(19) United States
(12) Patent Application Publication
(10) Pub. No.: US 2020/0376170 A1
(43) Pub. Date: Dec. 3, 2020

(54) BIOMIMETIC PRO-REGENERATIVE SCAFFOLDS AND METHODS OF USE THEREOF

(71) Applicant: President and Fellows of Harvard College, Cambridge, MA (US)

(72) Inventors: Seungkuk Ahn, Somerville, MA (US); Christophe Chantre, Geneva (CH); Grant Michael Gonzalez, Cambridge, MA (US); Kevin Kit Parker, Cambridge, MA (US)

(21) Appl. No.: 16/762,384

(22) PCT Filed: Nov. 8, 2018
(86) PCT No.: PCT/US18/59722

§ 371 Date: May 7, 2020

Related U.S. Application Data
(60) Provisional application No. 62/583,409, filed on Nov. 8, 2017.
(60) Provisional application No. 62/596,187, filed on Dec. 8, 2017.
(60) Provisional application No. 62/596,178, filed on Dec. 8, 2017.
(60) Provisional application No. 62/674,800, filed on May 22, 2018.

(51) Int. Cl. A61L 27/60; A61L 27/36; A61L 27/24; A61L 27/22; A61L 27/26

(52) U.S. Cl. CPC A61L 27/60; A61L 27/3691; A61L 27/24; A61L 2400/12; A61L 27/26; A61L 27/225; A61L 27/227

(57) ABSTRACT

The present invention provides polymeric fiber scaffolds, methods and devices suitable for fabricating such polymeric fiber scaffolds, and uses thereof for wound healing.

### RELATED APPLICATIONS

This application claims the benefit of priority to U.S. Provisional Application No. 62/583,409, filed on Nov. 8, 2017, U.S. Provisional Application No. 62/596,178, filed on Dec. 8, 2017, U.S. Provisional Application No. 62/596,187, filed on Dec. 8, 2017, and U.S. Provisional Application No. 62/674,800, filed on May 22, 2018. The entire contents of each of the foregoing applications are incorporated herein by reference.

### GOVERNMENT SUPPORT

This invention was made with government support provided by the National Science Foundation under grant number 1541959; the National Science Foundation-Division of Materials research under grant number DMR-1420570. The government has certain rights in the invention.

### BACKGROUND OF THE INVENTION

Developing dressings that restore cutaneous wounds to their original, healthy state remains a clinical challenge that impacts millions of people every year (Sen, C. K. et al. Wound Repair Regen 17, 763-771 (2009)). In the absence of external intervention, acute and chronic wounds and severe burns often result in collagen-dense scar formation as well as incomplete regeneration of hair follicles, sebaceous glands and cutaneous fat (Gurtner, G. C., Werner, S., Barrandon, Y. & Longaker, M. T. Nature 453, 314-321 (2008); Martin, P. Science 276, 75-81 (1997)). Adverse consequences can also include decreased tissue strength, elasticity, and impaired joint mobility (Corr, D. T., Gallant-Behm, C. L., Shrive, N. G. & Hart, D. Wound Repair Regen 17, 250-259 (2009); Tomasek, J. J., Gabbiani, G., Hinz, B., Chaponnier, C. & Brown, R. A. Nat Rev Mol Cell Biol 3, 349-363 (2002)), while changes in cosmetic appearance can lead to psychological sequelae.

Several therapeutic and cosmetic strategies have emerged over the last decades to improve the suboptimal outcome of normal wound healing. Although development of these strategies has led to reduction in infection rates and tissue morbidity, none of these strategies have been able to restore skin tissue to its native scarless configuration (Banyard, D. A., Bourgeois, J. M., Widgerow, A. D. & Evans, G. R. Plast Reconstr Surg 135, 1740-1748 (2015); Zhong, S. P., Zhang, Y. Z. & Lim, C. T. Tissue scaffolds for skin wound healing and dermal reconstruction. Wiley Interdiscip Rev Nanomed Nanobiotechnol 2, 510-525 (2010)). For example, a variety of skin substitutes and dermal analogs are already available and, although, these strategies have demonstrated some potential, the individual building-blocs (scaffolds, cell types, morphogens, etc.) that constitute these bioengineered constructs hamper their ability to direct tissue restoration. Indeed, these constituents are tailored to wound repair mechanisms that preferentially lead to fibrotic resolutions.

Accordingly, there is a need in the art for scaffolds, wound dressings, and methods to promote and accelerate cutaneous wound closure and to restore cutaneous wounds to their original native configuration without fibrosis.

### SUMMARY OF THE INVENTION

The present invention is based, at least in part, on the fabrication of polymeric fibers, e.g., micron, submicron or nanometer dimension polymeric fiber, scaffolds that have have physical and mechanical properties that mimic dermal skin extracellular matrix and/or fetal dermal skin extracellular matrix and that promote and accelerate cutaneous wound closure, promote cutaneous wound healing and/or cutaneous tissue regeneration and reduce fibrosis.

More specifically, the present invention is based, at least in part, on the fabrication of polymeric fibers, e.g., micron, submicron or nanometer dimension polymeric fiber, scaffolds comprising cellulose (CA) and soy protein hydrolysate (SPH), that have have physical and mechanical properties that mimic dermal skin extracellular matrix and that promote and accelerate cutaneous wound closure, promote cutaneous wound healing and/or cutaneous tissue regeneration and reduce fibrosis.

The present invention is also based, at least in part, on the fabrication of polymeric fiber, e.g., micron, submicron or nanometer dimension polymeric fiber, scaffolds comprising an extracellular matrix protein, e.g., hyaluronic acid, that have have physical and mechanical properties that mimic fetal dermal skin extracellular matrix, and that promote and accelerate cutaneous wound closure, promote cutaneous wound healing and/or cutaneous tissue regeneration and reduce fibrosis.

The present invention is further based, at least in part, on the fabrication of polymeric fiber, e.g., micron, submicron or nanometer dimension polymeric fiber, scaffolds comprising alfalfa and polycaprolactone (PCL), that have have physical and mechanical properties that mimic dermal skin extracellular matrix and that promote and accelerate cutaneous wound closure, promote cutaneous wound healing and/or cutaneous tissue regeneration and reduce fibrosis.

In addition, the present invention is based, at least in part, on the fabrication of polymeric fibers, e.g., micron, submicron or nanometer dimension polymeric fiber, scaffolds comprising hyaluronic acid (HA) and soy protein isolate (SPI), that have have physical and mechanical properties that mimic dermal skin extracellular matrix and that promote and accelerate cutaneous wound closure, promote cutaneous wound healing and/or cutaneous tissue regeneration and reduce fibrosis.

Methods and devices suitable for fabricating the polymeric fibers and polymeric fiber scaffolds of the invention having such superior and beneficial properties permit higher production rates and finer control over fiber morphology than standard electro-spinning methods and devices, and are less expresive to manufacture as high voltage is not required. Furthermore, in comparison to existing animal derived scaffolds for wound healing, the current polymeric fiber scaffolds may be free of animal derived proteins and/or synthetic polymers that may not be advantageous for wound healing.

In one aspect the present invention provides a polymeric fiber scaffold comprising a plurality of polymric fibers, each polymeric fiber independently comprising cellulose acetate and soy protein hydrolysate.

In one embodiment, each polymeric fiber independently comprises between about 60-70% w/w % cellulose acetate and between about 30-40 w/w % soy protein hydrolysate. In another embodiment, each polymeric fiber independently comprises between about 66.67% w/w % cellulose acetate and between about 33.33 w/w % soy protein hydrolysate.

In one embodiment, a solution forming the plurality of polymeric fibers comprises between about 8 w/v % and 12 w/v % cellulose acetate and between about 4 w/v % and 6 w/v % soy protein hydrolysate. In another embodiment, a solution forming the plurality of polymeric fibers comprises about 10 w/v % cellulose acetate and about 5 w/v % soy protein hydrolysate.

In one embodiment, each polymeric fiber independently comprises a cellulose acetate/soy protein hydrolysate weight ratio of about 2:1.

In one embodiment, each polymeric fiber independently has a diameter in a range of about 200 nm to 400 nm. In another embodiment, each polymeric fiber independently has a diameter in a range of about 300 nm to 400 nm.

In one embodiment, the polymeric fiber scaffold comprises a plurality of pores and the diameter of each pore independently is about 6 μm to 20 μm. In another embodiment, the polymeric fiber scaffold comprises a plurality of pores and the diameter of each pore independently is about 6 μm to 10 μm.

In one embodiment, the stiffness of the polymeric fiber scaffold is in the range of about 100 kPa to 200 kPa in the longitudinal direction and the stiffness of each of the fibers or the polymeric fiber scaffold is in the range of about 100 to 200 kPa in the transverse direction. In another embodiment, the stiffness of the polymeric fiber scaffold is in the range of about 150 kPa to 200 kPa in the longitudinal direction and the stiffness of each of the fibers or the polymeric fiber scaffold is in the range of about 100 to 150 kPa in the transverse direction.

In one embodiment, the polymeric fiber scaffold has physical properties that mimic extracellular matrix.

In one embodiment, the surface roughness (Rₐ) of each polymeric fiber is independently is about 50 to 100.

In one embodiment, the polymeric fiber scaffold exhibits a weight gain of at least 500% as a result of contact with water and water absorption.

In one embodiment, in the polymeric fiber scaffold has an initial water contact angle (at 0 s) of no higher than 60°.

In another aspect the present invention provides a polymeric fiber scaffold comprising a plurality of polymeric fibers, each polymeric fiber independently comprising a protein selected from the group consisting of collagen type I, fibrinogen, fibronectin, gelatin, chondroitin sulfate, and hyaluronic acid, and combinations thereof.

In one embodiment, each polymeric fiber independently comprises hyaluronic acid.

In one embodiment, each polymeric fiber independently comprises about 1% w/w to about 4% w/w hyaluronic acid.

In one embodiment, each polymeric fiber independently comprises fibronectin.

In one embodiment, each polymeric fiber independently comprises about 0.01% w/w to about 3.0% w/w fibronectin.

In one embodiment, each polymeric fiber independently comprises fibronectin and hyaluronic acid.

In one embodiment, each polymeric fiber independently comprises about 0.01% w/w to about 3.0% w/w fibronectin and about 1% w/w to about 2% w/w hyaluronic acid.

In one embodiment, each polymeric fiber independently comprises collagen type I.

In one embodiment, each polymeric fiber independently comprises about 2.0% w/w to about 10% w/w collagen type I.

In one embodiment, each polymeric fiber independently comprises fibrinogen.

In one embodiment, each polymeric fiber independently comprises about 4.0% w/w to about 12.5% w/w fibrinogen.

In one embodiment, each polymeric fiber independently comprises gelatin.

In one embodiment, each polymeric fiber independently comprises about 4.0% w/w to about 12% w/w gelatin.

In one embodiment, each polymeric fiber independently comprises chondroitin sulfate.

In one embodiment, each polymeric fiber independently comprises about 20% w/w chondroitin sulfate.

In one embodiment, each polymeric fiber independently comprises hyaluronic acid.

In one embodiment, each polymeric fiber independently comprises about 0.5% w/w to about 4% w/w hyaluronic acid.

In one embodiment, each polymeric fiber independently comprises hyaluronic acid and gelatin.

In one embodiment, each polymeric fiber independently comprises about 0.5% w/w to about 4% w/w hyaluronic acid and about 4% w/w to about 20% w/w gelatin.

In one embodiment, the polymeric fiber scaffold has a porosity greater than about 40%. In another embodiment, the polymeric fiber scaffold has a porosity of about 60% to about 80%.

In one embodiment, the polymeric fiber scaffold has a Young's modulus of about 400 Pascals to about 1,000 Pascals. In another embodiment, the polymeric fiber scaffold has a Young's modulus of about 400 Pascals to about 800 Pascals. In yet another embodiment, the polymeric fiber scaffold has a Young's modulus of about 400 Pascals to about 600 Pascals.

In one embodiment, the polymeric fiber scaffold has a compression modulus of about 10 kiloPascals to about 100 kiloPascals. In another embodiment, the polymeric fiber scaffold has a compression modulus of about 20 kiloPascals to about 50 kiloPascals.

In another embodiment, the polymeric fiber scaffold has about a 3000 fold to about a 6000 fold increase in absorption as determined by weight of the scaffold following the addition of water.

In one embodiment, each polymeric fiber independently has a diameter of about 500 nanometers to about 10 micrometers. In another embodiment, each polymeric fiber independently has a diameter of about 1 micrometer to about 5 micrometers.

In one embodiment, the plurality of polymeric fibers is covalently cross-linked.

In one embodiment, the plurality of polymeric fibers is covalently cross-linked via inter-polymeric fiber crosslinking and/or intra-polymeric fiber crosslinking.

In one embodiment, the plurality of polymeric fibers is covalently cross-linked via ester bond formation.

In one embodiment, the polymeric fiber scaffold has physical and mechanical properties that mimic fetal dermal skin extracellular matrix.

In one aspect, the present invention provides a polymeric fiber scaffold comprising a plurality of polymeric fibers, each polymeric fiber independently comprising polycaprolactone (PCL) and alfalfa.

In one embodiment, each polymeric fiber independently comprises between about 60-95% (w/w %) PCL and between about 5-35% (w/w %) alfalfa. In another embodiment, each polymeric fiber independently comprises about 85.71% (w/w %) PCL and about 14.29% (w/w %) alfalfa.

In one embodiment, a solution forming the plurality of polymeric fibers comprises about 6% (w/v %) PCL and between about 0.5% (w/v %) and 1% (w/v %) alfalfa. In another embodiment, a solution forming the plurality of polymeric fibers comprises about 6% (w/v %) PCL and about 1% (w/v %) alfalfa.

In one embodiment, each polymeric fiber independently comprises a PCL/alfalfa weight ratio of about 6:1.

In one embodiment, each polymeric fiber independently has a diameter in a range of about 200 nm to 500 nm. In another embodiment, each polymeric fiber independently has a diameter in a range of about 350 nm to 450 nm.

In one embodiment, the porosity of the polymeric fiber scaffold is about 50-80%.

In one embodiment, the stiffness of the polymeric fiber scaffold is in the range of about 5 kPa to 40 kPa.

In one embodiment, the specific stiffness of the polymeric fiber scaffold is in the range of about 10 kPa to 55 kPa.

In one embodiment, the polymeric fiber scaffold has a water contact angle at 25 seconds of less than 25°.

In one embodiment, the polymeric fiber scaffold comprises about 0.25% genistein.

In another aspect, the present invention provides a polymeric fiber scaffold comprising a plurality of polymeric fibers, each polymeric fiber independently comprising hyaluronic acid and soy protein isolate.

In one embodiment, each polymeric fiber independently comprises between about 2% w/w hyaluronic acid and about 2% w/w soy protein isolate.

In one embodiment, each polymeric fiber independently comprises a hyaluronic acid/soy protein isolate weight ratio of about 1:1.

In one embodiment, each polymeric fiber independently has a diameter in a range of about 1 micrometer to about 3 micrometers. In another embodiment, each polymeric fiber independently has a diameter in a range of about 1 micrometer to about 2 micrometers.

In one embodiment, the polymeric fiber scaffold has a porosity greater than about 40%. In another embodiment, the polymeric fiber scaffold has a porosity of about 60% to about 80%.

In one embodiment, the polymeric fiber scaffold has a Young's modulus of about 1 kiloPascal to about 10 kiloPascals. In another embodiment, the polymeric fiber scaffold has a Young's modulus of about 1 kiloPascal to about 7 kiloPascals.

In one embodiment, the plurality of polymeric fibers is covalently cross-linked.

In one embodiment, the plurality of polymeric fibers is covalently cross-linked via inter-polymeric fiber crosslinking and/or intra-polymeric fiber crosslinking.

In one embodiment, the plurality of polymeric fibers is covalently cross-linked via ester bond formation.

In one embodiment, the polymeric fiber scaffold comprises about 0.25% genistein.

In one embodiment, substantially all of the polymeric fibers in the scaffold are uniaxially aligned.

In one embodiment, the polymeric fiber scaffold promotes cutaneous wound healing.

In one embodiment, the polymeric fiber scaffold promotes cutaneous tissue regeneration.

In one embodiment, the polymeric fiber scaffold increases the closure of a cutaneous wound.

In one aspect, the present invention provides a method of forming a polymeric fiber scaffold comprising cellulose acetate and soy protein hydrosylate. The method includes providing a solution comprising a polymer comprising cellulose acetate; and soy protein hydrolysate; forming a plurality of polymeric fibers by ejecting or flinging the solution from a reservoir; and collecting the plurality of polymeric fibers on a collection surface to form the polymeric fiber scaffold.

In one embodiment, the solution comprises between about 8 w/v % and 12 w/v % acetate and between about 4 w/v % and 6 w/v % soy protein hydrolysate. In another embodiment, the solution comprises about 10 w/v % acetate and between about 5 w/v % soy protein hydrolysate.

In another aspect, the present invention provides a method of forming a polymeric fiber scaffold. The method includes providing a solution comprising an extracellular matrix protein selected from the group consisting of cola protein selected from the group consisting of collagen type I, fibrinogen, fibronectin, gelatin, and hyaluronic acid, and combinations thereof; rotating the polymer in solution about an axis of rotation to cause ejection of the polymer solution in one or more jets; and collecting the one or more jets of the polymer in a liquid to cause formation of one or more polymeric fibers, thereby forming the polymeric fiber scaffold.

In one embodiment, the solution comprises hyaluronic acid.

In one embodiment, the solution comprises about 1% w/v to about 3% w/v of hyaluronic acid.

In one embodiment, the solution comprises fibronectin.

In one embodiment, the solution comprises about 0.01% w/v to about 3.0% w/v fibronectin.

In one embodiment, the solution comprises fibronectin and hyaluronic acid.

In one embodiment, the solution comprises about 0.01% w/v to about 3.0% w/v fibronectin and about 1% w/v to about 2% w/v hyaluronic acid.

In one embodiment, the solution comprises collagen type I.

In one embodiment, the solution comprises about 2.0% w/v to about 10% w/v collagen type I.

In one embodiment, the solution comprises fibrinogen.

In one embodiment, the solution comprises about 4.0% w/v to about 12.5% w/v fibrinogen.

In one embodiment, the solution comprises gelatin.

In one embodiment, the solution comprises about 4.0% w/v to about 12% w/v gelatin.

In one embodiment, the solution comprises chondroitin sulfate.

In one embodiment, the solution comprises about 20% w/v chondroitin sulfate.

In one embodiment, the solution comprises hyaluronic acid.

In one embodiment, the solution comprises about 0.5% w/v to about 4% w/v hyaluronic acid.

In one embodiment, the solution comprises hyaluronic acid and gelatin.

In one embodiment, the solution comprises about 0.5% w/v to about 4% w/v hyaluronic acid and about 4% w/v to about 20% w/v gelatin.

In one embodiment, the polymeric fiber scaffold is soaked in a cross-linking bath.

In one embodiment, the cross-linking bath comprises ethyl(dimethylaminopropyl) carbodiimide (EDC)/N-hydroxysuccinimide (NHS).

In another aspect, the present invention provides a method of forming a polymeric fiber scaffold. The method includes providing a solution comprising a polymer comprising polycaprolactone (PCL); and alfalfa; forming a plurality of polymeric fibers by ejecting or flinging the solution from a reservoir; and collecting the plurality of polymeric fibers on a collection surface to form the polymeric fiber scaffold.

In one embodiment, the solution comprises about 6% (w/v %) PCL and between about 0.5% (w/v %) and 1% w/v % alfalfa. In another embodiment, the solution comprises about 6% (w/v %) PCL and between about 1% (w/v %) alfalfa.

In another aspect, the present invention provides a method of forming a polymeric fiber scaffold. The method includes providing a solution comprising hyaluronic acid and soy protein isolate; rotating the polymer in solution about an axis of rotation to cause ejection of the polymer solution in one or more jets; and collecting the one or more jets of the polymer in a liquid to cause formation of one or more polymeric fibers, thereby forming the polymeric fiber scaffold.

In one embodiment, the solution comprises about 2% w/v of hyaluronic acid and about 2% w/v soy protein isolate.

In one embodiment, the polymeric fiber scaffold is soaked in a cross-linking bath.

In one embodiment, the cross-linking bath comprises ethyl(dimethylaminopropyl) carbodiimide (EDC)/N-hydroxysuccinimide (NHS).

The present invention also provides a polymeric fiber scaffold produced from the method of the invention and a wound dressing comprising a polymeric fiber scaffold of the invention or a nanofiber scaffold produced by the methods of the invention.

In one aspect, the present invention provides a method for treating a subject having a cutaneous wound. The method includes providing the polymeric fiber scaffold of the invention or the polymeric fiber scaffold produced by the method of the invention; and disposing the polymeric fiber scaffold on, over, or in the wound, thereby treating the subject.

In one embodiment, the method further comprises keeping the polymeric fiber scaffold disposed on, over or in the wound during wound healing.

In one embodiment, the method promotes healing of the wound of the subject.

In one embodiment, the method accelerates closure of the wound.

In one embodiment, the method promotes tissue regeneration in the subject.

In one embodiment, at least a portion of the wound is in dermal tissue, in epidermal tissue, or in both and the method accelerates closure of at least the portion of the wound that is in dermal tissue, in epidermal tissue, or in both, and/or promotes dermal tissue regeneration, epidermal tissue regeneration, or both.

In one embodiment, the method promotes tissue regeneration in the subject.

In one embodiment, the method reduces fibrosis in the subject formed at the wound site.

In one embodiment, the method reduces fibrosis formation in dermal tissue of the subject, epidermal tissue of the subject, or both.

In one embodiment, the method is a method of reducing a size of a scar formed at the wound site in the subject.

### BRIEF DESCRIPTION OF THE DRAWINGS

FIG. 1 shows a schematic of polymeric nanofiber fabrication with a rotary jet spinning (RJS) system and a bright field image of a magnified portion of a cellulose acetate/soy protein hydrolysate (CA/SPH) nanofiber scaffold prepared using a solution comprising 10% w/v CA and 5% w/v SPH.

FIGS. 2A, 2B, 2C, 2D, 2E and 2F are scanning electron microscopy (SEM) images of polymeric CA and CA/SPH fibers spun using solutions comprising the indicated amounts of CA and SPH. Scales are 50 μm. Arrows indicate beading.

FIGS. 2G, 2H, 2I, 2J, 2K and 2L are scanning electron microscopy (SEM) images of dense polymeric nanofibrous scaffolds spun using solutions comprising the indicated amounts of CA and SPH. Scales are 50 μm. Arrows indicate beading.

FIG. 3 shows the FT-IR spectra of different CA and CA/SPH polymeric fibers and SPH powder.

FIG. 4 is a plot of peak area-to-peak area ratio (amide I peak (1600-1700 cm⁻¹) over acetyl peak (1700-1800 cm⁻¹)) for different CA/SPH nanofibers from the FT-IR spectrum of FIG. 3. Bars represent standard error, n=3 from 3 productions, R²=0.99967 for linear curve fit.

FIG. 5 shows the high-resolution XPS spectra of N₁ₛ for the indicated CA and CA/SPH nanofibers.

FIG. 6 is bar graph showing the nitrogen atomic percentages (%) in the indicated CA/SPH polymeric nanofibers that were calculated based on the peak areas of the N₁ₛ spectra in FIG. 5. The bars represent standard error, n=3 from 3 productions.

FIG. 7 shows the high-resolution XPS spectra of C₁ₛ for CA (10 wt/v %) and CA/SPH (10 wt/v %/5 wt/v %) nanofibers. The C₁ₛ peaks (in dotted lines) were deconvoluted to four peaks.

FIGS. 8A, 8B and 8C are images of the elemental analysis by energy-dispersive X-ray spectroscopy (EDS) for nitrogen (NK) and carbon (CK) together with corresponding secondary electron (SE2) images of CA (10 wt/v %) nanofibers. The white dots indicate the shape of nanofibers. Scales are 500 nm.

FIGS. 9A, 9B and 9C are images of the elemental analysis by energy-dispersive X-ray spectroscopy (EDS) for nitrogen (NK) and carbon (CK) together with corresponding secondary electron (SE2) images of CA/SPH (10 wt/v %/5 wt/v %) nanofibers. The white dots indicate the shape of nanofibers. Scales are 500 nm.

FIG. 10A is a bar graph showing the fiber diameter of CA (10 wt/v %) and CA/SPH (10 wt/v %/5 wt/v %) nanofibers. The diameter of CA (6% w/v) polymeric fibers is shown for comparison. Bars represent standard error, n=10 from 3 productions.

FIG. 10B is a bar graph showing the pore diameter of CA (10 wt/v %) and CA/SPH (10 wt/v %/5 wt/v %) polymeric fiber scaffolds. The pore diameter of CA (6% w/v) polymeric polymeric fiber scaffolds is shown for comparison. Bars represent standard error, n=10 from 3 productions.

FIG. 10C is a bar graph showing stiffness measurement for CA (10 wt/v %) and CA/SPH (10 wt/v %/5 wt/v %) nanofiber scaffolds in the wet state on the longitudinal and transverse directions. The stiffness measurement of PCL (6% w/v) polymeric polymeric fiber scaffolds is shown for comparison. Bars represent standard error, n=5 from 3 productions, * indicates p<0.05.

FIG. 10D is a bar graph showing fiber thickness within the scaffolds as a function of the different volumes of polymer solution. n=3 from 3 productions.

FIG. 10E is a bar graph showing pore diameters within the scaffolds as a function of the different volumes of polymer solution. n=3 from 3 productions.

FIGS. 11A and 11B are atomic force microscopy (AFM) images of CA (10 wt/v %) and CA/SPH (10 wt/v %/5 wt/v %) nanofiber scaffolds, respectively.

FIG. 12 is a bar graph showing roughness (Rₐ) of CA (10 wt/v %) and CA/SPH (10 wt/v %/5 wt/v %) nanofibers, n=3, FOV (field of view)=3 from 3 productions.

FIG. 13 is a line graph showing the contact angle analysis of brightfiled images of water droplets on CA (10 wt/v %) and CA/SPH (10 wt/v %/5 wt/v %) cast nanofiber films (see images in FIGS. 14A-14D), n=3 from 3 productions. Dots delimit water droplet and film. Scales are 5 mm.

FIGS. 14A and 14B are images of water droplets on scaffold samples at 0 s and 2 s, respectively, showing that contact angles on the scaffolds are highly time-dependent due to the rapid diffusion of water into the samples.

FIGS. 14C and 14D are bright field images of water droplets on CA (10 wt/v %) and CA/SPH (10 wt/v %/5 wt/v %) nanofiber scaffolds, respectively.

FIG. 15A is a bar graph showing the contact angle analysis of the water droplets on CA (10 wt/v %) and CA/SPH (10 wt/v %/5 wt/v %) nanofibers. Bars represent standard error, n=3 from 3 productions, * indicates p<0.05.

FIG. 15B is a line graph show in vitro biodegradation measured by weight loss (n=3 from 3 productions). Bars represent standard error, * indicates p<0.05.

FIG. 15C shows the in vitro release kinetics of soy protein from the CA/SPH (10 wt/v %/5 wt/v %) nanofibers. The line indicates a Boltzmann curve fitting (n=3 from 3 productions).

FIG. 16 is a bar graph showing in vitro water absorption measurements by weight gain, n=6 from 3 productions. Bars represent standard error, * indicates p<0.05.

FIGS. 17A and 17B are confocal microscopy images of human neonatal dermal fibroblasts (HNDF) on PCL (6 wt/v %) nanofiber scaffolds stained with Ki-67 and DAPI, and FIG. 17C is a merged image of FIGS. 17A and 17B.

FIGS. 17D and 17E are confocal microscopy images of human neonatal dermal fibroblasts (HNDF) on CA (10 wt/v %) nanofiber scaffold stained with Ki-67 and DAPI, and FIG. 17F is a merged image of FIGS. 17D and 17E.

FIGS. 17G and 17H are confocal microscopy images of human neonatal dermal fibroblasts (HNDF) on CA/SPH (10 wt/v %/5 wt/v %) nanofiber scaffolds stained with Ki-67 and DAPI, and FIG. 17I is a merged image of FIGS. 17G and 17H.

FIG. 18 is a bar graph showing analysis of Ki-67 positive cells on PCL (6 wt/v %), CA (10 wt/v %) and CA/SPH (10 wt/v %/5 wt/v %) nanofiber scaffolds. Scales are 100 μm. Bars represent standard error, n=5 for PCL and n=6 for CA and CA/SPH, FOV=25, * indicates p<0.05.

FIG. 19 is a bar graph showing cytotoxicity produced by calculating release of LDH from PCL (6 wt/v %), CA (10 wt/v %) and CA/SPH (10 wt/v %/5 wt/v %) nanofiber scaffold, n=17 in triplicates, box plot=25-75%, error bars=10-90%.

FIGS. 20A, 20B, 20C and 20D confocal microscopy images of GFP-expressing human neonatal dermal fibroblasts (HNDF) on PCL (6 wt/v %) nanofiber scaffolds on Day 0, 5, 10 and 15, respectively. Scales are 50 μm.

FIGS. 20E, 20F, 20G and 20H confocal microscopy images of GFP-expressing human neonatal dermal fibroblasts (HNDF) on CA (10 wt/v %) nanofiber scaffolds on Day 0, 5, 10 and 15, respectively. Scales are 50 μm.

FIGS. 20I, 20J, 20K and 20L confocal microscopy images of GFP-expressing human neonatal dermal fibroblasts (HNDF) on CA/SPH (10 wt/v %/5 wt/v %) nanofiber scaffolds on Day 0, 5, 10 and 15, respectively. Scales are 50 μm.

FIG. 21 is a bar graph showing analysis of surface area covered by cells at day 0, 5, 10, and 15 as in FIGS. 20A-20L. Scales are 50 μm. Bars represent standard error, n=5, FOV=5, * indicates p<0.05.

FIGS. 22A, 22B, 22C and 22D are binary images of tracking a single cell on PCL (6 wt/v %) nanofiber scaffolds at Day 0, 5, 10, and 15, respectively, and used for calculating the migration speed shown in the graph in FIG. 23.

FIGS. 22E, 22F, 22G and 22H are binary images of tracking a single cell on CA (10 wt/v %) nanofiber scaffolds at Day 0, 5, 10, and 15, respectively, and used for calculating the migration speed shown in the graph in FIG. 23.

FIGS. 221, 22J, 22K and 22L are binary images of tracking a single cell on CA/SPH (10 wt/v %/5 wt/v %) nanofiber scaffolds at Day 0, 5, 10, and 15, respectively, and used for calculating the migration speed shown in the graph in FIG. 23.

FIG. 23 is a bar graph showing migration speed of HNDF on PCL (6 wt/v %), CA (10 wt/v %) and CA/SPH (10 wt/v %/5 wt/v %) nanofiber scaffolds. Scales are 50 μm. Bars represent standard error, n=5, FOV=5.

FIGS. 24A, 24B and 24C are 3D-reconstructed confocal microscopy images of HNDF on PCL (6 wt/v %), CA (10 wt/v %) and CA/SPH (10 wt/v %/5 wt/v %) nanofiber scaffolds, respectively, after 15 days of cell culture.

FIG. 25 is a bar graph showing quantitative analysis of cell infiltration depth of HNDF on PCL (6 wt/v %), CA (10 wt/v %) and CA/SPH (10 wt/v %/5 wt/v %) nanofiber scaffolds. Bars represent standard error, n=5 for PCL and n=8 for CA and CA/SPH, FOV=3, * indicates p<0.05.

FIGS. 26A and 26B are immunostained images of HDNF on CA (10 wt/v %) nanofiber scaffolds and integrin β1 expressed on the HDNF, respectively. FIG. 26C is a merged image of FIGS. 26A and 26B. Scales are 100 μm.

FIGS. 26D and 26E are immunostained images of HDNF on CA/SPH (10 wt/v %/5 wt/v %) nanofiber scaffolds and integrin β1 expressed on the HDNF, respectively. FIG. 26F is a merged image of FIGS. 26D and 26E. Scales are 100 μm.

FIG. 27 is a Western blotting image for integrin β1 expressed in HDNFs on CA (10 wt/v %) and CA/SPH (10 wt/v %/5 wt/v %) nanofiber scaffolds.

FIG. 28 is a bar graph showing the quantitative analysis of Western blotting for integrin β1 expressed in HDNF on CA (10 wt/v %) and CA/SPH (10 wt/v %/5 wt/v %) nanofiber scaffolds. Bars represent standard error, n=6 for CA and n=7 for CA/SPH, * indicates p<0.05.

FIGS. 29A and 29B are cross-sectional view (yz plane) of dermal fibroblasts infiltrated in PCL (6 wt/v %) fiber scaffolds at Day 0 and Day 15, respectively. Scales are 100 μm.

FIGS. 29C and 29D are cross-sectional view (yz plane) of dermal fibroblasts infiltrated in CA (10 wt/v %) fiber scaffolds at Day 0 and Day 15, respectively. Scales are 100 μm.

FIGS. 29E and 29F are cross-sectional view (yz plane) of dermal fibroblasts infiltrated in CA/SPH (10 wt/v %/5 wt/v %) fiber scaffolds at Day 0 and Day 15, respectively. Scales are 100 μm.

FIG. 30 is a schematic representation of the in vivo wound healing experiment described herein.

FIGS. 31A-31D illustrate the various steps of the surgical procedure performed on the mouse excisional wound splinting model. FIG. 31A shows that a portion of the back of the mouse is shaved to reveal the animal's skin. FIG. 31B shows that two biopsy-punch articial wounds (6 mm in diameter) are introduced to the skin. FIG. 31C shows that suture silicon rings (8 mm in diameter) are applied onto the wounds. FIG. 31D shows that CA (10 wt/v %) or CA/SPH (10 wt/v %/5 wt/v %) nanofiber scaffolds are applied onto the wound sites which are then secured with Tegaderm™.

FIGS. 32A, 32B and 32C are images of a wound left untreated on Day 0, 7 and 14, respectively. Scales are 5 nm.

FIGS. 32D, 32E and 32F are images of a wound treated with CA (10 wt/v %) nanofiber scaffold on Day 0, 7 and 14, respectively. Scales are 5 nm.

FIGS. 32G, 32H and 321 are images of a wound treated with a CA/SPH (10 wt/v %/5 wt/v %) nanofiber scaffold on Day 0, 7 and 14, respectively. Scales are 5 nm.

FIG. 33 is a bar graph showing analysis of wound closures. Fiber wound dressings were prepared from 3 productions for each condition. Bars represent standard error, n=4 wounds and 3 mice for control, n=5 wounds and 3 mice for CA and CA/SPH. * indicates p<0.05.

FIG. 34A is an image of H & E staining of an untreated wound 14 days post-surgery. FIGS. 34B, 34C and 34D are magnified images of the sections highlighted in FIG. 34A. Scales are 500 μm for FIG. 34A and 200 μm for FIGS. 34B, 34C and 34D. Fiber wound dressings were prepared from 3 productions for each condition. The arrows indicate the edge of the epidermal layer and the white dots outline the scar area. The white outlines delimit the epidermal layer in the skin tissue.

FIG. 35A is an image of H & E staining of a wound treated with a CA (10 wt/v %) nanofiber scaffold 14 days post-surgery. FIGS. 35B, 35C and 35D are magnified images of the sections highlighted in FIG. 35A. Scales are 500 μm for FIG. 35A and 200 μm for FIGS. 35B, 35C and 35D. The arrows indicate the edge of the epidermal layer and the white dots outline the scar area. The white outlines delimit the epidermal layer in the skin tissue.

FIG. 36A is an image of H & E staining of a wound treated with a CA/SPH (10 wt/v %/5 wt/v %) nanofiber scaffold 14 days post-surgery. FIGS. 36B, 36C and 36D are magnified images of the sections highlighted in FIG. 36A. Scales are 500 μm for FIG. 36A and 200 μm for FIGS. 36B, 36C and 36D. The arrows indicate the edge of the epidermal layer and the white dots outline the scar area. The white outlines delimit the epidermal layer in the skin tissue.

FIG. 37A is an image of H & E staining of healthy skin harvested from Day 0. Scale is 500 μm. FIG. 37B is a magnified image of the section highlighted in FIG. 37A, with the white outlines delimiting the epidermal layer in the skin tissue. Scale is 100 μm.

FIG. 38 is a bar graph showing a quantitative analysis of epithelial gap of wounds that are left untreated, treated with a CA (10 wt/v %) nanofiber scaffold or treated with a CA/SPH (10 wt/v %/5 wt/v %) nanofiber scaffold. Bars represent standard error, n=3 wounds and 3 mice for control, n=4 wounds and 3 mice for CA and CA/SPH nanofibers, at least 3 sections per wound, * indicates p<0.05.

FIG. 39 is a bar graph showing a quantitative analysis of epithelial thickness of wounds that are left untreated, treated with a CA (10 wt/v %) nanofiber scaffold or treated with a CA/SPH (10 wt/v %/5 wt/v %) nanofiber scaffold. Bars represent standard error, n=3 wounds and 3 mice for control, n=4 wounds and 3 mice for CA and CA/SPH nanofibers, n=5 wounds and 5 mice for healthy tissue, at least 3 sections per wound, * indicates p<0.05.

FIG. 40 is a bar graph showing a quantitative analysis of scar index of wounds that are left untreated, treated with a CA (10 wt/v %) nanofiber scaffold or treated with a CA/SPH (10 wt/v %/5 wt/v %) nanofiber scaffold. Bars represent standard error, n=3 wounds and 3 mice for control, n=4 wounds and 3 mice for CA and CA/SPH nanofibers, at least 3 sections per wound, * indicates p<0.05.

FIG. 41 is a bar graph showing collagen alignment from the H&E staining images of FIGS. 35A-35D, 36A-36D and 37A-37B.

FIGS. 42(a)-42(e) depict that the hydrodynamic forces produced via rotary jet spinning (RJS) drove fibrillogenesis of fibronectin (Fn). (a) The RJS system consists of a perforated reservoir rotating at high speeds. (Insets) Soluble Fn contained in the reservoir is extruded through an orifice and unfolded via centrifugal forces produced by high-speed rotation. Insets 1 and 2 show the entry flow and channel flow loci, respectively. (b) Image of the perforated reservoir of the RJS system. (c) Extensional flow regime schematic (left) at the entry shows the Fn solution experiencing high acceleration and high strain rates, depicted with the computational fluid dynamics (CFD) simulations below. In contrast, the shear flow regime schematic (right) shows the Fn solution experiencing a high velocity and shear gradient across the channel, demonstrated with the CFD simulations below. (d) Scanning electron micrographs (SEM) of Fn spun at different rotation speeds with the RJS. Rotation speeds at 25 k rpm and above show formation of Fn nanofibers, whereas only partial fiber formation is observed at lower speeds. (e) Dual-labeling for FRET shows the reduction in acceptor to donor (IA/ID) ratio before (Fn solution) and after spinning at 28 k rpm. Intensity ratios were 0.95±0.02 and 0.58±0.01 for the Fn solution and the extended fibrillar Fn, respectively. n>20 measurements per condition.

FIGS. 43(a)-43(c) depict that Fn nanofibers extend 300% and exhibit a bimodal stress strain curve. (a) Differential interference contrast images of a single Fn nanofiber prepared for uniaxial tensile testing (top) and Fn nanofiber during uniaxial tensile test at −300% strain (bottom). Inset 1 shows Fn nanofiber (arrowhead) attached to tensile tester μ-pipettes at resting position, and inset 2 shows Fn nanofiber under uniaxial tension. (b) Stress-strain plot shows that Fn nanofibers produced by RJS have a non-linear behavior that can be characterized by two regimes and can extend up to three times their original length. (c) Results of molecular extension estimation by an eight-chain model.

FIGS. 44(a)-44(d) depict that Fn nanofiber scaffolds accelerated full-thickness wound closure in a C57BL/6 mouse model. (a) Schematic representation of (1) two full-thickness skin wounds on the back of a mouse using a biopsy punch and (2) application of a nanofiber wound dressing. To assure adhesion and stabilization of the nanofibers throughout the study, Tegaderm™ film dressings were applied over the wound (3). Control group was likewise covered with a Tegaderm™ film. (b) SEMs of the micro- and macro-structure of native dermal ECM inspired the design and fabrication of Fn scaffolds for optimal integration in the wound. (c) Representative images of the non-treated control group and wounds treated Fn nanofiber dressings at days 2, 8 and 16. Insets below show minimal scarring in Fn treatment compared to control (highlighted with the dashed line). (d) From these images, wound edge traces were established for each condition. (e) Normalized wound area over a 16 day period demonstrated that closure rate was significantly increased for Fn dressings compared to the control a from day 2 to day 14 after. Mean and standard error are shown. n=8 mice and 16 wounds; *p<0.05 and **p<0.01 vs. control in a Student's t-test.

FIGS. 45(a)-45(f) depict that Fn nanofiber scaffolds promoted native dermal and epidermal architecture recovery. (a) Masson's trichrome staining of healthy tissue sections was performed to establish the design criterion for successful skin tissue restoration. An epidermal thickness of ˜20 μm, a ECM fiber alignment of ˜0.7 (a.u.) as well as ˜7 hair follicles and ˜3.5 sebaceous glands per surface area of 500 μm² (c-e) was measured. (b) Representative stains of skin tissues with different treatment conditions 20 days post wounding. Black arrowheads indicate original wound edges. Insets demonstrate recovery of epidermal thickness and presence of skin appendages at the center of the wound in the Fn-treated tissue, in contrast with the control group. (c) Epidermal thickness measurements showed that Fn nanofiber dressings restored tissue close to its native state, whereas the control had a statistically significant increase in thickness. (d) ECM fibers alignment was used to quantify healthy tissue (characterized by a basket-woven structure) and scarred tissue (aligned fiber bundles) where 0 is perfectly isotropic and 1 is perfectly anisotropic. Analysis revealed that all recovering tissues were more aligned than native skin, with a closer value to native skin for the Fn. (e) Quantification of hair follicles and sebaceous glands per area demonstrated that Fn wound dressings promoted restoration of skin appendages close to the native state. This restoration was significantly higher than the control group for both hair follicles and sebaceous glands. Mean and standard error are shown. n=5-8 wounds; *p<0.05, **p<0.01 vs. Healthy and #p<0.05, ##p<0.01 vs. Fn in a oneway ANOVA on ranks with a post hoc multiple comparisons Dunn's test. (f) To quantify the regenerative potency of these treatments, the different parameters measured in c-e were compared to healthy tissues and scored from 0 to 100% match. Gray shaded boxes are used to represent % match to healthy skin (% match shown below the gray shaded boxes).

FIGS. 46(a)-46(c) depict that Fn nanofiber scaffolds supported recruitment of dermal papillae and basal epithelial cells. (a) Schematic representation of the hair follicle structure with specific markers used in (b-c) labelled. (b) Confocal fluorescent images of alkaline phosphatase (ALP) as well as immunostaining with Keratin 5 (K5), Keratin 14 (K14), Keratin 17 (K17) and DAPI confirmed the presence of dermal papillae (DP) and epithelial cells (EC) in healthy tissues of the mouse wound model. ECs were observed lining the interfollicular epidermis (IFE) and around the hair follicle shaft (light gray arrowheads). ECs with the K17 marker, specific to the outer root sheath (ORS), were observed in hair follicles only (dark gray arrowheads). White arrowheads highlight presence of DP (stained with ALP) in the follicle bulb, critical for hair growth and cycling. (c) At day 20 post wounding, tissue sections treated with Fn scaffolds demonstrated presence of K5/K14-positive cells in the IFE and around hair follicles. K17-positive cells were witnessed exclusively in the ORS. ALP-positive cells were observed in re-formed DP, supporting the potential for restoration of functional hair. For the two first panels (ALP/K5 staining), images close to the wound edge (top) and at the center of the wound (bottom) are shown.

FIGS. 47(a)-47(d) depict that Fn nanofiber scaffolds permitted restoration of a lipid layer in the wound. (a) Lysochrome staining (Oil-red-o) was performed to identify presence of lipid droplet-carrying adipocytes in skin of healthy uninjured mice. Oil-red-o revealed presence of a lipid layer in the hypodermis (Inset 1) and in sebum-secreting sebaceous glands (Inset 2). Oil-red-o-positive cells in the hypodermis only were used to quantify the lipid layer coverage. (b) Representative staining images showing presence of lipids in regenerating tissues treated with Fn and the control. (c) Quantitative analysis revealed that both conditions supported restoration of the lipid layer, with a higher trend for the Fn treatment. n=3 wounds; *p<0.05 vs. Healthy and #p<0.05 vs. Fn in a one-way ANOVA on ranks with a post hoc multiple comparisons Dunn's test. (d) As previously, treatment conditions were compared to healthy skin tissue (c) and scored from 0 to 100% match. Gray shaded boxes represent % match to healthy skin (% match shown below the gray shaded boxes).

FIGS. 48(a)-48(c) depict Fn scaffolds fabricated using the RJS. (a) Photograph of a sheet of Fn fibers (approx. 100-200 μm in thickness) spun at ˜30,000 rpm using the RJS, collected on a rotating mandrel and unrolled post-spinning (b) Fn nanofibers are then cut into 8 mm discs with a biopsy punch and used for imaging (right panel shows SEM image) or used subsequently for in vivo studies. (c) SEM images show fabrication of intact and smooth Fn nanofibers with an average diameter of 457 nm±138.

FIG. 49 depicts the chemical structure analysis of Fn fibers by Raman spectroscopy. Raman spectrum shows intact secondary structure of Fn fibers with the presence of Amide 1 (1649 cm-1) and Amide III (1249 cm-1) peaks. The absence of Amide II peak suggests that tertiary structures are in partially folded states.

FIGS. 50(a)-50(c) depict single fiber μ-pipette uniaxial tensile testing. (a), The testing setup consists of one calibrated pipette and one force applicator pipette to which a fiber is adhered by a droplet of epoxy. Tip deflection is measured as the fiber is pulled. (b) Force is measured based on calculated beam stiffness. A known force (F) will deflect the pipette tip a known distance (Δy). (c) Representative differential interference contrast (DIC) images of a single Fn nanofiber (black arrowheads) attached between two μ-pipette (gray arrowheads). DIC images represent different time points (0, 2 and 5 min) during uniaxial tensile testing (300% strain). DIC images show tip deflection as described in (a-b).

FIGS. 51(a)-51(b) depict epidermal thickness measurements and skin appendage density analysis. To determine if treated wounds had recovered original healthy epidermal structure, epidermal thicknesses of the different treated tissues were measured 20 days post wounding and compared to healthy uninjured tissue. To verify recovery of dermal architecture, density of hair follicles and sebaceous glands in the treated-wounds were calculated using the same tissue sections. (a) Masson's trichrome staining image of unwounded healthy tissue with black dashed lines delimiting the epidermal layer in the skin tissue. Black and gray arrowheads mark presence of hair follicles and sebaceous glands, respectively. (b) Representative images of wound centers 20 days post injury reveal epidermal thickness recovery for Fn treatments whereas control remains thicker. Arrowheads demonstrate strong presence of hair follicles and sebaceous glands in the Fn treatment. The control condition was void of any skin appendages at the center of the wounds.

FIGS. 52(a)-52(b) depict the establishment of wound edges for consistent measurements. To perform accurate and consistent measurements between our different treatment samples, wound edges were defined using the positions of the sectioned panniculus carnosus muscle tissue (black arrows). (a) Masson's trichrome images of a non-treated full-thickness wound two days post injury, demonstrating removal of the epidermis, the dermis, hypodermis and the underlying muscle tissue. Insets display position of original wound edges with position of muscle tissue. (b) Images of a full-thickness wound 20 post injury treated with a Fn nanofiber wound dressing. Insets display original position of wound edges.

FIGS. 53(a)-53(b) depict ECM fibers organization analysis. Skin tissue sections stained with H&E (left), color-coded image algorithms (center) and corresponding orientation order parameter (OOP) plots (right). H&E images were first manually preprocessed, discounting the epidermal layer and the underlying muscle tissue (black lines). Images were then converted to color-coded image algorithms to identify the orientation of ECM fibers in the dermis. Next, analysis of the OOP plots enabled to calculate an OOP value quantifying the organization of ECM fibers (with 0 being perfectly isotropic and 1 perfectly anisotropic). (a) Sample image of H&E and corresponding color-coded algorithm image and OOP plot of healthy/uninjured tissue. Data shows a distributed range of fiber orientation with an OOP value of 0.70. (b) Representative H&E images and corresponding gray scaled algorithm images and OOP plots of the different regenerating tissues 20 days post wounding. The OOP values for Fn and control were 0.83 and 0.93, respectively, in the samples showed.

FIGS. 54(a)-54(c) depict cell-mediated Fn unfolding and theoretical model of Fn unfolding in the RJS system. (a) Schematic of the Fn molecule structure with relevant domain sites labeled. Of specific interest are the FNI1-5 domains responsible for Fn assembly during fibrillogenesis, FNIII domains with embedded beta-sheet structures providing mechanical elasticity and the FNIII9-10 RGD and synergy sites necessary for cellular adhesion. (b) Mechanism of Fn fibrillogenesis in vivo. Globular Fn binds to cells via integrin-binding site, inducing actin cytoskeletal reorganization and cell contractility. This in turn enables unfolding of the Fn molecule, exposing N-terminal Fn-Fn binding sites (FNI1-5) and generating polymerization of Fn into insoluble fibrils. (c) Mechanism of flow-mediated Fn fibrillogenesis studied at the entry flow, where high extensional strain is experienced and the channel flow, where high shear is experienced. Insets show Fn molecules undergo stretching due to extensional strain (top) or shear (bottom) rates. (Top) An Fn molecule under a heterogeneous velocity field v can be modeled as a string of N modules, with a diameter a and separated by a center-to-center distance d, while the clusters have a radius r. (Bottom) Because of the heterogeneous velocity field perpendicular to the channel flow, the Fn molecule may either continue to stretch or become unstable and tumble.

FIGS. 55(a)-55(b) depict parameters for the CFD simulations. (a) Schematic representation of the RJS reservoir and orifice (top, and inset 1). Diagram bellow illustrates the reservoir section with the parameters relevant to the analytical model and CFD simulations. (b) Geometries of the Fn solution in the reservoir and the channel for the CFD simulations are constructed such that the centerline is aligned with the x axis and the yz plane for the symmetric boundary condition.

FIGS. 56(a)-56(c) depict Deborah (De) and Weissenberg (Wi) numbers for different rotation speeds by CFD simulations. (a) Maximum elongation strain rates and corresponding De numbers calculated for specific rotation speeds (0-3,000 s⁻¹) of the RJS reservoir. Results show an increase of De number with increasing rotation speeds. For the maximum rotation speed of 3,000 s⁻¹, a strain rate of 1.3×105 s⁻¹ and De number of 28.9 were calculated. (b) Elongation strain rates and corresponding De numbers along the centerline calculated for specific rotation speeds. For the maximum rotation speed, a strain rate of 0.76×105 s⁻¹ and De number of 16.6 were calculated. (c) Shear strain rates and corresponding Wi number calculated for different rotation speeds. For the maximum rotation speed, a shear rate of 2.9×105 s⁻¹ and Wi number of 79.0 were calculated.

FIG. 57 depicts immunostained Fn fibers. Images of two Fn nanofibers stained with an anti-human Fn antibody confirm molecular integrity of Fn post-spinning. The right-hand image is an iverted image of the left-hand image.

FIGS. 58(a)-58(b) depict the FRET sensitivity calibration for Fn unfolded via GdnHCl. (a) FRET fluorescence spectra of labeled Fn in solution, measured for increasing concentration of [GdnHCl]. FRET signal decreases with increasing concentration of [GdnHCl]. (b) The acceptor intensity (IA) and the donor intensity (ID) ratios (IA/ID) were calculated to show sensitivity of FRET measurements of Fn unfolding. FRET was lowest for exposure to 4M and 8M of [GdnHCl] with FRET signals of 0.688 and 0.5626, respectively.

FIGS. 59(a)-59(c) depict the conformational structure of Fn nanofibers by FRET analysis. (a) Schematic of FRET fluorescence, with a high FRET signal (close to 1) for the compact globular conformation and low FRET signal (close to 0) for the fully extended fibrillar conformation. (b) Confocal images at donor emission wavelength (520 nm) and acceptor emission wavelength (576 nm) were taken using the donor excitation wavelength (488 nm). Dual-labeled globular Fn adsorbed on glass coverslips shows strong FRET signal (compact conformation). (c) Dual-labeled Fn unfolded using the RJS shows a weak FRET signal (fibrillar conformation). Confocal images are also shown.

FIGS. 60(a)-60(b) depict that Fn nanofibers supported recruitment of dermal papillae and epithelial cells throughout wounded tissue. (a) Healthy tissue section stained for Alkaline Phosphatase (ALP), Keratin 5 (K5) and DAPI confirmed the presence of DPs and ECs. (b) Further histochemical stains of tissues treated with Fn nanofiber wound dressings and the control 20 days post injury. White arrowheads indicate original wound edges. Gray arrowheads in Fn-treated skin tissue highlights presence of ALP-positive cell niches, suggesting presence of dermal papillae (Inset 1). Images reveal lower density and distribution of ALP and K5-positive cells for the control, significant at the wound center (Inset 2).

FIGS. 61(a)-61(d) depict high-throughput production of biological nanofiber scaffolds using an immersion rotary jet spinning (iRJS) platform. a, Schematic of the iRJS system (left) with corresponding still images of the reservoir rotating at 15 k rpm and spinning an HA solution (right, panel 1 and 2). The iRJS is designed with a perforated rotating reservoir, capable of spinning at up to 40 k rpm, and a vortex precipitation bath positioned axially to the reservoir. The high centrifugal forces exerted by the spinning reservoir will drive extrusion of the polymer dope out of the reservoir through the two radial orifices (panel 2), forming a jet that will elongate across the air gap before hitting the vortex precipitation bath (panel 1). Jet precipitation and stabilization around a cylindrical collector will ensue. b, Side-view images of the whole iRJS setup at different spinning time-points (0 to 5 min), emphasizing the high throughput capabilities of the system, where fibers (in white) are collected on a collector (blue) in the precipitation vortex bath. c, Centimeter-wide sheet of fibers wrapped around a collector. Inset shows scanning electron micrograph (SEM) of fibers with a basket-weave alignment organization. d, Several different ECM molecules were spun to demonstrate the versatility of this manufacturing approach. The GAG chondroitine sulfate and the ECM proteins collagen, gelatin and fibrinogen were spun into micro- and nano-fiber scaffolds from aqueous solutions.

FIGS. 62(a)-62(c) depict HA disaccharide assembly confirmed by SEM images and FTIR. a, HA nanofiber fabrication and cross-linking schematic framework. (1) Lyophilized HA powder is dissolved in an aqueous solution of diH2O with 150 mM NaCl at RT, and stirred for 24 hrs for dissolution. (2) Spinning is then utilized to induce fiber formation, whereby HA disaccharides are assembled aligned structures. (see b, left) (3) Inter- and intra-fiber cross-linking is mediated via EDC/NHS to form ester bonds between carboxyls and primary amines b, SEM images depict ultrastructure of HA fibers with internal alignment polymer chains (left), and inter-fiber bonding created during cross-linking process. Inter-fiber can be avoided by shaking scaffold during cross-linking process. c, FTIR graph (top) and shows a small decrease of the C—O—C— and O—H groups of HA fibers compared to the raw lyophilized powder, while more market decreases are confirmed with the cross-linked fibers. This confirms fiber assembly and subsequent cross-linking, as the availability of these groups will decrease following these processes. n=3 different measurements on a sample.

FIGS. 63(a)-63(b) depict versatile material fabrication capabilities. a, To demonstrate the versatility of the manufacturing approach herein, the GAG chondroitine sulfate and the ECM proteins collagen, gelatin and fibrinogen were used to fabricate micro- and nano-fiber scaffolds from aqueous solutions. Insets. Close-up SEMs show distinctive morphologies and intra-fiber molecular packing. b, To support cellular adhesion in HA scaffolds, binding moieties were introduced by spinning HA/gelatin hybrid fibers. SEMs show two different hybrids, termed low and high protein content with respectively 1% w/v (1:1 HA/gelatin ratio), and 1.75% w/v (3:4 HA/gelatin ratio).

FIG. 64 depcits high throughput manufacturing of HA nanofibers using iRJS. Graph depicts the low production rate of previously published electrospinning (e-spinning) and electroblowing (e-blowing) techniques for HA nanofibers (empty bars), compared to our current iRJS setup with production-scale capabilities.

FIGS. 65(a)-65(b) depict flexible spinning conditions of porous nanofiber HA scaffolds. a, Large nanofiber scaffolds were produced in a single-step process using a wide range of concentrations (1-4% weight/volume) from aqueous solutions. Left. Macroscopic image shows a HA scaffold lyophilized into a cylindrical shape. Right. Scanning electron micrographs (SEM) depict the typical basket-woven structure produced using our collectors. Bottom. SEMs of different HA scaffolds produced using increasing concentrations (w/v) of HA in the starting aqueous solution. b, Left. Rheological measurements reveal Brigham pseudoplastic behaviors for HA dopes of different concentrations. Right. HA viscosity increased with increasing dope concentration, while individual curves decreased as a function of higher shear stresses. Significant decreases in viscosity can therefore be expected for all concentrations at iRJS spinning conditions, suggested by the convergent trajectories. n=3 per condition, errors presented as s.e.m. b, Large scaffolds could additionally be imaged using μCT, detailing the uniform fibrous structure throughout the scaffold.

FIGS. 66(a)-66(b) depict rheological measurements of HA dopes. Left. Measurements reveal Brigham pseudoplastic behaviors for HA dopes of different concentrations. Right. HA viscosity increased with increasing dope concentration, while individual curves decreased as a function of higher shear stresses. Significant decreases in viscosity can therefore be expected for all concentrations at iRJS spinning conditions, suggested by the convergent trajectories. n=3 per condition, errors presented as s.e.m.

FIG. 67 depcits SEM images of sectioned HA nanofiber scaffolds. Images at the center of the scaffold (enlarged on the right-hand image) reveal the uniformity and porosity of the engineered scaffolds.

FIGS. 68(a)-68(g) depict that HA scaffolds demonstrate structural and mechanical tenability. a, Fiber diameter increases from ˜1.0 μm for 1% (w/v) HA polymer dope to ˜3.0 μm for the 4% for fixed spinning at 15 k rpm. b, Fiber diameter conversely decreases with reservoir rotation speed increase, reaching average diameters below 1.0 μm at 30 k rpm. c, Porosity measurements reveal a decreasing trend with increasing polymer dope or fiber size as detailed in (a). d, Porosity can be modulated more significantly, and without relying on polymer dope, via dehydration post-spinning Non-dehydrated HA scaffolds (1% w/v) show a porosity of ˜75%, while scaffolds dried for 60 min exhibit a porosity of ˜55%. e, Corresponding SEM cross-section images of five different scaffold porosities that were enabled by dehydrating the scaffolds for 0, 15, 30, 45 and 60 min f, As-spun scaffolds demonstrate a strong water absorption capacity (calculated as the swelling ratio), reaching a ˜25-30 fold increase (2,500%-3,000%) in weight from their dry state. Water absorption capacity increased post-cross-linking, reaching 60 fold increase in weight (˜6,000%) for the 1% HA scaffolds. g, Young's modulus in compression and in extension (along fiber axis) scale with HA concentration, suggesting a correlation with fiber diameter detailed in (a). a-d, n=3 sample runs per condition with 4-6 field of views (FOVs) each. f, n=8 samples per condition. g, n=5-8 samples per condition. Errors bars are presented as s.e.m.

FIG. 69 depcits concentration-dependent fiber diameters. Histograms of fiber diameters show relatively normal distributions for the 0.5-2% w/v and become more negatively-skewed for the 3-4%. Fiber sizes range from 0.6 μm on average for 0.5% to 3.14 μm for the 4% w/v. Rotation speed was kept constant at 15 k rpm. n>100 fibers from several sample runs (>3).

FIGS. 70(a) and 70(b) depict rotation speed-dependent fiber diameters. a, Histograms of fiber diameter show relatively normal distribution for 5 k-15 k rpm and a more negatively-skewed distribution for the 30 k rpm. Fiber sizes range from 3.28 μm on average for lowest speed at 5 k rpm to 0.86 μm for the 30 k rpm. All solution dopes were kept constant at 1% w/v. n>100 fibers from several sample runs (>3). b, Representative SEM images of at low and higher magnification show decrease in fiber size with increasing reservoir rotation speed.

FIG. 71 depcits representative SEMs of varying scaffold porosities produced by nanofiber spinning platforms. (Top) Rotary jet spinning (RJS), previously described (Badrossamay, M. R., McIlwee, H. A., Goss, J. A. & Parker, K. K. Nano Lett 10, 2257-2261 (2010), is higher throughput dry-spinning nanofiber fabrication technique. Collection on mandrels can enable tunability over porosity and fiber alignment over a defined range. (Bottom) Immersed RJS, used in this study, enabled fabrication of highly porous nanofiber scaffolds. Wet rotating collection bath enables to significantly increase attainable porosity, when compared to dry-spinning techniques exemplified an RJS technique.

FIG. 72 depcits water absorption and degradation of HA scaffolds. (Left) As-spun HA scaffolds show rapid water absorbance (quantified by swelling ratio), but degrade rapidly via hydrolysis, losing their structural properties within the first 100 min of incubation. (Right) To increase structural stability over time, cross-linking of the hydroxyl- and carboxyl-groups via ester bond formation is induced (see FIG. 62). Measurements of cross-linked scaffold weight over time reveal a gradual degradation. After ˜1 week (10,000 min), they still retained between 80 and 95% of their weight when incubated in PBS at 37° C. Their swelling ratio shows also an increase over non-cross-linked fibers. n=8 samples per condition. Errors bars are presented as s.e.m.

FIGS. 73(a)-73(d) depict cell infiltration improves with increasing HA scaffold porosity. a, Representative live-confocal microscope images of dermal fibroblasts (GFP-HNDFs) at the scaffold surface, 50 μm deep, and 100 μm deep for varying scaffold porosities (dense HA (dHA): 55%, standard HA (sHA): 65%, and porous HA (pHA): 75%). 1% w/v precursor solution spun at 15 k rpm was used for all fabricated scaffolds. b, Orthogonal views of 3D reconstruction, corresponding to images in (a). c, Intensity values (normalized) were plotted up 100 μm in depth, and confirm the decreased presence of cells deeper in the dHA and sHA scaffolds. d, Quantification of infiltration (intensity values) averaged over 100 μm (left) and measured at the 100 μm position (right) demonstrate significant differences between all groups tested. N=4 samples with 4-6 FOVs per sample. One way ANOVA with post hoc multiple comparisons Holm-Sidak's tests were performed. Significance was considered for p<0.05. Errors bars are presented as s.e.m.

FIGS. 74(a)-74(f) depict porous HA scaffolds support robust wound closure and tissue regrowth. a, Schematic of the full-thickness excisional splinting wound model procedure steps: (1) 6 mm full-thickness excisional wounds are inflicted on C57BL/6 male mice (8-10 weeks old), (2) silicon rings are sutured to the surrounding uninjured skin, (3) HA wound dressings are applied to the wound, and (4) silicon occlusive dressings (Tegaderm™) are used to cover the wounds. b, Representative SEM images of standard HA scaffold (sHA; ˜55% porosity) and the porous HA scaffold (pHA; ˜75% porosity). c, Representative macroscopic images of wounds at day 0 and at day 6 post-injury for the control (only covered with a Tegaderm™ film dressing), the sHA and the pHA dressings. HA-treated wounds reveal formation a scab across the entire wounded area, while controls appear still completely open. d, Percentage of original wound area 6 days after wounding. One way ANOVA on ranks with a post hoc multiple comparisons Dunn's test was used. e, Trichrome stained sections of control (top), sHA (center) and pHA (bottom) dressings. Controls revealed minimal wound closure, characterized by the lack of reepithelialization. Center of the wound exhibited almost no cellular presence (see inset image). By contrast, both HA scaffold demonstrated strong tissue regrowth, with the pHA group showing a significant difference in reepithelialization when compared to the control (see vertical arrowheads and inset images). Both HA scaffolds supported granulation tissue formation bellow the epidermis (in blue). Black dotted lines and arrows highlight formation of epithelial tongue and new epidermis. f, Quantification of reepithelialization length (top) and granulation tissue formation (bottom) 6 days after wounding. One way ANOVA on ranks with post hoc multiple comparisons Holm-Sidak's tests were performed. Significance was considered for p<0.05. Errors bars are presented as s.e.m.

FIGS. 75(a)-75(b) depict porous HA-treated tissues demonstrate reduction in scar size. a, Photographic images of wound specimen 28 days after wounding reveal the formation of scar tissues in both treatments (white line depicts the scar edge), with however a reduction in size for the pHA condition. b, Quantification of scar size as a percentage of original wound area measured scars at 19.5% and 11% for the control and pHA groups, respectively. n=4 wounds per condition; Student's t-test. Errors bars are presented as s.e.m.

FIGS. 76(a)-76(b) depict an exemplary pull spinning system: (a) representative image and (b) schematic diagram of the setup.

FIG. 77 depcits SEM images of spun a) alfalfa (1 wt/v %) solution, b) PCL/alfalfa (6 wt/v %/1.5 wt/v %), and c) PCL/alfalfa (6 wt/v %/2 wt/v %) fiber scaffolds. Scales are 100 μm.

FIGS. 78(a)-78(c) depict SEM images, FIGS. 78 (d)-78(ff) fiber diameter analysis, FIG. 78 (g) alignment analysis, and FIG. 78 (h) porosity analysis of PCL (6 wt/v %) nanofiber, PCL/Alfalfa (6 wt/v %/0.5 wt/v %) nanofiber, and PCL/Alfalfa (6 wt/v %/1 wt/v %) nanofiber. Scales of SEM images are 20 μm. For a statistical analysis in (d-h), n=4, field of view (FOV)≥4. For the fiber alignment analysis, Gaussian fits were applied to raw data to show the distribution of fiber directionality. (i) Young's modulus and (j) specific modulus of nanofiber scaffolds. For statistical analysis, n=12 and *p<0.05.

FIGS. 79(a)-79(h) depict chemical and mechanical properties of alfalfa fibers. (a) FT-IR spectra of nanofibers. Black arrows indicate amide peaks. (b-d) Representative images of (b) PCL (6 wt/v %) and (c) PCL/Alfalfa (6 wt/v %/0.5 wt/v %) nanofibers with (d) corresponding UV-vis absorption spectra. Black arrows indicate absorbance peaks specific to alfalfa (λₘₐₓ=435, 663 nm). (e-h) Hyperspectral imaging of (e) alfalfa film, (f) PCL nanofiber, and (g) PCL/alfalfa nanofiber with (h) the corresponding spectra. The color of spectra matches to the color of boxes in the images. Scales are 10 μm.

FIGS. 80(a)-80(d) depict contact angle measurements of (a-b) cast films and (c-d) nanofibers. For statistics, n=4 for (b) and n=3 for (d), error bars in (d) are SEM.

FIG. 81 depcits phytoestrogen (genistein) analysis by LC-MS. The grey box indicates the genistein-specific peak (m/z=269).

FIG. 82 depcits cytotoxicity measurement of HNDFs on nanofibers using LDH assay. n=4, triplicate.

FIGS. 83(a)-83(f) depcit in vitro fibroblast and neuron cultures. (a-c) GFP-expressing HNDFs cultured on (a) PCL and (b) PCL/Alfalfa nanofiber scaffolds at Day 7 with (c) analysis of cell coverage on nanofibers. n=10 (field of view>25). Scales are 50 μm. *p<0.05. (d-f) Neurons cultured on d) PCL nanofiber scaffolds and (e) PCL/Alfalfa nanofiber scaffolds at Day 7 with f) neurite outgrowth analysis. Scales are 1 mm. *p<0.05, n=6 for PCL and PCL/alfalfa nanofiber scaffolds for neurite outgrowth analysis.

FIGS. 84(a)-84(d) depcit in vitro cardiomyocyte culture. NRVMs cultured on (a) PCL/Alfalfa nanofiber scaffolds at Day 5. Blue=DAPI and red=α-actinin. Scale is 50 μm. 3D reconstruction of NRVMs cultured on (b) PCL/Alfalfa nanofiber scaffolds. Blue=DAPI and red=α-actinin. Electrophysiological property of channelrhodopsin (ChR2)-expressing NRVM tissues on PCL/alfalfa fiber scaffolds with (c) time-lapse images of Ca²⁺ wave propagation, calculated from the temporal derivative of fluorescent signal, and (d) Ca²⁺ signal traces at 1 Hz optical pacing. The Ca²⁺ signals were obtained from the white boxes from (c). Purple boxes denote the optical pacing points. Scale of (c) is 5 mm.

FIGS. 85(a)-85(h) depict in vivo tissue regeneration. a) Schematic animation of the excisional splinting wound model. b-c) Representative images of wounds at day 0 and 14 post surgery with wound closure analysis at day 14 post surgery. *p<0.05 and n=6. Scales are 1 mm. d-h) Masson's trichrome images of day 14 wounds with epithelial gap and granulation tissue formation analysis. The black arrows in the images indicate the edge of epithelial tongues in the wound sites. *p<0.05, n=6 for control and n=5 for PCL and PCL/Alfalfa nanofibers (2 sections per tissue).

FIG. 86 depcits hair follicle formation in wounds treated with the indicated polymeric scaffolds. The arrows in the Masson's trichrome and immunofluorescence images indicate new hair germ and follicle formation in the wound site. Scales are 100 μm.

FIG. 87a depict scanning electron micrographs of the steps of HA/SPI polymeric fiber formation and cross-linking.

FIG. 87b depict the chemical formulas of hyaluronic acid before formation of polymeric fibers comprising HA/SPI, after formation of polymeric fibers comprising HA/SPI, and polymeric fibers comprising HA/SPI after cross-linking with EDC/NHS.

FIG. 88a depicts scanning electron micrographs of fibers formed from the indicated solutions.

FIG. 88b depicts the chemical structure of genistein (top left), a full mass spectrometry spectra of genistein showing the major peak at 271 (m/z) (bottom left), and a graph depicting the results of selective ion monitoring (SIM) of liquid chromatography-mass specetromety analysis of the fibers formed from the indicated solutions to verify the existence of genistein in HA/SPI fiber scaffolds (right).

FIG. 89 provides the FT-IR spectra of the fibers formed from the indicated solutions.

FIG. 90a is a graph depicting the diameter of the fibers formed from the indicated solutions as well as SEM images of the formed fibers and scaffolds.

FIG. 90b provides SEM images of the fibers formed from the indicated solutions.

FIG. 91a is a graph depicting the mechanical strength of the fiber scaffolds formed from the indicated solutions.

FIG. 91b provides the stability of the fiber scaffolds in phosphate buffered saline (PBS) or Dulbecco's Modified Essential Medium (DMEM).

FIG. 92 is a graph depicting the porosity of the fiber scaffolds formed from the indicated solutions.

FIG. 93a depicts photographic images of the wounds treated as indicated at the indicated days.

FIG. 93b is a graph depicting the percent of wound closure over time using the scaffolds indicated.

FIG. 94(a) depicts microscopic images of Masson's trichrome stained wound samples to show the effect of treating the wounds with the indicated scaffolds on connective tissues (medium gray) as well as keratinocytes, hair follicles, and adipose tissues (dark gray) at Day 20 post-surgery.

FIG. 94(b) depicts a schematic of the wound healing study performed in mice (top) and the graphs below depict the effect of the indicated scaffolds on epithelial thickness (top), scar index (middle) and hair follicle counts (bottom) at Day 20 post-surgery.

FIG. 95 depicts immunofluorescence images of day 20 post-surgery tissues treated with the indicted scaffolds. The tissues were stained with DAPI (for nuclei), ER β, and K14 (for hair follicles) antibodies.

FIG. 96(a) depicts microscopic images of Masson's trichrome stained wound samples to show the effect of treating the wounds with the indicated scaffolds on connective tissues (medium gray) as well as keratinocytes, hair follicles, and adipose tissues (dark gray) at Day 7 post-wounding.

FIG. 9b depicts a schematic of the wound healing study performed in ex vivo human tissues (top) and the graph below depict the effect of the indicated scaffolds on epithelial gap size at Day 7 post-wounding.

### DETAILED DESCRIPTION

The present invention is based, at least in part, on the fabrication of polymeric fibers, e.g., micron, submicron or nanometer dimension polymeric fibers comprising one or more polymers, e.g., protein, and non-woven polymeric scaffolds comprising the polymeric fibers that have physical and mechanical properties that mimic dermal skin extracellular matrix and/or fetal dermal skin extracellular matrix and that promote and accelerate cutaneous wound closure, promote cutaneous wound healing and/or cutaneous tissue regeneration and reduce fibrosis.

In the following brief descriptions and throughout the specification, weight/volume percentages (w/v %) associated with the fibers and scaffolds of the invention mean that the related fibers and scaffolds are prepared using a solution containing such amounts expressed as w/v %. For example, “CA (10 wt/v %) nanofibers” means that the fibers are prepared using a solution containing 10 wt/v % CA. “CA/SPH (10 wt/v %/5 wt/v %) nanofibers” means that the fibers are prepared using a solution containing 10 wt/v % CA and 5 wt/v % SPH. “PCL (6 wt/v %) nanofibers” means that the fibers are prepared using a solution containing 6 wt/v % PCL. Accordingly, the fibers prepared with, for example, 10 wt/v % CA and 10 wt/v % SPH means that the formed fibers, themselves, are 50 wt/wt % CA and 50 w/w % SPH. Similarly, the fibers prepared with, for example, 10 wt/v % CA and 5 wt/v % SPH means that the formed fibers, themselves, are about 66.6 wt/wt % CA and about 33.3 w/w % SPH.

It should be noted that whenever a value or range of values of a parameter are recited, it is intended that values and ranges intermediate to the recited values are also intended to be part of this invention.

### A. Polymeric Fiber Scaffolds and Wound Dressings of the Invention

The present invention provides polymeric fibers and non-woven polymeric fiber scaffolds comprising a plurality of polymeric fibers fibers that promote wound healing and tissue regeneration, e.g., cutaneous wound healing and tissue regeneration. The scaffolds of the invention have been engineered to mimic the extracellular matrix of skin and/or the extracellular matric of fetal skin and, thus, also reduce or inhibit scar formation (fibrosis) during wound healing. The term “fiber” and “polymeric fiber” are used interchangeably herein, and both terms refer to polymeric fibers having micron, submicron, and nanometer dimensions. The term “scaffold” as used herein refers to a structure comprising a pluarailty of polymeric fibers that provides structure to a tissue and allows cells to adhere, proliferate, and differentiate.

Accordingly, in some aspects, the polymeric fiber scaffolds of the invention are incorporated into wound dressings, which include, for example, a backing material, an adhesive material, and additional agent, such as a clotting agent, an antibacterial agent, a pharmaceutically acceptable carrier, e.g., injection into a wound, e.g., for packing a wound. The scaffold in wound dressings comprising, e.g., a backing material, is, typically, in direct contact with the wound.

The polymeric fiber scaffolds of the invention may further include an additional therapeutic agent, such as an agent which, e.g., angiogenesis, granulation tissue formation, etc. For example, the polymeric fibers may be contacted with additional agents which will allow the agents to, for example, coat (fully or partially) the fibers. In some embodiments, the polymer solution is contacted with the additional agent during the fabrication of the polymeric fibers which allows the agents to be incorporated into the polymeric fibers themselves.

The polymeric fiber scaffolds may also be contacted with cells, e.g., seeded, with a plurality of living cells, such as epithelial cells, stem cells, e.g., embryonic stem cells or adult stem cells, progenitor cells), to allow the cells to intercalate between fibers.

In one embodiment, the additional therapeutic agent is a therapeutic cytokine, such as an interleukin. In another embodiment, the additional therapeutic agent is a therapeutic cytokine, such as growth e.g., platelet derived growth factor (PDGF), fibroblast growth factor (FGF), epidermal growth factor (EGF), connective tissue growth factor (CTGF), hepatocyte growth factor (HGF), insulin-like growth factor (IGF), stromal cell derived factor-1 (SDF-1), bone morphogenic proteins (BMPs), nerve growth factor (NGF) transforming growth factors (a,b), keratinocyte growth factor (KGF) or vascular endothelial growth factor (VEGF)

In yet another embodiment, the additional therapeutic agent is a bacteriostatic agent, an antibacterial agent, an antimicrobial agent, an antibiotic, and/or an antifungal agent

Exemplary antimicrobials include but are not limited to silver, copper, zinc, titanium oxide, chlorhexidine gluconate, polyhexamethylene biguanide, povidone iodine, cadexomer iodine, citric acid, hypochlorous acid, antimicrobial peptides, honey, glucose oxidase generated hydrogen peroxide, or hydrogen peroxide generated or held by other methods. Antimicrobial agents with selectivity for bacterial physiologic targets over eukaryotic cytotoxicity would be preferred.

In one embodiment, the additional therapeutic agent is an agent an anti-inflammatory agent. In another embodiment, the additional therapeutic agent is an anti-scarring agent. In yet another embodiment, the additional therapeutic agent is an analgesic. Such agents include, for example, opiods, steroids, steroidal anti-inflammatory drugs, inhibitors of cyclooxygenase (COX) 1 & 2, a non-steroidal anti-inflammatory drug (NSAIDs) including ibuprofen and naproxen sodium, and anti-oxidants such as ascorbic acid or carotenoids.

The scaffolds of the invention may also be, for example, used as extracellular matrix and, together with cells, may also be used in forming engineered tissue. Such tissue is useful not only regenerative medicine, but also for investigating tissue developmental biology and disease pathology, as well as in drug discovery and toxicity testing. The scaffolds of the invention may also be combined with other substances, such as, therapeutic agents (such as an agent which, e.g., enhances or augments tissue growth, cell migration, etc.) during or after fabrication of the polymeric fibers and scaffolds in order to deliver such substances to the site of application of the polymeric fiber scaffolds and/or wound dressings.

1. Polymeric Fiber Scaffolds Comprising Cellulose and Soy Protein Hydrosylate

In one aspect, the present invention provides polymeric fiber scaffolds which include a plurality of polymeric fibers, each polymeric fiber independently comprising cellulose (e.g., cellulose acetate) and soy protein hydrolysate. In a particular embodiment, the cellulose and soy protein hydrolysate are co-spun to form the scaffold (described below). The cellulose component serves as a soft and hydrophilic backbone similar to that of the collagen matrix in the dermal native tissue, while the protein component promotes wound healing by accelerating proliferation, growth, migration, infiltration, and recruitment of integrin β1 expressing fibroblasts and keratnocytes. In a particular embodiment, the soy protein hydrolysate is homogeneously distributed along the fibers (i.e., co-spinning of soy protein hydrolysate and cellulose results in an even districution of soy protein hydrosylate in the fibers and along the length of the fibers). Additionally, the scaffolds of the invention contain bioactive molecules, e.g., phytoestrogens that enhance skin regeneration. Furthermore, the scaffolds are moisture-retaining (or hydrating) due to the high hydrophilicity and swelling properties of CA/SPH nanofibers. Thus, the scaffolds of the invention are useful in methods of wound healing, since they provide both structural and biological cues for promoting wound healing.

Cellulose is a natural polymer, which is manufactured from purified natural cellulose. Natural cellulose of the appropriate properties is derived primarily from two sources, cotton linters and wood pulp. Cellulose acetate is an ester of cellulose. In the manufacturing of cellulose acetate, natural cellulose is reacted with acetic anhydride to produce cellulose acetate, which comes out in a flake form. This flake is then ground to a fine powder.

As used herein, the term “soy protein” refers to a type of peptide or protein (including phytoestrogens and isoflavones) that is derived from soybean. The term “soy protein” also refers to a soy protein concentrate that is an unpurified or crude mixture of amino acids, peptides, proteins (including phytoestrogens and isoflavones) that are derived from soybean. In one embodiment, soy protein in accordance to the latter definition is made from soybean meal that has been dehulled. In another embodiment, soy protein is made from soybean meal that has been dehulled and defatted. In some embodiments, soy protein is provided in the form of soy flour. The protein content in soy protein is no higher than 70% w/w, e.g., about 40% to 60%, about 40%, about 52%, about 55% and about 60%, etc.

As used herein, the term “soy protein isolate” (SPI) or “isolated soy protein” refers to soy protein (in accordance with the second definition given above) where the non-protein components, i.e., fat and carbohydrates, have been removed. The protein content in soy protein isolate is about 90% to 95% w/w.

As used herein, the term “soy protein hydrolysate” (SPH) or “hydrolyzed soy protein” refers to soy protein isolate that is hydrolyzed to further maximize the protein content. In one embodiment, the soy protein isolate is enzymatically hydrolyzed to produce soy protein hydrolysate. Suitable enzymes include proteases and peptidases, such as but not limited to alcalase and Flavourzyme™. In one embodiment, either the glycinin or β-conglycinin fractions in the soy protein isolate are selectively hydrolyzed to produce soy protein hydrolysate. The protein content in soy protein hydrolysate is typically higher that n 95% w/w, e.g., about 97%, about 98%, about 99%, about 99.5%, about 99.9%. Moreover, SPH has higher solubility (i.e., >60%) compared to SPI (i.e., 5%) at the isoelectric point.

In one embodiment, a solution used to form the cellulose acetate/soy protein hydrolysate (CA/SPH) polymeric fibers and the scaffolds of the invention comprises about 5% to 30% w/v of cellulose acetate (based on volume of the carrier during manufacturing of the fibers and scaffolds, i.e., w/v %), e.g., about 5% to 25%, about 5% to 20%, about 5% to 15%, about 5% to 10%, about 10% to 30%, about 10% to 25%, about 10% to 20%, about 10% to 15%, about 15% to 30%, about 15% to 25%, about 15% to 20%, about 20% to 30%, about 25% to 30%, about 5%, about 7.5%, about 10%, about 12.5%, about 15%, about 17.5%, about 20%, about 22.5%, about 25%, about 27.5%, about 30% w/v %. Preferably, the solution comprises about 5% to 20%, about 5% to 15%, about 5% to 10%, about 10% to 20%, about 10% to 15%, about 5%, about 7.5%, about 10%, about 12.5%, about 15%, about 17.5%, or about 20% w/v % of cellulose acetate. More preferably, the solution comprises about 5% to 15%, about 5% to 10%, about 10% to 15%, about 5%, about 10%, or about 15% w/v % of cellulose acetate. In one embodiment, the solution comprises about 5% to 15% w/v % of cellulose acetate. In another embodiment, the solution comprises about 8% to 12% w/v % of cellulose acetate. In another embodiment, the solution comprises about 9% to 10% w/v % of cellulose. In another embodiment, the solution comprises about 5% to 10% w/v % of cellulose. In another embodiment, the solution comprises about 10% w/v % of cellulose acetate. In yet another embodiment, the solution comprises about 15% w/v % of cellulose acetate.

In one embodiment, a solution used to form the cellulose acetate/soy protein hydrolysate (CA/SPH) fibers and the scaffolds of the invention comprises about 0.5% to 15% w/v (based on volume of the carrier during manufacturing of the fibers and scaffolds, i.e., w/v %), e.g., about 1% to 15%, about 2% to 15%, about 3% to 15%, about 5% to 15%, about 7.5% to 15%, about 10% to 15%, about 12% to 15%, about 1% to 12.5%, about 2% to 12.5%, about 3% to 12.5%, about 5% to 12.5%, about 7.5% to 12.5%, about 10% to 12.5%, about 1% to 10%, about 2% to 10%, about 3% to 10%, about 5% to 10%, about 7.5% to 10%, about 1% to 5%, about 2% to 5%, about 3% to 5%, about 4% to 5%, about 3% to 6%, about 4% to 6%, about 5% to 6%, about 1%, about 2%, about 3%, about 5%, or about 10% w/v %. Preferably, the solution comprises about 1% to 10%, about 3% to 10%, about 5% to 10%, about 1% to 5%, about 2% to 5%, about 3% to 5%, about 4% to 5%, about 3% to 6%, about 4% to 6%, about 5% or 6%, about 1%, about 2%, about 3%, or about 5% w/v % of soy protein hydrolysate. More preferably, the solution comprises about 1% to 5%, about 2% to 5%, about 4% to 5%, about 3% to 6%, about 4% to 6%, or about 5% or 6%, about 1%, about 2%, about 3%, or about 5% w/v % of soy protein hydrolysate. In one embodiment, the solution comprises about 4% to 6% w/v % of soy protein hydrolysate. In another embodiment, the solution comprises about 1% w/v % of soy protein hydrolysate. In another embodiment, the solution comprises about 3% w/v % of soy protein hydrolysate. In another embodiment, the solution comprises about 5% w/v % of soy protein hydrolysate.

In some embodiments, the carrier used during fabrication of the CA/SPH fibers and scaffolds of the invention is an organic solvent. Preferably, the organic solvent is a polar, protic solvent. Preferably, the organic solvent is an alcohol including a pure alcohol or a solvent system with an alcohol as the primary solvent, and non-limiting examples of a suitable alcohol are n-butanol, tert-butanol, methanol, ethanol, n-propanol and isopropanol. In one embodiment, the alcohol used as a carrier in the manufacturing of the CA/SPH fibers and scaffolds is a halogenated alcohol, such a halogenated C1-C4 alcohol. In one embodiment, the carrier used in the manufacturing of the CA/SPH fibers and scaffolds is hexafluoroisopropanol (HFIP).

Since the carrier solvent dissipates completely upon formation (e.g., solidification) of the fibers and scaffolds, the formed fibers and scaffolds of the invention, accordingly, contain CA and SPH at a CA/SPH weight ratio of about 1.5-3:1, e.g., about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, or about 3:1, preferably 1.8:1, about 1.9:1, about 2:1, about 2.1:1, or about 2.2:1, more preferably about 1.9:1, 2:1, or about 2.1:1. In one embodiment, the CA/SPH weight ratio is about 2:1.

Methods for forming polymeric fibers and scaffold comprising CA and SPH are described below.

Alternatively or additionally, when expressed as weight/weight percentages, the formed fibers and scaffolds of the invention contain about 60-70% w/w CA (based on total weight of CA/SPH fiber or CA/SPH scaffold), e.g., about 61-70%, about 62-70%, about 63-70%, about 64-70%, about 65-70%, about 66-70%, about 67-70%, about 68-70%, or about 69-70%, preferably about 64-70%, about 65-70%, about 66-70%, about 67-70%, or about 68-70%, more preferably about 65-70%, about 66-70%, about 67-70%. In one embodiment, the formed fibers and scaffolds of the invention contain about 66.67% w/w CA. As SPH, the formed fibers and scaffolds of the invention contain about 30-40% w/w SPH (based on total weight of CA/SPH fiber or CA/SPH scaffold), e.g., about 30-39%, about 30-38%, about 30-37%, about 30-36%, about 30-35%, about 30-34%, about 30-33%, about 30-32%, or about 30-31%, preferably about 30-35%, about 30-34%, about 30-33%, or about 30-32%, more preferably about 30-34%, or about 30-35%. In one embodiment, the formed fibers and scaffolds of the invention contain about 33.33% w/w SPH.

The scaffolds of the invention promote cutaneous wound healing and/or cutaneous tissue regeneration and have physical and mechanical properties that mimic dermal skin extracellular matrix, as elaborated in the following paragraphs.

In some embodiments, each CA/SPH fiber in the scaffold independently has a diameter of about 200 nm to 400 nm, e.g., about 250 nm to 400 nm, about 300 nm to 400 nm, about 350 nm to 400 nm, about 360 nm to 400 nm, about 370 nm to 400 nm, about 375 nm to 400 nm, about 380 nm to 400 nm, about 385 nm to 400 nm, about 390 nm to 400 nm, about 395 nm to 400 nm, about 300 nm, about 325 nm, about 350 nm, about 360 nm, about 370 nm, about 375 nm, about 380 nm, about 385 nm, about 390 nm, about 395 nm, or about 400 nm. Ranges and values intermediate to the above recited ranges and values are also contemplated to be part of the invention. Fiber diameters ranging from 200 nm to 400 nm, which are similar to native extracellular matrix, enhance adhesion and proliferation of human dermal fibroblasts.

Preferably, the fiber diameter is about 300 nm to 400 nm, about 350 nm to 400 nm, about 375 nm to 400 nm, about 380 nm to 400 nm, about 390 nm to 400 nm, about 395 nm to 400 nm, about 300 nm, about 350 nm, about 375 nm, about 380 nm, about 385 nm, about 390 nm, about 395 nm, or about 400 nm. More preferably, the fiber diameter is about 300 nm to 400 nm, about 350 nm to 400 nm, about 375 nm to 400 nm, about 390 nm to 400 nm, about 395 nm to 400 nm, about 300 nm, about 350 nm, about 375 nm, about 390 nm, about 395 nm, or about 400 nm. In one embodiment, the fiber diameter is about 390 nm. In another embodiment, the fiber diameter is about 395 nm. In yet another embodiment, the fiber diameter is about 400 nm. Comparatively, polycaprolactine (PCL) fibers typically have fiber diameters exceeding 600 nm. Ranges and values intermediate to the above recited ranges and values are also contemplated to be part of the invention. The scaffolds themselves may be of any desired size and shape and can be fabricated according to need and use. Methods for fabricating the polymeric fiber scaffold are described below.

In certain embodiments, the scaffold formed has a porosity greater than about 40%, e.g., a porosity of about 60% to about 80%, about 65% to about 80%, about 70% to about 80%, e.g., about 60, 61, 62, 63, 64, 65, 66, 67, 68, 69, 70, 71, 72, 73, 74, 75, 76, 77, 78, 79, or about 80%. Ranges and values intermediate to the above recited ranges and values are also contemplated to be part of the invention.

In some embodiments, the average pore diameter of the scaffold formed is about 6 μm to 20 μm, about 6 μm to 15 μm, about 6 μm to 12 μm, about 6 μm to 10 μm, about 6 μm to 8 μm, about 6 μm, about 8 μm, about 10 μm, about 12 μm, about 15 μm, or about 20 μm. Preferably, the average pore diameter is about 6 μm to 10 μm, about 6 μm to 8 μm, about 6 μm, about 8 μm, or about 10 μm. More preferably, the average pore diameter is about 6 μm to 8 μm, about 6 μm, or about 8 μm. In one embodiment, the average pore diameter is about 6 μm. Ranges and values intermediate to the above recited ranges and values are also contemplated to be part of the invention. Pore diameters ranging from 6 μm to 20 μm, which are similar to native extracellular matrix, enhance adhesion and proliferation of human dermal fibroblasts. Comparatively, polycaprolactine (PCL) scaffolds typically have pore diameters that are under 4 μm.

Fiber and scaffold stiffnessness also affects cell behavior. To encourage assembly of new estracellular matrix (ECM), the stiffness of wound dressing materials should mimic the stiffness of the native ECM microenvironment of about 5 kPa to 600 kPa in Young's modulus. In some embodiments, the Young's modulus of the scaffold, which indicates the stiffness of the scaffold, is about 5 kPa to 600 kPa in the longitudinal direction, about 50 kPa to 500 kPa, about 50 kPa to 400 kPa, about 50 kPa to 300 kPa, about 50 kPa to 250 kPa, about 50 kPa to 200 kPa, about 100 kPa to 500 kPa, about 100 kPa to 400 kPa, about 100 kPa to 300 kPa, about 100 kPa to 250 kPa, about 100 kPa to 200 kPa, about 150 kPa to 200 kPa, about 50 kPa, about 100 kPa, about 150 kPa, about 200 kPa, about 250 kPa, about 300 kPa, about 400 kPa, or about 500 kPa. Preferably, the Young's modulus of the scaffold in the longitudinal direction is about 100 kPa to 300 kPa, about 100 kPa to 250 kPa, about 100 kPa to 200 kPa, about 150 kPa to 200 kPa, about 100 kPa, about 200 kPa, about 250 kPa, or about 300 kPa. More preferably, the Young's modulus in the longitudinal direction is about about 100 kPa to 200 kPa, about 150 kPa to 200 kPa, about 100 kPa, about 150 kPa, or about 200 kPa. In one embodiment, the Young's modulus of the scaffold in the longitudinal direction is about 200 kPa. Ranges and values intermediate to the above recited ranges and values are also contemplated to be part of the invention. Comparatively, the stiffness of common synthetic polymer nanofiber scaffolds used as wound dressings, such as polycaprolactone (PCL) scaffolds, is usually one to several orders of magnitude higher, i.e., in the MPa range.

In some embodiments, the Young's modulus of the scaffold is about 5 kPa to 600 kPa in the transverse direction, about 50 kPa to 500 kPa, about 50 kPa to 400 kPa, about 50 kPa to 300 kPa, about 50 kPa to 250 kPa, about 50 kPa to 200 kPa, about 100 kPa to 500 kPa, about 100 kPa to 400 kPa, about 100 kPa to 300 kPa, about 100 kPa to 250 kPa, about 100 kPa to 200 kPa, about 100 kPa to 150 kPa, about 100 kPa to 120 kPa, about 120 kPa to 130 kPa, about 50 kPa, about 100 kPa, about 120 kPa, about 130 kPa, about 150 kPa, about 200 kPa, about 250 kPa, about 300 kPa, about 400 kPa, or about 500 kPa. Preferably, the Young's modulus of the scaffold in the transverse direction is about 100 kPa to 300 kPa, about 100 kPa to 250 kPa, about 100 kPa to 200 kPa, about 100 kPa to 150 kPa, about 100 kPa to 120 kPa, about 120 kPa to 130 kPa, about 100 kPa, about 120 kPa, about 130 kPa, about 200 kPa, about 250 kPa, or about 300 kPa. More preferably, the Young's modulus in the transverse direction is about about 100 kPa to 150 kPa, about 100 kPa to 120 kPa, about 120 kPa to 130 kPa, about 100 kPa, about 120 kPa, or about 130 kPa. In one embodiment, the Young's modulus of the scaffold in the transverse direction is about 120 kPa. In another embodiment, the Young's modulus of the fiber/scaffold in the transverse direction is about 126 kPa. In another embodiment, the compression modulus of the scaffold in the transverse direction is about 130 kPa. Ranges and values intermediate to the above recited ranges and values are also contemplated to be part of the invention.

The thickness of the CA/SPH fibrous scaffolds of the invention can be controlled. For example, if a rotary jet spinning (RJS) system is used to spin the fibers and to produce the scaffolds, the thickness of the scaffold can be controlled by the amount of the carrier or the polymer solution used. In another embodiment, the thickness of the scaffold can be controlled by the rotation speed. In some embodiments, the thickness of the scaffold ranges from about 0.1 mm to 5 mm, e.g., about 0.2 mm to 4 mm, about 0.2 mm to 3 mm, about 0.2 mm to 2.5 mm, about 0.2 mm to 2 mm, about 0.2 mm to 1.5 mm, about 0.2 mm to 1 mm, about 0.5 mm to 4 mm, about 0.5 mm to 3 mm, about 0.5 mm to 2.5 mm, about 0.5 mm to 2 mm, about 0.5 mm to 1.5 mm, about 0.5 mm to 1.0 mm, about 0.2 mm, about 0.5 mm, about 1 mm, about 1.5 mm, about 2 mm, about 2.5 mm, about 3 mm, or about 4 mm. Preferably, the thickness of the scaffold is from about about 0.2 mm to 3 mm, about 0.2 mm to 2.5 mm, about 0.2 mm to 2 mm, about 0.2 mm to 1 mm, about 0.5 mm to 2 mm, about 0.5 mm to 1.0 mm, about 0.2 mm, about 0.5 mm, about 1 mm, about 1.5 mm, about 2 mm, about 2.5 mm, or about 3 mm. Ranges and values intermediate to the above recited ranges and values are also contemplated to be part of the invention.

The surface roughness of scaffold fibers affect cellular behaviors at nano- or micro-scales since cells sense and react differently to various nano- or micro-topographies. For example, rough surfaces enhance cell adhesion, migration and growth by triggering expression of integrin receptors and product of growth factors and ECM proteins. In certain embodiments, the CA/SPH fibers in the scaffold or the scaffold itself has a surface roughness (Rₐ), calculated for example from atomic force microscopy (AFM) images of the fibers or scaffold of about 50 to 100, about 50 to 90, about 50 to 80, about 50 to 75, about 50 to 70, about 50 to 60, about 60 to 100, about 60 to 90, about 60 to 80, about 60 to 75, about 60 to 70, about 50, about 60, about 65, about 70, about 75, about 80, about 90, or about 100. Preferably, the surface roughness is about about 50 to 75, about 50 to 70, about 50 to 60, about 60 to 75, about 60 to 70, about 50, about 60, about 65, about 70, or about 75. More preferably, the surface roughness is about 60 to 75, about 60 to 70, about 60, about 65, about 70, or about 75. In one embodiment, the surface roughness is about 65 or about 70. Comparatively, CA fibers that do not include soy protein hydrolysate have a surface roughness (Rₐ) of less than 40. Ranges and values intermediate to the above recited ranges and values are also contemplated to be part of the invention.

In some embodiments, the CA/SPH fiber scaffolds of the invention exhibit excellent wettability, with an initial water contact angle (at 0 s) of no higher than 60°, e.g., about 50° to 60°, about 55° to 60°, about 50°, about 55°, or about 60°. Comparatively, CA scaffolds which do not include soy protein hydrolysate have an initial water contact angle of about 75°. Ranges and values intermediate to the above recited ranges and values are also contemplated to be part of the invention.

In some embodiments, the CA/SPH polymeric fiber scaffolds of the invention exhibit excellent water absorption capability, with a weight gain (as resulted from absorption of water) of at least 500%, e.g., higher than 700%, e.g., about 750% to 800%, about 700% to 750%, about 700%, or about 750%. In one embodiment, these weight gain percentages are obtained after immersing the scaffold in 3 ml of water or an aqueous solution for 24 hours, for example, at 37° C. Comparatively, unmodified CA fiber scaffolds show a weight gain of no higher than 600% and PCL fibers show a weight gain of about 150%. Ranges and values intermediate to the above recited ranges and values are also contemplated to be part of the invention.

2. Polymeric Fiber Scaffolds Comprising an Extracellular Matrix Protein

In some aspects, the scaffolds of the invention are composed of a plurality of polymeric fibers comprising a protein, such as an extracellular matrix protein mimicking matrix in the fetal dermal native tissue and promoting wound healing by accelerating proliferation, growth, migration, infiltration, and recruiting fibroblasts and keratinocytes. The scaffolds are moisture-retaining (or hydrating) due to the high hydrophilicity and swelling properties of polymeric fibers. Thus, the scaffolds are useful in methods of wound healing, since they provide both structural and biological cues for promoting wound healing.

Accordingly, in one aspect, the present invention provides polymeric fiber scaffolds which include a plurality of polymeric fibers, each polymeric fiber independently comprising a protein, such as, collagen type I, fibrinogen, fibronectin, chondroitin sulfate, gelatin, and hyaluronic acid, and combinations thereof.

In one embodiment, each polymeric fiber in the polymeric fiber scaffold independently comprises hyaluronic acid. In one embodiment, an aqueous solution (e.g., diH₂O) used to form the plurality of polymeric hyaluronic acid fibers comprises about 1% w/v to about 4% w/v of hyaluronic acid.

In another embodiment, each polymeric fiber in the polymeric fiber scaffold independently comprises fibronectin. In one embodiment, an aqueous solution (e.g., diH₂O) used to form the plurality of polymeric fibers comprises about 0.01% w/v to about 3.0% w/v fibronectin.

In yet another embodiment, each polymeric fiber in the polymeric fiber scaffold independently comprises fibronectin and hyaluronic acid. In one embodiment, an aqueous solution (e.g., diH₂O) used to form the plurality of polymeric fibers comprises about 0.01% w/v to about 3.0% w/v fibronectin and about 1% w/v to about 2% w/v hyaluronic acid. In one embodiment, the ratio (wt) of fibronectin:hyaluronic acid is about 1:1.

In another embodiment, each polymeric fiber in the polymeric fiber scaffold independently comprises collagen type I. In one embodiment, an aqueous solution (e.g., diH₂O) used to form the plurality of polymeric fibers comprises about 2.0% w/v to about 10% w/v collagen type I.

In yet another embodiment, each polymeric fiber independently comprises fibrinogen. In one embodiment, an aqueous solution (e.g., diH₂O) used to form the plurality of polymeric fibers comprises about 4.0% w/v to about 12.5% w/v fibrinogen.

In one embodiment, each polymeric fiber independently comprises gelatin. In one embodiment, an aqueous solution (e.g., diH₂O) used to form the plurality of polymeric fibers comprises about 4.0% w/v to about 12% w/v gelatin.

In another embodiment, each polymeric fiber independently comprises hyaluronic acid. In one embodiment, an aqueous solution (e.g., diH₂O) used to form the plurality of polymeric fibers comprises about 0.5% w/v to about 4% w/v hyaluronic acid.

In yet another embodiment, each polymeric fiber independently comprises hyaluronic acid and gelatin. In one embodiment, an aqueous solution (e.g., diH₂O) used to form the plurality of polymeric fibers comprises about 0.5% w/v to about 4% w/v hyaluronic acid and about 4% w/v to about 4% w/v to about 20% w/v gelatin. In one embodiment, the ratio (wt) of hyaluronic acid:gelatin is about 10:1 to about 1:10.

In one embodiment, each polymeric fiber independently comprises chondroitin sulfate. In one embodiment, an aqueous solution (e.g., diH₂O) used to form the plurality of polymeric fibers comprises about 20% w/v chondroitin sulfate.

In certain embodiments, each polymeric fiber in the polymeric fiber scaffold independently comprises hyaluronic acid. In one embodiment, an aqueous solution (e.g., diH₂O) used to form the plurality of polymeric hyaluronic acid fibers comprises about 1% w/v of hyaluronic acid.

In another embodiment, an aqueous solution (e.g., diH₂O) used to form the plurality of polymeric hyaluronic acid fibers comprises about 2% w/v of hyaluronic acid. In one embodiment, an aqueous solution (e.g., diH₂O) used to form the plurality of polymeric hyaluronic acid fibers comprises about 3% w/v of hyaluronic acid. In yet another embodiment, an aqueous solution (e.g., diH₂O) used to form the plurality of polymeric hyaluronic acid fibers comprises about 4% w/v of hyaluronic acid. In one embodiment, each polymeric fiber in the polymeric fiber scaffold independently comprises about 1% w/v to about 4% w/v hyaluronic acid and the plurality of polymeric fibers is covalently cross-linked, e.g., via inter-polymeric fiber crosslinking and/or intra-polymeric fiber crosslinking, e.g., via ester bond formation.

Since the polymer solution is solidified upon formation of the fibers and scaffolds in a liquid, such as ethanol (e.g., using an iRJS system described below), the formed fibers and scaffolds of the invention contain about 100% w/w of the protein in the dry state (when a single protein polymer is used to form the fibers and scaffolds). It is to be understood that the fibers and scaffolds of the invention are highly hydrophillic and, thus, when contacted with water, the polymer in the formed fibers and scaffolds may absorb water decreasing the content of polymer in the formed fibers and scaffolds. Decrease in polymer content can be calculated using the water absorption data (or swelling ratio) of HA described below (e.g. a 100% w/w HA fiber that swells 1000% (i.e. absorbs 10 times its weight) will have a polymer content of 10%).

Accordingly, in one embodiment, the formed fibers and scaffolds of the invention, comprise about 100% w/w hyaluronic acid in the dry state (based on total weight of protein scaffold).

In one embodiment, the formed fibers and scaffolds of the invention, comprise about 100% w/w fibronectin in the dry state (based on total weight of protein scaffold).

In one embodiment, the formed fibers and scaffolds of the invention, comprise about 100% w/w collagent type I in the dry state (based on total weight of protein scaffold).

In one embodiment, the formed fibers and scaffolds of the invention, comprise about 100% w/w fibrinogen in the dry state (based on total weight of protein scaffold).

In one embodiment, the formed fibers and scaffolds of the invention, comprise about 100% w/w gelatin in the dry state (based on total weight of protein scaffold).

In one embodiment, the formed fibers and scaffolds of the invention, comprise about 100% w/w chondroitin sulfate in the dry state (based on total weight of protein scaffold).

In one embodiment, the formed fibers and scaffolds of the invention, comprise about 100% w/w collagent type I in the dry state (based on total weight of protein scaffold).

In one embodiment, the formed fibers and scaffolds of the invention, comprise about 100% w/w collagent type I in the dry state (based on total weight of protein scaffold).

In one embodiment, the formed fibers and scaffolds of the invention, comprise about 0.99% w/w fibronection and about 99.01% w/w hyaluronic acid, about 75% w/w fibronection and about 25% w/w hyaluronic acid, about 0.49% w/w fibronection and about 99.51% w/w hyaluronic acid, or about 60% w/w fibronection and about 40% w/w hyaluronic acid in the dry state (based on total weight of protein scaffold).

In one embodiment, the formed fibers and scaffolds of the invention, comprise about 89% w/w gelatin and about 11% w/w hyaluronic acid, about 97.6% w/w gelatin and about 2.4% w/w hyaluronic acid, about 50% w/w gelatin and about 50% w/w hyaluronic acid, or about 83.33% w/w gelatin and about 16.66% w/w hyaluronic acid in the dry state (based on total weight of protein scaffold).

In one embodiment, substantially all of the plurality of polymeric fibers in the scaffold is covalently cross-linked to at least one of the plurality, e.g., covalently cross-linked via inter-polymeric fiber crosslinking and/or intra-polymeric fiber crosslinking, e.g., via ester bond formation.

In particular embodiments, substantially all of the plurality of polymeric fibers comprising a protein, such as hyaluronic acid, in the scaffold are covalently cross-linked to at least one of the plurality, e.g., covalently cross-linked via inter-polymeric fiber crosslinking and/or intra-polymeric fiber crosslinking, e.g., via ester bond formation, e.g., using EDC/NHS (described below).

The polymeric fiber scaffolds of the invention comprising an extracellular matrix protein promote cutaneous wound healing and/or cutaneous tissue regeneration and have physical and mechanical properties that mimic fetal dermal skin extracellular matrix, as elaborated in the following paragraphs. It should be noted that the following applies to polymeric fibers and scaffolds that are cross-linked as well as to polymeric fibers and scaffolds that are not cross-linked.

In some embodiments, each polymeric fiber in the polymeric fiber scaffold independently has a diameter of about 500 nanometers to about 10 micrometers, e.g., a diameter of about 1 micrometer to about 5 micrometers. Fiber diameters ranging from 200 nm to 400 nm, which are similar to native extracellular matrix, enhance adhesion and proliferation of human dermal fibroblasts. Accordingly, in some embodiments, each polymeric fiber in the scaffold independently has a diameter of about 200 nm to 400 nm, e.g., about 250 nm to 400 nm, about 300 nm to 400 nm, about 350 nm to 400 nm, about 360 nm to 400 nm, about 370 nm to 400 nm, about 375 nm to 400 nm, about 380 nm to 400 nm, about 385 nm to 400 nm, about 390 nm to 400 nm, about 395 nm to 400 nm, about 300 nm, about 325 nm, about 350 nm, about 360 nm, about 370 nm, about 375 nm, about 380 nm, about 385 nm, about 390 nm, about 395 nm, or about 400 nm. Preferably, the fiber diameter is about 300 nm to 400 nm, about 350 nm to 400 nm, about 375 nm to 400 nm, about 380 nm to 400 nm, about 390 nm to 400 nm, about 395 nm to 400 nm, about 300 nm, about 350 nm, about 375 nm, about 380 nm, about 385 nm, about 390 nm, about 395 nm, or about 400 nm. More preferably, the fiber diameter is about 300 nm to 400 nm, about 350 nm to 400 nm, about 375 nm to 400 nm, about 390 nm to 400 nm, about 395 nm to 400 nm, about 300 nm, about 350 nm, about 375 nm, about 390 nm, about 395 nm, or about 400 nm. Comparatively, polycaprolactine (PCL) fibers typically have fiber diameters exceeding 600 nm. Ranges and values intermediate to the above recited ranges and values are also contemplated to be part of the invention. The polymeric fiber scaffolds themselves may be of any desired size and shape and can be fabricated according to need and use. Methods for fabricating the polymeric fiber scaffold are described below.

In certain embodiments, the polymeric fiber scaffold has a porosity greater than about 40%, e.g., a porosity of about 60% to about 80%, about 65% to about 80%, about 70% to about 80%, e.g., about 60, 61, 62, 63, 64, 65, 66, 67, 68, 69, 70, 71, 72, 73, 74, 75, 76, 77, 78, 79, or about 80%. Ranges and values intermediate to the above recited ranges and values are also contemplated to be part of the invention.

The compression modulus of the polymeric fiber scaffolds may be about 400 Pascals to about 1,000 Pascals, e.g., about 400 Pascals to about 975 Pascals, about 400 Pascals to about 950 Pascals, about 400 Pascals to about 925 Pascals, about 400 Pascals to about 900 Pascals, about 400 Pascals to about 875 Pascals, about 400 Pascals to about 850 Pascals, about 400 Pascals to about 825 Pascals, about 400 Pascals to about 800 Pascals, about 400 Pascals to about 775 Pascals, about 400 Pascals to about 750 Pascals, about 400 Pascals to about 725 Pascals, about 400 Pascals to about 700 Pascals, about 400 Pascals to about 675 Pascals, about 400 Pascals to about 650 Pascals, about 400 Pascals to about 625 Pascals, about 400 Pascals to about 600 Pascals, e.g., about 425, 450, 475, 500, 525, 550, 575, or about 600 Pascals. Ranges and values intermediate to the above recited ranges and values are also contemplated to be part of the invention.

Fiber and scaffold stiffnessness also affects cell behavior. To encourage assembly of new estracellular matrix (ECM), the stiffness of wound dressing materials should mimic the stiffness of the native fetal dermal skin microenvironment of about 5 kPa to 150 kPa in Young's modulus. The Young's modulus of the polymeric fiber scaffolds may be about 10 kiloPascals to about 100 kiloPascals, e.g., about 15 kiloPascals to about 100 kiloPascals, about 20 kiloPascals to about 100 kiloPascals, about 25 kiloPascals to about 100 kiloPascals, about 30 kiloPascals to about 100 kiloPascals, about 15 kiloPascals to about 75 kiloPascals, about 20 kiloPascals to about 75 kiloPascals, about 25 kiloPascals to about 75 kiloPascals, about 30 kiloPascals to about 75 kiloPascals, about 15 kiloPascals to about 50 kiloPascals, about 20 kiloPascals to about 50 kiloPascals, about 25 kiloPascals to about 50 kiloPascals, about 30 kiloPascals to about 50 kiloPascals, about 15 kiloPascals to about 45 kiloPascals, about 20 kiloPascals to about 45 kiloPascals, about 25 kiloPascals to about 45 kiloPascals, about 30 kiloPascals to about 50 kiloPascals, about 30 kiloPascals to about 45 kiloPascals. Ranges and values intermediate to the above recited ranges and values are also contemplated to be part of the invention.

In some embodiments, the extracellular matrix protein, e.g., hyaluronic acid, polymeric fiber scaffolds of the invention exhibit excellent water absorption capability, with a weight gain (as resulted from absorption of water) of at least 500%, e.g., higher than 1000%, e.g., about 2000% to 6000%, about 3000 to about 6000%, about 3500 to about 6000%. In one embodiment, these weight gain percentages are obtained after immersing the scaffold in 3 ml of water or an aqueous solution for 24 hours, for example, at 37° C. Comparatively, uncrosslinked HA fiber scaffolds show a weight gain of no higher than 2000-3000%. Ranges and values intermediate to the above recited ranges and values are also contemplated to be part of the invention.

In some embodiment, the extracellular matrix protein, e.g., hyaluronic acid, polymeric fiber scaffolds of the invention may exhibit a water absorption capability, with a weight gain of about 4000% to about 6000% at about 10 minutes post-addition of water.

The thickness of the polymeric fiber scaffolds comprising an extracellular matrix protein. For example, if an iRJS system is used to spin the fibers and to produce the scaffolds, the thickness of the scaffold can be controlled by the amount of the polymer solution used. In another embodiment, the thickness of the scaffold can be controlled by the rotation speed. In some embodiments, the thickness of the scaffold ranges from about 0.1 mm to 5 mm, e.g., about 0.2 mm to 4 mm, about 0.2 mm to 3 mm, about 0.2 mm to 2.5 mm, about 0.2 mm to 2 mm, about 0.2 mm to 1.5 mm, about 0.2 mm to 1 mm, about 0.5 mm to 4 mm, about 0.5 mm to 3 mm, about 0.5 mm to 2.5 mm, about 0.5 mm to 2 mm, about 0.5 mm to 1.5 mm, about 0.5 mm to 1.0 mm, about 0.2 mm, about 0.5 mm, about 1 mm, about 1.5 mm, about 2 mm, about 2.5 mm, about 3 mm, or about 4 mm. Preferably, the thickness of the scaffold is from about about 0.2 mm to 3 mm, about 0.2 mm to 2.5 mm, about 0.2 mm to 2 mm, about 0.2 mm to 1 mm, about 0.5 mm to 2 mm, about 0.5 mm to 1.0 mm, about 0.2 mm, about 0.5 mm, about 1 mm, about 1.5 mm, about 2 mm, about 2.5 mm, or about 3 mm. Ranges and values intermediate to the above recited ranges and values are also contemplated to be part of the invention.

3. Polymeric Fiber Scaffolds Comprising Polycaprolactone (PCL) and Alfalfa

In one aspect, the present invention provides polymeric fiber scaffolds which include a plurality of polymeric fibers, each polymeric fiber independently comprising polycaprolactone (PCL) and alfalfa. In a particular embodiment, the PCL and alfalfa are co-spun to form the scaffold (described below). The PCL component serves as a soft and hydrophilic backbone similar to that of the collagen matrix in the dermal native tissue, while the protein (alfalfa) component promotes wound healing by accelerating proliferation, growth, migration, infiltration. In a particular embodiment, the alfalfa is homogeneously distributed along the fibers (i.e., co-spinning of alfalfa and PCL results in an even districution of alfalfa in the fibers and along the length of the fibers). Additionally, the scaffolds of the invention contain bioactive molecules, e.g., phytoestrogens that enhance skin regeneration. Furthermore, the scaffolds are moisture-retaining (or hydrating) due to the high hydrophilicity and swelling properties of PCL/alfalfa nanofibers. Thus, the PCL/alfalfa scaffolds of the invention are useful in methods of wound healing, since they provide both structural and biological cues for promoting wound healing.

In one embodiment, a solution used to form the polycaprolactobne/alfalfa (PCL/alfalfa) polymeric fibers and the scaffolds of the invention comprises about 4% to about 8% w/v of PCL (based on volume of the carrier during manufacturing of the fibers and scaffolds, i.e., w/v %), e.g., about 4% to 8%, about 4% to 7%, about 4% to 6%, about 5% to 8%, about 6% to 8% w/v % PCL. In one embodiment, the solution comprises about 6% w/v % of PCL.

In one embodiment, a solution used to form the polycaprolactobne/alfalfa (PCL/alfalfa) fibers and the scaffolds of the invention comprises about 0.5% (w/v %) and 2% (w/v %) (based on volume of the carrier during manufacturing of the fibers and scaffolds, i.e., w/v %), e.g., about 0.5% to 2%, about 0.6% to 2%, about 0.7% to 2%, about 0.8% to 2%, about 0.9% to 2%, about 1% to 2%, about 1.1% to 2%, about 1.2% to 2%, about 1.3% to 2%, about 1.4% to 2%, about 1.5% to 2%, about 1.6% to 2%, about 1.7% to 2%, about 1.8% to 2%, about 1.9% to 2%, about 0.5% to 1.5%, about 0.6% to 1.5%, about 0.7% to 1.5%, about 0.8% to 1.5%, about 0.9% to 1.5%, about 1% to 1.5%, about 1.1% to 1.5%, about 1.2% to 1.5%, about 1.3% to 1.5%, about 1.4% to 1.5% w/v %. Preferably, the solution comprises about 1% w/v alfalfa.

In some embodiments, the carrier used during fabrication of the PCL/alfalfa fibers and scaffolds of the invention is an organic solvent. Preferably, the organic solvent is a polar, protic solvent. Preferably, the organic solvent is an alcohol including a pure alcohol or a solvent system with an alcohol as the primary solvent, and non-limiting examples of a suitable alcohol are n-butanol, tert-butanol, methanol, ethanol, n-propanol and isopropanol. In one embodiment, the alcohol used as a carrier in the manufacturing of the PCL/alfalfa fibers and scaffolds is a halogenated alcohol, such a halogenated C1-C4 alcohol. In one embodiment, the carrier used in the manufacturing of the PCL/alfalfa fibers and scaffolds is hexafluoroisopropanol (HFIP).

Since the carrier solvent dissipates completely upon formation (e.g., solidification) of the fibers and scaffolds, the formed fibers and scaffolds of the invention, accordingly, contain PCL and alfalfa at a PCL:alfalfa weight ratio of about 3-12:1, e.g., about 3:1, about 4:1, about 5:1, about 6:1, about 7:1, about 8:1, about 9:1, about 10:1, about 11:1, or about 12:1. In one embodiment, the PCl:alfalfa weight ratio is about 6:1.

Methods for forming polymeric fibers and scaffold comprising PCL and alfalfa are described below.

Alternatively or additionally, when expressed as weight/weight percentages, the formed fibers and scaffolds of the invention contain about 60-95% w/w PCL (based on total weight of PCL/alfalfa fiber or PCL/alfalfa scaffold), e.g., about 61-95%, about 62-95%, about 63-95%, about 64-95%, about 65-95%, about 66-95%, about 61-90%, about 62-90%, about 63-90%, about 64-90%, about 65-90%, about 66-90%, about 61-85%, about 62-85%, about 63-85%, about 64-85%, about 65-85%, about 66-85%, about 61-80%, about 62-80%, about 63-80%, about 64-80%, about 65-80%, or about 66-80% w/w %. In one embodiment, the formed fibers and scaffolds of the invention contain about 85.71% w/w PCL. As alfalfa, the formed fibers and scaffolds of the invention contain about 5-35% w/w alfalfa (based on total weight of PCL/alfalfa fiber or PCL/alfalfa scaffold), e.g., about 5-35%, about 5-34%, about 5-33%, about 5-32%, about 5-31%, about 5-30%, 5-29%, 5-28%, 5-27%, 5-26%, 5-25%, about 5-24%, about 5-23%, about 5-22%, about 5-21%, about 5-20%, 5-19%, 5-18%, 5-17%, 5-16%, 5-15%, 10-35%, about 10-34%, about 10-33%, about 10-32%, about 10-31%, about 10-30%, 10-29%, 10-28%, 10-27%, 10-26%, 10-25%, about 10-24%, about 10-23%, about 10-22%, about 10-21%, about 10-20%, 10-19%, 10-18%, 10-17%, 10-16%, or about 10-15% w/w % alfalfa. In one embodiment, the formed fibers and scaffolds of the invention contain about 14.29% w/w alfalfa.

The scaffolds of the invention promote cutaneous wound healing and/or cutaneous tissue regeneration and have physical and mechanical properties that mimic dermal skin extracellular matrix, as elaborated in the following paragraphs.

In some embodiments, each PCL/alfalfa fiber in the scaffold independently has a diameter of about 200 nm to 500 nm, e.g., about 200 nm to 500 nm, about 250 nm to about 500 nm, about 300 nm to 500 nm, about 350 nm to 500 nm, about 360 nm to 500 nm, about 370 nm to 500 nm, about 375 nm to 500 nm, about 380 nm to 500 nm, about 385 nm to 500 nm, about 390 nm to 500 nm, about 395 nm to 500 nm, about 200 nm to 450 nm, about 250 nm to about 450 nm, about 300 nm to 450 nm, about 350 nm to 450 nm, about 360 nm to 450 nm, about 370 nm to 450 nm, about 375 nm to 450 nm, about 380 nm to 450 nm, about 385 nm to 450 nm, about 390 nm to 450 nm, about 395 nm to 450 nm, e.g., about about 300 nm, about 325 nm, about 350 nm, about 360 nm, about 370 nm, about 375 nm, about 380 nm, about 385 nm, about 390 nm, about 395 nm, about 400 nm, about 410 nm, about 15 nm, about 420 nm, about 425 nm, about 430 nm, about 435 nm, about 440 nm, about 445 nm, or about 450 nm. Ranges and values intermediate to the above recited ranges and values are also contemplated to be part of the invention.

Fiber diameters ranging from 200 nm to 500 nm, which are similar to native extracellular matrix, enhance adhesion and proliferation of human dermal fibroblasts. Comparatively, polycaprolactine (PCL) fibers typically have fiber diameters exceeding 600 nm. Ranges and values intermediate to the above recited ranges and values are also contemplated to be part of the invention. The scaffolds themselves may be of any desired size and shape and can be fabricated according to need and use. Methods for fabricating the polymeric fiber scaffold are described below.

In certain embodiments, the scaffold formed has a porosity greater than about 40%, e.g., a porosity of about 50% to about 80%, about 55% to about 80%, about 60% to about 80%, about 65% to about 80%, about 70% to about 80%, about 75% to about 80%, e.g., about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or about 80%. Ranges and values intermediate to the above recited ranges and values are also contemplated to be part of the invention.

Fiber and scaffold stiffnessness also affects cell behavior. To encourage assembly of new estracellular matrix (ECM), the stiffness of wound dressing materials should mimic the stiffness of the native ECM microenvironment of about 5 kPa to 600 kPa in Young's modulus. In some embodiments, the Young's modulus of the scaffold, which indicates the stiffness of the scaffold, is about 5 kPa to 100 kPa, about 5 kPa to 95 kPa, about 5 kPa to 90 kPa, about 5 kPa to 85 kPa, about 5 kPa to 80 kPa, about 5 kPa to 75 kPa, about 5 kPa to 70 kPa, about 5 kPa to 65 kPa, about 5 kPa to 60 kPa, about 5 kPa to 55 kPa, about 5 kPa to 50 kPa, about 5 kPa to 45 kPa, e.g., about 5 kPa to 10 kPa, about 15 kPa to 20 kPa, about 25 kPa, about 30 kPa, about 35 kPa, or about 40 kPa. In some embodiments, the specific stiffness (which accounts for any effect of scaffold density on stiffness) of the fiber and scaffolds is about 10 kPa to about 55 kPa, e.g., about 0 kPa, about 15 kPa to 20 kPa, about 25 kPa, about 30 kPa, about 35 kPa, about 40 kPa, about 45 kPa, about 50 kPa, or about 55 kPa. Ranges and values intermediate to the above recited ranges and values are also contemplated to be part of the invention. Comparatively, the stiffness of common synthetic polymer nanofiber scaffolds used as wound dressings, such as polycaprolactone (PCL) scaffolds, is usually one to several orders of magnitude higher, i.e., in the MPa range.

As described herein and known in the art, phytoestrogen is a chemical in plants that is structurally and functionally similar to estrogen. Once delivered to a target organ, phytoestrogens bind to estrogen receptors (ERs; ER α or ER β) in cells with higher affinity to ER β than ER α. By triggering the ER β signaling pathways, phytoestrogens benefit human health (such as wound healing). One of the major phytoestrogens that are advantageous to human health is genistein, which is known to be present in alfalfa. As described below, the formed fibers and scaffolds comprising PCL/alfalfa were shown to contain biologically active genistein, e.g., about 0.25% w/w genistein.

The thickness of the PCL/alfalfa fibrous scaffolds of the invention can be controlled. For example, if a rotary jet spinning (RJS) system is used to spin the fibers and to produce the scaffolds, the thickness of the scaffold can be controlled by the amount of the carrier or the polymer solution used. In another embodiment, the thickness of the scaffold can be controlled by the rotation speed. In some embodiments, the thickness of the scaffold ranges from about 0.1 mm to 5 mm, e.g., about 0.2 mm to 4 mm, about 0.2 mm to 3 mm, about 0.2 mm to 2.5 mm, about 0.2 mm to 2 mm, about 0.2 mm to 1.5 mm, about 0.2 mm to 1 mm, about 0.5 mm to 4 mm, about 0.5 mm to 3 mm, about 0.5 mm to 2.5 mm, about 0.5 mm to 2 mm, about 0.5 mm to 1.5 mm, about 0.5 mm to 1.0 mm, about 0.2 mm, about 0.5 mm, about 1 mm, about 1.5 mm, about 2 mm, about 2.5 mm, about 3 mm, or about 4 mm. Preferably, the thickness of the scaffold is from about about 0.2 mm to 3 mm, about 0.2 mm to 2.5 mm, about 0.2 mm to 2 mm, about 0.2 mm to 1 mm, about 0.5 mm to 2 mm, about 0.5 mm to 1.0 mm, about 0.2 mm, about 0.5 mm, about 1 mm, about 1.5 mm, about 2 mm, about 2.5 mm, or about 3 mm. Ranges and values intermediate to the above recited ranges and values are also contemplated to be part of the invention.

In some embodiments, the PCL/alfalfa fiber scaffolds of the invention exhibit excellent wettability, with a water contact angle (at 25 s) of no higher than 60°, e.g., about 20° to 60°, about 20° to 55°, about 20° to 50°, about 20° to 45°, about 20° to 40°, about 20° to 35°, about 20° to 30°, e.g., about 60°, about 55°, or about 50°, about 45°, about 40°, about 35°, about 30°, or about 25°. Comparatively, PCL scaffolds which do not include alfalfa have an initial water contact angle of about 85°. Ranges and values intermediate to the above recited ranges and values are also contemplated to be part of the invention.

4. Polymeric Fiber Scaffolds Comprising Hyaluronic Acid and Soy Protein Isolate

In one aspect, the present invention provides polymeric fiber scaffolds which include a plurality of polymeric fibers, each polymeric fiber independently comprising hyaluronic acid (HA) and soy protein isolate (SPI). In a particular embodiment, the HA and SPI are co-spun to form the scaffold (described below). The HA component serves as a soft and hydrophilic backbone similar to that of the collagen matrix in the dermal native tissue, while the protein (SPI) component promotes wound healing by accelerating proliferation, growth, migration, infiltration. In a particular embodiment, the alfalfa is homogeneously distributed along the fibers (i.e., co-spinning of SPI and HA results in an even districution of SPI in the fibers and along the length of the fibers). Additionally, the scaffolds of the invention contain bioactive molecules, e.g., phytoestrogens that enhance skin regeneration. Furthermore, the scaffolds are moisture-retaining (or hydrating) due to the high hydrophilicity and swelling properties of HA/SPI nanofibers. Thus, the HA/SPI scaffolds of the invention are useful in methods of wound healing, since they provide both structural and biological cues for promoting wound healing.

Accordingly, in one aspect, the present invention provides polymeric fiber scaffolds which include a plurality of polymeric fibers, each polymeric fiber independently comprising hyaluronic acid (HA), soy protein isolate (SPI).

In one embodiment, a solution used to form the HA/SPI polymeric fibers and the scaffolds of the invention comprises about 1% to about 3% w/v of HA (based on volume of the carrier during manufacturing of the fibers and scaffolds, i.e., w/v %), e.g., about 1%, about 1.25, about 1.5, about 1.75, about 2, about 2.25, about 2.5, and 2.75, or about 3% w/v % of HA. In one embodiment, the solution comprises about 2% w/v % of HA.

In one embodiment, a solution used to form the HA/SPI fibers and the scaffolds of the invention comprises about about 1% to about 3% w/v of SPI (based on volume of the carrier during manufacturing of the fibers and scaffolds, i.e., w/v %), e.g., about 1%, about 1.25, about 1.5, about 1.75, about 2, about 2.25, about 2.5, and 2.75, or about 3% w/v % of SPI. In one embodiment, the solution comprises about 2% w/v % of SPI.

In one embodiment, each polymeric fiber in the polymeric fiber scaffold independently comprises HA and SPI. In one embodiment, an aqueous solution (e.g., diH₂O) used to form the plurality of polymeric fibers comprises about 2% w/v HA and about 2% w/v SPI. In one embodiment, the ratio (wt) of HA to SPI is about 1:1.

In one embodiment, each polymeric fiber in the polymeric fiber scaffold independently comprises about 2% w/v HA and 2% SPI and the plurality of polymeric fibers is covalently cross-linked, e.g., via inter-polymeric fiber crosslinking and/or intra-polymeric fiber crosslinking, e.g., via ester bond formation.

Since the polymer solution is solidified upon formation of the fibers and scaffolds in a liquid, such as ethanol (e.g., using an iRJS system described below), the formed fibers and scaffolds of the invention contain about 100% w/w of the protein in the dry state (when a single protein polymer is used to form the fibers and scaffolds). It is to be understood that the fibers and scaffolds of the invention are highly hydrophillic and, thus, when contacted with water, the polymer in the formed fibers and scaffolds may dissolve decreasing the content of polymer in the formed fibers and scaffolds.

Accordingly, in one embodiment, the formed fibers and scaffolds of the invention, comprise about 50% w/w HA and about 50% SPI in the dry state (based on total weight of protein scaffold).

In one embodiment, substantially all of the plurality of polymeric fibers in the scaffold is covalently cross-linked to at least one of the plurality, e.g., covalently cross-linked via inter-polymeric fiber crosslinking and/or intra-polymeric fiber crosslinking, e.g., via ester bond formation.

In particular embodiments, substantially all of the plurality of polymeric fibers comprising a protein, such as hyaluronic acid, in the scaffold are covalently cross-linked to at least one of the plurality, e.g., covalently cross-linked via inter-polymeric fiber crosslinking and/or intra-polymeric fiber crosslinking, e.g., via ester bond formation, e.g., using EDC/NHS (described below).

The polymeric fiber scaffolds of the invention comprising HA and SPI promote cutaneous wound healing and/or cutaneous tissue regeneration and have physical and mechanical properties that mimic fetal dermal skin extracellular matrix, as elaborated in the following paragraphs. It should be noted that the following applies to polymeric fibers and scaffolds that are cross-linked as well as to polymeric fibers and scaffolds that are not cross-linked.

In some embodiments, each polymeric fiber in the polymeric fiber scaffold independently has a diameter of about 1 μm nanometers to about 3 μm, e.g., a diameter of about 1 μm to about 2 μm, e.g., 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 μm. Ranges and values intermediate to the above recited ranges and values are also contemplated to be part of the invention. The polymeric fiber scaffolds themselves may be of any desired size and shape and can be fabricated according to need and use. Methods for fabricating the polymeric fiber scaffold are described below.

In certain embodiments, the polymeric fiber scaffold has a porosity greater than about 40%, e.g., a porosity of about 60% to about 80%, about 65% to about 80%, about 70% to about 80%, e.g., about 60, 61, 62, 63, 64, 65, 66, 67, 68, 69, 70, 71, 72, 73, 74, 75, 76, 77, 78, 79, or about 80%. Ranges and values intermediate to the above recited ranges and values are also contemplated to be part of the invention.

The Young's modulus of the polymeric fiber scaffolds may be about 1 kiloPascals to about 10 kiloPascals, e.g., about 1, 1.25, 1.5, 2, 2.5, 3, 3.5, 4, 4.5, 5, 5.5, 6, 6.5, 7, 7.5, 8, 9.5, or about 10 kiloPascals. Ranges and values intermediate to the above recited ranges and values are also contemplated to be part of the invention.

The thickness of the polymeric fiber scaffolds comprising an extracellular matrix protein. For example, if an iRJS system is used to spin the fibers and to produce the scaffolds, the thickness of the scaffold can be controlled by the amount of the polymer solution used. In another embodiment, the thickness of the scaffold can be controlled by the rotation speed. In some embodiments, the thickness of the scaffold ranges from about 0.1 mm to 5 mm, e.g., about 0.2 mm to 4 mm, about 0.2 mm to 3 mm, about 0.2 mm to 2.5 mm, about 0.2 mm to 2 mm, about 0.2 mm to 1.5 mm, about 0.2 mm to 1 mm, about 0.5 mm to 4 mm, about 0.5 mm to 3 mm, about 0.5 mm to 2.5 mm, about 0.5 mm to 2 mm, about 0.5 mm to 1.5 mm, about 0.5 mm to 1.0 mm, about 0.2 mm, about 0.5 mm, about 1 mm, about 1.5 mm, about 2 mm, about 2.5 mm, about 3 mm, or about 4 mm. Preferably, the thickness of the scaffold is from about about 0.2 mm to 3 mm, about 0.2 mm to 2.5 mm, about 0.2 mm to 2 mm, about 0.2 mm to 1 mm, about 0.5 mm to 2 mm, about 0.5 mm to 1.0 mm, about 0.2 mm, about 0.5 mm, about 1 mm, about 1.5 mm, about 2 mm, about 2.5 mm, or about 3 mm. Ranges and values intermediate to the above recited ranges and values are also contemplated to be part of the invention.

### B. Devices and Methods for the Fabrication of the Polymeric Fiber Scaffolds of the Invention

Suitable devices and methods of use of such devices for fabricating the polymeric fiber (micron, submicron or nanometer dimension polymeric fiber) scaffolds of the present invention are described in U.S. Pat. Nos. 9,410,267 and 9,738,046, and U.S. Patent Publication Nos. 2013/0312638 and 2015/0354094, the entire contents of each of which are incorporated herein by reference. Exemplary fiber formation devices do not employ a nozzle for ejecting the liquid material, a spinneret or rotating reservoir containing and ejecting the liquid material, or an electrostatic voltage potential for forming the fibers. The exemplary devices described herein are simplified as they do not employ a spinneret or an electrostatic voltage potential. In addition, the lack of a nozzle for ejecting the liquid material in exemplary devices avoids the issue of clogging of the nozzle.

For example, as described in U.S. Pat. No. 9,410,267 and U.S. Patent Publication No. 2013/0312638, in some embodiments, suitable devices for fabricating the polymeric fiber scaffolds of the invention which may, in some embodiments, be configured in a desired shape, may include a reservoir for holding a polymer, the reservoir including one or more orifices for ejecting the polymer during fiber formation, and a collection device, e.g., a mandrel, for accepting the formed polymeric fiber, wherein at least one of the reservoir and the collection device employs rotational motion during fiber formation, and the device is free of an electrical field, e.g., a high voltage electrical field. Such devices may be referred to as rotary jet spinning (RJS) devices.

The device may include a rotary motion generator for imparting a rotational motion to the reservoir and, in some exemplary embodiments, to the collection device. In some embodiments, a flexible air foil is attached to a shaft of the motor above the reservoir to facilitate fiber collection and solvent evaporation.

Rotational speeds of the reservoir in exemplary embodiments may range from about 1,000 rpm-60,000 rpm, about 1,000 rpm-50,000 rpm, about 1,000 rpm to about 40,000 rpm, about 1,000 rpm-30,000 rpm, about 1,000 rpm to about 20,000 rpm, about 1,000 rpm-10,000 rpm, about 5,000 rpm-60,000 rpm, about 5,000 rpm-50,000 rpm, about 5,000 rpm to about 40,000 rpm, about 5,000 rpm-30,000 rpm, about 5,000 rpm-20,000 rpm, about 5,000 rpm to about 15,000 rpm, about 5,000 rpm-10,000 rpm, about 10,000 rpm-60,000 rpm, about 10,000 rpm-50,000 rpm, about 10,000 rpm to about 40,000 rpm, about 10,000 rpm-30,000 rpm, about 10,000 rpm-20,000 rpm, about 10,000 rpm to about 15,000 rpm, about 20,000 rpm-60,000 rpm, about 20,000 rpm-50,000 rpm, about 20,000 rpm to about 40,000 rpm, about 20,000 rpm-30,000 rpm, or about 50,000 rpm to about 400,000 rpm, e.g., about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500,10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000 rpm, about 200,000 rpm, 250,000 rpm, 300,000 rpm, 350,000 rpm, or 400,000 rpm. Ranges and values intermediate to the above recited ranges and values are also contemplated to be part of the invention.

In certain embodiments, rotational speeds of the reservoir of about 50,000 rpm-400,000 rpm are intended to be encompassed by the invention. In one embodiment, devices employing rotational motion may be rotated at a speed greater than about 50,000 rpm, greater than about 55,000 rpm, greater than about 60,000 rpm, greater than about 65,000 rpm, greater than about 70,000 rpm, greater than about 75,000 rpm, greater than about 80,000 rpm, greater than about 85,000 rpm, greater than about 90,000 rpm, greater than about 95,000 rpm, greater than about 100,000 rpm, greater than about 105,000 rpm, greater than about 110,000 rpm, greater than about 115,000 rpm, greater than about 120,000 rpm, greater than about 125,000 rpm, greater than about 130,000 rpm, greater than about 135,000 rpm, greater than about 140,000 rpm, greater than about 145,000 rpm, greater than about 150,000 rpm, greater than about 160,000 rpm, greater than about 165,000 rpm, greater than about 170,000 rpm, greater than about 175,000 rpm, greater than about 180,000 rpm, greater than about 185,000 rpm, greater than about 190,000 rpm, greater than about 195,000 rpm, greater than about 200,000 rpm, greater than about 250,000 rpm, greater than about 300,000 rpm, greater than about 350,000 rpm, or greater than about 400,000 rpm. Ranges and values intermediate to the above recited ranges and values are also contemplated to be part of the invention.

Rotational speeds of the collection device in exemplary embodiments may range from about 1,000 to about 10,000 rpm. Ranges and values intermediate to the above recited range and values are also contemplated to be part of the invention.

Exemplary devices employing rotational motion may be rotated for a time sufficient to form a desired polymeric fiber, such as, for example, about 1 minute to about 100 minutes, about 1 minute to about 60 minutes, about 10 minutes to about 60 minutes, about 30 minutes to about 60 minutes, about 1 minute to about 30 minutes, about 20 minutes to about 50 minutes, about 5 minutes to about 20 minutes, about 5 minutes to about 30 minutes, or about 15 minutes to about 30 minutes, about 5-100 minutes, about 10-100 minutes, about 20-100 minutes, about 30-100 minutes, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 minutes, or more. Times and ranges intermediate to the above-recited values are also intended to be part of this invention.

In some embodiments, the reservoir may not be rotated, but may be pressurized to eject the polymer material from the reservoir through one or more orifices. For example, a mechanical pressurizer may be applied to one or more surfaces of the reservoir to decrease the volume of the reservoir, and thereby eject the material from the reservoir. In another exemplary embodiment, a fluid pressure may be introduced into the reservoir to pressurize the internal volume of the reservoir, and thereby eject the material from the reservoir.

An exemplary reservoir may have a volume ranging from about one nanoliter to about 1 milliliter, about one nanoliter to about 5 milliliters, about 1 nanoliter to about 100 milliliters, or about one microliter to about 100 milliliters, for holding the liquid material. Some exemplary volumes include, but are not limited to, about one nanoliter to about 1 milliliter, about one nanoliter to about 5 milliliters, about 1 nanoliter to about 100 milliliters, one microliter to about 100 microliters, about 1 milliliter to about 20 milliliters, about 20 milliliters to about 40 milliliters, about 40 milliliters to about 60 milliliters, about 60 milliliters to about 80 milliliters, about 80 milliliters to about 100 milliliters, but are not limited to these exemplary ranges. Exemplary volumes intermediate to the recited volumes are also part of the invention. In certain embodiment, the volume of the reservoir is less than about 5, less than about 4, less than about 3, less than about 2, or less than about 1 milliliter. In other embodiments, the physical size of a polymer and the desired number of polymers that will form a fiber dictate the smallest volume of the reservoir.

The reservoir includes one or more orifices through which one or more jets of the fiber-forming liquid (e.g., polymer solution) are forced to exit the reservoir by the motion of the reservoir during fiber formation. One or more exemplary orifices may be provided on any suitable side or surface of the reservoir including, but not limited to, a bottom surface of the reservoir that faces the collection device, a side surface of the reservoir, a top surface of the reservoir that faces in the opposite direction to the collection device, etc. Exemplary orifices may have any suitable cross-sectional geometry including, but not limited to, circular, oval, square, rectangular, etc. In an exemplary embodiment, one or more nozzles may be provided associated with an exemplary orifice to provide control over one or more characteristics of the fiber-forming liquid exiting the reservoir through the orifice including, but not limited to, the flow rate, speed, direction, mass, shape and/or pressure of the fiber-forming liquid. The locations, cross-sectional geometries and arrangements of the orifices on the reservoir, and/or the locations, cross-sectional geometries and arrangements of the nozzles on the orifices, may be configured based on the desired characteristics of the resulting fibers and/or based on one or more other factors including, but not limited to, viscosity of the fiber-forming liquid, the rate of solvent evaporation during fiber formation, etc.

Exemplary orifice lengths that may be used in some exemplary embodiments range between about 0.001 m and about 0.05 m, e.g., 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, or 0.05. In some embodiments, exemplary orifice lengths that may be used range between about 0.002 m and 0.01 m. Ranges and values intermediate to the above recited ranges and values are also contemplated to be part of the invention.

Exemplary orifice diameters that may be used in some exemplary embodiments range between about 0.1 μm and about 10 μm, about 50 μm to about 500 μm, about 200 μm to about 600 μm, about 200 μm to about 1,000 μm, about 500 μm to about 1,000 μm, about 200 μm to about 1,500 μm, about 200 μm to about 2,000 μm, about 500 μm to about 1,500 μm, or about 500 μm to about 2,000 μm, e.g., about 10, 20, 30, 40, 50, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,550, 1,600, 1,650, 1,700, 1,750, 1,800, 1,850, 1,900, 1,950, or about 2,000 μm. Ranges and values intermediate to the above recited ranges and values are also contemplated to be part of the invention.

In other embodiments, a suitable device for the formation of a polymeric fibers includes a reservoir for holding a polymer, the reservoir including one or more orifices for ejecting the polymer during fiber formation, a collection device, e.g., a mandrel, and an air vessel for circulating a vortex of air around the formed fibers to wind the fibers into one or more threads.

In yet other embodiments, a suitable device for the formation of a micron, submicron or nanometer dimension polymeric fiber includes a reservoir for holding a polymer, the reservoir including one or more orifices for ejecting the polymer during fiber formation, thereby forming a polymeric fiber, a collection device, e.g., a mandrel, one or more mechanical members disposed or formed on or in the vicinity of the reservoir for increasing an air flow or an air turbulence experienced by the polymer ejected from the reservoir, and a collection device for accepting the formed micron, submicron or nanometer dimension polymeric fiber.

In one embodiment, a suitable device further comprises a component suitable for continuously feeding the polymer into the rotating reservoir (or a platform), such as a spout or syringe pump.

An exemplary method to fabricate the scaffolds of the invention comprising a plurality of polymeric fibers (which may be configured in a desired shape) may include imparting rotational motion to a reservoir holding a polymer, the rotational motion causing the polymer to be ejected from one or more orifices in the reservoir and collecting a plurality of formed polymeric fibers, e.g., on a collection surface, e.g., a surface of a mandrel, thereby forming a scaffold comprising a plurality of polymeric fibers.

In one embodiment, a polymer is fed into a reservoir as a fiber-forming liquid. In this embodiment, the methods may further comprise dissolving the polymer in a solvent prior to feeding the solution into the reservoir.

In one embodiment, the methods include feeding a polymer into a rotating reservoir of a device of the invention and providing motion at a speed and for a time sufficient to form a plurality of polymeric fibers, and collecting the formed fibers, e.g., on a collection surface, e.g., a surface of a collection device, such as a mandrel having a desired shape, to form a scaffold comprising a plurality of polymeric fibers, e.g., a scaffold comprising a plurality of polymeric fibers having the desired shape.

In another embodiment, the methods include feeding a polymer solution into a rotating reservoir of a device of the invention and providing an amount of shear stress to the rotating polymer solution for a time sufficient to form a plurality of polymeric fibers, and collecting the formed fibers e.g., on a collection surface, e.g., a surface of a collection device, such as a mandrel having a desired shape, to form a scaffold comprising a plurality of polymeric fibers, e.g., a scaffold comprising a plurality of polymeric fibers having the desired shape.

In another embodiment, suitable devices for fabricating the polymeric fiber scaffolds of the invention which may, in some embodiments, be configured in a desired shape, include those described in U.S. Patent Publication No. 2015/0354094, the entire contents of which are incorporated herein by reference. Such devices, which may be referred to as immersed rotary jet spinning (iRJS) devices, are suitable for preparing polymeric fiber scaffolds from polymers that, e.g., require on-contact cross-linking, that cannot be readily dissolved at a high enough concentrations to provide sufficient viscosity for random entanglement and solvent evaporation to form polymeric fibers, and that require precipitation,

Suitable iRJS devices include, a reservoir for holding a polymer and including a surface having one or more orifices for ejecting the polymer for fiber formation; a motion generator configured to impart rotational motion to the reservoir, the rotational motion of the reservoir causing ejection of the polymer through the one or more orifices; and a collection device holding a liquid, the collection device configured and positioned to accept the polymer ejected from the reservoir; wherein the reservoir and the collection device are positioned such that the one or more orifices of the reservoir are submerged in the liquid in the collection device during rotation of the reservoir to eject the polymer; and wherein the ejection of the polymer into the liquid in the collection device causes formation of one or more polymeric fibers. In some embodiment, the device may include a second motion generator couplable to the collection device, the second motion generator configured to impart rotational motion to the liquid in the collection device.

Suitable rotational speeds of the rotating reservoir and the collection device, suitable rotational times, suitable reservoir volumes, suitable orifice diameters, and suitable orifice lengths in the iRJS devices are the same as those of the RJS device described supra.

Use of such devices for preparation of scaffolds comprising a plurality of polymeric fibers of the invention include using the motion generator to rotate the reservoir about an axis of rotation to cause ejection of the polymer in one or more jets; and collecting the one or more jets of the polymer in the liquid held in the collection device to cause formation of the plurality of polymeric fibers, thereby forming the scaffold.

In another embodiment, a suitable device for formation of the polymeric fiber scaffolds of the invention includes a reservoir for holding a polymer and including an outer surface having one or more orifices for ejecting the polymer for fiber formation; a first motion generator couplable to the reservoir, the first motion generator configured to impart rotational motion to the reservoir to cause ejection of the polymer through the one or more orifices; and a collection device holding a liquid, the collection device configured and positioned to accept the polymer ejected from the reservoir; a second motion generator couplable to the collection device, the second motion generator configured to impart rotational motion to the liquid in the collection device to generate a liquid vortex including an air gap; wherein the reservoir and the collection device are positioned such that the one or more orifices of the reservoir are positioned in the air gap of the liquid vortex in the collection device; and wherein the ejection of the polymer into the air gap and subsequently into the liquid of the liquid vortex in the collection device causes formation of one or more micron, submicron or nanometer dimension polymeric fibers.

Use of such devices for preparation of scaffolds comprising a plurality of polymeric fibers include using the first motion generator to rotate the reservoir about an axis of rotation to cause ejection of the polymer in one or more jets; using the second motion generator to rotate the liquid in the collection device to generate the liquid vortex; and collecting the one or more jets of the polymer in the air gap of the liquid vortex and subsequently in the liquid of the liquid vortex of the collection device to cause formation of the plurality of polymeric fibers, thereby forming the scaffold

In another embodiment, suitable devices for fabricating the polymeric fiber scaffolds of the invention which may, in some embodiments, be configured in a desired shape, include those described in U.S. Pat. No. 9,738,046, the entire contents of which are incorporated herein by reference. Such devices may be referred to as pull-spinning devices which include a platform for supporting a deposit of a liquid polymer material. In an exemplary embodiment, the platform is stationary. In another exemplary embodiment, the platform is movable and/or moving. In an exemplary embodiment, the deposit may be a one-time deposit. In another exemplary embodiment, the deposit may be a continual or intermittently replenished deposit. The exemplary fiber formation device may include a component suitable for continuously feeding the liquid material onto the platform, such as a spout or syringe pump. The devices also include a rotating structure disposed vertically above the platform and spaced from the platform along a vertical axis, the rotating structure comprising: a central core rotatable about a rotational axis, and one or more blades affixed to the rotating core; wherein the rotating structure is configured and operable so that, upon rotation, the one or more blades contact a surface of the polymer to impart sufficient force in order to: decouple a portion of the polymer from contact with the one or more blades of the rotating structure, and fling the portion of the polymer away from the contact with the one or more blades and from the deposit of the polymer, thereby forming a polymeric fiber.

In another embodiment, suitable devices for fabricating the polymeric fiber scaffolds of the invention which may, in some embodiments, be configured in a desired shape, include a platform for supporting a stationary deposit of a polymer; and a jet nozzle disposed in the vicinity of the platform and spaced from the platform, the jet nozzle configured to generate a gas jet directed at the polymer so that the gas jet contacts a surface of the polymer to impart sufficient force in order to fling a portion of the polymer away from the contact with the gas jet and from the deposit of the polymer, thereby forming a polymeric fiber.

Use of such devices for preparation of scaffolds comprising a plurality of polymeric fibers include providing a stationary deposit of a liquid material comprising a polymer solution or a polymer melt; and making a contact with a surface of the liquid material in the stationary deposit to impart sufficient momentary force thereto in order to: decouple a portion of the liquid material from the deposit, and fling the portion of the liquid material away from the contact and from the deposit of the liquid material, wherein the force is applied substantially parallel to the surface of the liquid material by a rotating structure that penetrates the stationary deposit of the liquid material during its rotation, thereby forming a scaffold comprising a plurality of polymeric fibers.

### C. Uses of the Scaffolds of the Invention

The scaffolds of the invention may be used in a broad range of applications, including, but not limited to, use in wound healing, drug delivery and drug discovery. The scaffolds of the invention, which may be incorporated into wound dressings, are good candidates for wound healing due to their structural and mechanical properties mimicking extracellular matrix of dermal skin, such as high porosity, e.g., for breathability and to allow cell infiltration, water absorption capabilities, and degradation characteristics, and because the structures can be easily formed into different sizes and shapes. In addition, because of the ability of the scaffolds described herein to remain moist and intact, the scaffolds of the invention are useful for, e.g., exudate removal.

Accordingly, in one aspect, the present invention provides methods of treating a subject having a wound. The methods include providing a polymeric fiber scaffold of the invention and disposing the scaffold on, over, or in the wound, thereby treating the subject. Such use of the polymeric fiber scaffolds may be combined with other methods of treatment, debridement, repair, and contouring.

The scaffolds and wound dressings of the invention may promote healing of the wound and/or accelerate closure of the wound by, for example, providing a substrate that does not have to be synthesized by fibroblasts and other cells, thereby decreasing healing time and reducing the metabolic energy requirement to synthesize new tissue at the site of the wound. In addition, since the scaffolds and wound dressings of the invention mimic extracellular matrix, tissue regeneration, in the absence of fibrosis is promoted.

Wounds that may be treated in the methods of the invention include cutaneous wounds. Cutaneous wounds include dermal tissue wounds, epidermal tissue wounds, and both dermal and epidermal tissue wounds. Wounds may be chronic non-healing wounds, e.g., pressure ulcers or bed sores, diabetic wounds, e.g., foot ulcers, burns, hypertrophic scars, infected wounds, incisional wounds, and excisional wounds, e.g., superficial excisional wounds, partial-thickness excisional wounds, and full-thickness excisional wounds.

In further embodiments, the scaffolds of the present invention can be used to study functional differentiation of stem cells (e.g., pluripotent stem cells, multipotent stem cells, induced pluripotent stem cells, and progenitor cells of embryonic, fetal, neonatal, juvenile and adult origin) into cutaneous phenotypes. Indeed, the scaffolds of the invention are able to mature skin cells, e.g., fibroblasts and keratinocytes, cells that play a crucial role in skin function.

This invention is further illustrated by the following examples, which should not be construed as limiting. The entire contents of all references, patents and published patent applications cited throughout this application, as well as the Figures, are hereby incorporated herein by reference.

### EXAMPLES

### Example 1: Soy Protein/Cellulose Polymeric Fiber Scaffold Mimicking Skin Extracellular Matrix for Enhanced Would Healing

Polymeric fiber scaffolds, such as nanofibrous scaffolds, have emerged as a promising approach to develop wound dressings, as they can replicate the fibrous dermal ECM microenvironment that provides structural support for wound healing and functional cues for directing tissue regeneration.

Biodegradable synthetic polymers such as polycaprolactone (PCL) have been widely used to produce nanofibers due to their versatile spinning capabilities. Yet, PCL polymeric fibers are poorly suited for developing wound dressings as they are much stiffer than natural skin. Furthermore, they are hydrophobic, limiting their ability to keep wounds hydrated. Synthetic polymers also lack cell binding domains and therefore cannot enhance cellular attachment or functionality. Nanofibers spun from animal-sourced ECM proteins, such as gelatin and collagen in combination with synthetic polymers, have been previously reported in literature to contain bioactive molecules which support healing. Whilst adding ECM proteins to a nanofibrous scaffold enhances its biological and mechanical properties, ECM proteins are costly and susceptible to common liabilities of animal-derived products: immunogenicity, antigenicity, disease transmission, and pathogen contamination. Furthermore, the utilization of collagen alone, the most common ECM protein used in wound dressings, has been shown to cause extensive wound contraction and scarring.

Soy protein is a dietary protein extracted from soy beans. Historically, soy protein and extracts have been used extensively in foods due to their high protein and mineral content. More recently, soy protein has received considerable attention for a variety of its potential health benefits. Epidemiological and clinical studies supporting this claim ultimately enabled US Food and Drug Administration (FDA) approval in 1999 of soy protein for protective effects on coronary heart disease. Alternatively, soy protein has also been explored more recently as a “green” and renewable substitute for petroleum- or animal-derived polymers in biomedical applications.

It has been found that soy protein has bioactive peptides similar to extracellular matrix (ECM) proteins, present in human tissues. Specifically in cutaneous wound healing, it has been shown that cryptic peptides in soy protein improved wound healing by increasing dermal ECM synthesis and stimulating re-epithelialization. Soy phytoestrogens have demonstrated to accelerate the healing process via ER-mediated signaling pathways. They also possess anti-bacterial, anti-inflammatory, and anti-oxidant properties that support and enhance wound healing. It has also been reported that oral intake of soy (both protein and phytoestrogens) accelerates skin regeneration in aged women and burn patients.

Because of these pro-regenerative traits, soy protein-based nanofiber wound dressings have recently been developed in an effort to deliver soy protein to the wound sites. By mimicking the fibrous dermal ECM microenvironment, they can provide potent structural and functional cues for directing tissue regeneration. However, current methods for engineering soy protein nanofibers require the use of synthetic polymers as carriers, due to the low molecular weight of soy protein that inhibits the production of nanofibers alone, and high-voltage for use in electrospinning to prepare the fibers. Moreover, soy protein hydrogels necessitate additional crosslinking agents that can be toxic and can alter the original structure of soy peptides.

As described in this example, plant hybrid cellulose acetate (CA)/soy protein hydrolysate (SPH) nanofibers for wound healing applications have been fabricated. It has been shown that such CA/SPH nanofibers recapitulate the dermal ECM microenvironment and maintain a moist environment while delivering soy protein to potentiate skin regeneration. Cellulose acetate was selected as a co-spinning polymer because it readily dissolves in various solvents and self-assembles into nanofibers, enabling recapitulation of the native ECM fibrous structure and high water retention ability. It is also abundant and exhibits low immunogenicity to humans because of its non-animal origins. Dermal ECM-mimetic CA and SPH nanofibers were manufactured via rotary jet spinning (RJS) system that utilizes centrifugal forces to extrude fibers in the nanometer range. The physicochemical properties of the spun nanofibers were optimized by functionalizing the CA nanofibers with SPH. The RJS-spun CA/SPH nanofibers have higher production rate and better control of fiber morphology without an additional modification or high-voltage electric fields in the system, when compared to the existing electro-spun soy-based nanofibers. Lastly, in vitro and in vivo functionalities of our dressings were tested by investigating dermal fibroblast behaviors and then further assessing wound closure rate and skin regeneration in an excisional wound splinting mice model, respectively. In comparison with the current fibrous scaffolds, the CA/SPH nanofibers described herein have a healing ability similar to or better than other fibrous dressings, but the scaffolds of the invention are free of animal-derived proteins or synthetic polymers that are suboptimal.

### Example 1A: Materials and Methods

The materials and methods used in Example 1 are described below.

### Materials

Polycaprolactone PCL (Mₙ 70,000-90,000; Sigma-Aldrich), cellulose acetate CA (M 50,000; Sigma-Aldrich), soy protein hydrolysate SPH (Amisoy™; Sigma-Aldrich), and hexafluoroisopropanol (HFIP, Oakwood Chemical) were used as received.

### Fiber Fabrication by Rotary Jet Spinning

Nanofibers were spun by using rotary jet spinning (RJS) system as described in U.S. Patent Publication No. 2012/0135448, U.S. Patent Publication No. 2013/0312638, U.S. Patent Publication No. 2014/0322515, which are each incorporated herein by reference in their entireties. Briefly, CA and CA/SPH with different compositions and concentrations (weight per volume percent, wt/v %) were dissolved in HFIP and stirred for overnight. As a reference group, PCL (6 wt/v %) was also dissolved in HFIP. After mixing, solutions were flowed to the rotating reservoir through polyfluoroalkoxy alkane tubing (Saint-Gobain) at 2 mL/min by using an automatic syringe pump (Harvard Apparatus). Then, the solutions were ejected from the reservoir at 60,000 rpm for 5 min, elongating polymers into nanofibers and evaporating HFIP rapidly in the air from the orifice (diameter of 360 μm). The spun nanofibers were dried overnight in a desiccator to fully remove excess solvent. For cell culture, the spun nanofibers were collected on coverslips and sterilized overnight under UV-light.

### Scanning Electron Microscopy (SEM)

Fiber samples were imaged by using a field emission scanning electron microscopy (FESEM, Carl Zeiss). The fiber samples were mounted on sample stubs, sputter-coated with 5 nm thickness of Pt/PD (Denton Vacuum), and imaged by using FESEM.

### Characterization of Chemical Compositions

Attenuated Total Reflectance-Fourier Transform Infrared spectroscopy (ATR-FTIR, Bruker) was used to obtain FT-IR spectra of nanofibers over 600-4000 cm at a resolution of 2 cm with 16 scans. The samples were mounted on sample stage and contacted with ATR-crystal for measurement. The FT-IR spectrum of the dried samples were measured and normalized from 0 to 1. For Gaussian curve fitting and area analysis, OriginPro 9.0 (Origin Lab Corporation) was used. For statistical analysis, n=3 from 3 productions for each condition. X-ray photoelectron spectrometer (XPS, K-Alpha XPS system, Thermo Scientific) was used to further evaluate fiber surface composition. Fibrous test samples were prepared on silicon wafer substrates. Survey and high resolution elemental spectra were obtained using monochromatized aluminum K_{α} radiation (pass energy 200 eV). An argon flood gun was applied to offset sample charging. Peak detection and high resolution C₁ₛ peaks were deconvoluted using Lorentzian/Gaussian product mix (30% L) functions. For statistical analysis, n=3 from 3 productions for each condition. Energy-dispersive X-ray spectroscopy (EDS) in FESEM was used to investigate elemental mapping of nitrogen (N_{K} near 0.392 eV) and carbon (C_{K} near 0.277 eV) atoms, together with corresponding type II secondary electron (SE2) images. The fiber sample was also sputter-coated with Pd/Pt on sample stub and imaged by using EDS.

### Characterization of Fiber and Pore Diameters and Fiber Thickness

Fiber and pore diameters and fiber thickness were analyzed by using SEM images of the nanofibers and ImageJ (NIH) with the plug-in DiameterJ. For fiber thickness analysis, nanofiber scaffolds were prepared from different injection volume (10, 30, and 60 mL in total) and the cross-sectioned scaffolds were imaged and analyzed. DiameterJ was used to determine fiber and pore diameters by using algorithm as described in previous study. Here, the pore diameters refer to the pores of the fibrous scaffolds (between fibers). For statistical analysis, =10 from 3 productions for each condition.

### Biaxial Tensile Test for Stiffness Measurement

The stiffness in the wet state was determined by using biaxial tensile tester (CellScale). The spun fiber scaffolds were loaded by using clamps to hold the samples and immersed in phosphate buffered saline (PBS, ThermoFisher Scientific) at 37° C. Sample was loaded equibiaxially at a strain rate of 5% per second to 20% strain. Loaded samples were biaxially pulled to 80% strain. A built-in software (CellScale) was used to record force/displacement measurements and images at 15 Hz. By using these measurements and the thickness of the samples, stress-strain curves were then produced by OriginPro 9.0. Stiffness was determined by calculating the slope of the stress-strain curves. For statistical analysis, n=5 from 3 productions for each condition.

### Atomic Force Microscopy (AFM) for Roughness Measurement

Roughness (average deviation, Rₐ) was calculated by using built-in software in atomic force microscopy (AFM, MFP-3D™, Asylum). The fiber samples were mounted on sample stage and imaged with tapping mode.

### Contact Angle and Water Absorption Measurements

The cast film samples were prepared on coverslips using spin coater (at 2000 rpm for 1 min). The nanofiber samples were directly spun onto coverslips. A camera was used to record water droplet formation on the surfaces of the substrates. Contact angle was calculated by using ImageJ with the plug-in drop shape analysis. For statistical analysis, n=3 from 3 productions for each condition. Water absorbency was measured as % mass gain like a standard method reported before. First, dry weight of the samples was recorded. The samples were immersed in PBS for 24 h at 37° C. The excess PBS on the wet samples was removed by placing it on a paper towel. Then, weight of the water-absorbing samples was measured. The water absorption ability was defined as described below:

$$A = \frac{100 \times ( W 2 - W 1 )}{W 1}$$

where A is the water absorption ability (%), W1 is the weight before wet, and W2 is the weight after wet. For statistical analysis, n=3 from 3 productions for each condition.

### Biodegradation Measurement

In vitro biodegradation was measured as % mass loss as detailed in previous studies. The initial weight of the scaffold was measured, after which the samples were immersed in PBS at 37° C. and 5% CO2. At day 5, 10, and 15, the samples were washed three times with fresh PBS and dried in an oven at 60° C. overnight. After complete dehydration, the weight of the dried samples was measured. The in vitro biodegradation was defined as follows:

$$D = \frac{100 \times ( W 3 - W 1 )}{W 1}$$

where D is the in vitro biodegradation (%), W1 is the initial weight, and W3 is the final weight after degradation. For statistical analysis, n=3 from 3 productions for each condition.

### Soy Protein Release Kinetics

In vitro release profile of soy protein from the nanofibers was measured as % loss of amide I peaks. The samples were immersed in PBS at 37° C. and 5% CO₂. At Day 0, 3, 5, 7, and 15, the samples were washed three times with fresh PBS and freeze-dried. The FT-IR spectrum of the dried samples were measured and normalized from 0 to 1. The relative areas of amide I peaks were analyzed from the normalized spectrum to calculate the % release of soy protein from the scaffolds. For statistical analysis, n=3 from 3 productions for each condition.

### Cell Culture

Green fluorescent protein (GFP)-expressing human neonatal dermal fibroblasts (HNDFs, Angio-Proteomie) were properly treated as described in protocol from the manufacturer (Angio-Proteomie) for cell culture. Briefly, HNDFs were delivered at passage 3 in a frozen vial and stored in a liquid nitrogen tank before use. Cells were subcultured to passage 7 with Dulbecco's modified eagle medium (DMEM, ThermoFisher Scientific) containing 5% Fetal Bovine Serum (FBS) and 1% antibiotics (penicillin-streptomycin, ThermoFisher Scientific) in a T25 flask at 37° C. incubator with 5% CO₂ and 21% O₂. Once the cells reach passage 7, 2 mL of trypsin/ethylenediaminetetraacetic acid solution (trypsin/EDTA, Lonza) was added to the T25 flask. Seeding density was fixed at 30,000 cells per sample. Cell media was changed every 2 days before imaging and fixation.

### Analysis of Growth, Migration, and Infiltration of Dermal Fibroblasts

GFP-expressing HNDFs on the fibers were imaged by using confocal microscopy (Zeiss LSM 5 LIVE) at 37° C. in a temperature controlled chamber. 2.5% of 4-(2-hydroxyethyl)-1-piperazineethanesulfonic acid (HEPES, ThermoFisher Scientific) buffer was added to the media during imaging in an effort to keep the pH constant. For cellular growth study, the intensity of GFP-expressing HNDF per area was calculated from the confocal images by using ImageJ. For cellular migration study, the migration of GFP-expressing cells on fibers was tracked (1 frame/10 min for at least 40 frames). Once all images were collected, ImageJ plug-in StackReg was used to correct the center of each image. For statistical analysis, n=5 (field of view (FOV)=5) from 3 productions for each condition. Migration of each cell was analyzed by using the plug-in Mtrack2 in ImageJ. The Mtrack2 calculates the total distance each cell has migrated. Migration speed of cells was calculated by dividing the total distance by total imaging time. For statistical analysis, n=5 (FOV=5) from 3 productions for each condition. In cellular infiltration studies, z-stack confocal images of GFP-expressing cells on fibers were captured at 15 days of cell culture. The cell infiltration depth from the z-stack images was calculated using the z-axis profile function in ImageJ. The cross-sectional view (in yz plane) of cells was processed from ImageJ by

using the orthogonal view function. For statistical analysis, n=5 for PCL and n=8 for CA and CA/SPH nanofibers (FOV=3) from 3 productions for each condition.

### Cytotoxicity Measurement

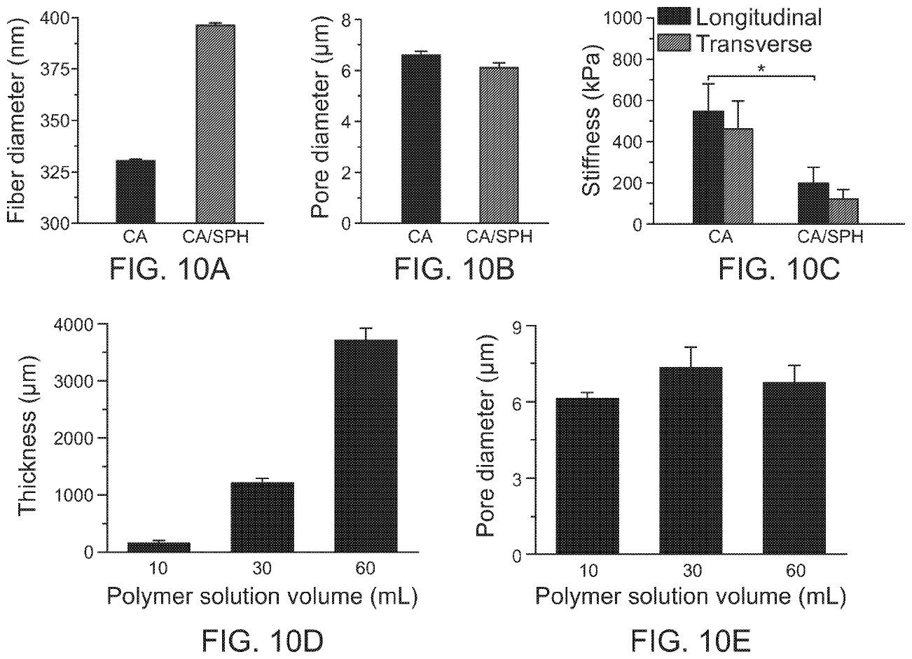

In vitro cytotoxicity of cells on the fibers was measured by using lactate dehydrogenase (LDH) cytotoxicity assay (Promega) as described previously. Briefly, HNDFs were cultured on nanofibers for 15 days and successively incubated with reaction solution and stop solution (1 M acetic acid) from the assay kit. A commercial plate reader was used to measure absorbance at 490 nm. The % cytotoxicity was defined as follows:

$$\% Cytotoxicity = \frac{100 \times ( S - C )}{M - C}$$

where S is the readout from the sample, C is the readout from the control (medium only without cell), and M is the readout from maximum LDH release. For statistical analysis, n=17 in triplicate from 3 productions for each condition. For the box plot in FIGS. 10D and 10#, the box range is 25-75%, the whisker range is 10-90% using OriginPro 8.6 software.

### Immunocytochemical Analysis

After 15 days of culture, HNDFs grown on nanofibers were fixed in 4% paraformaldehyde (PFA) and 0.05% Triton-X for 10 min. Following fixation, samples were incubated with primary antibody (rabbit polyclonal anti-Ki67 with 4′,6-diamidino-2-phenylindole dihydrochloride (DAPI) for proliferation study or rabbit monoclonal anti-integrin β1 antibody, Abcam) and with secondary antibody (goat anti-rabbit IgG (H+L) secondary antibody with Alexa Fluor® 546, Invitrogen) during 1 h at room temperature for both primary and secondary antibody incubation. Following immunostaining, samples were mounted on glass slides by using Prolong Gold anti-fade agent (Invitrogen) and imaged on the confocal microscopy. Cell proliferation was calculated by dividing the number of Ki-67 positive cells by the number of DAPI-positive cells. For statistical analysis, n=5 for PCL and n=6 for CA and CA/SPH (FOV=25) from 3 productions for each condition.

### Western Blot Analysis

HNDFs were cultured on nanofibers for 15 days and were lysed at 4° C. using radioimmunoprecipitation assay (RIPA) lysis buffer (SLBG8489, Sigma) with Complete Mini (11836153001, Roche Diagnostic) and Halt-Protease and Phosphotase Inhibitor (1861281, ThermoFisher Scientific). A capillary-based Wes Simple Western (ProteinSimple) was used to detect and quantify the expression of integrin β1 in cell lysates following the manufacturer's protocol. In brief, each capillary loaded 5 μg of sample lysates and separated proteins by size. The samples were incubated with primary antibodies for Integrin 131 and Glyceraldehyde 3-phosphate dehydrogenase (GAPDH) as a loading control (ab52971 and ab9485 respectively, ABCAM). Target proteins were labeled with secondary antibodies and chemiluminescent reagents provided by the manufacturer (ProteinSimple). Signals were detected and quantified using CompassSoftware (ProteinSimple). Expression of integrin β1 was normalized to GAPDH loading control and compared across sample conditions. For statistical analysis, n=6 for CA and n=7 for CA/SPH from 3 productions for each condition.

### Mouse Excisional Wound Splinting Model

All mouse wound healing experiments were performed using IACUC approved protocols (Protocol ID 11-11). Based on the previous publications, the mouse excisional splinting model was carried out in order to analyze cutaneous wound closure in murine skin by excluding wound contraction. Briefly, splinting rings were prepared by cutting 8 mm holes in a 0.5 mm-thick silicon sheet (Grace Bio-Labs) using a sterile biopsy punch)(Integra® Miltex®). The prepared rings were washed and sterilized by 70% (vol/vol) ethanol, and then were air-dried in a sterile culture hood before surgery. C57BL/6 male mice (Charles River Laboratories, 52 days old) were anesthetized with isofurane through the duration of procedure. Once anesthesia was confirmed by a toe pinch test, the dorsal side of mice was shaved using electric and manual razor. After hair removal, the skin was cleaned with betadine (Santa Cruz Biotechnology) and 70% (vol/vol) ethanol. The full-thickness excisional wounds were created on the midline by punching through the skin with a 6-mm-diameter sterile biopsy punch. The punched tissues were used for histological analysis of healthy skin (Day 0). An instant-bonding adhesive (Krazy glue) was put on one side of a splint. The splints were fixed into place around the wound with instant bonding adhesive followed by suturing with nylon suture (Ethicon). Nanofiber wound dressings were applied to the wound and covered with Tegaderm™ (Nexcare™) patches to keep the scaffolds in place and the surgical area clean. Control wounds received no nanofibers and were covered with Tegaderm™ patches only. Tegaderm™ is a clinical standard wound dressing. The mice were monitored daily. Before tissue harvest on Day 7 and 14, mice were sacrificed via IACUC approved methods.

### In Vivo Wound Closure Analysis

Wound areas were photographed with a digital camera on Day 0, 7, and 14. The wound area was manually quantified using ImageJ. Wound closure was defined as described below:

$$Wound closure ( \% ) = 100 \times \frac{( \begin{matrix}Area of original wound - \\ Area of actual wound\end{matrix} )}{Area of actual wound}$$

### Histological Analysis

Histological analysis was preformed based on previously published methods. Tissues were harvested from Day 0 and 14 and fixed with 4% PFA at 4° C. overnight. The fixed tissue was washed using PBS five times for 30 min each. The tissue was incubated with 20% and 40% (wt/vol) sucrose (Sigma) in PBS at room temperature for 2 h each. Then, the tissue was embedded in O.C.T. compound (Electron Microscopy Science) with cryomold (Tissue-Tele). The frozen wound tissues were sectioned with 10 μm thickness, stained with hematoxylin and eosin (H&E), and imaged by slide scanner (Olympus VS120). Re-epithelialization was analyzed by manually calculating distance among the newly synthesized epithelial layers from H&E staining tissue sections (marked with arrows in FIGS. 34A, 34B, 34D, 35A, 35B, 35C, 36A, 36C). Epithelial thickness was also manually measured using ImageJ. Scar index was quantified by using a previously published method. Briefly, scar area (areas surrounded by dotted lines in FIGS. 34A, 35A and 36A) and dermal thickness were manually measured using ImageJ. Then, scar index was defined as described below:

$$Scar index ( µm ) = \frac{Scar area ( µm \times µm )}{Average dermal thickness ( µm )}$$

Dermal collagen alignment in the wounds was calculated by using OrientationJ in ImageJ as previously published. The OrientationJ computes the coherency that is between 0 (isotropic) and 1(anisotropic). Fiber wound dressings were prepared from 3 productions for each condition. For statistical analysis, n=3 wounds and 3 mice for control, n=4 wounds and 3 mice for CA and CA/SPH nanofibers, n=5 wounds and 5 mice for healthy tissue, at least 3 sections per wound.

### Statistical Analysis

All data is displayed as mean±standard error (SEM). One-way analysis of variance (ANOVA) in OriginPro 9.0 was used for statistical comparisons. Statistical significance was determined at * p<0.05.

### Example 1B: Fabrication of Cellulose Acetate-Soy Protein Hydrolysate (CA/SPH) Nanofibers

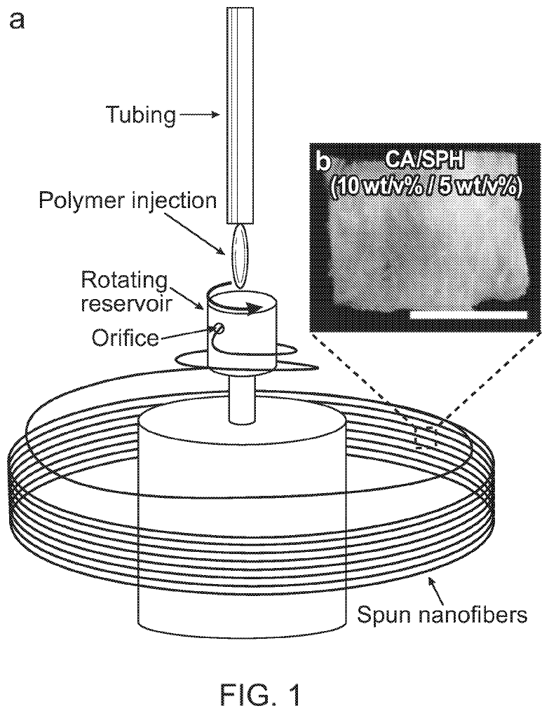

Plant-based hybrid nanofibers were fabricated by co-spinning cellulose acetate (CA) and soy protein hydrolysate (SPH) in hexafluoroisopropanol (HFIP) using a rotary jet spinning (RJS) system, which produces apparently defect-free nanofibers under centrifugally induced shear forces (FIG. 1). CA was chosen to supplement the low molecular weight of soy protein, and SPH was chosen as the soy protein source. As depicted in FIG. 1, continuous CA and CA/SPH nanofibers were spun at a centimeter scale by extruding polymer solution from the rotating reservoir.

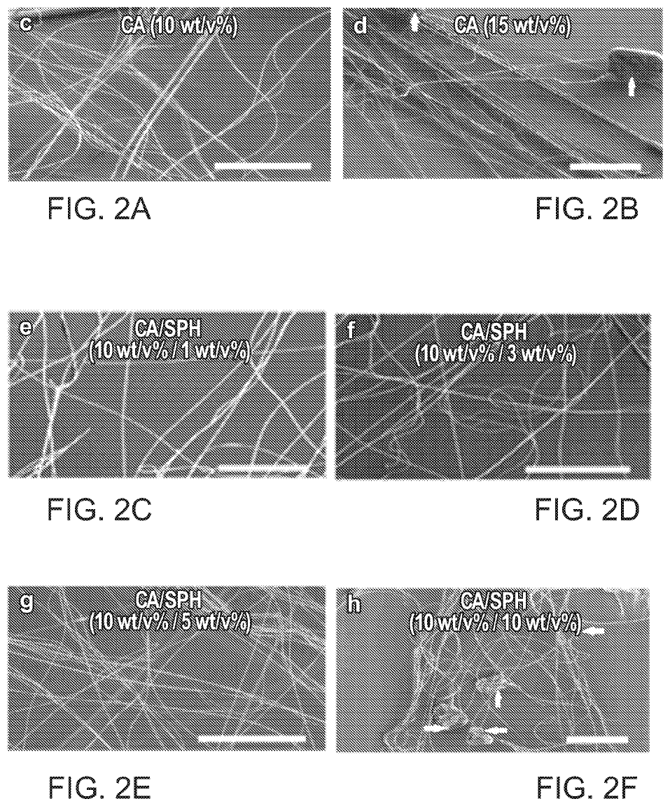

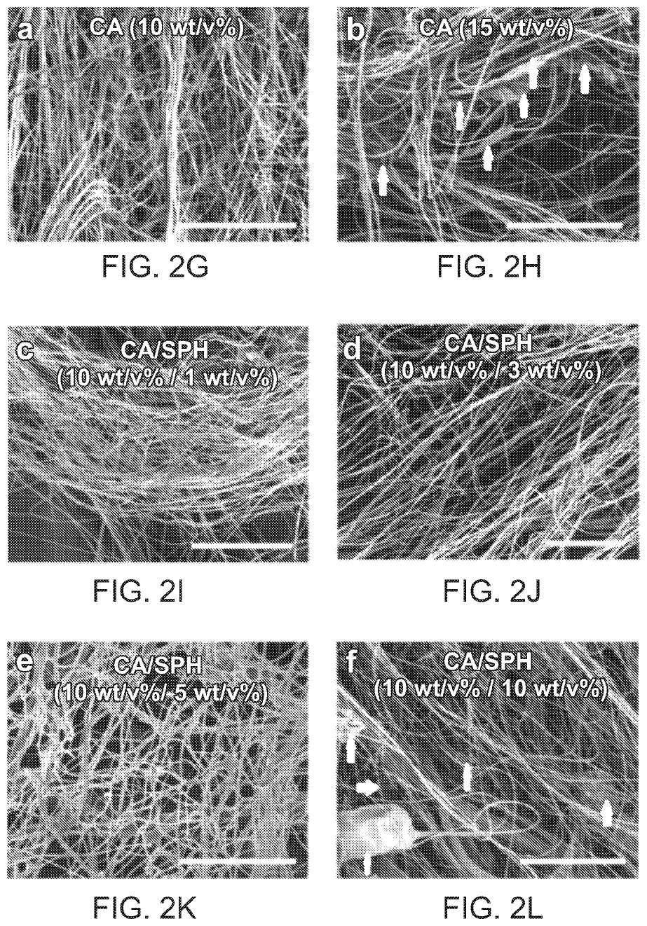

For the RJS system, the spinnability and beading of CA and SPH nanofibers were significantly influenced by their polymer concentrations (w/v %). Table 1 shows that SPH alone could not be spun into nanofibers because its molecular weight is too low. The short chains of SPH molecules cannot overlap and entangle, suggesting that SPH would require a co-spinning polymer with longer chains. Experimentation with fixed rotation and injection speeds showed that adding 10 w/v % of CA to various concentrations of SPH (1, 3, 5 w/v %) resulted in continuous nanofiber formation without beading (Table 1 and FIGS. 2E and 2G). A higher concentration of SPH (10 w/v %) in contrast showed beading in fibers (Table 1 and FIG. 2H). Moving forward, 10 w/v % of CA was therefore selected as the carrier polymer for SPH. The developed continuous nanofibers had an intercalated nanofibrous structure that resembles the native extracellular matrix. This morphological similarity supports cell-fiber interactions that promote wound healing.

TABLE 1
Spinnability of CA and SPH in HFIP

| Material | | | |
|---|---|---|---|
| Carrier polymer | Soy protein | | Corresponding |
| (w/v %) | (w/v %) | Morphology | image |
| CA (5) | None | No fiber | N/A |
| CA (10) | None | Continuous fibers | FIGS. 2A, 2G |
| CA (15) | None | Continuous fibers with beads | FIGS. 2B, 2H |
| CA (10) | SPH (1) | Continuous fibers | FIGS. 2C, 2I |
| CA (10) | SPH (3) | Continuous fibers | FIGS. 2D, 2J |
| CA (10) | SPH (5) | Continuous fibers | FIGS. 2E, 2K |
| CA (10) | SPH (10) | Continuous fibers with beads | FIGS. 2F, 2L |
| None | SPH (10) | No fiber | N/A |

### Example 1C: Chemical Composition Analysis of CA/SPH Nanofibers by ATR-FTIR Spectroscopy

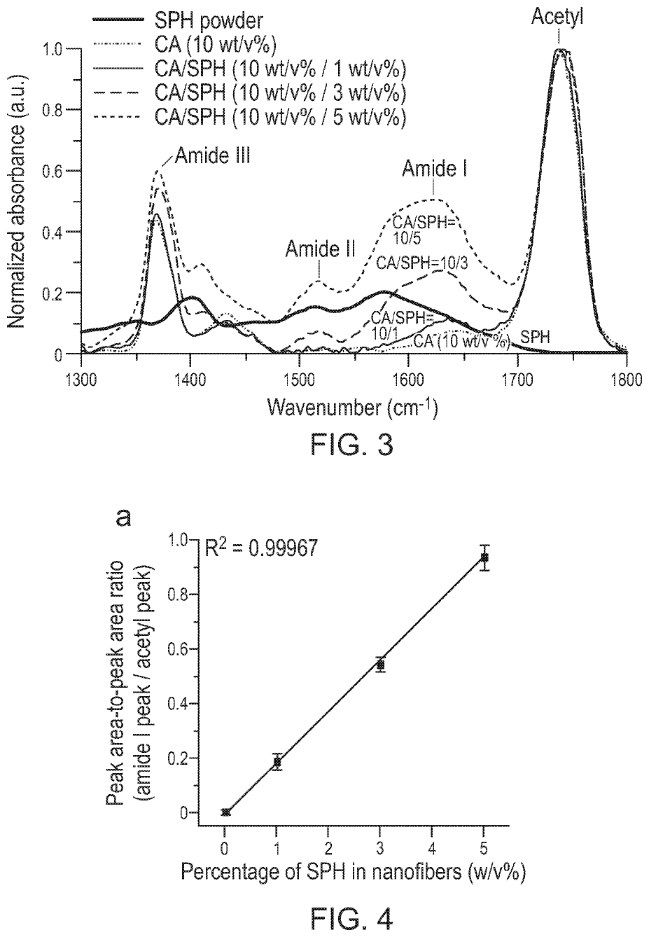

To ensure a uniform structure, elements must be homogenously dispersed at the nanofiber surface. ATR-FTIR (attenuated total reflectance-Fourier transform infrared) spectroscopy was performed to determine the relative amounts of proteins in the spun nanofibers. In the FTIR spectrum shown in FIG. 3, amide I peaks (1600-1700 cm⁻¹) are representative of the secondary structure of amino acids in SPH, and acetyl peaks (1700-1800 cm⁻¹) are representative of C═O stretching of acetyl groups in CA. Soy phytoestrogens can also attributed to peaks in 1600-1700 cm⁻¹ range due to C═O and C═C stretching in phytoestrogen molecules. After subtracting background intensity from CA in the amide I peak, the peak area-to-peak area ratios (amide I peak over acetyl peak) were linearly related to the amounts of SPH (FIG. 4), showing that SPH can be added into fibers in an amount up to 5 w/v % without causing the loss of soy protein molecules.

### Example 1D: Elemental Composition Analysis of CA/SPH Nanofibers by XPS

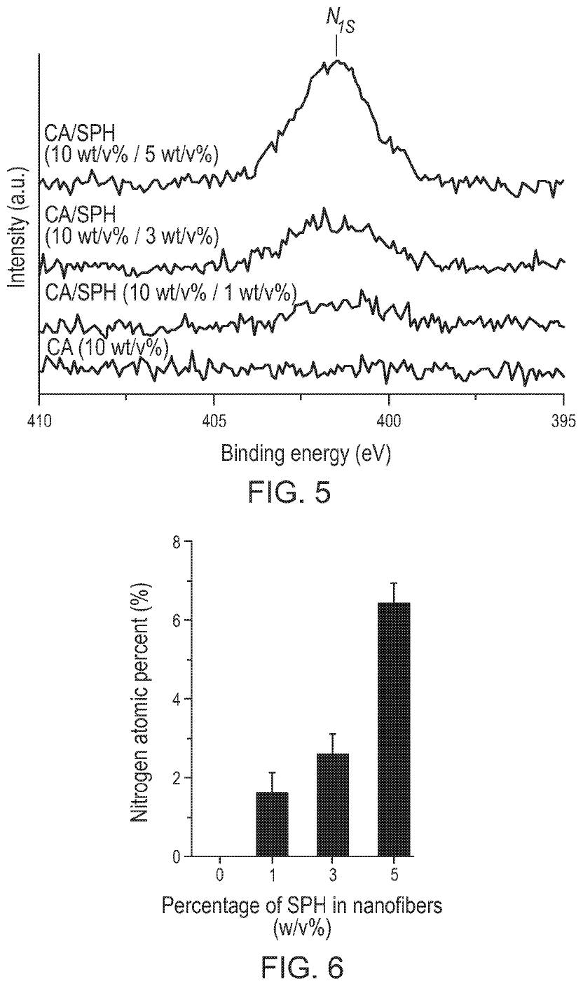

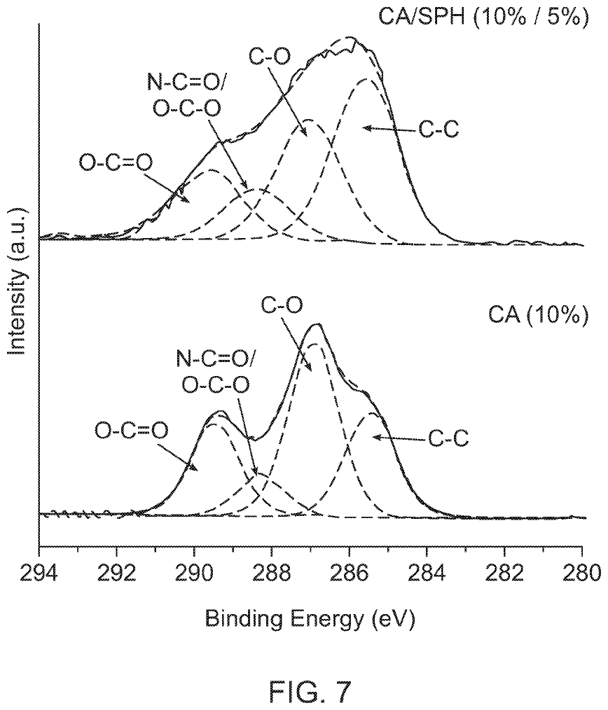

XPS (X-ray photoelectron spectroscopy) was performed to confirm the elemental composition of the nanofiber surfaces. The nitrogen content gradually increased as the concentration of SPH increased (FIGS. 5 and 6), confirming that SPH was incorporated into CA nanofibers. High resolution analysis of the C₁ₛ peaks additionally confirmed the increasing protein content on the nanofiber surface. This peak was deconvoluted, into four peaks corresponding to the following chemical bonds: C—C, C—O, O—C—O/N—C═O, and O—C═O (FIG. 7). Increasing SPH content thus led to relatively higher concentrations of C—C and O—C—O/N—C═O bonds (FIG. 7 and Table 2). More amino acids and phytoestrogens in higher concentration of SPH were ascribed to the increase of C—C and O—C—O/N—C═O bonds. These results demonstrated that SPH was successfully integrated with CA.

TABLE 2
Relative atomic concentration of XPS spectra of deconvoluted C₁ₛ

Deconvolution of C₁ₛ

| | | | O═C—N or | | |
|---|---|---|---|---|---|
| | C—C | C—O | O—CO | O═C—O | |
| Material | (285.5- | (287.0- | (288.3- | (289.5- | |
| (w/v %) | 285.6 eV) | 287.1 eV) | 288.4 eV) | 289.6 eV) | Total |
| CA (10) | 25.74% | 42.46% | 9.4% | 22.39% | 100% |
| CA/SPH | 40.43% | 29.03% | 13.02% | 17.53% | 100% |
| (10/5) | | | | | |

### Example 1E: Component Distribution Analysis of CA/SPH Nanofibers by EDS

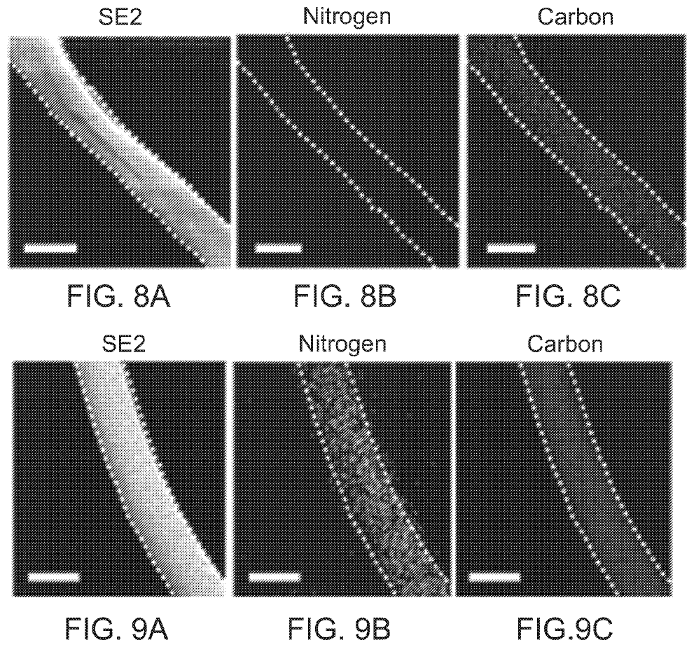

To analyze the distribution of CA and SPH in individual fibers, EDS (energy-dispersive X-ray spectroscopy) was performed to obtain an elemental mapping of nitrogen and carbon atoms (FIGS. 8A-8C, 9A-9C). Carbon mapping showed uniform distribution of carbon atoms on the spun nanofibers, matching the corresponding secondary electron (SE2) images. Nitrogen atoms appeared exclusively on CA/SPH nanofibers owing to the presence of SPH and were homogeneously distributed throughout individual fibers (FIGS. 9A-9C). This confirms and concludes that spinning CA at 10 w/v % and SPH at 5 w/v % improved fiber spinnability and yielded fibers with high concentrations of uniformly distributed protein. In the following studies, CA (10 w/v %) and CA/SPH (10 w/v % /5 w/v %) nanofibers were selected as pure CA nanofibers and CA/SPH nanofibers, respectively.

### Example 1F: Characterization of Mechanical Properties and Surface Chemistry of Nanofibers

The physico-mechanical properties of nanofibers—fiber diameter, pore diameter, and stiffness—influence wound healing. It has been shown that fiber diameter (200-400 nm) and pore diameter (6-20 μm), similar to the native ECM, enhance adhesion, proliferation and infiltration of human dermal fibroblasts, while minimizing bacterial infiltration. Fiber stiffness has also been shown to affect cell behavior. To encourage assembly of new ECM, the stiffness of wound dressing materials should mimic the stiffness of the native ECM microenvironment (5-600 kPa), although the stiffness of common synthetic polymer nanofiber scaffolds is usually one to several orders of magnitude higher.

### Fiber and Pore Diameters

FIGS. 10A and 10B respectively indicate that fiber diameter ranges from 300.30±0.76 nm in CA nanofibers and to 396.66±0.90 nm in CA/SPH nanofibers. In contrast, PCL nanofibers showed thicker fiber diameter (644.04±5.20 nm) than CA-based nanofibers. Pore diameter ranges from 6.63±0.14 μm in CA scaffolds to 6.13±0.17 μm in CA/SPH nanofiber scaffolds, while PCL scaffold pore size decreased to 3.82±0.38 μm.

### Stiffness

Next, the scaffold thickness can be controlled by spinning a different amount of polymer solution. FIGS. 10D and 10E showed that the RJS system was able to produce fiber scaffolds with thickness ranging from a couple hundred micrometers to several millimeters, However, scaffold thickness does not significantly change pore diameters of nanofiber scaffolds. The stiffness of the CA and the CA/SPH nanofibers was between 100 and 600 kPa in the longitudinal and transverse directions respectively (see FIG. 10C and Table 3). On the other hand, the stiffness of the PCL fibers was in a MPa range, which is much stiffer when compared to native skin or CA-based nanofibers. These results suggest that fiber and pore diameter of both CA and CA/SPH nanofibers are well suited to support growth and migration of human dermal fibroblasts and that their stiffness resembles that of human skin ECM.

TABLE 3
Modulus of nanofiber scaffolds

| Material (w/v %) | Direction | Modulus (mean ± SEM) |
|---|---|---|
| PCL(6) | Longitudinal | 8.64 ± 0.93 MPa |
| PCL (6) | Transverse | 5.12 ± 0.82 MPa |
| CA (10) | Longitudinal | 549 ± 131 kPa |
| CA (10) | Transverse | 464 ± 131 kPa |
| CA/SPH (10/5) | Longitudinal | 197 ± 74 kPa |
| CA/SPH (10/5) | Transverse | 126 ± 40 kPa |

### Surface Roughness

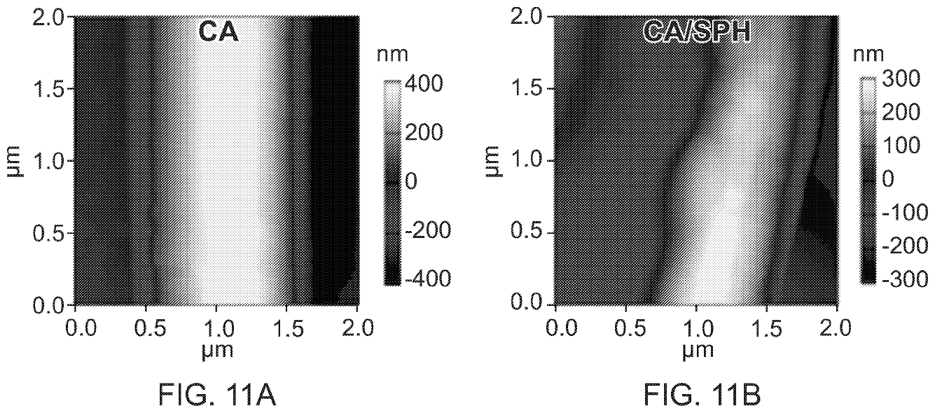

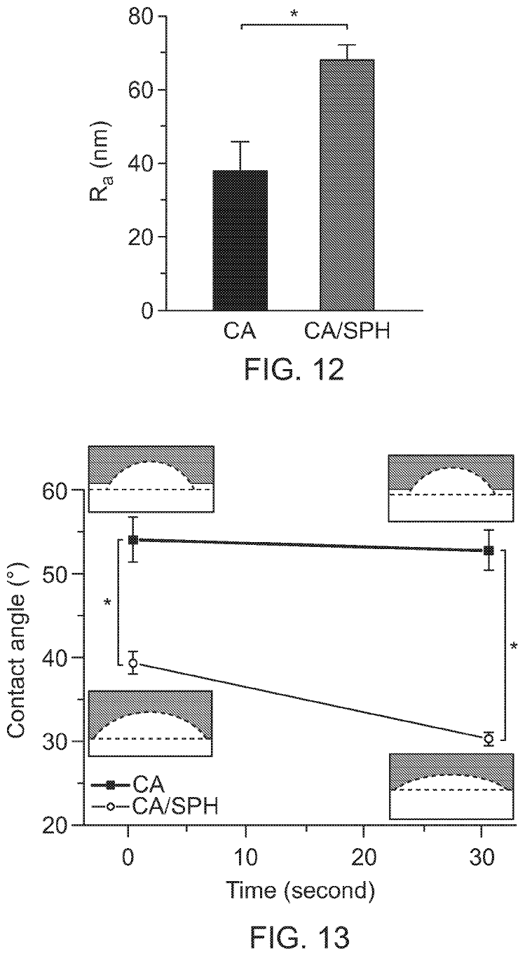

The surface roughness of the nanofibers, which affects cellular behaviors at both nano- and micro-scales since cells sense and react differently on various micro-topographies. It has been reported that rough surfaces enhance cell adhesion, migration, and growth by triggering expression of integrin receptors and production of growth factors and ECM proteins. To estimate the effect of the addition of SPH on the surface roughness of CA nanofibers, the average deviation (Rₐ) of the surface roughness was calculated from atomic force microscopy (AFM) images (FIGS. 11A, 11B). FIG. 12 shows that the Rₐ value for the CA/SPH nanofibers (68.19±4.13 nm) was significantly higher than that of the CA nanofibers (38.06±7.98 nm). Several factors may account for the effect of SPH on fiber roughness: the distribution of proteins throughout the surface and inside the nanofibers (FIGS. 3, 5, 8A-8C, 9A-9C), the aggregation of different materials within the nanofibers, and the short peptides that SPH carries.

### Hydrophilicity and Water Absorbing/Retaining Capabilities

The incorporation of SPH introduces polar moieties such as hydroxyl, amino, and carboxylic groups into the fibers. This increases the hydrophilicity as well as improves cell attachment by providing cell-binding functional groups. High hydrophilicity and water retaining properties are vital for removing wound exudates and providing a moist environment for cell growth.

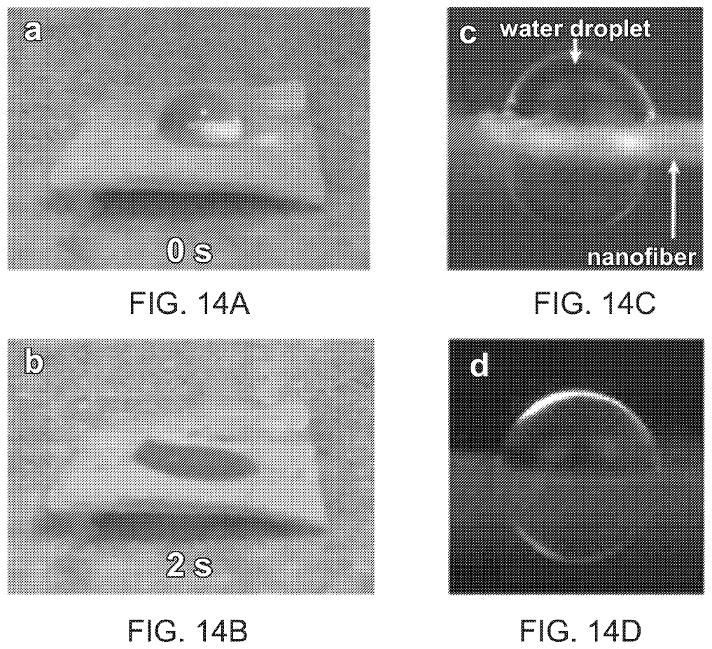

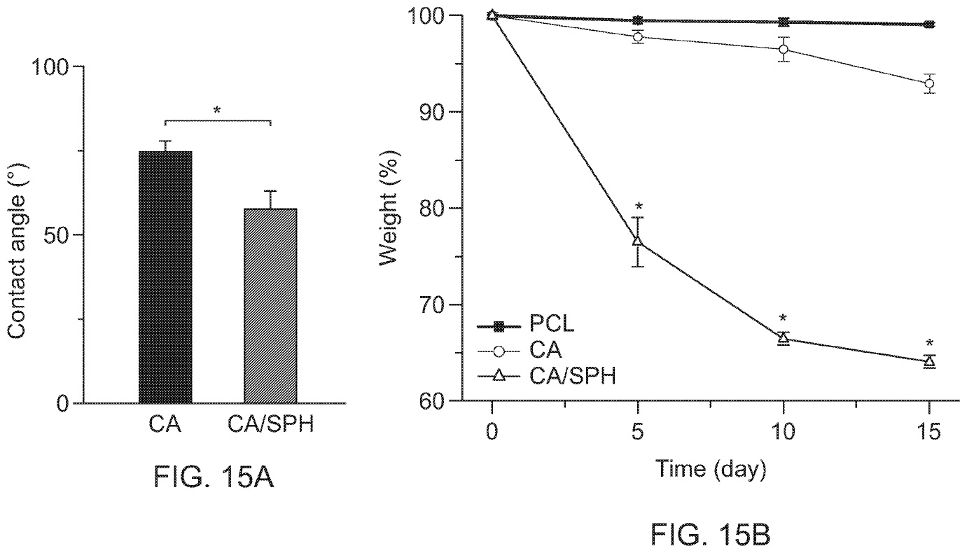

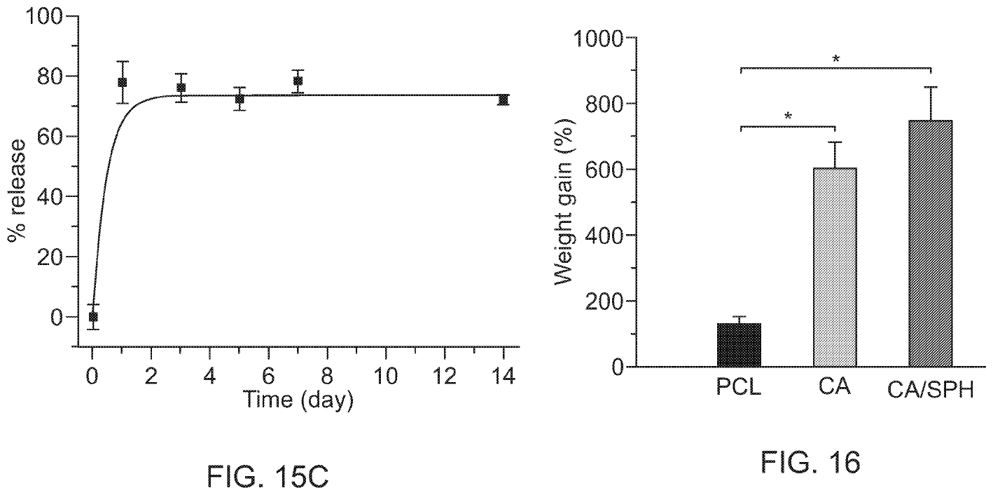

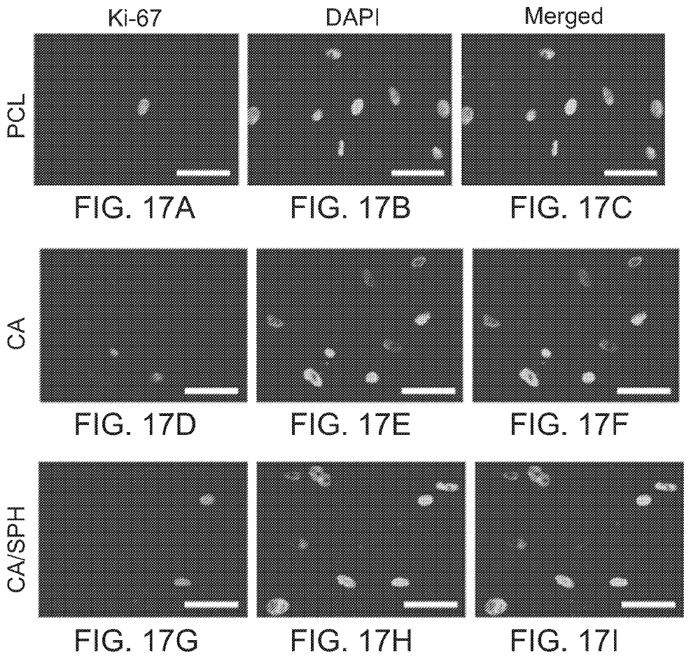

To evaluate the chemical composition influence on the hydrophilicity of the materials, contact angle measurement of uniform cast films was performed (FIGS. 13, 14A-14D, 15A15C, 16). The contact angles were significantly reduced by raising the ratio of SPH in the films, indicative of increased hydrophilicity. A similar trend was seen for fibrous samples, though rapid diffusion of water into the samples was seen for all samples (FIGS. 14-14D, 15A). The increased hydrophilicity was reflected by an increased water absorption capacity (FIG. 16). When CA was used as a backbone in nanofibers, their water-absorbing capabilities were significantly greater than that of hydrophobic polycaprolactone (PCL) nanofibers which are frequently used as a backbone polymer to spin nanofiber scaffolds. Also, the CA/SPH nanofiber had higher water uptake than that of pure CA fibers.

An ideal nanofibrous scaffolds should be highly biodegradable so that it is gradually replaced by natural tissues during wound healing. FIG. 15B shows that over a 15-day period CA/SPH nanofibers lost significantly more mass than CA or PCL nanofibers due to hydrolysis of soy proteins. The rate of soy protein hydrolysis within the hybrid nanofibers resulted in the degradation, which correlates with the rate of protein breakdown. The lower mechanical strength and higher surface wettability of the hybrid nanofibers also contributed to their rate of degradation. In addition, the release kinetics of soy protein from CA/SPH nanofiber scaffolds resulted in a burst release of soy protein within 24 hours due to the fast hydrolysis of soy protein and high hydrophilicity (FIG. 15C). After the initial burst release, a sustained soy release over 2 weeks was observed. The two phases of in vitro release (the initial burst and the sustained release over a long period) are typical release profiles of nanofiber-loaded molecules. Therefore, a dressing made from plant-based hybrid nanofibers could provide structural cues until wound healing is completed and be naturally replaced by native tissue.

### Example 1G: In Vitro Fibroblast Study

The inventors of the present application hypothesize that the addition of SPH into CA nanofiber could promote wound healing-relevant cellular activity of human neonatal dermal fibroblasts (HNDF) via the presence of bioactive molecules, increased roughness, and enhanced water-retaining capabilities. As an effort to test this hypothesis, several indicative markers for wound closure and tissue regeneration were analyzed, including in vitro proliferation, surface coverage, migration, and infiltration of HNDFs (FIGS. 17A-17I, 20A-20L, 29A-29F).The behaviors of dermal fibroblasts were tested in vitro because they are a critical skin cell type that remodels the dermal ECM, communicates with other skin cells (such as keratinocytes), and thus regulates dermal function. Cytotoxicity tests of the nanofiber scaffolds were likewise conducted as a standard pre-clinical experiment. PCL nanofibers were used as a reference since it is one of the most common Cytotoxicity tests of the nanofiber scaffolds were likewise conducted as a standard pre-clinical experiment. PCL (6 wt/v %) nanofibers were used as a reference since it is one of the most common biocompatible and biodegradable synthetic polymers in nanofiber fabrication for biomedical applications.

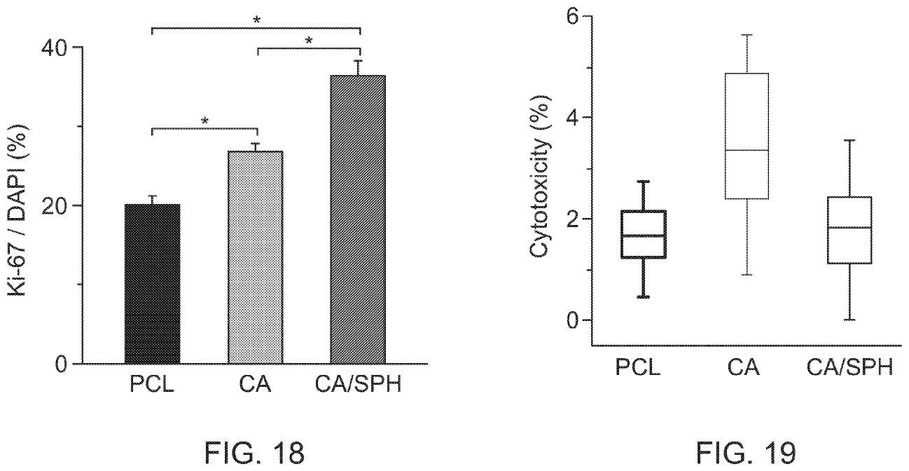

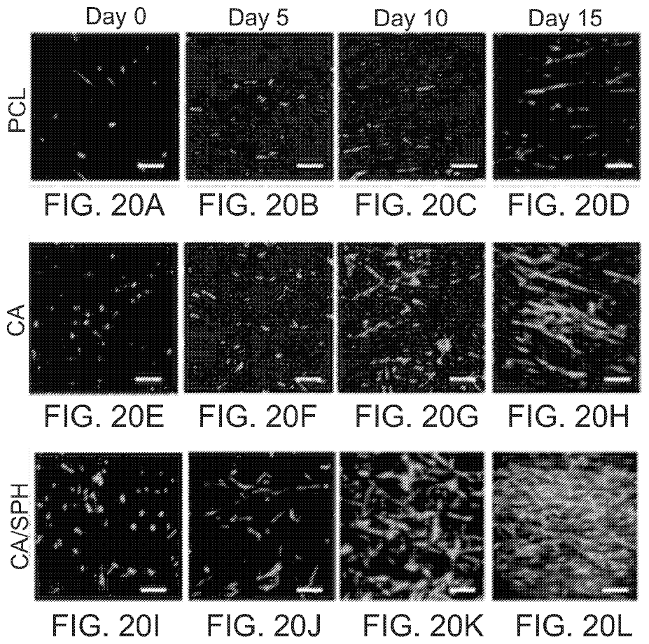

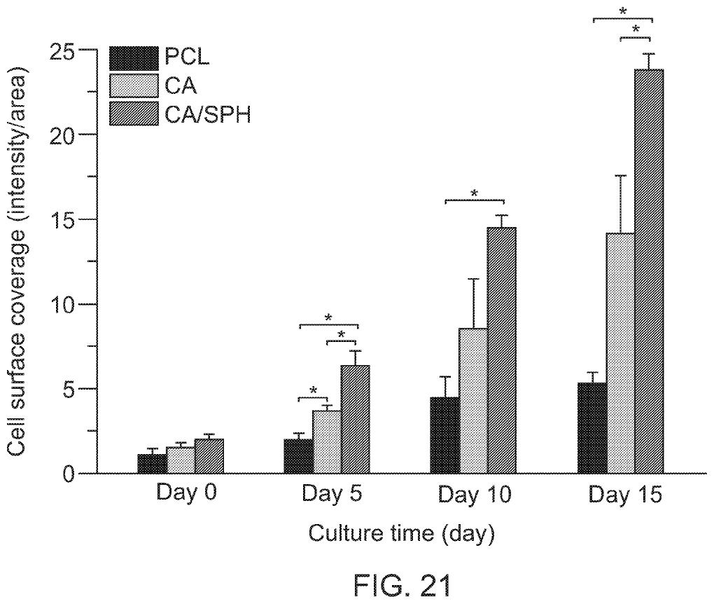

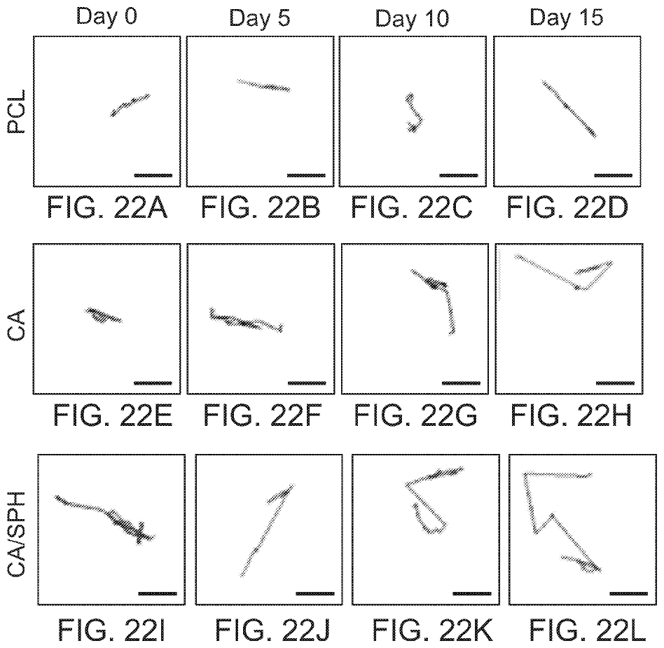

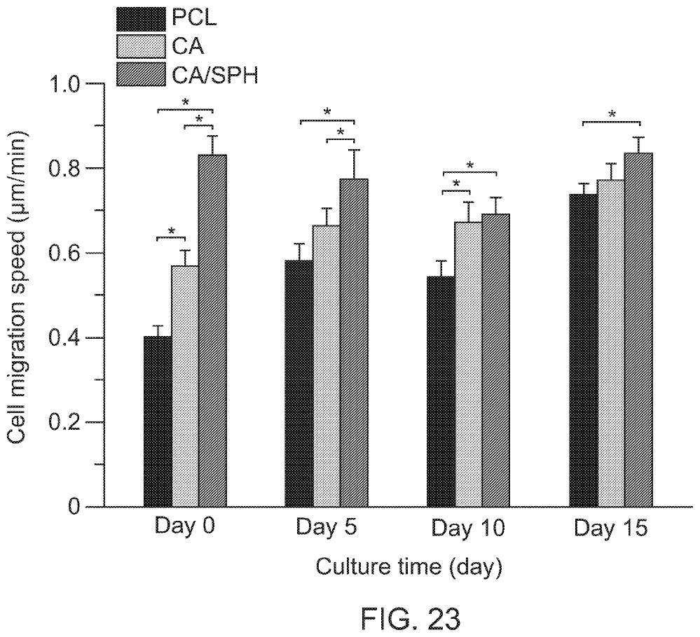

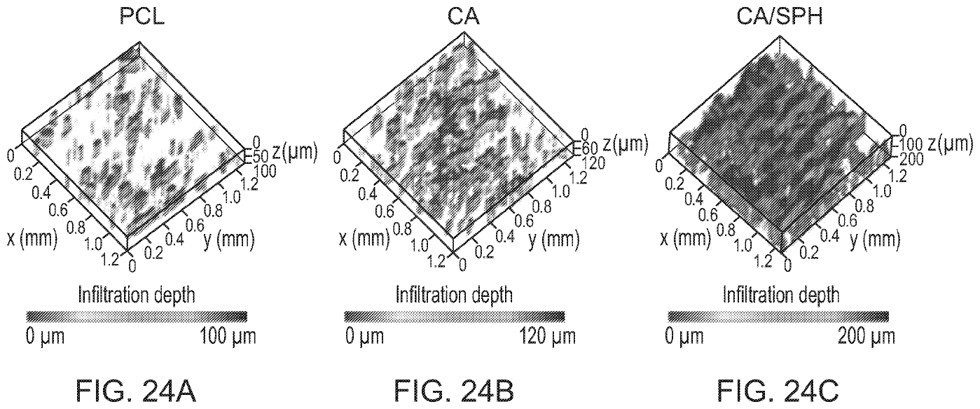

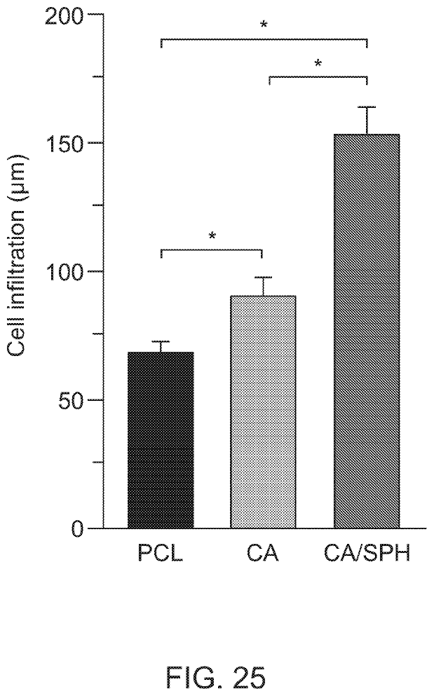

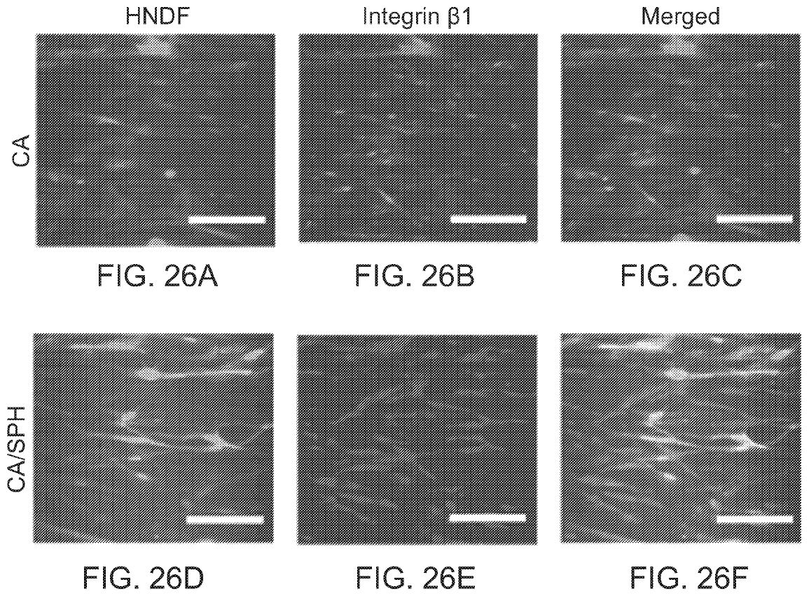

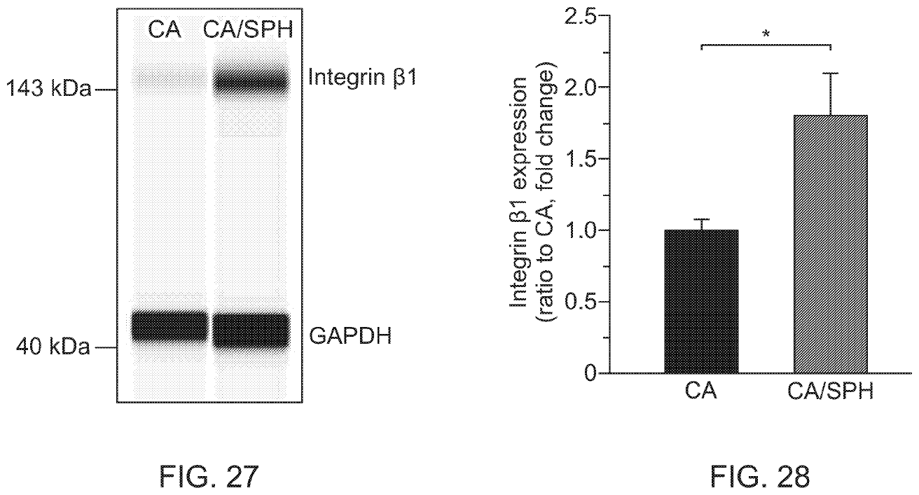

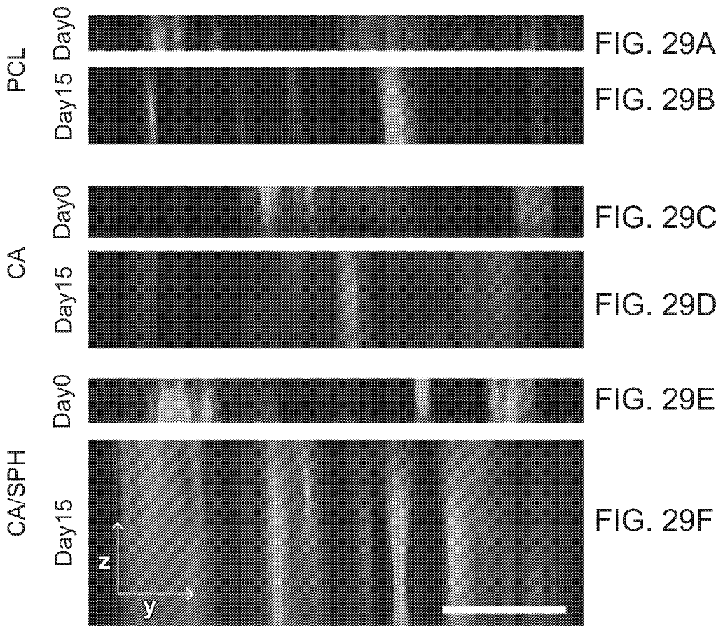

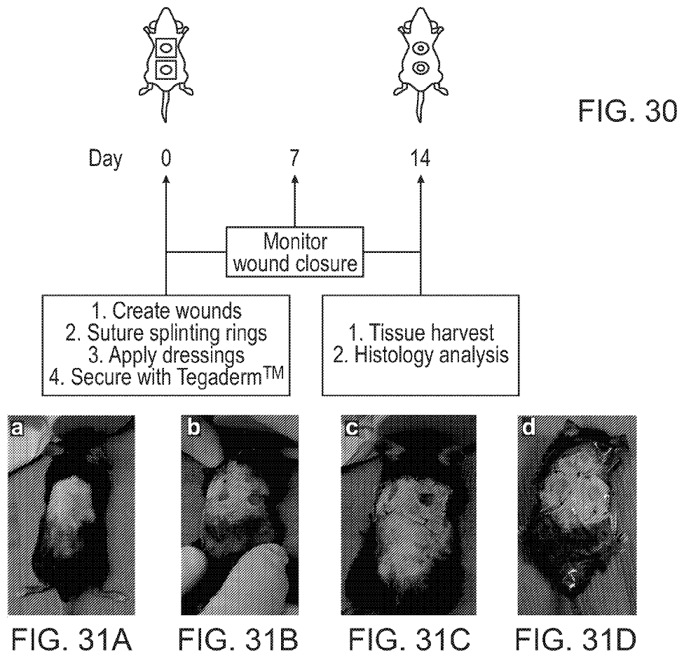

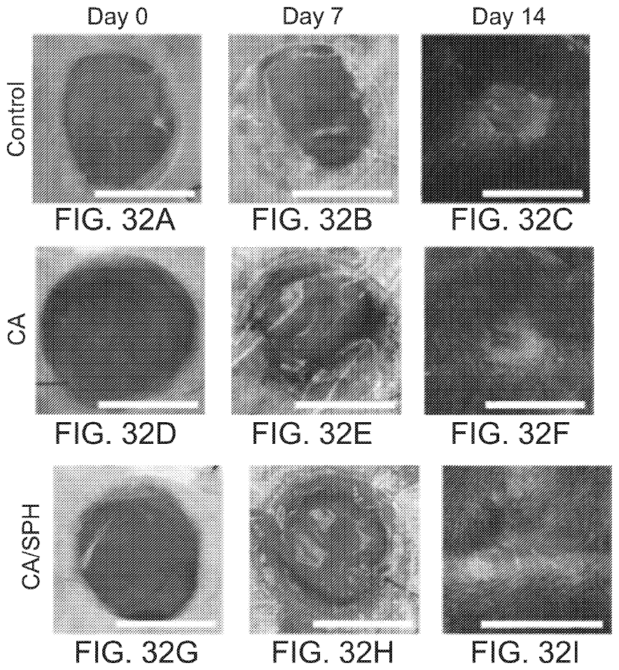

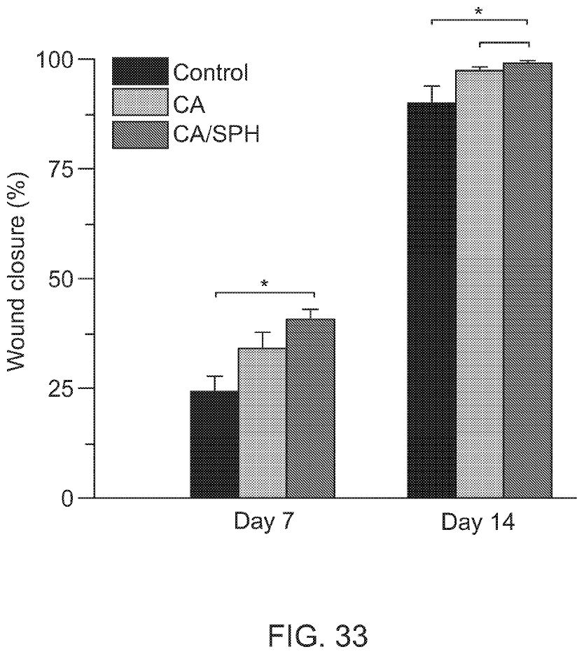

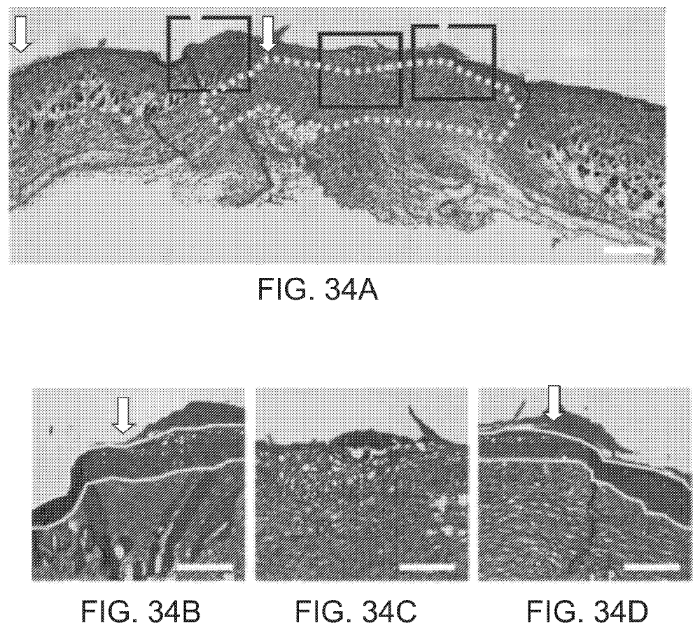

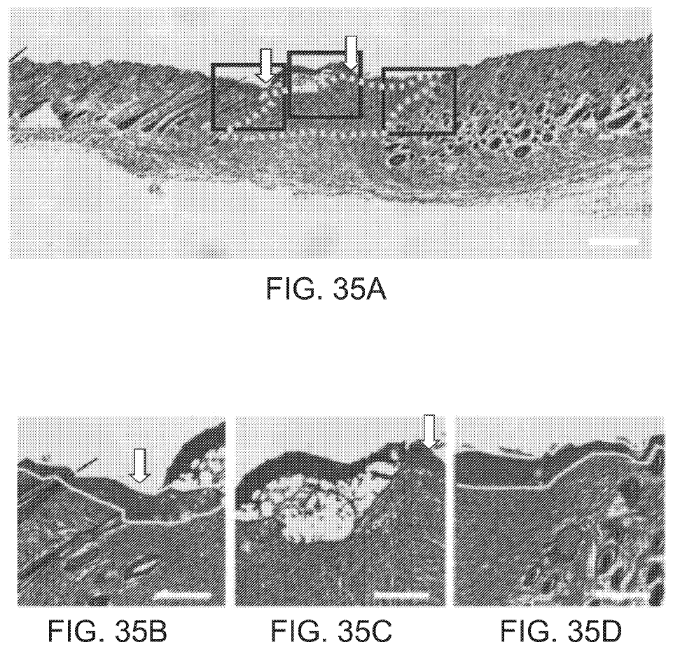

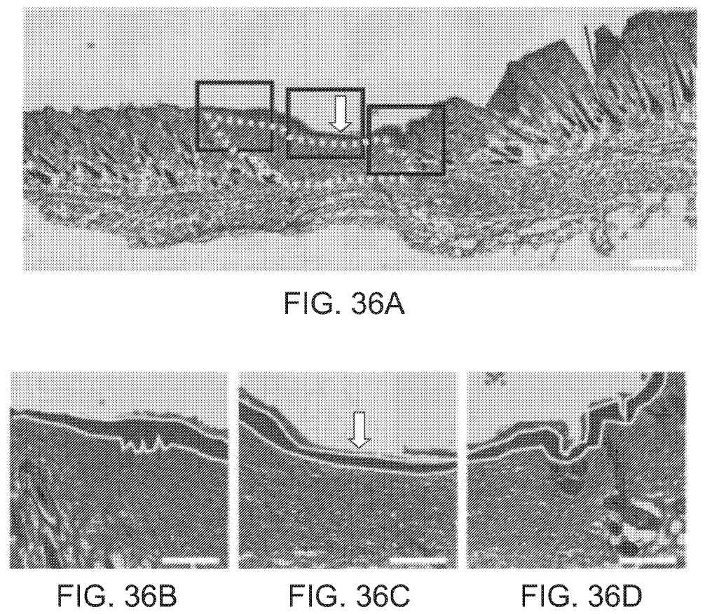

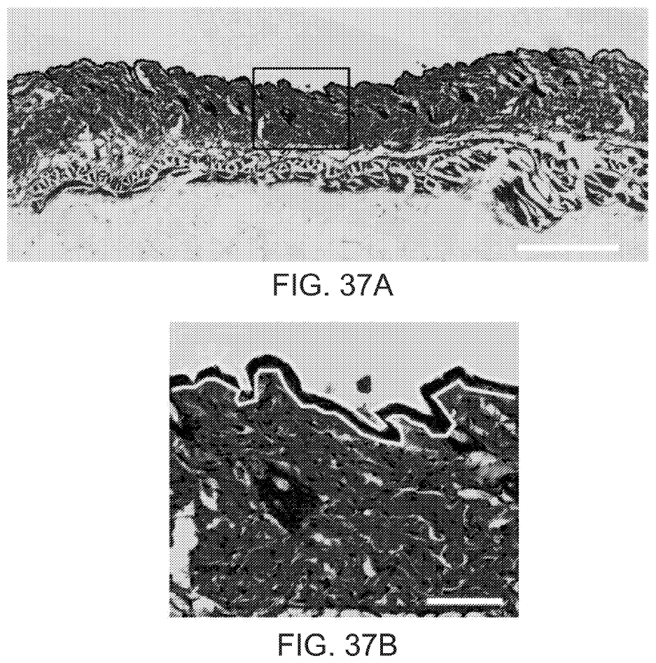

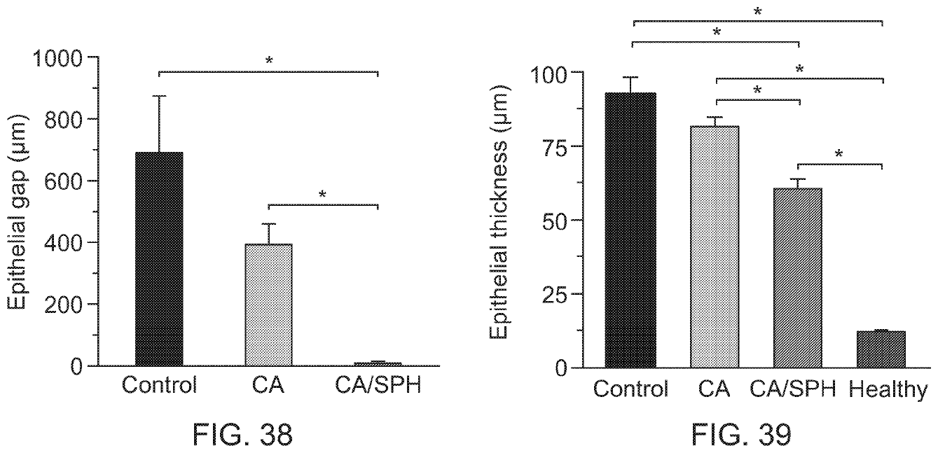

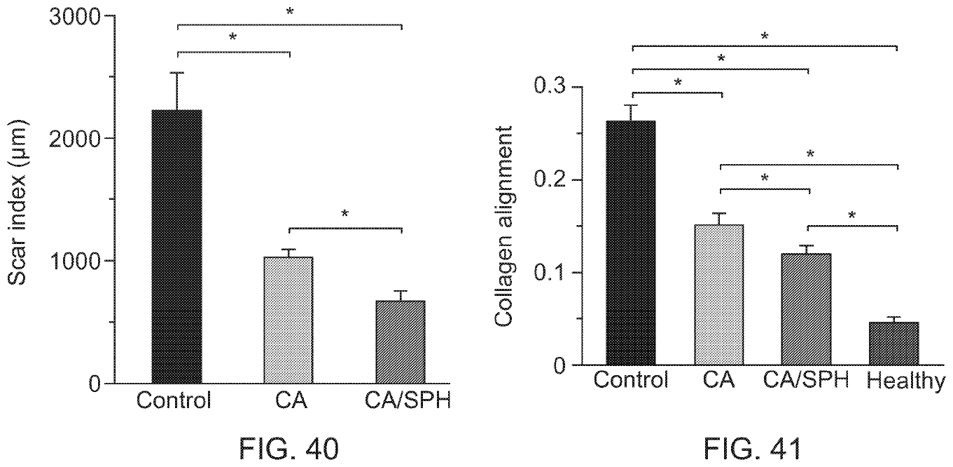

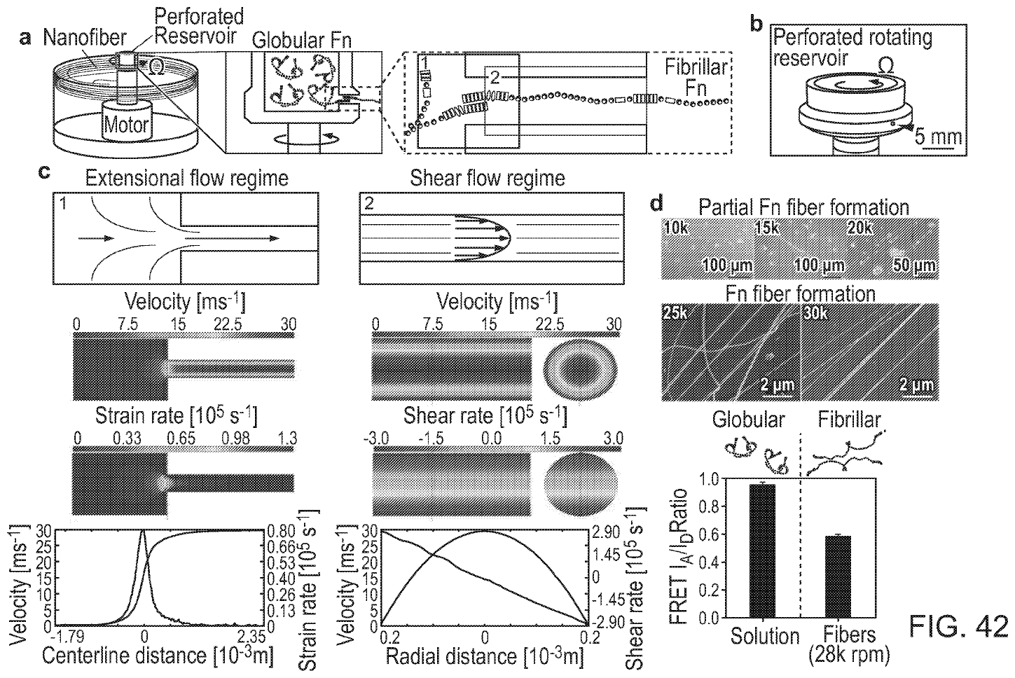

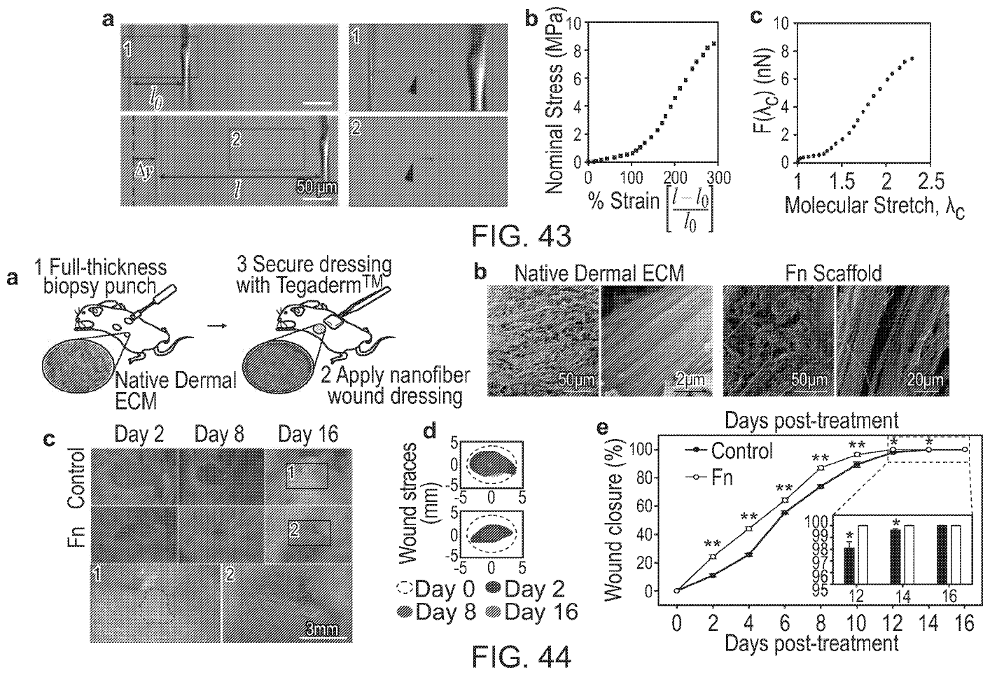

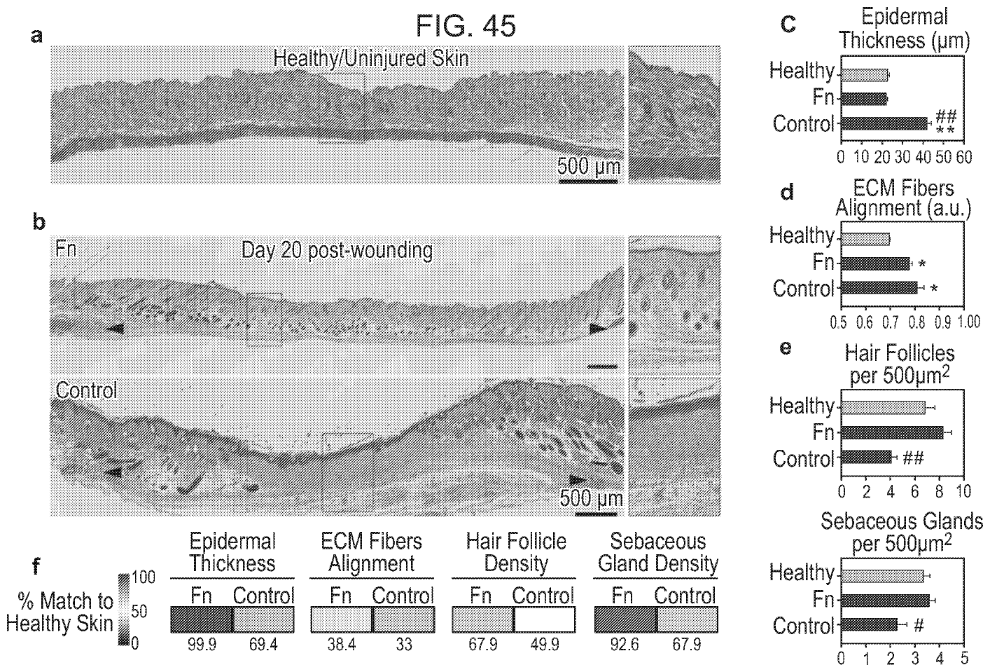

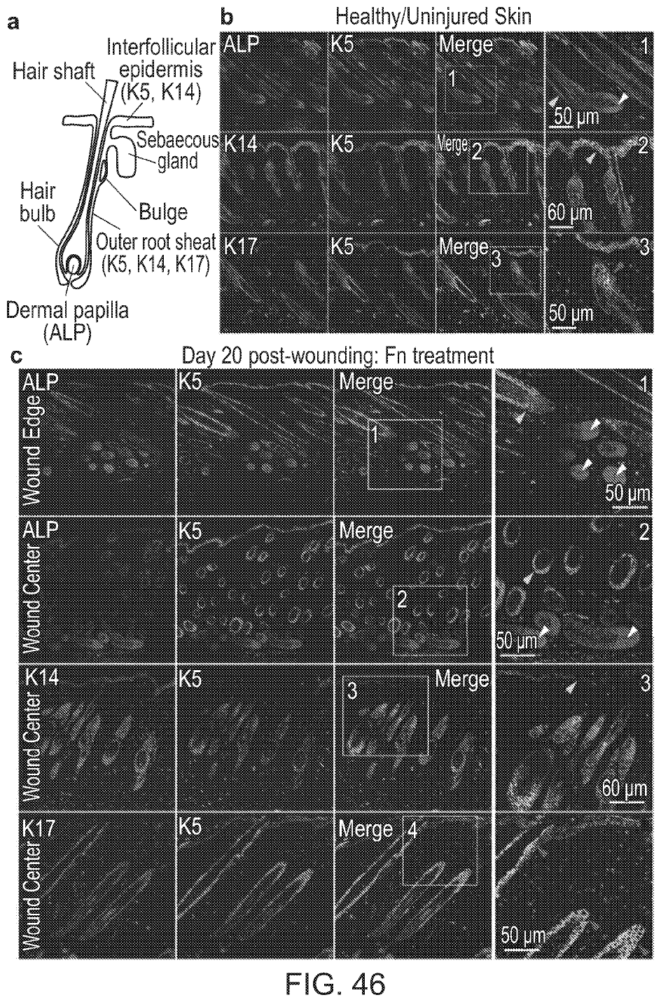

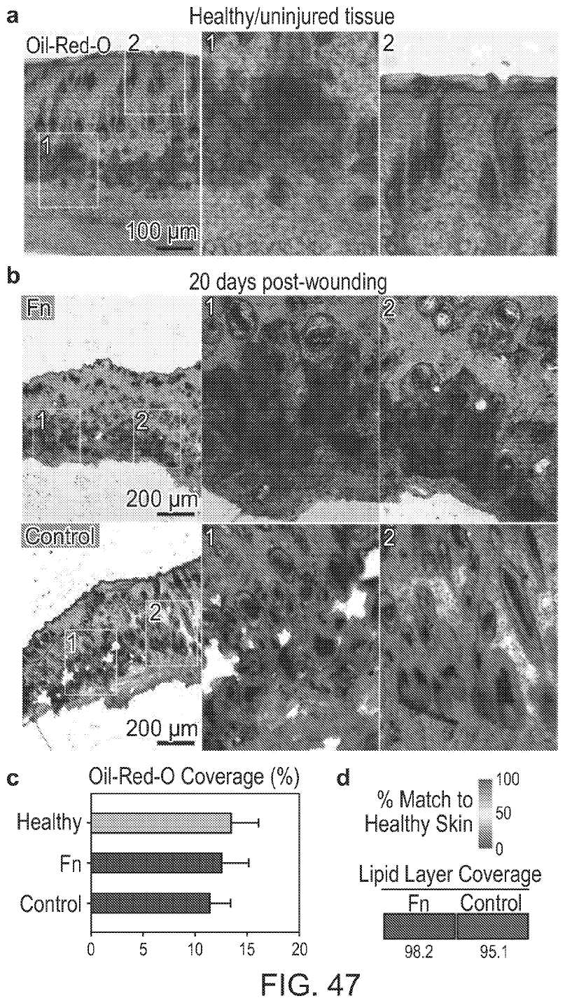

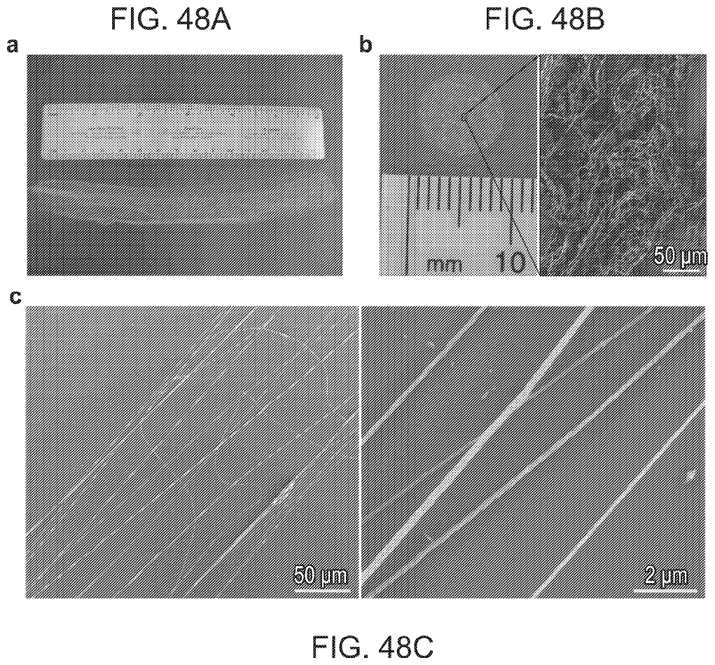

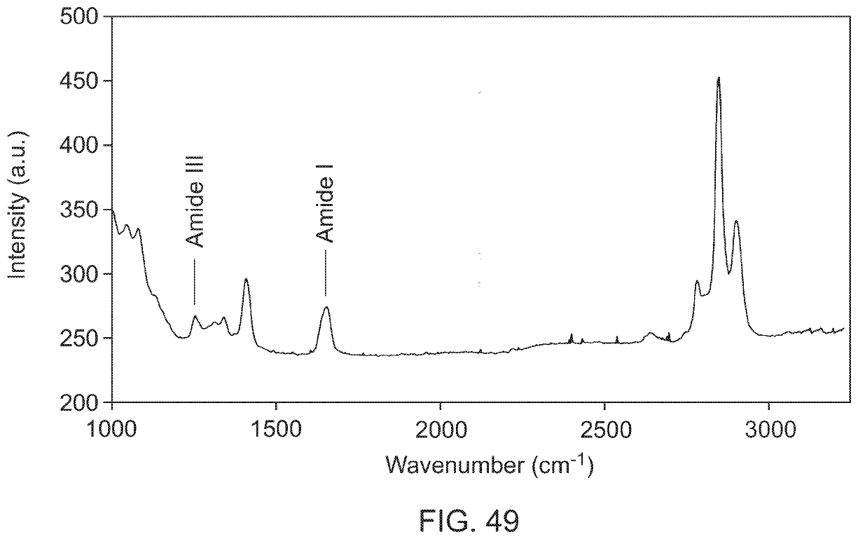

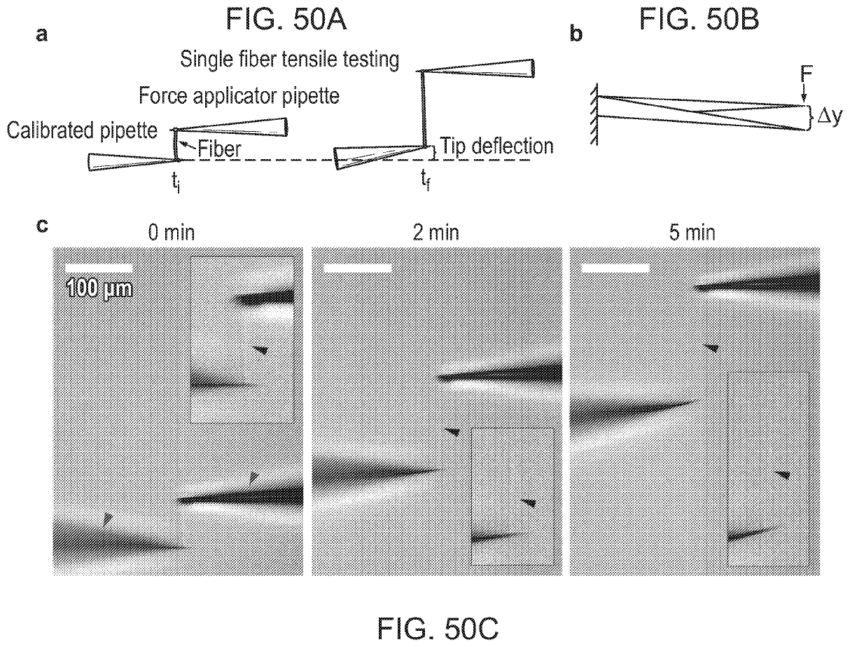

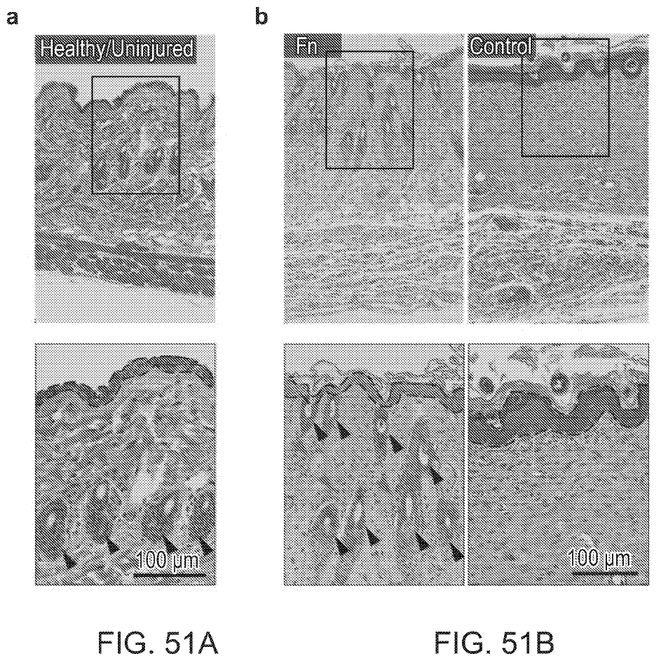

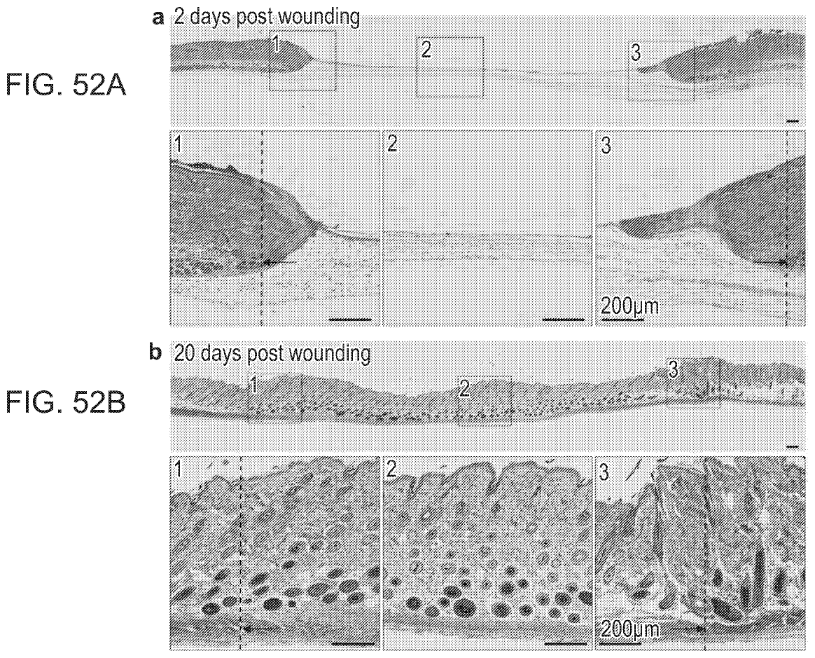

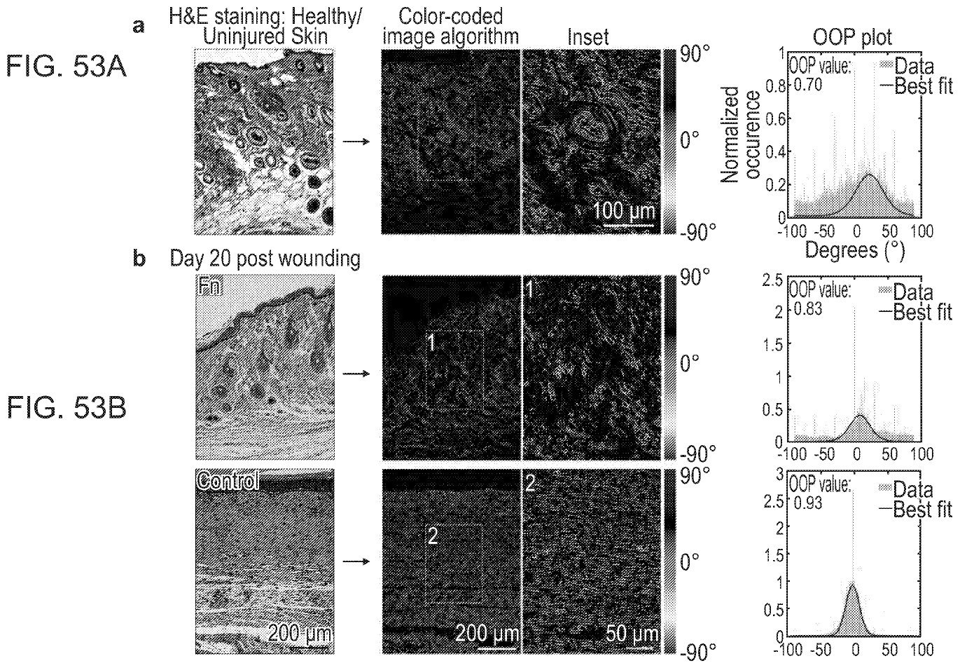

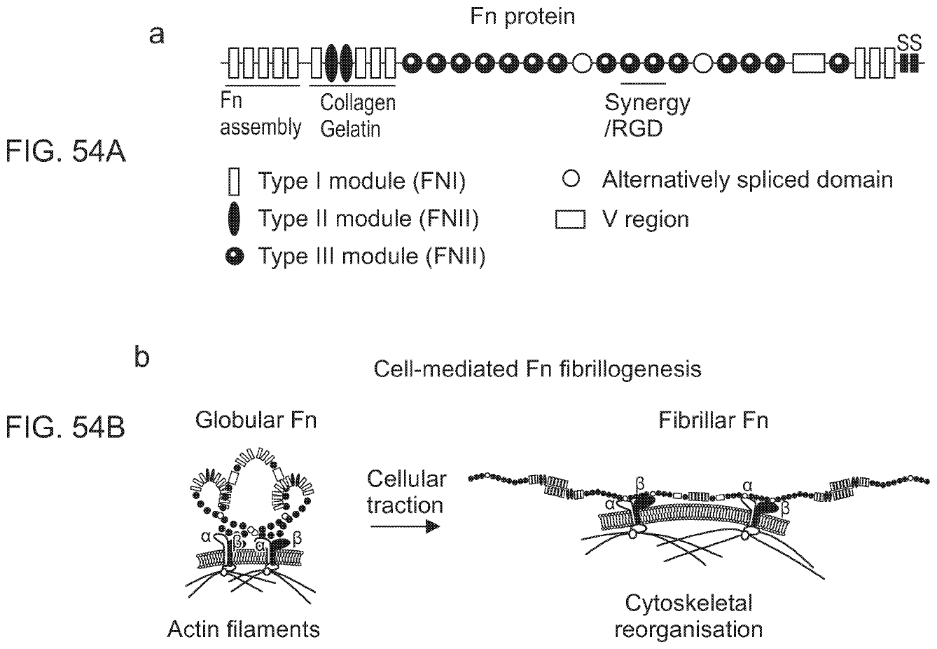

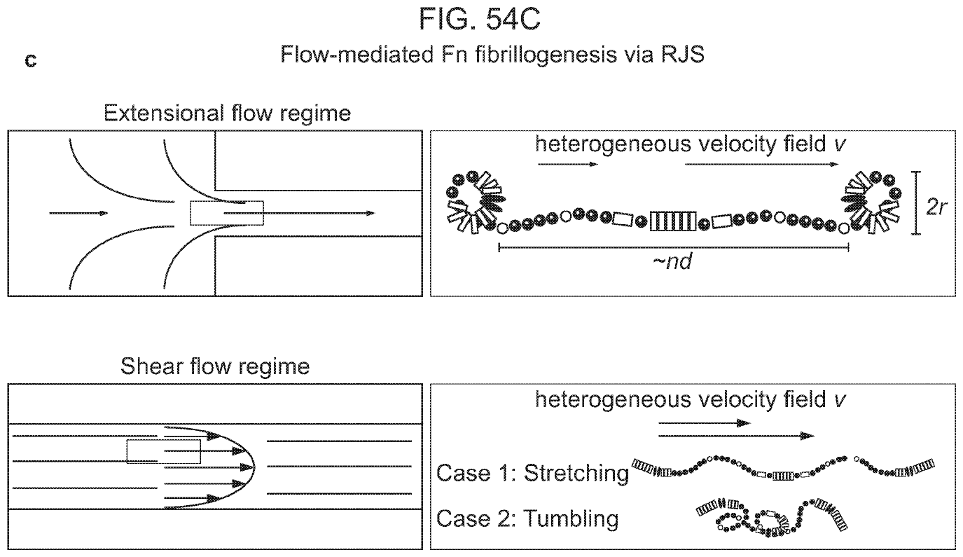

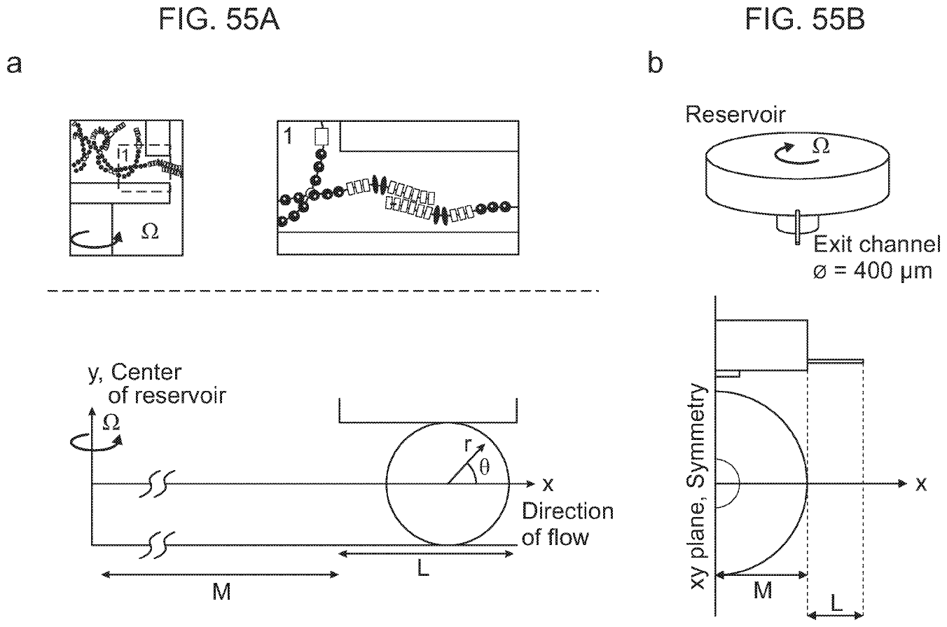

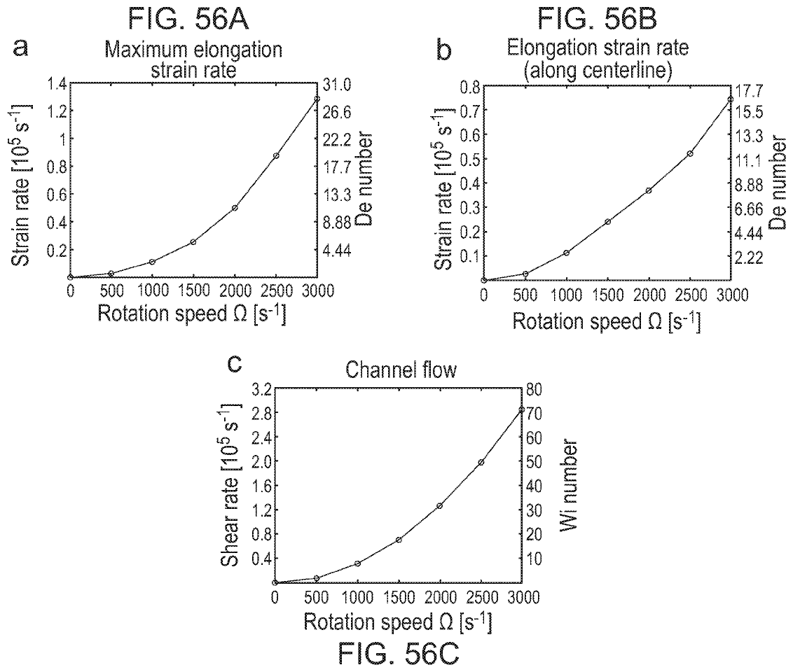

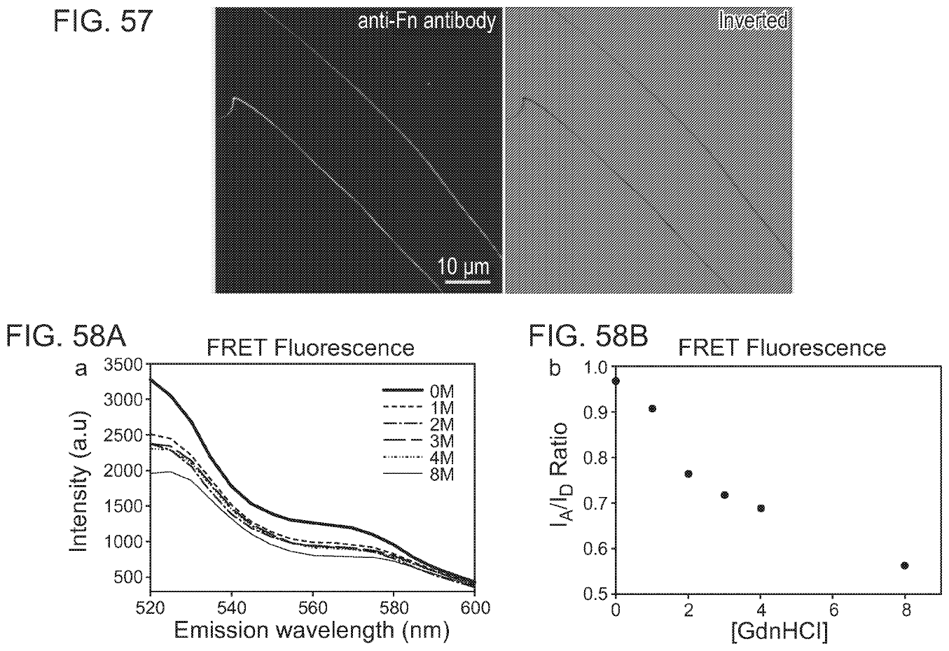

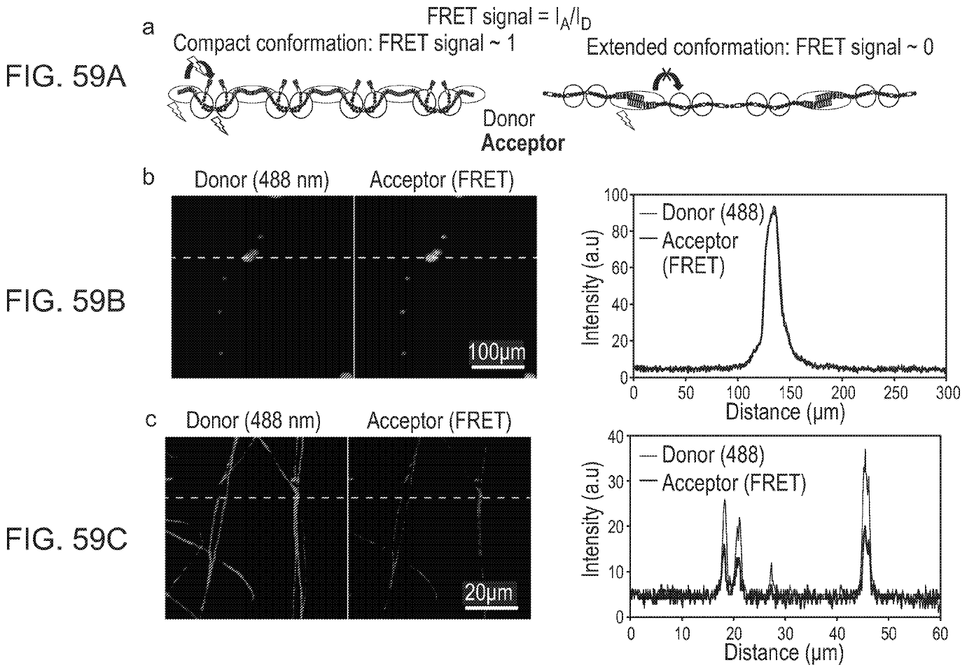

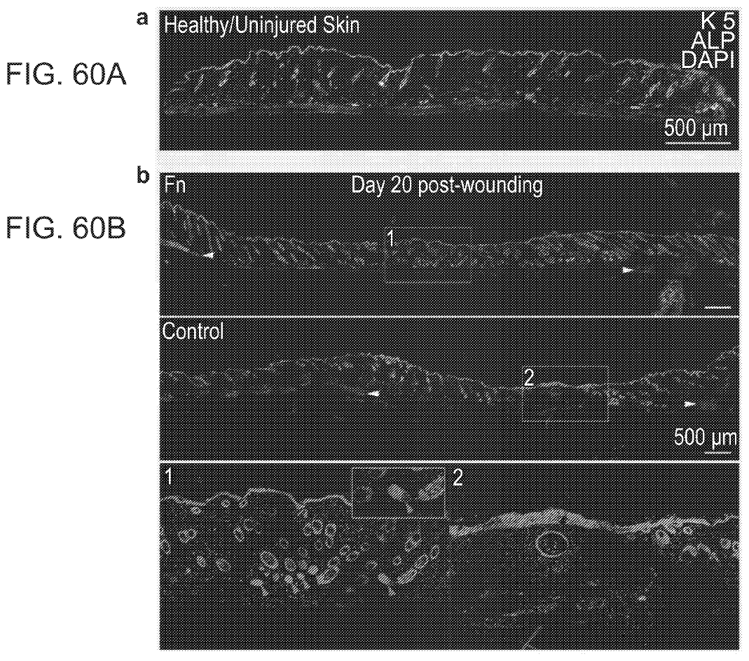

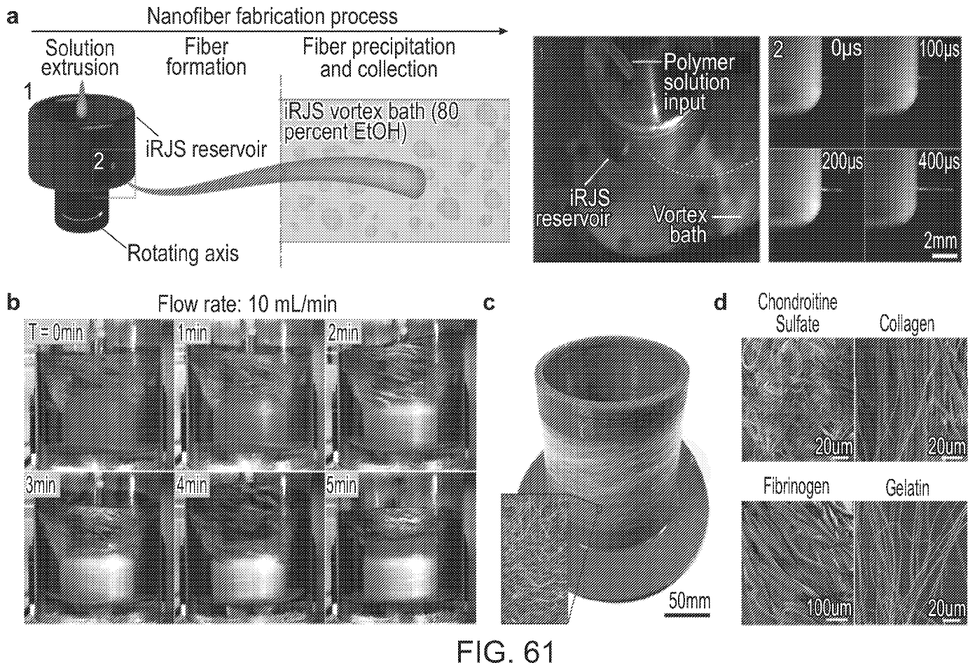

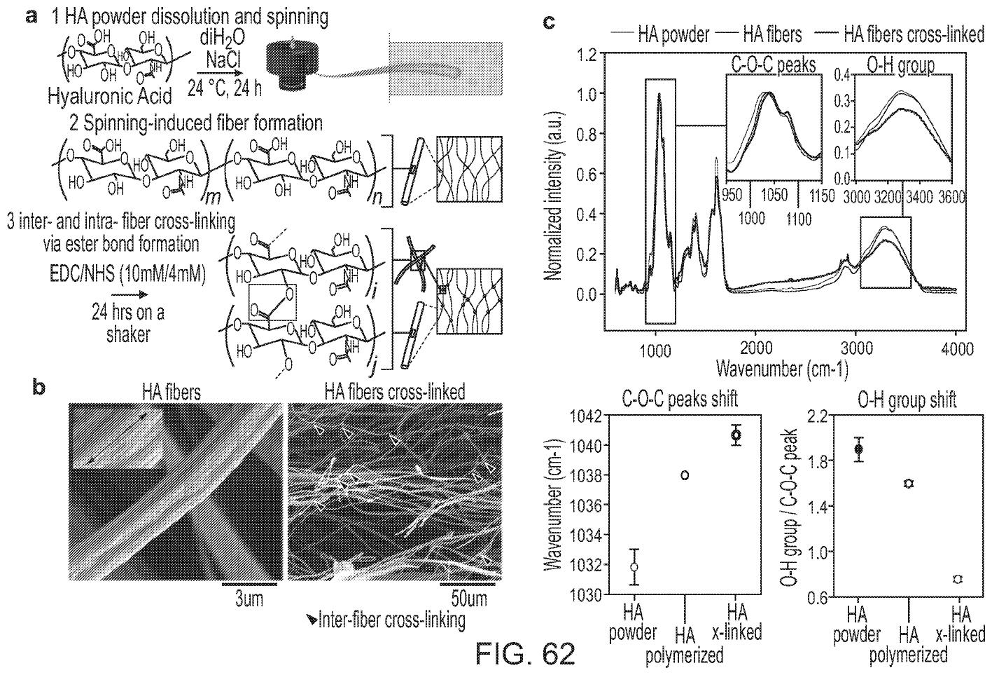

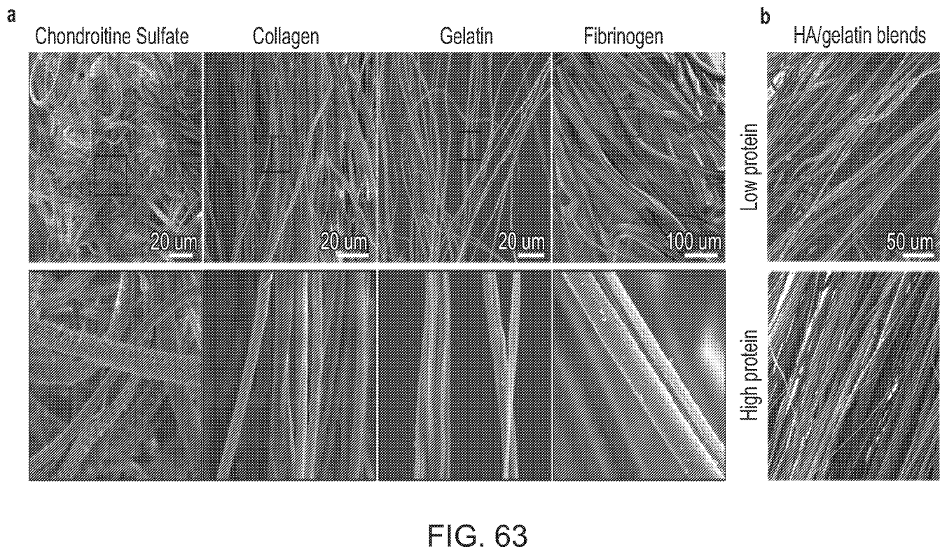

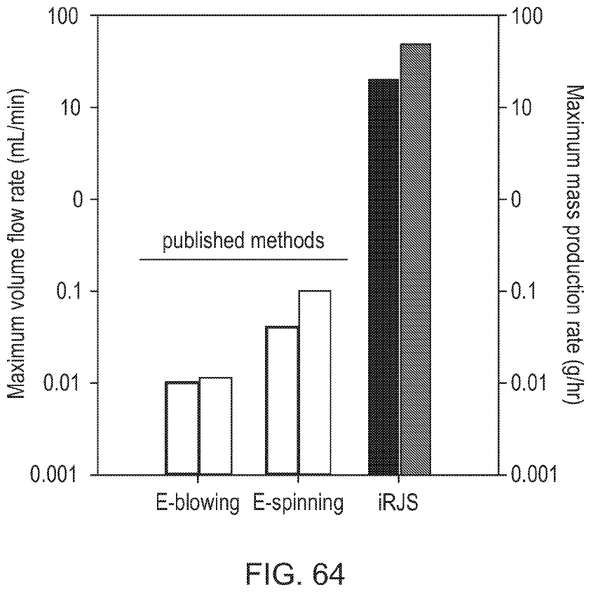

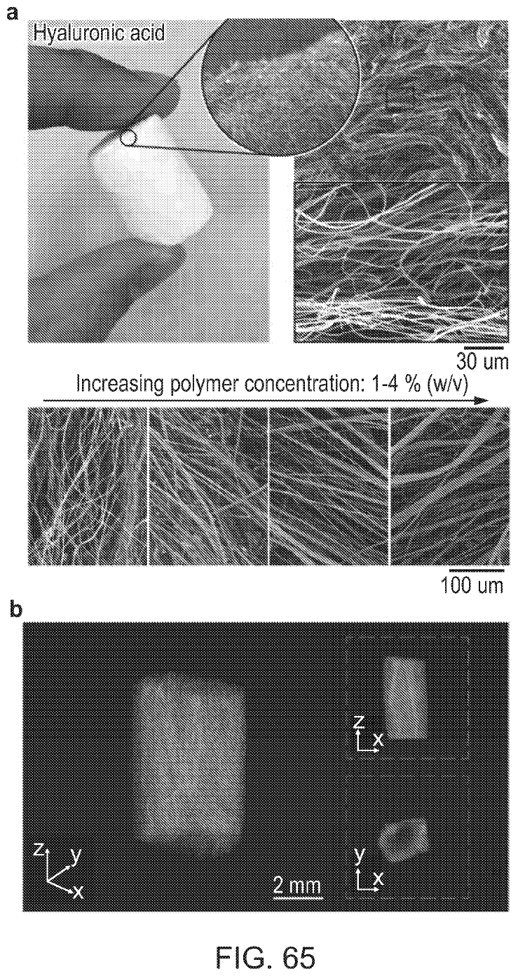

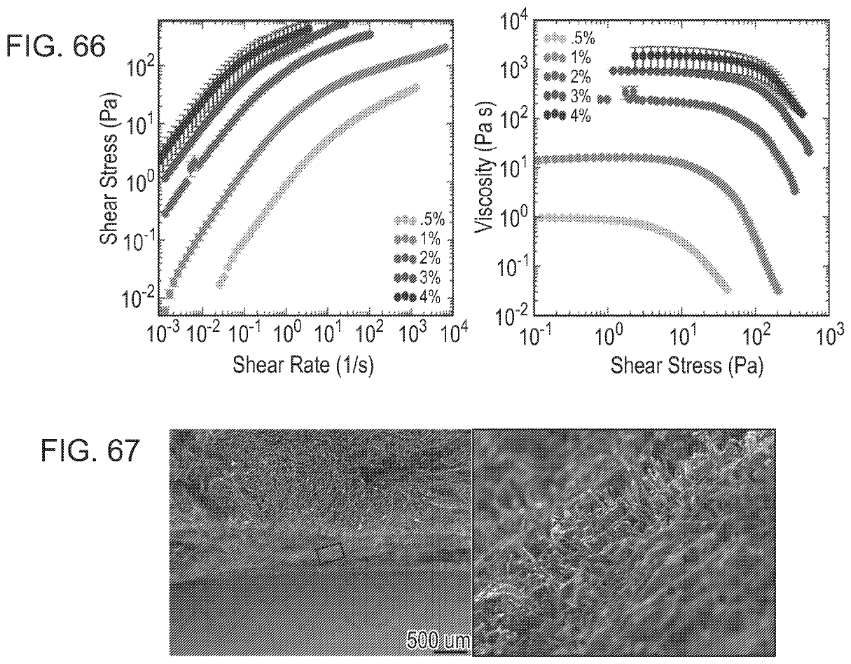

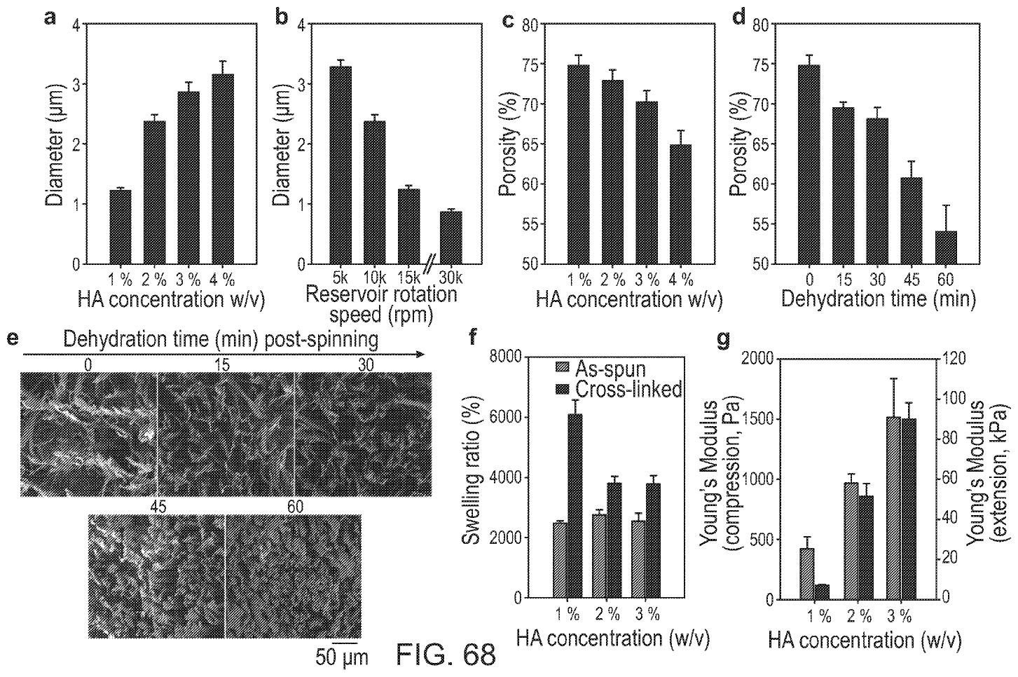

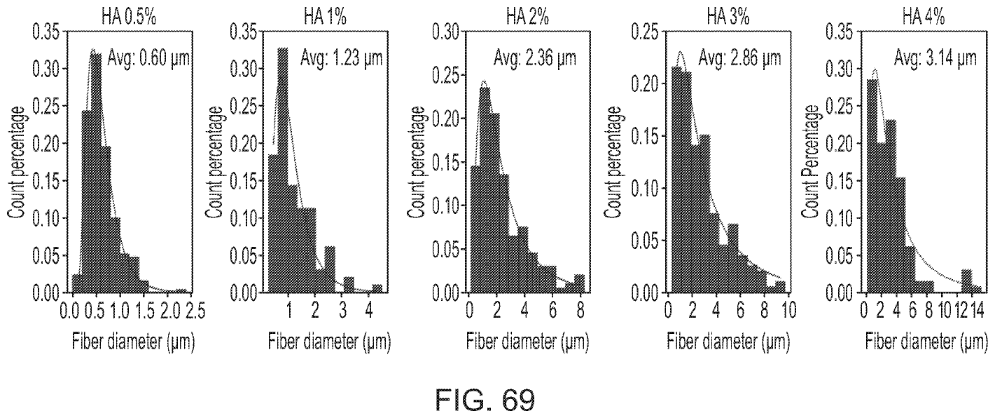

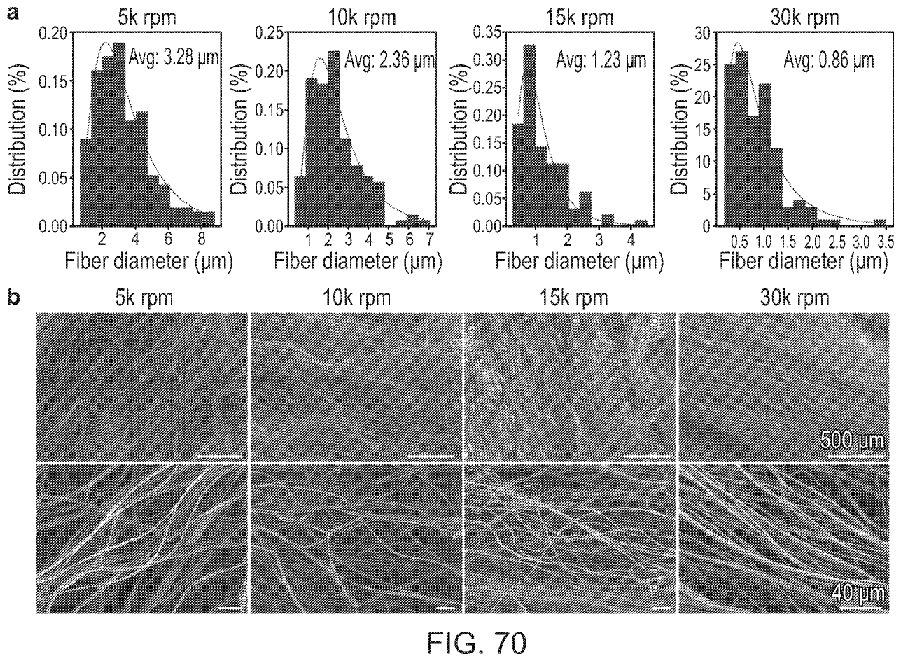

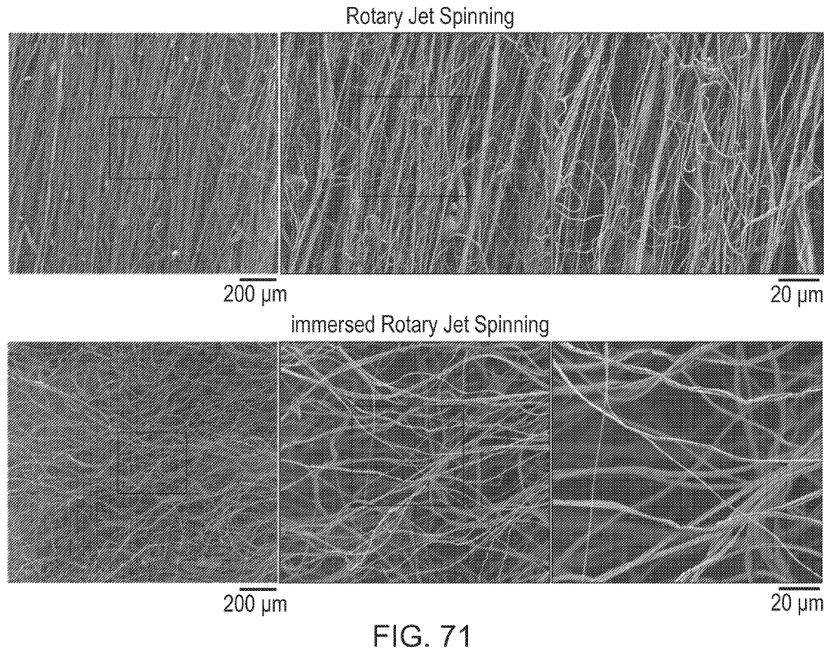

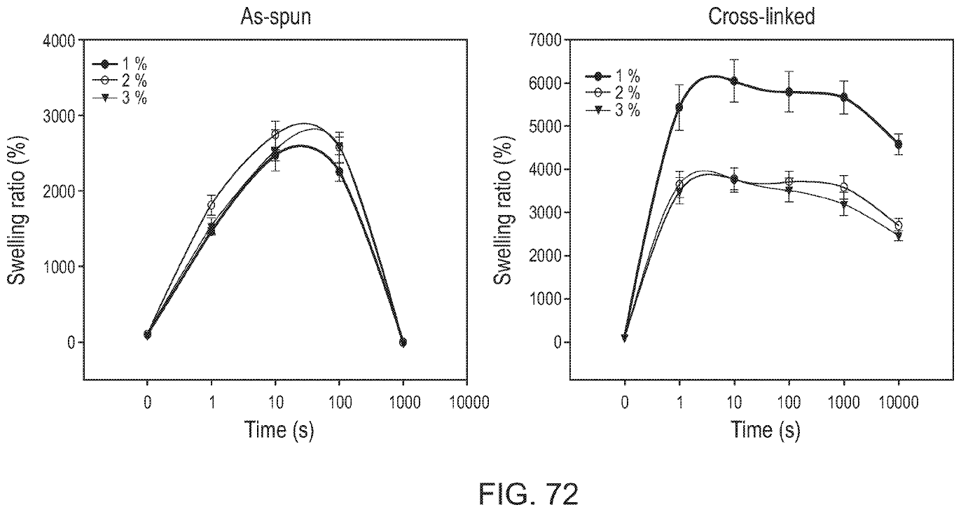

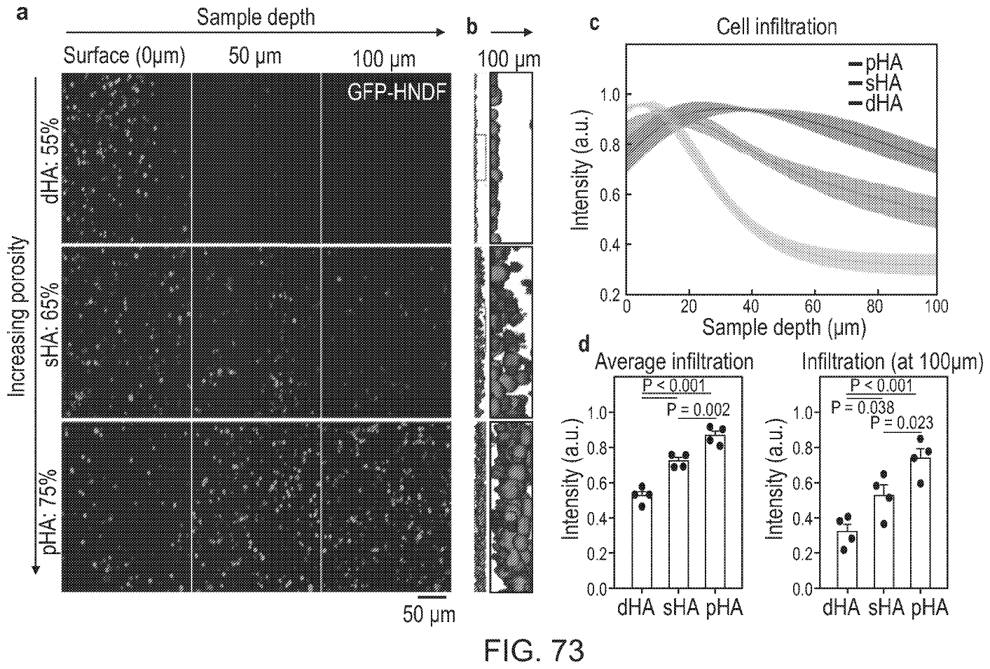

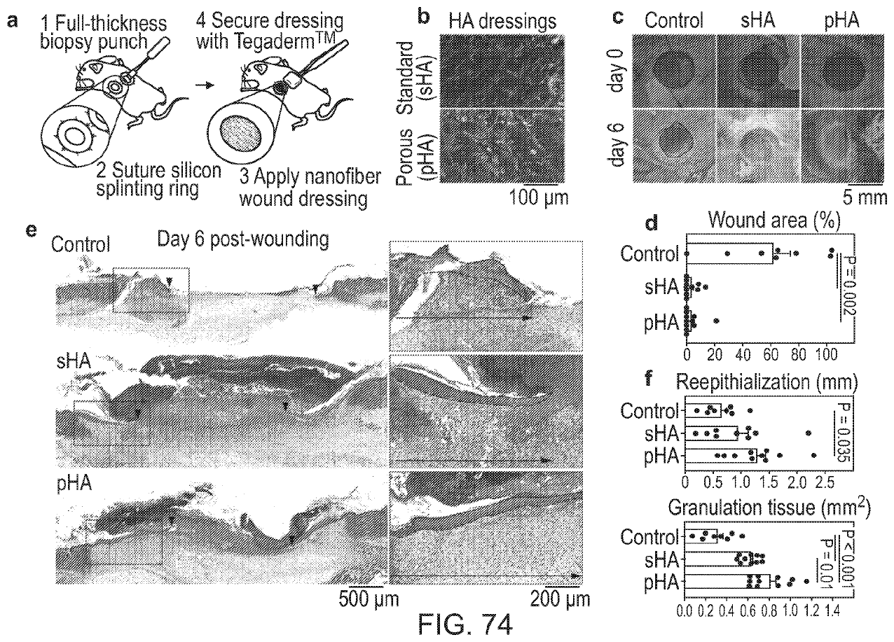

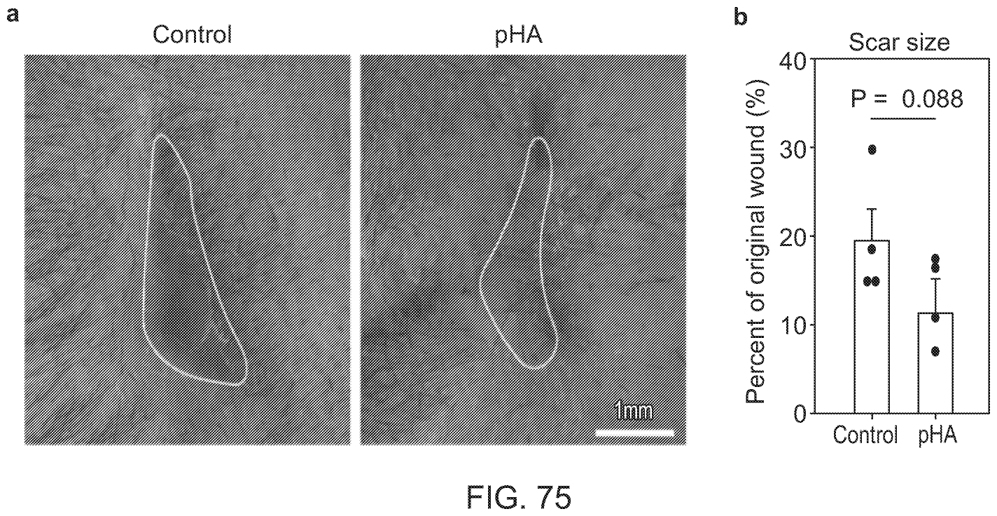

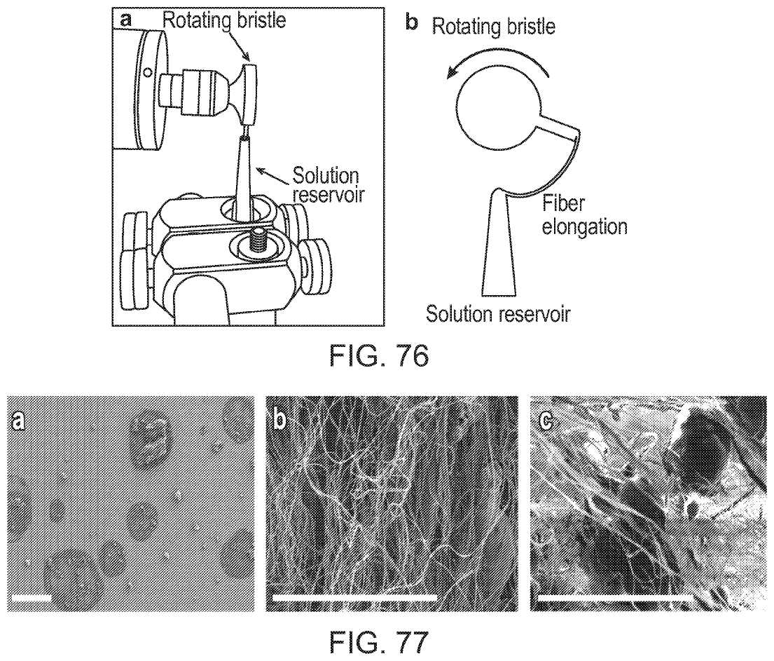

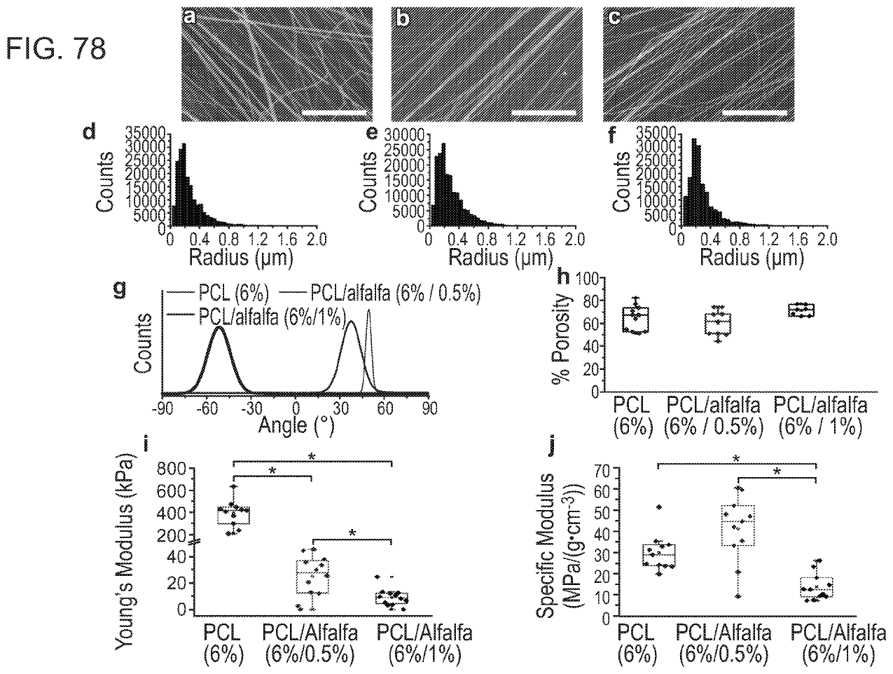

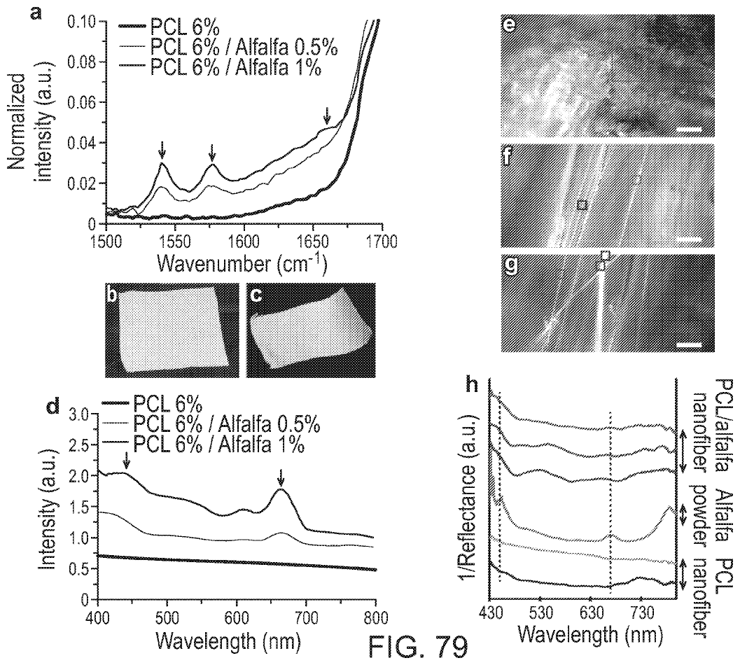

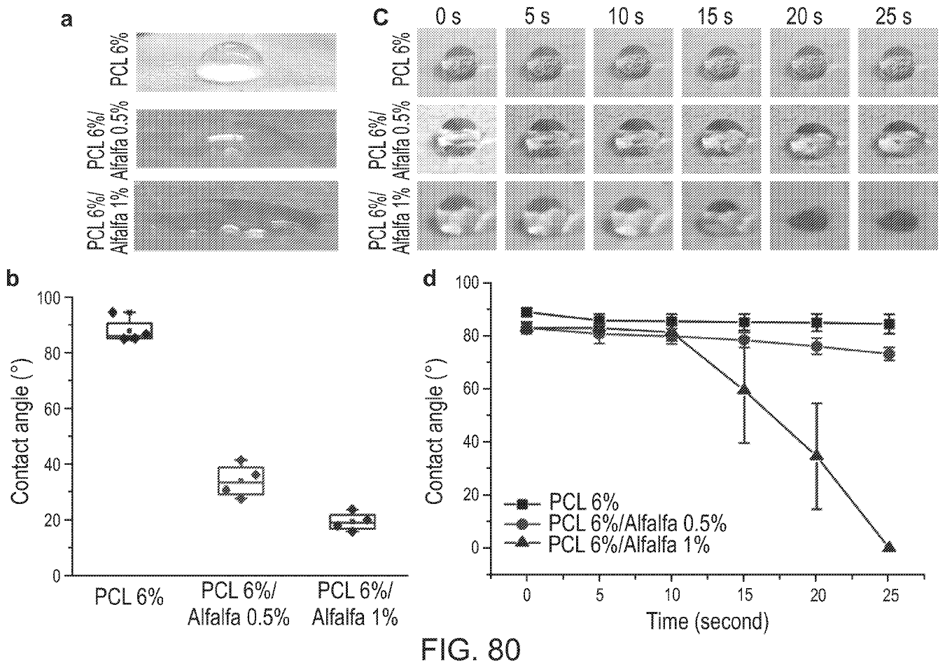

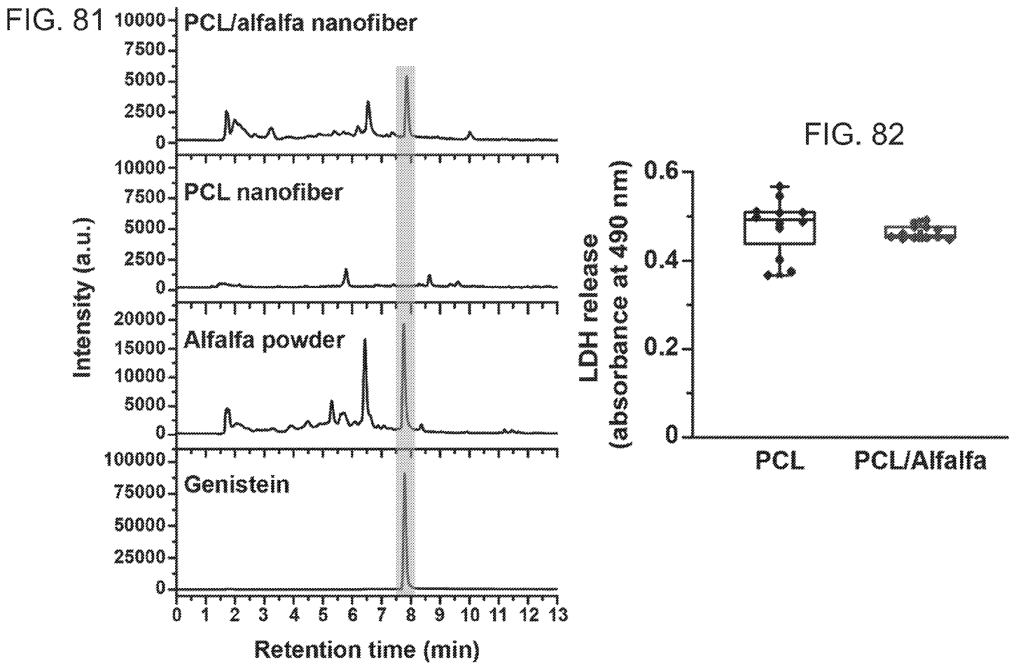

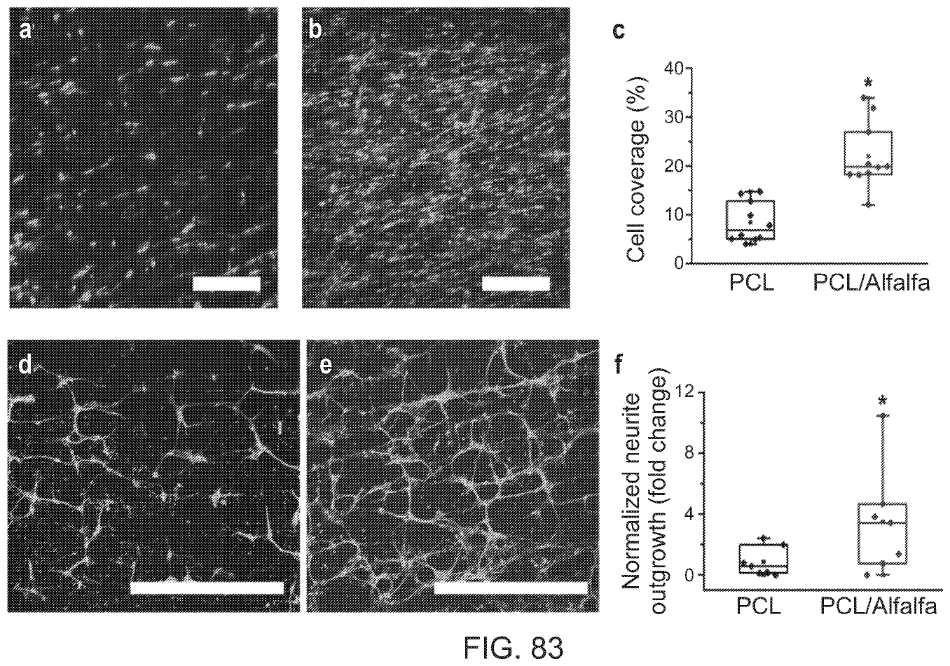

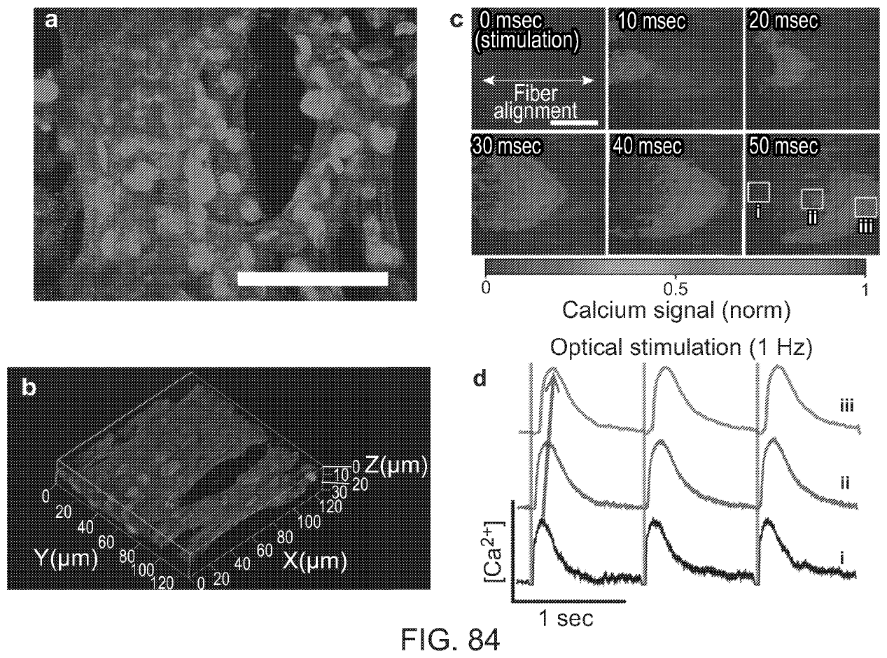

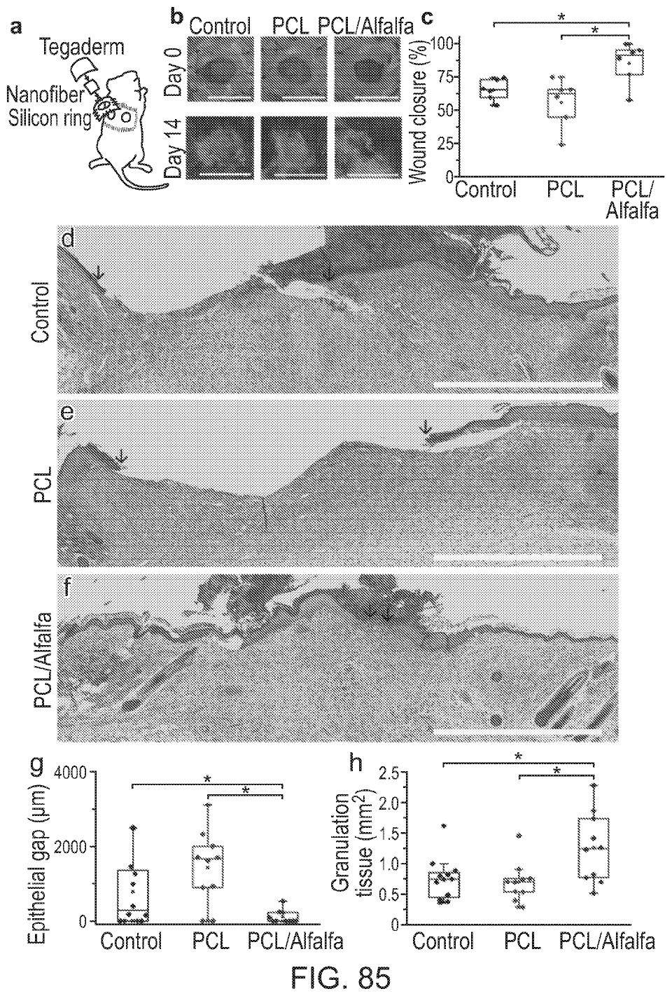

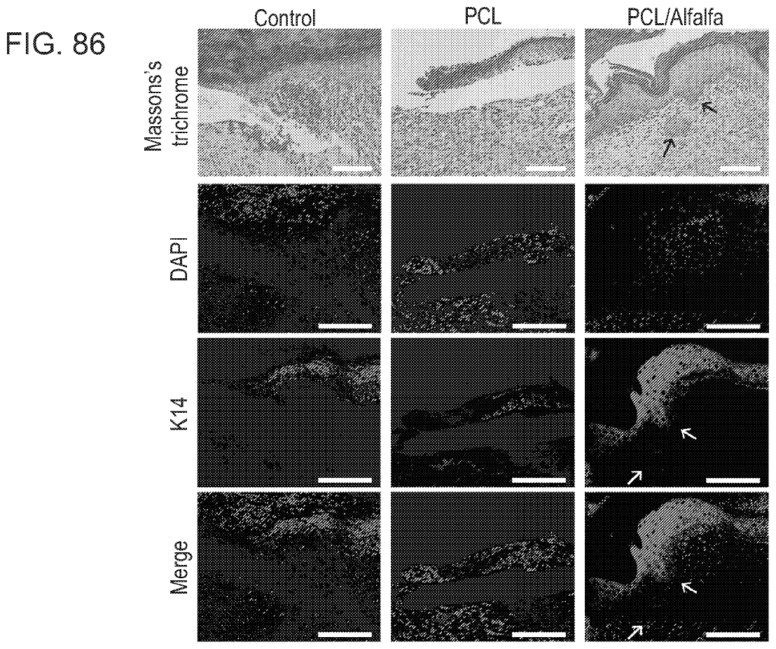

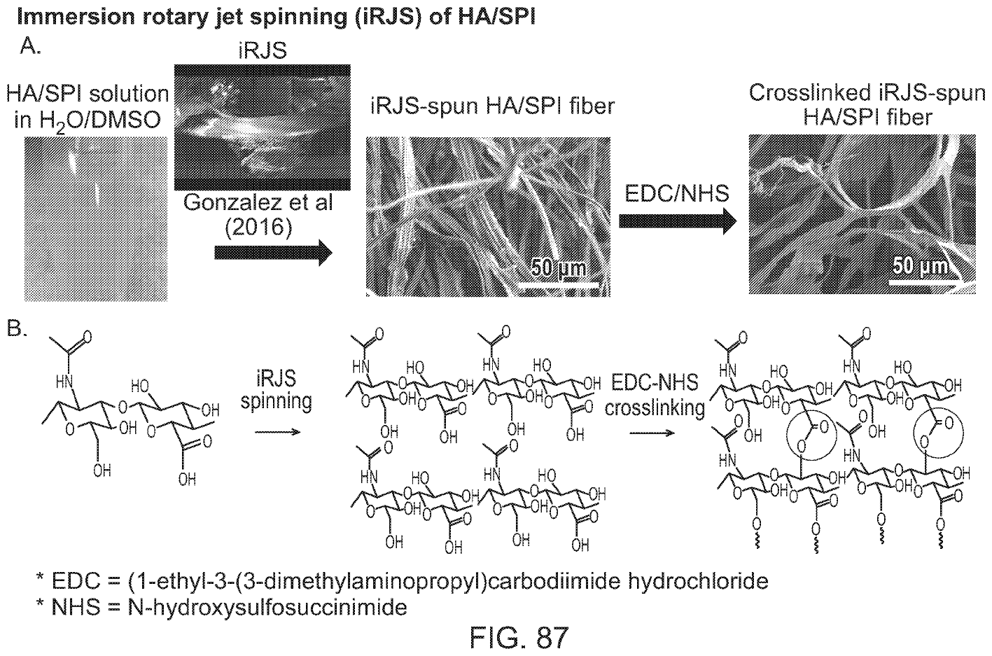

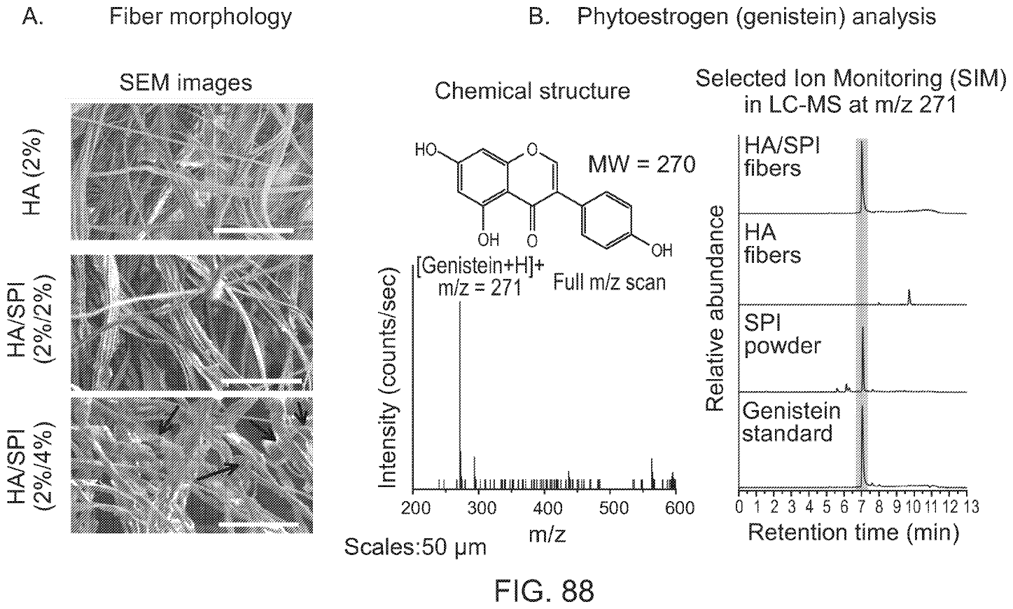

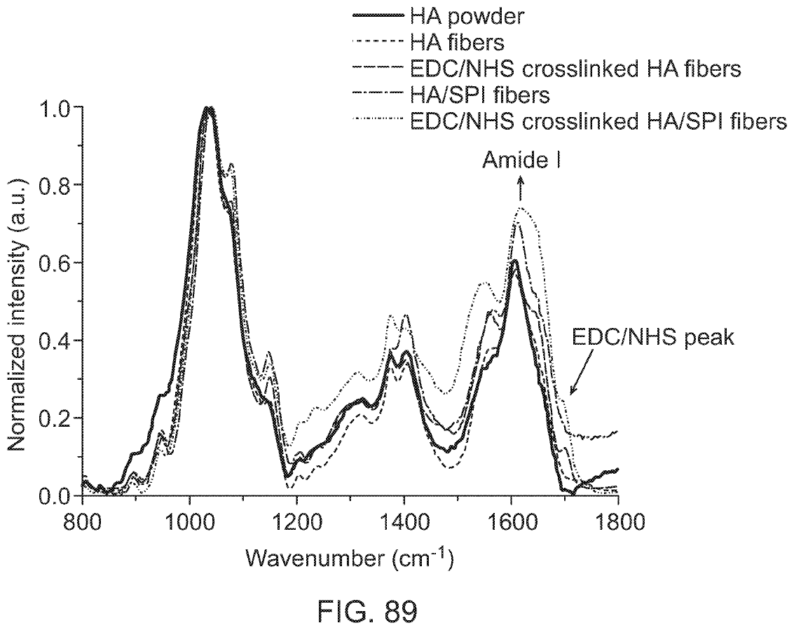

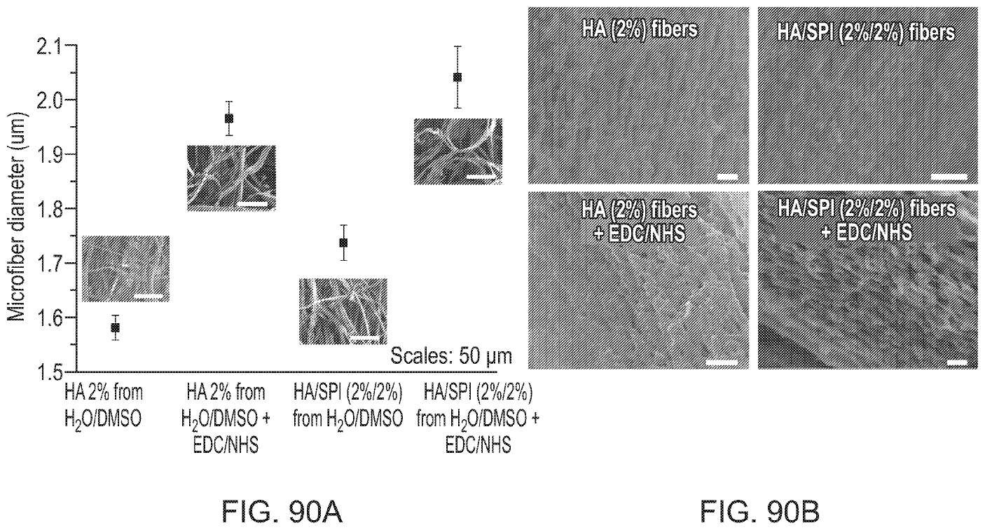

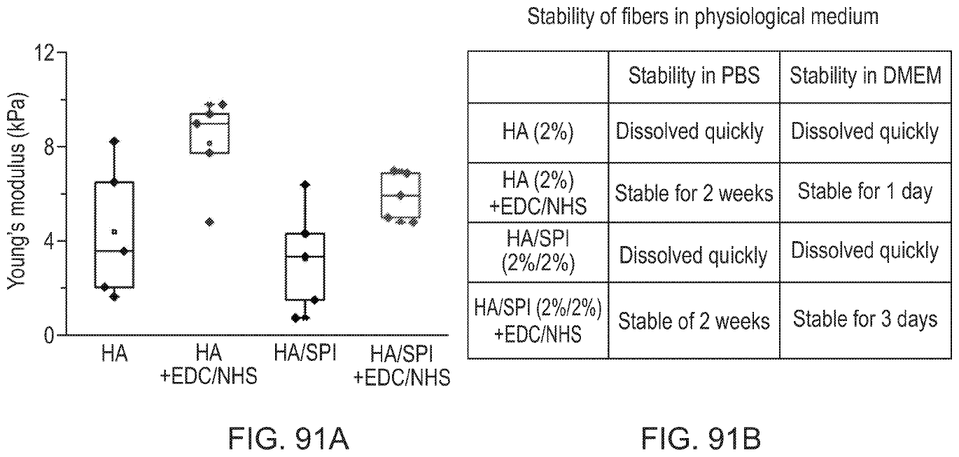

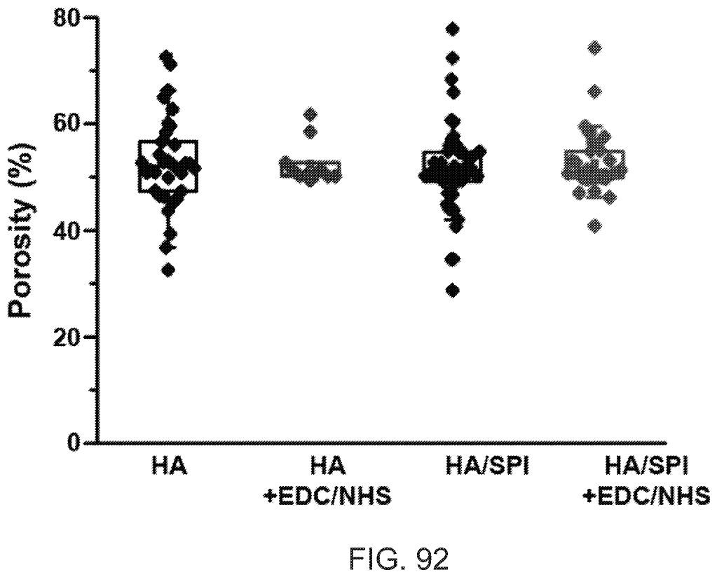

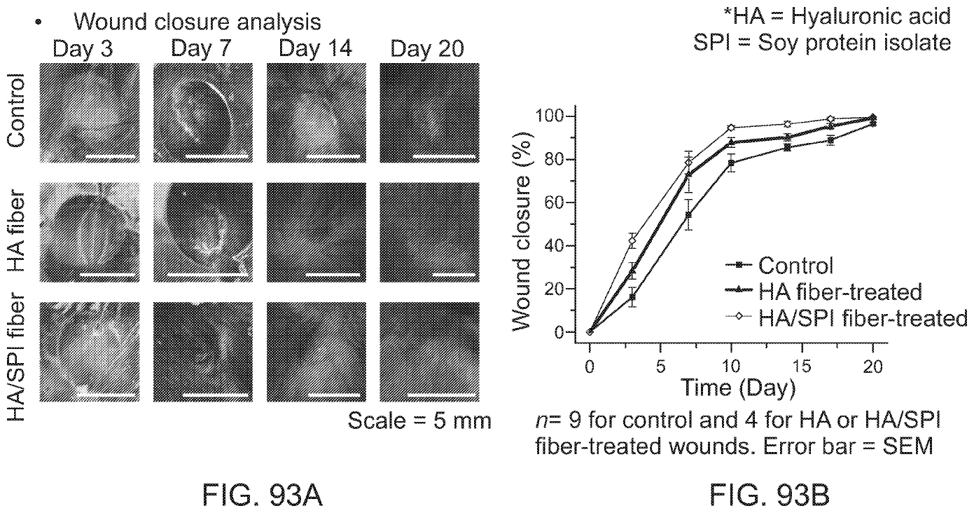

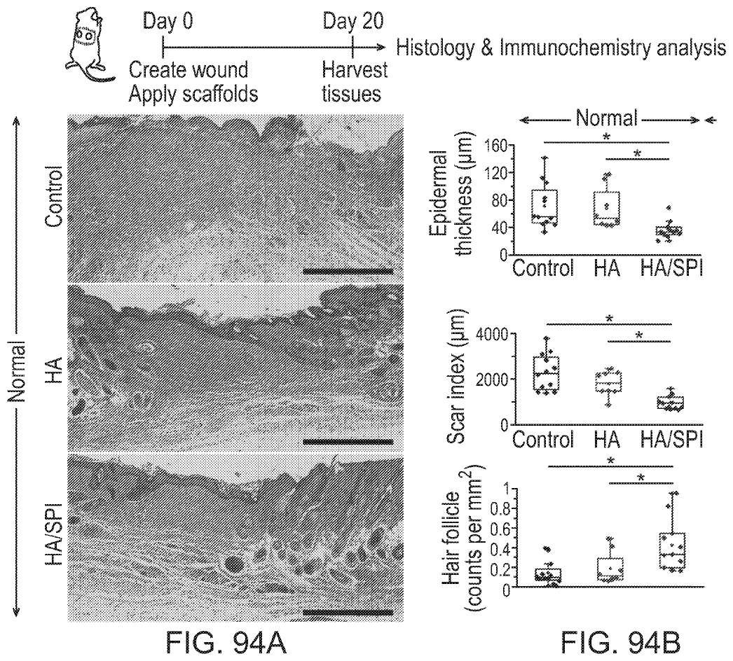

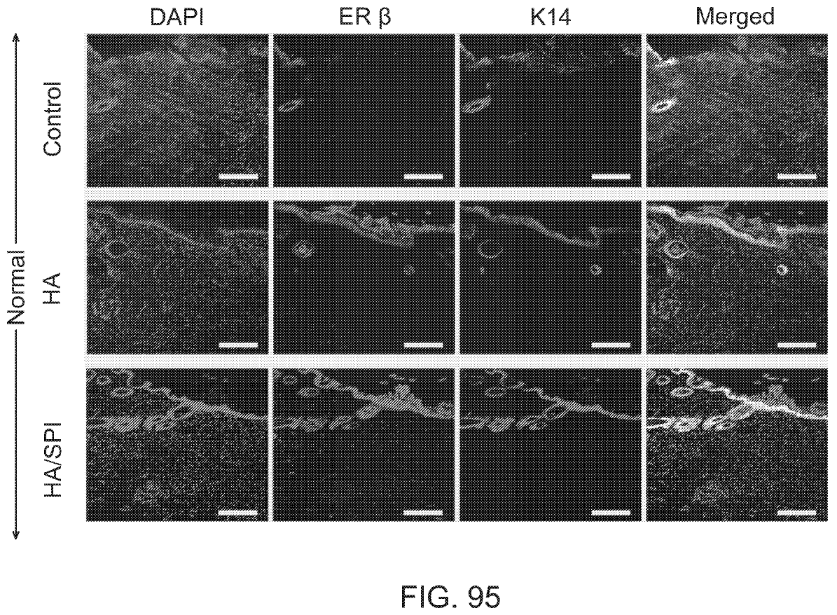

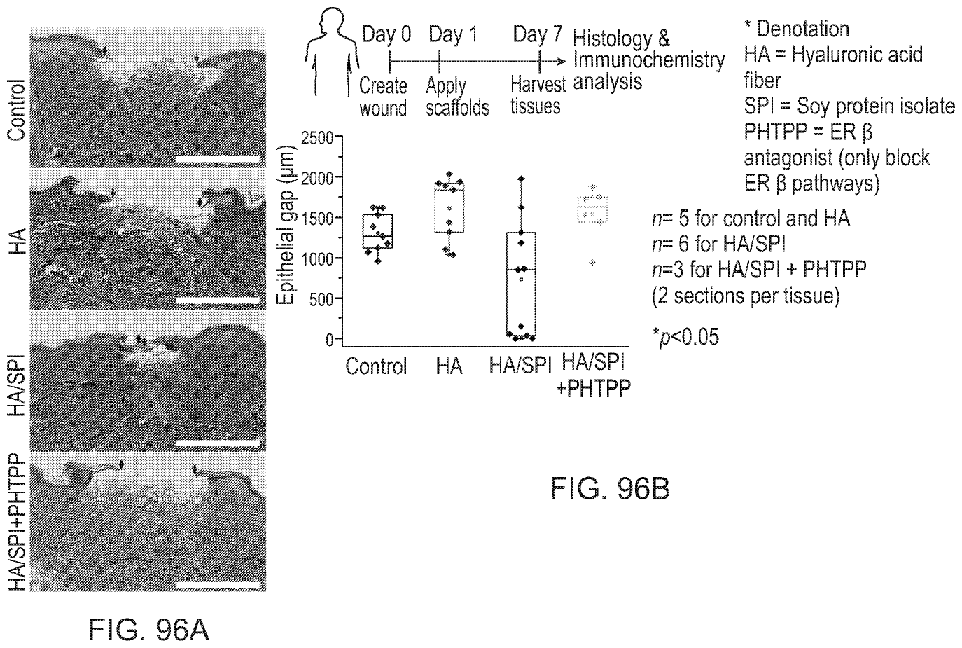

Immunostaining analysis with the Ki-67 antibody—a marker specific to proliferative nuclei—showed that CA/SPH nanofibers induced higher cell proliferation than PCL or CA nanofibers (FIGS. 17A-17I, 18). Nanofiber cytotoxicity was calculated by using a common lactate dehydrogenase (LDH) assay. Both CA and CA/SPH nanofiber scaffolds exhibited low cytotoxicity, with similar values to PCL nanofibers (FIG. 19). It was furthermore observed that the cell surface coverage on the CA/SPH nanofibers was significantly higher than on the PCL and CA nanofibers after 5 days in culture (FIGS. 20A-20L, 21). The CA nanofibers showed greater cell coverage at day 5 and day 15 versus the PCL nanofibers. HNDFs migrated faster on CA-based nanofibers than on PCL nanofibers (FIGS. 22A-L, 23), whilst the addition of bioactive SPH into CA nanofibers resulted in increased cell migration compared to pure CA nanofibers. These results reflect the preferential properties of dermal ECM-mimetic CA-based nanofibers (fiber diameter, pore diameter, and stiffness as shown in FIGS. 10A-10E, 11A, 11B, 12), and underscore the suboptimal properties of PCL. In addition, soy protein has been reported to trigger the expression of extracellular signal-regulated kinase (ERK), transforming growth factor (TGF β1), and integrin β1 that promote cell migration. In an effort to assess cell infiltration, cells were seeded on the surface of nanofiber scaffolds. Cells adhered to nanofibers and started to grow. At day 0, there is no significant difference in cell infiltration between different nanofibers (FIGS. 29A-29F). After 15 days of cell culture, CA-based nanofibers showed an increase in cell infiltration depth compared to PCL nanofibers (FIGS. 24A-24C, 25, 29A-29F) which was again further increased by co-spinning CA with SPH to form CA/SPH nanofibers. As CA-based nanofiber scaffolds have higher pore diameters than PCL nanofibers (FIG. 10B), cells infiltrate faster on CA-based nanofibers. However, there is no significant difference in pore diameters between CA and CA/SPH nanofiber scaffolds, suggesting that the existence of SPH promoted cell migration (FIGS. 22A-22L, 23) and thus cells on CA/SPH nanofibers penetrated faster than CA nanofibers.

Next, immunocytochemical and western blot analysis for integrin β1 were performed to understand the effect of SPH on cell growth and migration. The integrin β1 is ECM protein receptors which regulates the behavior of ECM proteins and cells. It also enables crosstalk with other growth factors and plays a crucial role in tissue repair. During wound healing, dermal fibroblasts migrate to the wound site and express integrin β1 to mature the developing matrix. It has been found that decreased expression of integrin β1 reduces the ability of fibroblasts and keratinocytes to migrate, lay down a collagen matrix, and ultimately enable a wound closure. After 15 days of cell culture, immunocytochemical (FIGS. 26A-26F) and western blot (FIGS. 27 and 28) analyses indicated that the integrin β1 expression was significantly increased on CA/SPH nanofibers, compared to CA nanofibers. These results indicate that soy protein in the CA/SPH scaffolds can trigger the expression of integrin β1 that in turn accelerates the cell migration and the production of new ECM proteins for wound closure. The increased integrin β1 expression by co-spinning CA with SPH (to form CA/SPH nanofibers) is in line with previously published work that reported that soy protein peptides up-regulated the expression of integrin β1 in fibroblasts.

In summary, the in vitro fibroblast studies described herein demonstrated that CA nanofibers supported stronger cell growth, proliferation, migration, and infiltration than PCL nanofibers. These enhanced cellular activities occurred because CA provides a soft and hydrophilic backbone similar to that of a collagen matrix found in native dermal tissue for cell growth. Co-spinning of CA and SPH to form CA/SPH nanofibers accelerated proliferation, growth, migration, infiltration, and integrin β1 expression of HNDFs. Accordingly, it can be extrapolated that CA/SPH nanofibers possess the ability to provide structural and biological cues for promoting wound healing in vivo.

### Example 1H: In Vivo Wound Healing Study in a Rodent Model

To investigate the potency of CA/SPH in vivo, the nanofiber scaffolds synthesized herein were tested on a mouse excisional wound splinting model. Wound contraction was inhibited by suturing a silicon splint to the peripheral edge of the wound in an effort to study the healing process via re-epithelialization and thus improving recapitulation of the wound healing process of humans (FIGS. 30, 31A-31D). Nanofiber scaffolds were held in place with a Tegaderm™ transparent medical dressing film. The control group wounds received no nanofiber treatment and were only covered with the Tegaderm™ transparent medical dressing film. It was observed that CA/SPH nanofibers significantly accelerated in vivo wound closure (FIGS. 32A-32I, 33). On Day 7 after surgery, CA nanofibers showed 42% faster wound closure than the control. The addition of SPH in the CA nanofibers further accelerated wound closure by 21% and showed an overall 72% increase when compared to the non-treated control. After 14 days, the wounds treated with CA/SPH nanofibers were fully closed. Moreover, the wound closure potentiated by CA/SPH nanofibers significantly higher than both the control and CA nanofibers. (FIGS. 32A-32I, 33).

In an effort to further assess the regenerative capacity of the aforementioned treatment conditions, histological analysis of healed tissues was performed at Day 14 post surgery (FIGS. 34A-34D, 35A-35D, 36A-36D). Restoration of the dermal and epidermal layers are key parameters for evaluating wound healing and tissue regeneration. It is commonly analyzed by quantifying the epithelial gap, epithelial thickness, and scar size. H&E (hematoxylin and eosin) staining revealed that CA/SPH nanofiber-treated wounds were re-epithelialized at day 14 post-surgery (FIGS. 35A-35D, FIG. 37). However, wounds from the control and CA nanofiber-treated groups remained open, resulting in epithelial gaps a few hundred micrometers in diameter after 14 days of treatment (FIGS. 34A-34D, FIG. 37). In addition, the control or CA nanofibers-treated wounds exhibited significantly thicker epidermis layers than CA/SPH nanofiber-treated wounds, indicating slower regeneration of the epidermis (FIGS. 33A-33D, FIG. 37). However, it should be noted that the epidermal thicknesses of CA/SPH nanofibers-treated wounds was still higher than that of healthy tissues (FIGS. 36A-36B, 37). The scar sizes were measured using a quantitative scar index (FIG. 38). It was found that CA/SPH nanofibers significantly reduced the scar size compared to control or CA nanofibers after 14 days of treatment. Lastly, the alignment of the newly synthesized collagen in the dermis was calculated (FIG. 6e). The dermal collagen was significantly less aligned in CA/SPH nanofiber-treated wounds than control or CA nanofiber-treated wounds. However, the alignment of CA/SPH nanofiber-treated wounds was still higher than that of healthy tissues that possess typically basket-woven fiber organization. In line with the in vitro results described herein, the in vivo data supported the inventors' hypothesis that both a nanofibrous architecture and bioactive soy protein accelerated wound closure and supported regeneration of the dermal and epidermal layers. These observation also corroborate previously published results in which ECM-mimetic peptide and phytoestrogens in soy protein promoted re-epithelialization and dermal tissue regeneration.

The studies described above represent the first fabrication and optimization of cellulose acetetate/soy protein hydrolysate (CA/SPH) nanofibers. The studies described herein also represent the first of these nanofibers produced using a rotary jet spinning (RJS) system. CA and SPH molecules were homogeneously distributed along the nanofibers for equal functionality at the fiber surface. Using CA as a co-spinning polymer enabled recapitulation of fiber morphology, fiber diameter, pore diameter, and stiffness of the native extracellular matrix (ECM) thus creating optimal conditions for dermal fibroblasts to thrive. Co-spinning of CA nanofibers with SPH enhanced surface roughness, hydrophilicity, and water absorption capacity. The in vitro study indicated that CA/SPH nanofibers increased proliferation, growth, migration, and infiltration of fibroblasts and exhibited low cytotoxicity, compared to both PCL and CA nanofibers. The addition of SPH into CA nanofibers further up-regulated the expression of integrin β1, which has been attributed to enhanced cell migration and tissue regeneration. Finally, the in vivo mouse studies revealed that CA/SPH nanofibers accelerated in vivo wound closure and tissue regeneration in comparison to CA nanofibers or the non-treated control. Both ECM-mimetic peptides and phytoestrogens in soy protein may play a role in facilitating the healing process, potentially via multiple mechanisms including integrin β1 signaling, estrogen-mediated pathways, and/or anti-inflammatory activity.

Altogether, the findings of the studies described herein confirmed the utility of CA/SPH nanofibers for enhanced wound healing. These data demonstrate that phytoestrogens in soy protein-based nanofibers may also play a role in facilitating wound healing via estrogen-mediated pathways. The inventors have also surprisingly discovered RJS-spun CA/SPH nanofibers have higher production rate and better control of fiber morphology without an additional modification or high-voltage electric fields in the system, when compared to the existing electro-spun soy-based nanofibers.

### Example 2: Engineered Fetal-Inspired Regenerative Polymeric Fiber Scaffolds and Methods of Use Thereof—Production-Scale Fibronectin Nanofibers Promote Regeneration of Hair Follicles and Enhance Wound Healing in a Dermal Mouse Model

During embryogenesis, scarless wound healing is a regularly occurring process observed through the end of the second trimester (Rowlatt, U. Virchows Arch A Pathol Anat Histol 381, 353-361 (1979)). Although the mechanisms that regulate this regenerative phenotype are not fully understood, several spatiotemporal differences of the extracellular microenvironment, including differences in extracellular matrix proteins, such as fibronectin, collagen type I, and hyaluronic acid, have been observed in fetal and postnatal wounds (Coolen, N. A., Schouten, K., Middelkoop, E. & Ulrich, M. M. W. Arch Dermatol Res. 2010 January; 302(1):47-55. Epub 2009 Aug. 23 doi:10.1007/s00403-009-0989-8; Longaker, M. T. et al. J Pediatr Surg 24, 799-805 (1989)). Consequently, biomaterials that attempt to recapitulate the biophysical and biochemical properties of fetal skin have emerged as promising pro-regenerative strategies. The extracellular matrix (ECM) protein fibronectin (Fn) in particular is involved in gestational wound healing in contrast to adults

Fn exists in two distinct conformations in vivo: a globular, soluble state and an extended fibrillary state. While globular Fn has been shown to stimulate angiogenesis and reduce the inflammatory response, resulting in an increase in wound closure rate (Qiu, Z., Kwon, A. H. & Kamiyama, Y. J Surg Res 138, 64-70 (2007); Hamed, S. et al. J Invest Dermatol 131, 1365-1374 (2011)), there is limited information on how fibrillar Fn—the highly upregulated form in fetal wound microenvironments—can be leveraged as a material for wound healing. Fibrillar Fn is critical during tissue repair (To, W. S. & Midwood, K. S. Tissue Repair 4, 1755-1536 (2011)), and its structural stability in a proteolytic environment, characteristic of cutaneous wounds (Clark, R. A., Ghosh, K. & Tonnesen, M. G. J Invest Dermatol 127, 1018-1029 (2007)) suggest advantages for promoting robust cellular ingrowth and directing pro-regenerative cell function in the wound. However, manufacturing fibrillar Fn remains an engineering challenge, as the available chemical (Williams, E. C., Janmey, P. A., Johnson, R. B. & Mosher, D. F. J Biol Chem 258, 5911-5914 (1983); Sakai, K., Fujii, T. & Hayashi, T. J Biochem 115, 415-421 (1994)), mechanical (Ejim, O. S., Blunn, G. W. & Brown, R. A. Biomaterials 14, 743-748 (1993); Smith, M. L. et al. PLoS Biol 5 (2007)) or extrusion (Raoufi, M. et al. Nano Lett 15, 6357-6364, doi:10.1021/acs.nanolett.5b01356 (2015)) methods of producing fibers are limited to small (˜mm) scales. In order to recapitulate the Fn-rich fetal microenvironment at a scale suitable for clinical applications, new methods are required for the production and assembly of fibrillar Fn networks. It was reasoned that nanofiber manufacturing techniques such as rotary jet spinning (RJS) could be employed for the bulk production of Fn scaffolds. The RJS is indeed distinct among other nanofiber manufacturing techniques, as it utilizes centrifugal forces, instead of electric field gradients or high solution temperatures (Reneker, D. H. & Yarin, A. L. Polymer 49, 2387-2425, doi:http://dx.doi.org/10.1016/j.polymer.2008.02.002 (2008); Huang, Z.-M., Zhang, Y.-Z., Kotaki, M. & Ramakrishna, S. Composites science and technology 63, 2223-2253 (2003)), to eject a biopolymer jet from a micron-sized orifice to produce nanoscale fibers (Badrossamay, M. R., Mcllwee, H. A., Goss, J. A. & Parker, K. K. Nano Lett 10, 2257-2261 (2010); Badrossamay, M. R. et al. Biomaterials 35, 3188-3197 (2014)). Its process parameters such as nozzle diameter and spinning velocity can be tuned for different material types, improving morphological quality of fibers (Mellado, P. et al. Applied Physics Letters 99, 203107, doi:10.1063/1.3662015 (2011); Golecki, H. M. et al. Langmuir: the ACS journal of surfaces and colloids 30, 13369-13374, doi:10.1021/la5023104 (2014)). It was thus hypothesized that the centrifugal forces of the RJS could be used to generate fluid strains necessary to unfold the soluble, globular Fn molecule, facilitating fibrillogenesis and protein network formation. The bulk production capability of the RJS could enable assembly of large sheets of fibrillar Fn, required for the development of regenerative materials.

As described in this example and in U.S. Patent Publication No. 2013/0312638, it has been demonstrated that RJS can serve as a platform to fabricate centimeter-wide thick (>100 μm) wound dressings out of pure fibrillar Fn. Analytical and computational simulations developed in parallel validate how the extensional and shear flow regimes in the rotating reservoir are sufficient to extend the globular conformation, thus enabling flow-induced fibrillogenesis. Using fluorescence resonance energy transfer (FRET), it was confirmed that Fn molecular unfolding induced by the hydrodynamic forces applied to the protein. Fn scaffolds were then investigated as a bioactive material strategy for accelerating wound closure and promoting skin tissue restoration in a full-thickness wound mouse model. To evaluate the regenerative potency of the Fn dressings, treated-skin tissues are systematically compared to healthy skin by assessing restoration of basic structural components like hair follicles and sebaceous glands. Non-treated wounds are added as a comparison control group. A skin tissue architecture quality (STAQ) index, developed to respond to the paucity in regenerative performance standards in pre-clinical experiments, is furthermore utilized as a quantitative metric for comparing the different treatments. It highlights Fn nanofiber scaffolds capacity to achieve a skin architecture closest to healthy skin. Taken together, these data show that synthetic fibrillogenesis was effective in manufacturing fibrillar Fn nanofiber wound dressings, which subsequently demonstrated use as a pro-regenerative material strategy, elicited by the accelerated wound closure and enhanced tissue restoration.

### Example 2A: Materials and Methods

The following materials and methods were used in Example 2.

### Rotary Jet Spinning (RJS)

The RJS set-up consists of a custom machined aluminum reservoir with an inner diameter of 20 mm and volume of 3.5 ml perforated with two cylindrical orifices (D=400 μm, L=0.75 cm) (FIGS. 42a and 42b). The perforated reservoir was attached to the shaft of a brushless motor (Maxon motors, Fall River, Mass.) and rotation speed, ranging from 10 k rpm to 35 k rpm, was controlled by circuit board.

### Fn Nanofiber Fabrication

Fn was obtained (Human, BD Biosciences) as a 5 mg lyophilized powder in its unreduced form with a molecular weight of 440 kDa. To facilitate dissolution of Fn and appropriate solvent evaporation to form nanofibers, a 2:1 mixture of 1,1,1,3,3,3-Hexafluoro-2-propanol (HFIP) (Sigma Aldrich, St. Louis, Mo.) and millipore H₂O was used as a solvent. 2% weight/volume (w/v) Fn was first dissolved in millipore H₂O for 24 hours at 4° C. and prior to spinning HFIP was added. After the motor reached target speed, Fn solution was loaded by pipette at a rate of ˜10 mL/min into the perforated reservoir. The resulting fibers were collected on a stationary round collector of radius=13.5 cm. The collector was lined with 25 mm glass coverslips to collect fibers. Alternatively, samples were collected on a rotating mandrel, forming sheets of Fn nanofibers (FIG. 48A).

### Nanofiber Diameter Measurements

Fiber coated coverslips were removed from the collector and sputter coated with 5 nm Pt/Pd (Denton Vacuum, Moorestown, N.J.) to minimize charging during imaging. The samples were imaged using a Zeiss SUPRA 55 field-emission scanning electron microscope (Carl Zeiss, Dresden, Germany). Images were analyzed using image analysis software (ImageJ, NIH). A total of 100-200 fibers were analyzed (3-6 random fields of view per sample) to calculate the fiber diameter. The fiber diameter distribution was reported as mean fiber diameter±standard error of the mean (SEM).

### Protein Structural Integrity

To ensure that Fn proteins remained intact after dissolution in HFIP solvent (for a period of 5 hours maximum) and subsequent unfolding into nanofibers, Raman spectroscopy analysis was performed, suggesting Amide stretching regions (FIG. 49). Briefly, spectral scans were collected using a WITec Confocal Raman microscope/SNOM/AFM (WITec, Alpha300) with a 532 nm laser. Three spectral scans (Integration time=25 sec) were collected for n=10 fibers per sample.

### Fn Immunostaining

Fn fibers were stained by incubating fiber coated coverslips in a solution of PBS containing a 1:200 dilution of anti-human Fn polyclonal antibody (Sigma) for 1 hour at room temperature. Samples were rinsed in PBS (3×15 minutes). Samples were then incubated in a 1:200 dilution of Alexa Fluor 546 goat anti-rabbit IgG (H+L) secondary antibody (Invitrogen, Eugene, Oreg.) for 1 hr. After staining, samples were rinsed and mounted on glass slides for imaging. Images were then acquired on the LSM 5 LIVE Confocal Microscopy (Carl Zeiss) using a 40×/1.3 Oil Differential Interference Contrast (DIC) objective lens. Fn labeled with Alexa Fluor 488 was imaged with a λ=488 nm wavelength emission laser. Nanofibers immunofluorescently-labeled were imaged using a λ=546 nm wavelength emission laser.

### Fn FRET Measurements

Fn molecules were FRET-labeled according to previously published protocols (Baneyx, G., Baugh, L. & Vogel, V. Proc Natl Acad Sci USA 98, 14464-14468 (2001); Little, W. C., Smith, M. L., Ebneter, U. & Vogel, V. Matrix Biol 27, 451-461 (2008); Vogel, V. Annual Review of Biophysics and Biomolecular Structure 35, 459-488, doi:10.1146/annurev.biophys.35.040405.102013 (2006); Deravi, L. F. et al. Nano Lett 12, 5587-5592 (2012)). Briefly, Fn was denatured in 4M guadinidinium hydrochloride [GdnHCl] for 15 minutes, then incubated with tetramethylrhodamine-5-maleimide (TMR) (Molecular Probes, Invitrogen) at room temperature for 2 hours to covalently bind TMR to cryptic cysteines by maleimide coupling. Fn was then refolded and separated from unreacted TMR fluorophore by size exclusion chromatography (Quick Spin G-25 Sephadex Protein Columns, Roche). TMR labeled Fn was then incubated with Alexa Fluor 488 carboxylic acid, 2,3,5,6-tetrafluorophenyl ester (Molecular Probes, Invitrogen) for 1 hour at room temperature. The dual-labeled Fn was separated from unreacted fluorophore using size exclusion chromatography. Dual-labeled Fn was then lyophilized and used immediately. Using confocal microscopy, samples were excited at λ=488 nm and emission spectra was collected at λ=520 nm and 576 nm. Fluorescent images were analyzed using ImageJ image analysis software.

### Fn Nanofiber Tensile Testing

Mechanical testing of Fn nanofibers was performed according to previously published methods (Deravi, L. F. et al. Nano Lett 12, 5587-5592 (2012)) using glass micropipette beam bending. Solid borosilicate glass rods (#BR-100-10, diameter: 1.0 mm, length: 10 cm, Sutter Instrument Co., Novato, Calif.,) were pulled into tapered pipettes using a Flaming/Brown Micropipette Puller (Sutter Instrument Co.) by the following parameter settings: Heat=730, Pull=50, Velocity=100, Time=250. Calibrated pipettes were then used to measure force generated during single fiber tensile tests (Deravi, L. F. et al. Nano Lett 12, 5587-5592 (2012)). Fn nanofibers were attached at one end to a calibrated pipette and at the other to a force applicator pipette via nonspecific adhesive forces. Samples were then pulled uniaxially at a constant strain rate of 1 μm s-1 (FIG. 50).

### In Vivo Wound Healing Studies

All animal experiments were performed following a procedure approved by the Harvard University Institutional Animal Care and Use Committee (IACUC). C57B/L6 male mice (52 days old) (Charles River Laboratories, Wilmington, Mass.) were anesthetized and maintained on surgical plane of anesthesia with isoflurane. Once a toe pinch test confirmed anesthesia, dorsal side of mice prepared by shaving with an electric razor, then manual razor. Surgical area was cleaned three times with betadine and alcohol to sterilize the area. Two full thickness wounds were made on the midline of the back and nanofiber dressings were applied to the wound. Following previous wound healing protocols that studied de novo regeneration of hair follicles, no splinting model was used in these experiments (Ito, M. Stem cells in the hair follicle bulge contribute to wound repair but not to homeostasis of the epidermis. Nature Med. 11, 1351-1354 (2005); Ito, M. Wnt-dependent de novo hair follicle regeneration in adult mouse skin after wounding. Nature 447, 316-320 (2007)). To keep the area clean, free of debris and stabilized, Tegaderm™ patches were applied above all treatment conditions. Mice were monitored daily. After 20 days, mice were sacrificed via IACUC approved methods and tissue harvested for further testing. To confirm mouse health for the duration of the study, the mice were weighed at the beginning and ending of the study and were all shown to gain on an average 2.7 g over a 3 week study. There was no significant weight or health difference in any test or control group.

### Wound Closure Measurements

Wound area was measured from digital photographs of wounds taken every two days throughout the study. Area was measured by tracing leading edge of the epithelial layer using ImageJ image analysis software.

### Histological and Immunofluorescent Staining

Tissues were harvested from healthy and injured mice and fixed with 4% paraformaldehyde for 5 minutes. To stain and image tissues, a cryostat operation to prepare thin slices from the harvested tissues was used. Whole tissues were first embedded in either a 50% Paraffin and 50% Tissue-Tek O.C.T Compound embedding medium solution (Electron Microscopy Sciences, Hatfield, Pa.) or in a 100% Tissue-Tek O.C.T Compound embedding medium solution for 24 hours, after which samples were flash frozen in liquid nitrogen and stored at −20° C. Thin slices were then prepared with a Leica CM 1950 cryostat and collected with Super Frost Plus slides, after which they were replaced in a freezer at −20° C. before staining. Next, staining and imaging were performed according to standard protocols.

### Epidermal Thickness Quantification

Recovery of healthy epidermal structure in the treated tissues was assessed by measuring epidermal thickness from H&E and Masson's Trichrome staining tissue sections. Thickness was measured manually using ImageJ image analysis software. FIG. 51 illustrates measurements of different treated tissue samples, calculated between the black dashed lines. Lower dashed black lines were drawn at the interface of the dermis and the stratum basal, and upper dashed black line was positioned above the stratum granulosum, disregarding the stratum corneum as it flaked off during staining.

### Hair Follicle and Sebaceous Gland Quantification

Regeneration of the skin appendages in the treated tissues was quantified by counting hair follicles and sebaceous glands in Masson's trichrome stained tissue sections (FIG. 51). As wound closure in mice is strongly promoted by contraction compared to humans (Sullivan, T. P., Eaglstein, W. H., Davis, S. C. & Mertz, P. Wound Repair Regen 9, 66-76 (2001)), consistency in measurements was maintained by establishing wound edges. The wound edges were defined by determining the position where the underlying panniculus carnosus muscle tissue was sectioned as illustrated in FIG. 52. Hair follicle and sebaceous gland amounts were quantified per area and compared to healthy tissues (FIG. 51).

### ECM Fiber Alignment Quantification

Organization of ECM fiber alignment was quantified using an orientation order parameter (OOP) metric (0≤OOP≤1), representing perfect anisotropy with a value of 1 and perfect isotropy with a value of 0 (Grosberg, A. et al. PLoS Comput Biol 7, 24 (2011)). To calculate OOPs, angle-color image algorithms, using a custom ImageJ macro, were first derived from H&E images of treated tissues at day 20. Values of ECM fiber orientation were then extracted from the image algorithm, using a custom Matlab code, and subsequently calculating an OOP value for the tissue (FIG. 53).

### Development of a Skin Tissue Architecture Quality (STAQ) Index

To quantitatively assess the efficacy of nanofiber wound dressings to promote tissue restoration, a Skin Tissue Architecture Quality (STAQ) index was developed. This rubric utilizes a modified form of the Hellinger distance metric used previously to assess the therapeutic outcome of cardiopoetic stem cell repair of myocardial infarction (Emmert, M. Y. et al. Biomaterials 122, 48-62, doi:10.1016/j.biomaterials.2016.11.029 (2017)) to calculate the overlap in values from 5 experimentally-measured parameters (e.g. epidermal thickness, ECM fibers alignment, hair follicle density, sebaceous gland density, and percent lipid coverage) between healthy/unwounded skin and wounded skin that has been treated with a wound dressing. The STAQ index (Eq. 1) uses the mean (0 and standard deviation (a) values of the experimental measurements from healthy and wounded skin to calculate the degree of separation between the probability distributions for each experimental parameter.

$$S T A Q = 100 \times \sqrt{\frac{2 {σ}_{h e a l t h y} {σ}_{wounded}}{{σ}_{h e a l t h y}^{2} + {σ}_{wounded}^{2}}} {e}^{- \frac{1 {( {μ}_{h e a l thy} - {μ}_{wounded} )}^{2}}{4 {σ}_{h e a l t h y}^{2} + {σ}_{wounded}^{2}}} \qquad ( 1 )$$

The STAQ score output by this equation falls within the interval [0, 100], where a score of zero indicates that the population distributions are completely different (i.e. no match between healthy and wounded skin), and a value of 100 indicates that they are completely identical (i.e. perfect match between healthy and wounded skin). Combined scores for each wound dressing were calculated as the mean absolute deviation (MAD) between the healthy and wounded STAQ scores (Eq. 1) for the set of 5 experimental parameters measured, according to the following equation:

### Statistical Analysis

Statistical analyses were conducted using SigmaPlot (v12.0, Systat Software, Inc., CA). One-way ANOVA on ranks with post hoc multiple comparisons Dunn's test or Student's t-test were used where appropriate, for wound closure and histological data analyses. Quantitative data are presented as mean±SEM and significance was considered for p<0.05.

### Haematoxylin and Eosin Staining (H&E)

H&E staining was performed as described previously (Abaci, H. E., Gledhill, K., Guo, Z., Christiano, A. M. & Shuler, M. L. Lab Chip 15, 882-888 (2015)). De-paraffinized sections were stained with Mayers Haematoxylin (Sigma) at room temperature for 3 minutes. Blue staining was performed by rinsing in tap water while differentiation was performed by rinsing in 1% acid ethanol. Samples were counterstained by rinsing with eosin (Sigma) for 30 seconds and dehydrated by sequential washing with 95% ethanol, 100% ethanol and Histo-Clear (National Diagnostics, Atlanta, Ga.). Slides were covered with cover-slips using DPX (Agar Scientific, UK) and examined by light microscopy using a Zeiss Axioplan 2 microscope.

### Masson's Trichrome Staining

Masson's Trichrome was performed using Sigma's HT15 Trichrome staining kit according to the manufacturer's instructions (Sigma). Briefly, paraffin embedded tissues were de-paraffinized and rehydrated gradually in graded ethanol. The samples were then fixed in Bouin's solution and incubated in Weigert's Iron Hematoxylin solution. The slides were stained with Biebrich Scarlet-Acid Fuchsin and Aniline Blue, followed by dehydration in ethanol and xylene. The collagen fibers were stained light gray, the cell nuclei were stained dark gray, and keratin and muscle fibers were stained medium gray. Samples were then monitored under a Olympus VS120 Whole Slide Scanner.

Oil-Red-O staining and Quantification

Frozen sections of 7-12 μm thick were air dried for 2 hours at room temperature, and then stained with Oil-Red-O dye to detect the presence of lipids. Sections were washed in PBS, fixed in 4% formaldehyde (Sigma) and 1% calcium chloride (Sigma) at room temperature for 1 hour. Samples then were incubated in 60% isopropanol (Sigma) for 15 minutes and stained with Oil-Red-O solution (Sigma) for 15 minutes. Samples were then briefly rinsed in 60% isopropanol, rinsed with diH₂O, and counterstained in Mayers Hematoxylin solution (Fluka) before mounting with coverslips in DPX (Agar Scientific). The amount of adipose tissue was assessed by the ratio of the area covered by the oil-red-o positive tissue to the total area of interest. The area of interest was selected as the total area below the sebaceous gland of the hair follicles to exclude the fat tissue in the sebaceous glands from our calculations. CellProfiler software (Carpenter, A. E. et al. Genome Biol 7, 31 (2006)) was used to manually select the tissue of interest and determine the pixels on the image with red staining. Image stitching was performed using a previously published ImageJ plugin (Preibisch, S., Saalfeld, S. & Tomancak, P. Bioinformatics 25, 1463-1465, doi:10.1093/bioinformatics/btp184 (2009)).

### Alkaline Phosphatase Staining

Alkaline phosphatase activity was monitored using VectorLab SK-5100 kit (Vector Laboratories, Burlingame, Calif.) according to manufacturer's instructions. Briefly, frozen tissue sections were rinsed with PBS/0.05% Tween 20 (PBST) shortly and fixed again with 4% formaldehyde for 3-5 min Samples were then rinsed with PBST and incubated for 20 minutes in the staining mixture composed of two drops of reagents 1, 2 and 3 in 5 ml of Tris 150 mM solution with a pH of 8.3. Samples were then monitored under a Olympus VS120 Whole Slide Scanner.

### Immunostaining and Quantification

Tissue samples were first de-paraffinized and rehydrated gradually in graded ethanol. Heat-induced antigen retrieval was then performed by bathing samples in a solution of Sodium Citrate 0.01M and 0.01% tween at pH=6 in diH₂O at a temperature of 98° C. for 20 min, followed by a cooling for 10 min at the bench. Samples were then blocked in NGS (Normal Goat Serum) and 0.3% Tween in PBS for 2 hrs, after which they were incubated for 24 hours at 4° C. in primary antibody solutions of PBS with:
Keratin 5 (mouse) 1: 100 dilution (Invitrogen: MA5-17057)
Keratin 14 (rabbit) 1: 500 dilution (Biolegend: 905301)
Keratin 17 (rabbit) 1:100 dilution (Abcam: ab109725).
   Samples were then washed (2×10 min) and stained with secondary antibodies for 1 hr:
Alexa Fluor 488 goat anti-rabbit secondary antibody 1:1000 dilution (Invitrogen)
Alexa Fluor 594 goat anti-mouse secondary antibody 1:1000 dilution (Invitrogen).

After staining, samples were rinsed, mounted on glass slides and imaged under confocal microscopy using a Zeiss LSM 5 LIVE microscope and an Olympus microscope.

Fluid Mechanics Model

As the reservoir rotates at constant angular speed Ω, the fluid escapes through two small circular channels connecting the bottom of the reservoir to the exterior. Within the RJS system two flow regimes of interest are expected. First, there is a transition region as the fluid travels from the reservoir and into the channel. This entry flow, similar to flow through circular-circular contractions (Dobson, J. et al. Proc Natl Acad Sci USA 114, 4673-4678, doi:10.1073/pnas.1702724114 (2017)), presents high elongational strain rates. The second type of flow occurs inside the channel as the solution travels outwards before being ejected. Shear is dominant in this second flow regime (FIG. 42C). When the jet exits the RJS system sudden lateral forces and fiber extension ensue as the fiber travels from the reservoir to the collector (Mellado, P. et al. Applied Physics Letters 99, 203107, doi:10.1063/1.3662015 (2011)).

Here, the focus is on the flow inside the RJS system. Extensional flow has been shown to enable protein unfolding and assembly (Dobson, J. et al. Proc Natl Acad Sci USA 114, 4673-4678, doi:10.1073/pnas.1702724114 (2017); Perkins, T. T., Smith, D. E. & Chu, S. Science 276, 2016 (1997); Larson, R. G., Hu, H., Smith, D. E., & Chu, S. Journal of Rheology 43, 267-304 (1999); Sing, C. E. & Alexander-Katz, A. Biophysical Journal 98, L35-37, doi:10.1016/j.bpj.2010.01.032 (2010); Paten, J. A. et al. ACS nano 10, 5027-5040, doi:10.1021/acsnano.5b07756 (2016)). Polymers in shear flow in contrast experience fluctuations between folded and stretched configuration, so that extremely high shear flows are usually required to unfold globular proteins (Smith, D. E., Babcock, H. P. & Chu, S. Science 283, 1724-1727 (1999); Hur, J. S., Shaqfeh, E. S. G. & Larson, R. G. Journal of Rheology 44, 713-742, doi:10.1122/1.551115 (2000); Jaspe, J. & Hagen, S. J. Do Biophysical Journal 91, 3415-3424, doi:10.1529/biophysj.106.089367 (2006)). The extensional strain rates in the system's entry flow were first estimated using Computational Fluid Dynamics (CFD) simulations (FIG. 42C). These values were then be utilized to investigate the propensity of fibronectin (Fn) to unfold using established models of protein dynamics under extensional flow. These models are based on calculations of work (J) (Jaspe, J. & Hagen, S. J. Biophysical Journal 91, 3415-3424, doi:10.1529/biophysj.106.089367 (2006)), force (N) (Larson, R. G., Hu, H., Smith, D. E., & Chu, S. Journal of Rheology 43, 267-304 (1999)) and the dimensionless Deborah number (Perkins, T. T., Smith, D. E. & Chu, S. Science 276, 2016 (1997)) and can then be compared to published literature on Fn properties. In a second step, the possibility that shear may also affect Fn's molecular conformation as it travels through the reservoir channel was also examined. As previously, shear rates were estimated and subsequently interpreted using an established model that calculates the dimensionless Weissenberg number, descriptive of protein unfolding in shear flow (Smith, D. E., Babcock, H. P. & Chu, S. Science 283, 1724-1727 (1999)).

Models of Fn Unfolding in Extensional Flow (Entry Flow)

Extensional Strain Rates Calculation in Entry Flow:

CFD simulations allow calculation of the flow profiles. The finite element software COMSOL 5.2a was used. The reservoir has a radius M=0.0125 m and the channel has length L=0.0075 m and radius R=200×10⁻⁶ m. Half of the domain was modeled. While the flow inside of the channel is axisymmetric, the entry flow might be affected by the three-dimensional (3D) geometry. Moreover, modeling a smaller domain with only a fraction of the entire fluid domain would require additional assumptions regarding boundary conditions in the vicinity of the channel entry. These considerations were avoided by solving the Navier-Stokes equations in the 3D domain corresponding to half of the reservoir. The geometry is constructed such that the channel centerline is aligned with the x axis and the yz plane is used for the symmetric boundary condition (FIG. 55). The body force due to centrifugal force in this coordinate system is:

b=(xρΩ²,yΩ²,0) (S1)

At the top of the reservoir an inlet boundary condition is specified with zero pressure. An outlet boundary condition is prescribed at the end of the channel also with zero pressure. The plane yz has a symmetry boundary condition. The rest of the walls have no slip conditions. The fluid density is taken as ρ=1400 kg/m³, and the viscosity as μ=0.1 Pa·s (Golecki, H. M. et al. Langmuir: the ACS journal of surfaces and colloids 30, 13369-13374, doi:10.1021/1a5023104 (2014)). A typical rotation speed for the reservoir is Ω=3000 s⁻¹. The flow was assumed laminar. The resulting finite element mesh was composed of tetrahedral elements inside of the domain and quadrilaterals near the boundary. The element size was chosen to be extremely fine leading to a system of 4,297,332 degrees of freedom. Simulations were run until they converged to a relative error of 10⁻⁷. The computational cost of each simulation was approximately 1.5 hr in a machine equipped with an Intel Xeon E5-1630 v4 processor consisting of four cores operating at 3.7 GHz, and 16 GB RAM.

The velocity in the majority of the reservoir is close to zero, followed by a region of high acceleration as the fluid is pushed into the channel. Once the fluid enters the channel, the velocity profile gradually resembles that of a Poiseuille flow. Even though there is a body force that increases away from the center of the reservoir, the speed inside of the channel does not change significantly (FIG. 42C). The velocity u in the x direction has a maximum of 29.6 m/s. The strain rate in the x direction is \dot{ϵ}=∂u/∂x and has a peak value of 0.76×10⁵ s⁻¹ along the axis of the channel and a maximum of 1.3×10⁵ s⁻¹ overall.

To verify that the assumption of laminar flow is consistent, the Reynolds number is calculated:

$$R e = \frac{ρ \overline{u} R}{μ} \qquad ( S 2 )$$

Where ū=14.8 m/s is the mean velocity in the channel. In this case, Re=41.44 and laminar flow can be assumed.

1^{st} Model of Fn Unfolding in Extensional Flow:

Next the extensional force on Fn due to the strain rate is estimated. Fn can be modeled as a string of 56 globular modules or spherical beads of a=2.5 nm diameter with a contour length of L_{c}=160 nm⁶⁶⁻⁶⁹. The main assumption is that under the influence of the strong extensional flow the molecule begins to unfold just enough such that two spherical sub-clusters are formed separated by a small string of beads⁶⁵. The two spherical sub-clusters consist of n beads and the volume of each sub-cluster is (FIG. 54):

v=nv_{b}=4/3πr³ (S3)

Where v_{b} is the volume of an individual bead and r is the radius of the sub-cluster. The distance between the two sub-clusters can be estimated as (N−2n)d with d the distance between any two beads. The difference in velocity between the two sub-clusters is:

v₂−v₁=(N−2n)d\dot{ϵ} (S4)

The corresponding tension that is created due to the difference in drag force between front and back is:

T=T₂−T₁=3πμr\dot{ϵ}(N−2n)d (S5)

The value of the tension changes from the initial point in which there is a single bead between the two sub-clusters, to the final fully extended conformation. The integral of the tension as the molecule is completely unfolded yields the total work that will be done on the molecule by the fluid (Jaspe, J. & Hagen, S. J. Do Biophysical Journal 91, 3415-3424, doi:10.1529/biophysj.106.089367 (2006)):

$$W = \frac{27}{28} π μ {d}^{2} {{N}^{7 / 3} ( \frac{3 {v}_{b}}{8 π} )}^{1 / 3} \dot{ϵ} \qquad ( S 6 )$$

For the rotation speed of Ω=3000 s⁻¹ (˜28,000 rpm), the calculated work done on a single molecule with this model along the centerline is W=0.00235 fJ and at its maximum is W=0.00382 fJ. Conversely, in previous experiments on Fn nanotextiles (Deravi, L. F. et al. Nano Lett 12, 5587-5592 (2012)), the force required to unfold a single molecule was estimated. From the corresponding force-strain relationship the work needed to unfold a single molecule from 15 nm to 60 nm is calculated to be 0.00399 fJ. These values are remarkably close. The analysis suggests that the elongational strain rate produced by the RJS system at rotation speeds of Ω=3000 s⁻¹ would transfer energy to the Fn molecule in sufficient amount to induce at least partial unfolding.

2^{nd} Model of Fn Unfolding in Extensional Flow:

Alternatively, following the analysis of DNA stretching in extensional flow previously described⁵⁰, the tension in the stretched polymer should balance the drag forces at equilibrium:

ξ\dot{ϵ}x−F(x)=0 (S7)

Where ξ is the drag coefficient, x is the length of the polymer in the current configuration, and F(x) is the force in the molecule due to unfolding or stretching. The force can be calculated based on a worm-like chain model for flexible molecules (Larson, R. G., Hu, H., Smith, D. E., & Chu, S. Journal of Rheology 43, 267-304 (1999)):

$$\frac{F ( x ) {l}_{p}}{{k}_{B} T} = \frac{1}{4} {( 1 - \frac{x}{{L}_{c}} )}^{- 2} - \frac{1}{4} + \frac{x}{{L}_{c}} \qquad ( S8 )$$

Where lₚ is the persistence length. For Fn lₚ=7 to 14 nm (Pelta, J., Berry, H., Fadda, G. C., Pauthe, E. & Lairez, D. Biochemistry 39, 5146-5154 (2000)). The drag coefficient can be approximated using Batchelor's theory of slender bodies in Stokes flow (Saeidi, N., Sander, E. A. & Ruberti, J. W. Biomaterials 30, 6581-6592, doi:http://doi.org/10.1016/j.biomaterials.2009.07.070 (2009)):

$$ξ = \frac{μ 2 π {L}_{c}}{ln ( \frac{{L}_{c}}{a} )} \qquad ( S 9 )$$

Solving Eq. (S7) the stretch value of x/L_{c}=0.98 is obtained, and a tension of 494 pN is the one that satisfies equilibrium. This very high force is obtained because the force in the worm-like chain model increases sharply near full extension. According to the literature, forces ranging from 50 to 200 pN have demonstrated unfolding of globular Fn (Erickson, H. P. Current opinion in structural biology 42, 98-105, doi:10.1016/j.sbi.2016.12.002 (2017)), suggesting that the strain rate of 1.3×10⁵ s⁻¹ should generate enough tension to keep a Fn molecule in a fully extended configuration.

3^{rd} Model of Fn Unfolding in Extensional Flow:

Finally, the Weissenberg (Wi) number, also called Deborah (De) number, is a nondimensional number that relates elastic and viscous forces or the times scales of relaxation and observation, and is defined as:

De=τᵣ\dot{ϵ}(=wi) (S10)

Where τᵣ is the longest relaxation time of the polymer, and can be estimated from the Rouse model as⁷⁰:

$${τ}_{r} = \frac{ξ {〈 {r}_{e e}^{2} 〉}_{0}}{6 {π}^{2} {k}_{B} T} \qquad ( S11 )$$

Where ξ is the drag coefficient, [symbol] rₑₑ² [symbol] ₀ is the chain end-to-end distance, k_{B} is the Boltzmann constant, and T is the temperature. The end-to-end distance can be calculated based on the persistence and contour lengths as:

[symbol]

rₑₑ² [symbol] ₀=2lₚL_{c (S}12)

The relaxation time of Fn is then estimated to be 222 μs. Thus, at the centerline strain rate the Deborah number is De=16.6 and at the maximum strain rate it is De=28.9. It has been determined that a coil-stretch transition occurs at De=0.5, such that for De>0.5 there will be at least some unfolding of the polymer (Perkins, T. T., Smith, D. E. & Chu, S. Science 276, 2016 (1997)). As the strain rate and, consequently, the Deborah (or Weissenberg) number increases, the polymer is stretched more. For instance, with DNA, which is a flexible chain, a stretch of x/L_{c}=0.82 is achieved at De=4.1 (Perkins, T. T., Smith, D. E. & Chu, S. Science 276, 2016 (1997); Larson, R. G., Hu, H., Smith, D. E., & Chu, S. Journal of Rheology 43, 267-304 (1999)). Thus we expect that De values between 16.6 and 28.9 will be enough to induce Fn unfolding.

Additionally, it must also be noted that the relaxation time and the corresponding critical strain rate are determined for dilute concentrations. Fn has an intrinsic viscosity of 10 mg/L at low ionic strengths and pH 7.4 (Williams, E. C., Janmey, P. A., Ferry, J. D. & Mosher, D. F. J Biol Chem 257, 14973-14978 (1982)). Thus, the critical concentration to reach a semi-dilute solution is 77 mg/mL (C. Clasen, J. P. P., and W.-M. Kulicke. Journal of Rheology 50 (2006)), whereas the solutions used in this study have a Fn concentrations of 20 mg/mL. Nonetheless, it has been shown that even small deformation of polymer chains in extensional flow fields sharply lower the critical concentration needed for coil-coil interactions between molecules—indicative of semi-dilute regimes (Hur, J. S., Shaqfeh, E. S. G. & Larson, R. G. Journal of Rheology 44, 713-742, doi:10.1122/1.551115 (2000)). Transitioning to a non-dilute regime can have significant effect on the relaxation time as the molecules aggregate, consequently increasing the local De and Wi values in the flow system. Recent investigations on collagen assembly also showed that solutions in the semi-dilute regime increased the relaxation time by orders of magnitude or, equivalently, reduced the critical strain rate needed for unfolding (Paten, J. A. et al. ACS nano 10, 5027-5040, doi:10.1021/acsnano.5b07756 (2016)). In the case of Fn, where fibrillogenesis has even been demonstrated with relatively low strain rates (Raoufi, M. et al. Nano Lett 15, 6357-6364, doi:10.1021/acs.nanolett.5b01356 (2015); Little, W. C., Smith, M. L., Ebneter, U. & Vogel, V. Matrix Biol 27, 451-461 (2008)), this dynamic flow regime should be largely sufficient to prompt molecular unfolding and assembly.

Model of Fn Unfolding in Shear Flow (Channel Flow):

Shear Rates Calculation in Channel Flow:

While extensional flow at the channel entry might be the strongest contribution to the initial unfolding of Fn (Dobson, J. et al. Proc Natl Acad Sci USA 114, 4673-4678, doi:10.1073/pnas.1702724114 (2017)), shear flow has been shown to also influence the conformation of flexible polymers (Smith, D. E., Babcock, H. P. & Chu, S. Science 283, 1724-1727 (1999); Hur, J. S., Shaqfeh, E. S. G. & Larson, R. G. Journal of Rheology 44, 713-742, doi:10.1122/1.551115 (2000)). Therefore the shear flow in the channel is now considered (FIG. 42C).

Based on the numerical simulation, even though there is a body force that increases as the fluid moves along the channel, the velocity stays nearly constant. This is consistent with Poiseuille flow through the channel. Let x remain the coordinate along the channel, and r and θ be the radial and circumferential coordinates respectively. Under the assumption of Poiseuille flow the velocity field is at steady state, is axisymmetric, and only has a nonzero component in the x direction which depends solely on the radial coordinate (FIG. 55):

$${u}_{x} ( r ) = - \frac{1}{4 μ} ( \frac{\partial p}{\partial x} - b ) ( {R}^{2} - {r}^{2} ) \qquad ( S13 )$$

where p is the pressure, R is the radius of the orifice, μ denotes dynamic viscosity, and b is a body force. In this coordinate system the centrifugal force is:

b=ρΩ²x (S14)

The force of gravity is neglected since for the channel the centripetal acceleration is dominant g<<Ω²x, x∈[M, M+L]. To determine the pressure distribution, a quadratic dependence on x is proposed:

p(x)=a₁+a₂x+a₃x² (S15)

The quadratic dependence is needed in order to satisfy the continuity equation in Poiseuille flow ∂uₓ/∂x=0. Taking the derivative of Equation (S13) with respect to x and setting the expression equal to zero leads to:

a₃=½ρΩ² (S16)

And the velocity profile becomes:

$${u}_{x} ( r ) = - \frac{1}{4 μ} ( {a}_{2} ) ( {R}^{2} - {r}^{2} ) \qquad ( S17 )$$

Next, to determine the constant a₂, the pressure drop along the pipe is determined. Inside of the reservoir the fluid is rotating at constant angular speed and therefore it pushes on the inside walls of the reservoir according to the centripetal acceleration a=Ω²x. The pressure distribution of the rotating fluid inside the reservoir ignoring gravity is (Lubarda, V. A. The shape of a liquid surface in a uniformly rotating cylinder in the presence of surface tension. Acta Mechanica 224, 1365-1382, doi:10.1007/s00707-013-0813-6 (2013)):

p=½ρΩ²x^{2 (S}18)

Therefore, the pressure at the inner wall of the reservoir is:

pᵢₙ=½ρΩ²M²=a₁+a₂M+½ρΩ²M²=p(M) (S19)

The pressure at the outlet of the channel is zero.

pₒᵤₜ=0=a₁+a₂(M+L)+½ρΩ²(M+L)²=p(M+L) (S20)

From Eq. (S19) and (S20) the missing constant is determined:

$${a}_{2} = - \frac{ρ {{Ω}^{2} ( M + L )}^{2}}{2 L} \qquad ( S 21 )$$

And the final expression for the velocity is obtained:

$${u}_{x} ( r ) = \frac{1}{4 μ} ( \frac{ρ {{Ω}^{2} ( M + L )}^{2}}{2 L} ) ( {R}^{2} - {r}^{2} ) \qquad ( S 22 )$$

And the shear rate is readily calculated as

$$\dot{γ} = - \frac{ρ {{Ω}^{2} ( M + L )}^{2} r}{4 μ L} \qquad ( S23 )$$

Thus the shear rate depends linearly on the radial coordinate and is quadratic on the angular velocity. This analytical result is in very good agreement with the numerical simulation. For a rotation speed of Ω=3000 s⁻¹ the maximum velocity using Eq. S22 is 31.9 m/s whereas the simulation predicts 29.5 m/s. Similarly, the shear rate using S23 is \dot{γ}=3.19×10⁵ s⁻¹ while the numerical result is 2.9×10⁵ s⁻¹.

Model for Fn Unfolding in Shear Flow:

The conformational changes of polymers in shear flow are governed by the Weissenberg (Wi) number, which is a nondimensional measure that relates elastic and viscous forces:

Wi=\dot{γ}τᵣ (S24)

The Weissenbergh (Wi) number at the wall can be calculated using (S18) and gives Wi=79.0. The Wi number is expected to have significant effect on the stretching of the molecules in shear flow⁵¹, measuring the strength of the shear force relative to the relaxation time of the polymer. As the Wi number increases the polymer molecules are expected to present more frequent and larger extensions. When Wi is below 1, the molecules will have Brownian motion and oscillate between coiled and stretched conformations but the effect of the flow remains weak⁵². As Wi increases, oscillation will persist, but it will become more likely to find the molecules in their extended conformation.

For Fn in the rotating reservoir channel, fluctuations of the molecule between different conformational extensions should still be expected. Nonetheless, non-dimensional simulations on wormlike chain models and Kramer bead and rod models for different polymer flexibilities show that the expected mean of relative elongation is in the range of 0.2 to 0.6 (Hur, J. S., Shaqfeh, E. S. G. & Larson, R. G. Journal of Rheology 44, 713-742, doi:10.1122/1.551115 (2000)). Moreover, this behavior is representative of smaller values of Wi, and for the simulations with Wi approaching 80 the elongation achieves an asymptotic limit close to 0.5 for flexible molecules. Considering these Brownian dynamic simulations, it is expected that the Wi numbers in the RJS system are thus large enough to further contribute to conformational changes of the Fn molecules in addition to the extensional flow at the channel entry.

Models Discussion

Using CFD simulations of the extensional and shear rates (FIG. 42C) as well as established analytical models for predicting protein unfolding under flow, the propensity of Fn to undergo fibrillogenesis in RJS was assessed.

The models described above allow comparison of previous work on protein unfolding in different flow regimes with literature on Fn mechanical behavior. However, to elucidate the detailed mechanisms of Fn unfolding and assembly, a more thorough understanding of the spinning process would likely be required. In particular, Brownian dynamic simulations similar to those described previously (Hur, J. S., Shaqfeh, E. S. G. & Larson, R. G. Journal of Rheology 44, 713-742, doi:10.1122/1.551115 (2000)) could provide additional insights into this process. Simpler models can nonetheless provide insight into the physics of the process. The equilibrium model to estimate the force on the molecule disregards the unfolding process (Larson, R. G., Hu, H., Smith, D. E., & Chu, S. Journal of Rheology 43, 267-304 (1999)), yet it supports the idea that an unfolded Fn molecule can be kept in the extended configuration in the RJS′ extensional flow field. The work calculation provides a simple characterization of the unfolding process (Jaspe, J. & Hagen, S. J. Biophysical Journal 91, 3415-3424, doi:10.1529/biophysj.106.089367 (2006)) and allows comparison with previous data on Fn force-strain curves (Deravi, L. F. et al. Nano Lett 12, 5587-5592 (2012)) also supporting the idea of Fn unfolding in elongational flow. The dimensionless Deborah number or the Weissenberg number enable a wider comparison to theory and experiments done with other flexible polymers such as DNA (Perkins, T. T., Smith, D. E. & Chu, S. Science 276, 2016 (1997); Smith, D. E., Babcock, H. P. & Chu, S. Science 283, 1724-1727 (1999)) and collagen (Paten, J. A. et al. ACS nano 10, 5027-5040, doi:10.1021/acsnano.5b07756 (2016)) and further suggests that the strain or shear rates in the RJS system are large enough to induce Fn unfolding.

### Example 2B: Synthetic Fibrillogenesis of Fn Nanofibers

Analytical and computational models were first used to estimate whether the strain and shear rates generated in the RJS could induce Fn unfolding and fibrillogenesis, thus testing the initial hypothesis (FIG. 42A and FIG. 54). To establish these models, the system into was separated into its two distinct flow regimes: the transitory entry flow, where the fluid travels from the reservoir to the channel, and the channel flow, where the fluid travels through the channel and is ejected out of the system (FIGS. 42A-42C and FIG. 54). Once the fluid exits the reservoir channel, it will be exposed to sudden lateral forces while the solvent gradually evaporates. Fiber formation and extension will ensue, enabling assembly of nanofiber sheets on a collector (timescale ˜0.01 s) (Badrossamay, M. R., McIlwee, H. A., Goss, J. A. & Parker, K. K. Nano Lett 10, 2257-2261 (2010); Badrossamay, M. R. et al. Biomaterials 35, 3188-3197 (2014); Mellado, P. et al. Applied Physics Letters 99, 203107, doi:10.1063/1.3662015 (2011); Golecki, H. M. et al. Langmuir: the ACS journal of surfaces and colloids 30, 13369-13374, doi:10.1021/1a5023104 (2014)).

Focus on a first step on the entry flow where the Fn solution will experience acceleration as it is constricted into the channel (FIG. 42C, top schematic). This acceleration, characterized by high extensional strain rates, was recently described to enable and drive protein aggregation in a similar system (Dobson, J. et al. Proc Natl Acad Sci USA 114, 4673-4678, doi:10.1073/pnas.1702724114 (2017)). Thus, to evaluate the propensity of Fn to undergo fibrillogenesis in this flow regime, the velocity profile and extensional strain rates were calculated using computational fluid dynamics (CFD) simulations (FIG. 42C and FIG. 55). The strain rates for a rotation speed of ˜28,000 rpm were estimated at 0.76×10⁵ s⁻¹ along the center line and at 1.28×10⁵ s⁻¹ proximal to the entry flow edges. Next, the Deborah (De) number, used to explain the conformational changes of proteins under elongation flow, was calculated:

De=τᵣ\dot{ϵ} (2)

Equation (2) expresses the dimensionless number De that quantifies the strain rate \dot{ϵ} and the protein relaxation time scale ratio, with τᵣ the longest relaxation time—estimated at 222 μs for Fn. From this, a De number of 28.9 (FIG. 56) was calculated. In contrast, previous experiments showed that stretching of DNA was achieved with a De as low as 4.1 (Perkins, T. T., Smith, D. E. & Chu, S. Single Science 276, 2016 (1997); Larson, R. G., Hu, H., Smith, D. E., & Chu, S. Journal of Rheology 43, 267-304 (1999)). This suggests that the elongation strain rates in RJS should be sufficient to initiate unfolding of Fn. Alternatively, calculating the total work applied to an Fn molecule also demonstrated comparable values to previously described methods of Fn nanotextile fabrication (Deravi, L. F. et al. Nano Lett 12, 5587-5592 (2012)). Balancing the drag forces and the tension in the molecule modeled as a worm-like chain also revealed that equilibrium of a single chain could be achieved for a 0.98 stretch.

Although, elongation flow described above is likely the strongest contributor to Fn unfolding (Dobson, J. et al. Proc Natl Acad Sci USA 114, 4673-4678, doi:10.1073/pnas.1702724114 (2017)), shear has likewise been demonstrated to impact protein conformation (Smith, D. E., Babcock, H. P. & Chu, S. Science 283, 1724-1727 (1999)). To determine the shear rate produced in the RJS channel, a Poiseuille flow was assumed and the pressure gradient along the channel as a function of the centrifugal force exerted by the rotating reservoir was calculated (FIG. 42C). Shear rates achievable within the RJS system therefore range from 0 to ˜3×10⁵ s⁻¹. CFD simulations in the channel paralleled these calculations (FIG. 42C). Next, as varying shear rates of polymer chains can have a significant effect on molecular extension dynamics (Smith, D. E., Babcock, H. P. & Chu, S. Science 283, 1724-1727 (1999)), the Weissenberg (Wi) number that is used to explain conformational changes in such conditions was calculated:

Wi=\dot{γ}τᵣ (3)

Equation (3) shows the nondimensional Wi number dependent on the shear rate γ and is readily calculated at 79.0 for the maximum rotation speed at the channel wall (FIG. 56). From simulations previously described (Hur, J. S., Shaqfeh, E. S. G. & Larson, R. G. Journal of Rheology 44, 713-742, doi:10.1122/1.551115 (2000)), normalized molecular extension reaches an asymptotic limit close to 0.5 for a Wi number approaching 80, thus suggesting that shear-induced conformational changes of Fn should be achievable within the system.

Experimentally, it was observed that fibers composed of Fn formed at speeds above 25 k rpm with an average fiber diameter of 427±138 nm, while partial fiber formation was noticed for speeds of 15 k to 20 k rpm (FIG. 42D and FIG. 48). To determine how RJS processing affected the conformation state of Fn, Raman spectroscopy was used and showed an intact secondary structure with defined Amide I and III peaks (FIG. 49). The absence of Amide II peak suggests that Fn tertiary structure was in a partially folded state (Deravi, L. F. et al. Nano Lett 12, 5587-5592 (2012)). To further verify molecular integrity, immunstaining was performed, using an amine-specific fluorophore as well as an antibody against human Fn (FIG. 57). The ability to perform staining of Fn fibers in aqueous solution also confirmed their insolubility-distinctive of fibrillar Fn matrices (To, W. S. & Midwood, K. S. Fibrogenesis Tissue Repair 4, 1755-1536 (2011)). Together, these data demonstrate that the RJS system produces sufficient shear forces to unfold and polymerize Fn, and the spinning parameters described herein are amenable to form fiber scaffolds.

To support these data, Fn was dual-labeled for fluorescence resonance energy transfer (FRET) imaging as previously described (Baneyx, G., Baugh, L. & Vogel, V. Proc Natl Acad Sci USA 98, 14464-14468 (2001); Little, W. C., Smith, M. L., Ebneter, U. & Vogel, V. Matrix Biol 27, 451-461 (2008); Ahn, S. et al. Adv Mater 27, 2838-2845 (2015)), and measured changes in FRET intensity during fiber formation. A high acceptor to donor fluorescence ratio (0.95±0.02) in solution was observed, suggesting Fn in solution is in a compact, folded conformation prior to RJS processing.

After spinning, the FRET signal decreased by ˜39 percent to 0.58±0.01 for a rotation speed of 28,000 rpm (FIG. 42E and FIG. 58). As a mean of comparison, Fn unfolding using 4 M and 8 M guanidinium chloride [GdnHCl] demonstrated FRET intensities of 0.69 and 0.56, respectively (FIG. 59). The lower FRET signals (I_{A}/I_{D}<0.6) demonstrates a flow-induced unfolding event, producing insoluble Fn fibers. Collectively, these data demonstrate that Fn molecules are unfolding—a prerequisite for exposure of Fn-Fn binding sites and induction of fibrillogenesis (To, W. S. & Midwood, K. S. Fibrogenesis Tissue Repair 4, 1755-1536 (2011)—and thus validates RJS as a method for producing fibrillar Fn nanofiber scaffolds.

### Example 2C: Fn Nanofibers Tensile Testing

It was then determined how these molecular changes impacted the mechanical properties of these fibers. Previously, it was shown that extended Fn proteins exhibited bi-modal stress strain curves when pulled under uniaxial tension (Deravi, L. F. et al. Nano Lett 12, 5587-5592 (2012)). To determine whether the same was true in the Fn networks produced with the RJS system, uniaxial tensile testing was used to measure the stiffness of these fibers (FIG. 43A and FIG. 50). Single fibers were attached to force-calibrated pipette tips and deflection at the tip-fiber interface was measured to generate stress-strain curves for ˜400 nm fibers. A 300% strain before failure was observed in the fibers (FIGS. 43A and 43B). To understand how the conformational state of Fn influenced its bulk mechanical properties, a two-state eight-chain model to estimate the force-extension profile of a single molecule according to previously reported methods was used (Deravi, L. F. et al. Nano Lett 12, 5587-5592 (2012)). A plateau and a sharp force increase were observed, suggestive of molecular straightening and domain unfolding, respectively (FIG. 43C). Together with the chemical analysis, the information extrapolated from single fiber mechanics demonstrates that Fn undergoes conformational unfolding during spinning to yield continuous fibers and that their ability to undergo a 300% strain is largely due to domain unfolding during uniaxial tensile testing.

### Example 2D: In Vivo Wound Closure Acceleration

To evaluate the effect of Fn nanofibers on wound healing, full-thickness dorsal wounds in a murine model were studied. Two full-thickness dermal wounds were made with an 8 mm biopsy punch on the flanks of C57BL/6 male mice (FIG. 44A). For optimal integration into cutaneous wounds, the structural architecture of native murine dermal ECM was mimicked (FIG. 44B, left panel) by replicating the basketwoven fiber appearance on the macroscale and the anisotropic structure on the microscale (FIG. 44B, right panel). In this study, fibronectin nanofiber scaffolds (Fn) were compared to a control group with no fibers. Both groups were covered with Tegaderm™ to secure the wounds and provide support for scaffolds integration. Tegaderm™ was chosen as it is a widely used film dressing, known for its moist retention and protection against pathogens (Murphy, P. S. & Evans, G. R. Plast Surg Int 190436, 22 (2012)), and was therefore added to support both tested conditions. Mice were photographed daily throughout the study to determine wound closure rate (FIG. 44C). Wound traces revealed that Fn nanofibers significantly accelerated wound closure (closed by ˜day 11) compared to the control (˜day 14) (FIGS. 44D and 44E). In addition, by day 16, Fn-treated wounds showed closer morphological appearance to native unwounded tissue (FIG. 44C), demonstrating enhanced cutaneous wound healing.

### Example 2E: Dermal and Epidermal Tissue Architecture Restoration

Epithelial cells enable de novo regeneration of hair follicles in adult mice after wounding, recapitulating to some extent the embryonic developmental process (Ito, M. Nature 447, 316-320 (2007)). It was, therefore, determined whether mimicking the Fn-rich fetal dermal microenvironment in humans was promoting restoration of epidermal and dermal architecture, and more specifically if it could enhance neogenesis of skin appendages by stimulating the recruitment of these cells. Tissue sections stained for Masson's trichrome at day 20 revealed that Fn-treated wounds had strong appendage regeneration capabilities, recovering comparable structures to healthy skin (FIGS. 45A and 45B).

Quantitative analysis of skin tissue architecture demonstrated that original, healthy epidermal thickness was recovered for Fn within 20 days, whereas the non-treated wounds still had significantly thicker epidermises, characteristic of ongoing healing (Martin, P. Science 276, 75-81 (1997)) (FIG. 45C and FIG. 51). Organization of ECM fibers in the dermis, commonly depicted as a basket-woven structure in healthy tissue and aligned bundles in scar tissue, was used as a metric to assess fibrosis (FIG. 53). These analyses revealed that both conditions had higher ECM fiber alignment than native skin, with closer values to native skin for the Fn condition (FIG. 45D). Finally, hair follicle and sebaceous gland density confirmed that Fn-based wound dressings promoted stronger restoration of skin appendages, and showed similar organization to the native state. In contrast, the control group exhibited significantly lower restoration (FIG. 45E and FIGS. 51 and 53). To facilitate the assessment of the regenerative potency of the Fn scaffolds fabricated herein, treatments were compared to healthy skin tissues and scored from 0 to 100% match based on the data from the different testing parameters (FIG. 45F). This analysis demonstrated the regenerative potency of fibrillar Fn with the closest match to native skin for all tested parameters.

### Example 2F: Dermal Papillae and Basal Epithelial Cell Recruitment

To support these findings, it was determined whether dermal papillae (DP), critical for hair follicle neogenesis (Reynolds, A. J., Lawrence, C., Cserhalmi-Friedman, P. B., Christiano, A. M. & Jahoda, C. A. Nature 402, 33-34 (1999); Oshima, H., Rochat, A., Kedzia, C., Kobayashi, K. & Barrandon, Y. Cell 104, 233-245 (2001)), and epidermal cells (EC), which fuel epidermal homeostasis (Blanpain, C. & Fuchs, E. Nat Rev Mol Cell Biol 10, 207-217 (2009)) and repair (Ito, M. Nature 447, 316-320 (2007)), were present in Fn-treated wounds. Sectioned tissue samples were stained with alkaline phosphatase (ALP) to determine presence of DP in the bulb of hair follicles, as well as keratin 5 (K5)/keratin 14 (K14) to highlight ECs that constitute the interfollicular epidermis (IFE) and surround hair follicles, and keratin 17 (K17) to mark ECs specific to the outer root sheath (ORS) of hair follicles (FIGS. 46A and 46B). After wounding, ECs are recruited from the surrounding IFE and the hair follicle bulge and migrate towards the injury to repair the epidermis and its skin appendages (Ito, M. Nature 447, 316-320 (2007)). By day 20, Fn-treated tissues demonstrated widespread presence of K5/K14 in the epidermis and around the regenerating hair follicles, while K17 remained specific to the ORS. Remarkably, DPs were discernable in the dermis at the wound edge and at the center of wounds (FIG. 46C). As centers of wounds treated with the non-treated control prompted minimal presence of skin appendages, K5-positive cells were only observed in the IFE while DPs were altogether absent. Although wound contraction, typical in mouse wound healing (Wang, X., Ge, J., Tredget, E. E. & Wu, Y. Nat Protoc 8, 302-309 (2013)), may be hindering the ability to image full structures of hair follicles, presence of DPs and ECs in Fn-treated tissues is compelling and demonstrates restoration of functional hair follicles.

### Example 2G: Lipid Layer Restoration

It was next determined whether intradermal adipocyte cells, known to contribute to the stem cell niche that directs hair follicle growth (Festa, E. et al. Cell 146, 761-771 (2011)), were also regenerating in the treated tissues. To verify this, the presence of lipids was examined in the tissues using a lysochrome dye (oil-red-o) to stain the lipid droplet in adipocytes. In healthy tissues, lipids were observed in the sebumsecreting sebaceous glands and in the adipose tissue of the hypodermis (FIG. 47A). In both conditions, adipocytes were re-forming a lipid layer in the hypodermis, (FIG. 47B). A quantitative analysis of the oilred-o coverage revealed comparable levels of adipose tissue in the healthy and the tested conditions, with closer values for the Fn treatment (FIG. 47C). As previously, treatments were compared to healthy skin tissues to assess their regenerative potency, and highlighted the advantage of Fn fibers over the other treatments with a 98.2% match (FIG. 47D).

### Example 2H: Skin Tissue Architecture Quality Index

As novel wound healing therapeutics do not only promote wound closure but also attempt to improve tissue regeneration (Gurtner, G. C., Werner, S., Barrandon, Y. & Longaker, M. T. Nature 453, 314-321 (2008)), metrics to evaluate the efficacy of these products is becoming critical. Comparative effectiveness analyses are being developed to improve the understanding of different available wound dressings, helping the clinician in choosing the ideal treatment (Sood, A., Granick, M. S. & Tomaselli, N. L. Wound Dressings and Comparative Effectiveness Data. (Adv Wound Care (New Rochelle). 2014 Aug. 1; 3(8):511-529.)). Yet, standardized metrics to assess regenerative potency at a preclinical stage are still lacking. Therefore, a skin tissue architecture quality (STAQ) index, inspired by previously described statistical methods (Emmert, M. Y. et al. Biomaterials 122, 48-62, doi:10.1016/j.biomaterials.2016.11.029 (2017); Sheehy, S. P. et al. Stem Cell Reports 2, 282-294 (2014)), to assess functional and structural recovery of the treated skin tissues was developed. The parameters collected during this study (epidermal thickness, ECM fibers alignment, hair follicle density, sebaceous gland density, lipid layer coverage) were compared to a design criterion—healthy/uninjured skin tissue—and scored from 0 to 100 percent, where 0 designates the baseline outcome with no distribution overlap and 100 designates the optimal outcome with perfect overlap. STAQ calculations confirmed the recovery of skin structure and functionality using the Fn nanofiber scaffolds with 79.4% match to healthy skin. In contrast, the non-treated control displayed a lower overlap with 63.1% (FIG. 48).

Development of Fn nanofiber scaffolds was inspired by the distinct biochemical and biophysical properties of the fetal wound healing microenvironment, and tailored to replicate the multi-scale architecture of native dermis, with a basket-woven scaffold organization, an anisotropic fiber alignment and fibers in the nanometer range. Fabrication of Fn nanofibers was achieved by applying sufficient extensional and shear strain rates to the protein, thus inducing fibrillogenesis at a production-scale level (Capulli, A. K. et al. JetValve: Biomaterials 133, 229-241, doi:10.1016/j.biomaterials.2017.04.033 (2017)). FRET analysis was further used to confirm the conformational change instated by RJS. When pulled under uniaxial tension, individual fibers showed a bimodal stress-strain curve that the two-state 8-chain model indicated was due to domain unfolding of extended Fn molecules. These fibers, arranged into 8 mm wide wound dressings, accelerated wound closure and enhanced skin appendage neogenesis, ultimately leading to tissue restoration in full-thickness wounds, highlighting their use as building blocks for wound care products.

Towards a mechanistic understanding of this regenerative phenotype, it was shown that Fn wound dressings supported epithelial cell recruitment, promoting skin appendage, dermal and hypodermal epithelium neogenesis. STAQ scored the Fn-potentiated tissue restoration at 79.4%. In contrast, the control group (Tegaderm™ only) showed a delayed epidermal thinning, and decreased dermal restoration, elicited by the lower presence of hair follicles and sebaceous glands, and characterized by a more anisotropic dermal ECM structure. STAQ score for the non-treated control was measured at 63.1%.

Ultimately, this study improved tissue restoration by emulating a single constituent in the fetal wound healing microenvironment—the ubiquitous presence of fibrillar Fn. Providing this instructive milieu that recapitulates the multi-scale structural properties of skin ECM, delivering functional and protein-binding domains inherent to the Fn molecule, demonstrated strong efficacy for stimulating wound healing and tissue restoration. The ability to support widespread regeneration of skin appendages in full thickness wounds as well as recover skin architectures addresses a fundamental challenge in the field.

### Example 3: Versatile Extracellular (ECM) Protein Nanofiber Scaffold Fabrication for Regenerative Medicine Applications

Currently available wound dressings and regenerative scaffolds are typically composed of one or two main components—commonly referred to as ‘seed and soil’—that denote the cells (such as, keratinocytes or fibroblast) and the scaffolds, respectively. Several approaches are currently being advanced to design the optimal material, whether natural, synthetic or biologic, to establish the principal building-block of these instructive systems. Biologics, such as extracellular matrix (ECM) proteins, present a unique advantage in that regard, as they are inherently designed to interact and function with cells and tissues. In vivo, these ECM proteins are organized as fibrillar structures surrounding cells, with individual fibrils characterized by diameters ranging from 5 to 50 nanometers and that assemble into larger micron-wide fiber bundles and networks. Their large and complex secondary and tertiary structures can furthermore bestow these molecules with significant plasticity, capable of extending to several times their full length, or adopting different conformational shapes under certain stimuli. This in turn can render their manufacturability challenging. Fiber scaffolds are an interesting approach in this space and, at the present time, several competing methods exist for manufacturing such fiber scaffolds with relative versatility.

For example, biological materials, that constitute the extracellular matrix (ECM), present a unique advantage for designing wound dressings as they evolved to directly interact and function with cells and tissues. In vivo, these ECM materials are found as proteins and glycosaminoglycans (GAGs), weaved into fibrillar structures and meshes, and provide physical support and regulatory function (Hynes, R. O. Science (New York, N.Y.) 326, 1216-1219, doi:10.1126/science.1176009 (2009)). Their structural and mechanical properties can furthermore bestow these molecules with significant influence over specific cell behaviors, critical to homeostasis, wound healing and regeneration (Frantz, C., Stewart, K. M. & Weaver, V. M. Journal of cell science 123, 4195-4200, doi:10.1242/jcs.023820 (2010)). The GAG hyaluronic acid (HA) in particular has received considerable attention for its regulatory roles during development (Dicker, K. T. et al. Acta biomaterialia 10, 1558-1570, doi:10.1016/j.actbio.2013.12.019 (2014)) and in several regenerative phenomena observed in mice (Iocono, J. A., Ehrlich, H. P., Keefer, K. A. & Krummel, T. M. Journal of pediatric surgery 33, 564-567 (1998)), fish (Ouyang, X. et al. Hyaluronic acid synthesis is required for zebrafish tail fin regeneration. PloS one 12, e0171898, doi:10.1371/journal.pone.0171898 (2017)), amphibians (Calve, S., Odelberg, S. J. & Simon, H. G. A Developmental biology 344, 259-271, doi:10.1016/j.ydbio.2010.05.007 (2010)), and human fetal skin (Longaker, M. T. et al. Journal of pediatric surgery 25, 430-433 (1990)). Its inherent biocompatibility, mechanical and structural tenability, and water retention properties has in addition made HA a promising candidate for tissue engineering applications (Highley, C. B., Prestwich, G. D. & Burdick, J. A. Current opinion in biotechnology 40, 35-40, doi:10.1016/j.copbio.2016.02.008 (2016)). The availability of reactive functional groups along its disaccharide chain have also been leveraged for functionalization with morphogenic compounds (Jha, A. K. et al. Biomaterials 47, 1-12, doi:10.1016/j.biomaterials.2014.12.043 (2015)), matrix-metalloproteinases (Purcell, B. P. et al. Nat Mater 13, 653-661, doi:10.1038/nmat3922 (2014)) and cell binding moieties (Bian, L., Guvendiren, M., Mauck, R. L. & Burdick, J. A. Proceedings of the National Academy of Sciences of the United States of America 110, 10117-10122, doi:10.1073/pnas.1214100110 (2013)).

To improve recapitulation of the structural and topographical features of the native ECM, micro- and nano-fiber scaffolds have emerged as an efficacious approach, and have contributed to the development of a variety of biomimetic pro-regenerative materials (Wang, X., Ding, B. & Li, B. Mater Today 16, 229-241 (2013)). Their characteristic pervious architecture and fiber directionality can further facilitate integration and remodeling within the host tissue. To date, several spinning methods (such as, electrospinning (Reneker, D. H. & Yarin, A. L. Polymer 49, 2387-2425, doi:https://doi.org/10.1016/j.polymer.2008.02.002 (2008)) and wet-spinning (Dario, P. & Federica, C. Polymer International 66, 1690-1696, doi:doi:10.1002/pi.5332 (2017))) exist for manufacturing these fibrous scaffolds with relative versatility. They present however limitations when it comes to producing pure ECM fibers (Zeugolis, D. I. et al. Biomaterials 29, 2293-2305 (2008)) and at scales that can foster further development and subsequent clinical translation (Capulli, A. K., MacQueen, L. A., Sheehy, S. P. & Parker, K. K. Advanced drug delivery reviews 96, 83-102, doi:10.1016/j.addr.2015.11.020 (2016)). For example, electrospinning has for example enabled the fabrication of collagen and fibrinogen nanofibers by dissolving these proteins into organic volatile solvents and spinning them using jet-elongating electrical fields. However, these fabrication conditions lead to the denaturation of the proteins' secondary structures, thereby inhibiting its functionality. Conversely, methods for fabricating ECM polysaccharides such as hyaluronic acid have required carrier polymers to facilitate fiber formation, as electrical fields interfere with their polyelectrolyte backbones. These limitations have constrained innovation in fiber manufacturing of ECM proteins as their bio-chemical and—physical properties were degraded or modified—involuntarily or as a sine qua none condition. In addition, small pore sizes resulting from tight packing of nano-scale fibers has also emerged as a common hindrance of these fibrous scaffolds (Pham, Q. P., Sharma, U. & Mikos, A. G. Biomacromolecules 7, 2796-2805, doi:10.1021/bm060680j (2006)). They typically offer limited cellular ingress as well as low gas and nutrient diffusion (Telemeco, T. A. et al. Regulation of cellular infiltration into tissue engineering scaffolds composed of submicron diameter fibrils produced by electrospinning Acta biomaterialia 1, 377-385, doi:10.1016/j.actbio.2005.04.006 (2005)), critical in the absence of an embedded vasculature (Novosel, E. C., Kleinhans, C. & Kluger, P. J. Advanced drug delivery reviews 63, 300-311, doi:10.1016/j.addr.2011.03.004 (2011)). Complementary strategies focused on increasing the porosity via enzymatically-controlled degradation (Wade, R. J., Bassin, E. J., Rodell, C. B. & Burdick, J. A. Nature communications 6, 6639, doi:10.1038/ncomms7639 (2015)), sacrificial components (Baker, B. M. et al. Proceedings of the National Academy of Sciences of the United States of America 109, 14176-14181, doi:10.1073/pnas.1206962109 (2012)) or expansion methods (Jiang, J. et al. Advanced healthcare materials 5, 2993-3003, doi:10.1002/adhm.201600808 (2016)) have therefore become necessary, but present additional steps and complexity in the fabrication process. In the context of HA fiber manufacturing, spinning methods have also typically required carrier polymers (Li, J., He, A., Zheng, J. & Han, C. C. Biomacromolecules 7, 2243-2247, doi:10.1021/bm0603342 (2006)), high temperatures (Li, J. et al. Macromolecular Rapid Communications 27, 114-120, doi:doi:10.1002/marc.200500726 (2006)) or added air-blowing systems to facilitate fiber formation (Um, I. C., Fang, D., Hsiao, B. S., Okamoto, A. & Chu, B. Biomacromolecules 5, 1428-1436, doi:10.1021/bm034539b (2004)), because high viscosity, hydrophilicity and surface tension can hinder manufacturability(Lee, K. Y., Jeong, L., Kang, Y. O., Lee, S. J. & Park, W. H Advanced drug delivery reviews 61, 1020-1032, doi:10.1016/j.addr.2009.07.006 (2009)).

In this example, high-throughput manufacture of pure full-length ECM proteins from aqueous solutions, that do not rely on polymeric carrier adjuvants is demonstrated. Importantly, it is demonstrated herein that pure protein nanofibers enable fabrication of ultra-soft (˜0.5-1.5 kPa) and robust, tissue-mimetic scaffolds and wound dressings unattainable using traditional spinning methods. The scaffolds fabricated herein are also highly porous (>60%) and water absorbent. These data exemplify how more optimal pro-regenerative properties can be obtained using a simple one-step process system.

Experiments performed in vitro highlighted in particular the advantage of such high porosity, illustrated by the rapid and in-depth cellular infiltration of dermal fibroblasts. Full-thickness excisional wound splinting experiments then enabled to investigate the regenerative potency of these HA scaffolds in mice. Remarkably, without functionalization, these scaffolds supported significantly faster granulation tissue formation and reepithelialization than non-treated controls, and long-term assessments further revealed a decreased trend in scar formation. Comparing scaffolds of varying porosity additionally reaffirmed the importance of appropriately tailoring structural properties for such indications. Altogether, this study demonstrated the use of a simple process for fabricating HA and other ECM molecules into nanofiber scaffolds, and how their assembly into biomimetic and porous structures supported tissue repair.

### Example 3A: Materials and Methods

The following materials and methods were used in Example 3.

The iRJS System

The immersed rotary jet spinning device used to fabricate the polymeric scaffolds is described in U.S. Patent Publication No. 2015/0354094, the entire contents of which are incorporated herein by reference. Briefly, the iRJS set-up consists of six main components: (1) a custom-machined 7075 aluminum reservoir coated with AMS 2482 Type 1 anodized hard coat, Teflon with 1 mil build up (25 um), an inner diameter of 40 mm, and two cylindrical orifices of 300 microns; (2) a remote-controlled electric motor with rotation speeds ranging from 1,000 rpm to 80,000 rpm; (3) a custom-built chemical resistant epoxy-coated cylindrical polycarbonate precipitation bath container with an inner diameter of 28 cm and a working volume of ˜5 L; (4) a custom-built aluminum rotating vortex generator connected via rotary sealed shaft to a pulley driven by motor with a spinning range of 1 to 500 rpm; (5) 3D-printed cylindrical sample collectors of variable diameters (from 8 cm to 20 cm) and height (from 5 to 20 cm), for tailored fiber sheet sizes, and (6) a remote-controlled syringe pump (PHD Ultra, Harvard Apparatus), providing working extrusion rates of 0.1 ml/min to 20 ml/min. The iRJS system was further placed in a humidity-controlled chamber.

### Protein Solution Preparation and Spinning

All full-length proteins described in this study were dissolved in aqueous solutions and spun into solvent-miscible precipitations baths, thus enabling rapid carrier solvent dissolution, and precipitation and stabilization of the protein in their fibrous physiological structures. Briefly, specific protein solution preparation and spinning methods are described:

Hyaluronic Acid (HA):

HHA was obtained (Hyaluronic acid sodium salt from Streptococcus equi, ˜1500-1800 kDa MW, Sigma) as a powder, dissolved in diH₂O and NaCl at various concentrations (from 1-4% weight/volume (w/v) and 0-600 mM, respectively) for 24-48 hrs at room temperature. See Table 1 for details. A precipitation bath of 80 percent ethanol was used.

Chondroitin Sulfate (CS):

CS was obtained (Chondroitin sulfate sodium salt from shark cartilage Sigma) as a powder, dissolved at 20% w/v in diH₂O for 24-48 hrs at room temperature. See Table 1 for details. A precipitation bath of 80 percent ethanol was used.

Collagen Type I (ColI):

ColI was supplied (Solution from rat tail, Sigma) in an aqueous solution of 20 mM acetic acid at a concentration of ˜4-4.5% w/v. ColI was either spun directly from the purchased solution, or purified through dialysis for 24 hrs in 10% Poly(ethylene glycol) (PEG) to reach a final concentration of ˜10% w/v. A precipitation bath of 80 percent ethanol was used.

Gelatin (Gel):

Gel was obtained (Bovine tendon, Bloom 300, Sigma) as a powder and dissolved at various concentrations in diH₂O (see Table 1) at 37° C. for 24 hrs. Because concentrated Gel solution form solid-like gels at RT, dope solutions were kept at or above 30° C., thus maintaining low enough viscosity to allow extrusion in the rotating reservoir of the iRJS. A bath of 95 percent ethanol was used to precipitate Gel fibers.

Fibrinogen (Fb):

Fb was purchased (Bovine plasma, Type I-S, Sigma) as a powder and dissolved at various concentrations (see Table 1) in DMEM (Thermofisher) at 37° C. for 3-4 days. Fb solution was then brought to RT and spun in a bath of 95 percent ethanol.

Fibronectin (Fn): Fn was obtained (Human protein, Plasma, Thermofisher) as a lyophilized powder containing 100 mM CAPS, 0.15 M NaCl and 1 mM CaCl2, for a pH of 11.5 when dissolved at 1 mg/ml. Here, Fn was first dissolved at 1 mg/ml in diH₂O for 1 hr, and subsequently concentrated via dialysis for 8 hrs in 10% PEG, 100 mM CAPS, 0.15 M NaCl and 1 mM CaCl2, for a final concentration of 5 mg/ml. pH was kept at ˜11. To facilitate fibrillogenesis of Fn via mechanical extension, Fn was first unfolded in solution by adding 10% w/v sodium dodecyl sulfate (SDS). Fn solution was then spun in a bath of 95 percent ethanol.

All solution dopes were loaded into a syringe and extruded in the iRJS rotating reservoir. Fibers were then collected in the precipitation bath. Different speeds were used for different protein solutions (see Table 1 for detailed specifications). Unless otherwise specified, air-gap distance was set at ˜5 cm. After spinning, fiber samples were briefly stored in their respective precipitation baths at −80° C., and subsequently lyophilized before use.

TABLE 1

| | | | | Reservoir | | |
|---|---|---|---|---|---|---|
| | Aqueous | Concentration | Dissolution | speed | Precipitation | Aditional |
| Protein | Solvent | (weight/vol) | method | rotation (rpm) | bath | comment |
| Collagen | Acedic | 2-10% | Stirred at | 2-30 k | 70-95% | Solutions can be spun |
| Type I | Acid (10- | | RT (2-6%) | (optimal | Ethanol | directly from supplier's |
| | 100 mM) | | Dialysis (5- | 15 k) | | aqueous |
| | | | 10%) | | | solution (2-6% w/v) |
| Fibrinogen | DMEM | 4-12.5% | 37° C. for 3- | 2 k-30 k | 70-95% | Fb solution |
| | | | 4 days | (optimal | Ethanol | was then |
| | | | | 12 k) | | brought to RT |
| | | | | | | and preloaded |
| | | | | | | in reservoir |
| Fibronectin | diH₂O and | 1-3% | Dialysis | 2-30 k | 70-95% | Variable |
| | salts | | | (optimal | Ethanol | concentrations |
| | | | | 15 k) | | of salts can be |
| | | | | | | used to improve |
| | | | | | | solution |
| | | | | | | viscosity and |
| | | | | | | dissolvability |
| Gelatin | diH₂O | 4-20% | 37° C. for | 2-30 k | 70-95% | Solution was |
| | | | 24 hrs | (optimal | Ethanol | spun at 30° C. to avoid |
| | | | | 15 k) | | gelling before |
| | | | | | | spinning |
| Hyaluronic | diH₂O and | 0.5-4% | Stirred at | 2-50 k | 70-95% | Variable |
| Acid | salts | | RT | (optimal | Ethanol | concentrations |
| | | | | 15 k) | | of salts can be |
| | | | | | | used to improve |
| | | | | | | solution viscosity and |
| | | | | | | dissolvability |
| Chondroitin | diH₂O | 20% | Stirred at | 15 k rpm | 70-95% | N/A |
| Sulfate | | | RT | | Ethanol | |
| Hyaluronic | diH₂O | HA: 0.5-4% | HA: Stirred | 2-50 k | 70-95% | Separate |
| Acid/ | salts | and Gel: 4-20% | at RT | (optimal | Ethanol | solutions are |
| Gelatin | | (ratios 10:1 to | 37° C. for | 15 k) | | mixed before |
| | | 1:10) | 24 hrs | | | spinning |

### Scaffold Dehydration, Lyophilization and Cross-Linking

To increase density of HA nanofiber scaffolds, dehydration was performed by removing sample from the precipitation bath and positioned between two holders, hanging horizontally. Sample sizes were kept identical when dehydration was performed. Dehydration times of 5-30 min were used. Alternatively, samples were directly placed in a −80° C. and subsequently lyophilized. If cross-linked, samples were placed in a solution of 80% ethanol with 10 mM EDC and 4 mM NHS for 24 hrs on a shaker. Samples were then washed several times in diH₂O and DMEM, before lyophilizing again and stored in 4° C.

### Scanning Electron Micrography (SEM) and Characterization

Fiber samples were mounted on SEM stubs and coated with 5-20 nm of platinum/palladium (Pt/Pd) using an EMS 300T Sputter Coater (Quorum Technologies) to minimize charge accumulation during imaging. Thin samples were coated with 5 nm of Pt/Pd, while thick and porous samples were coated with up to 20 nm. SEM imaging was then performed using a field emitting (FESEM Ultra55, Zeiss) at a voltage of 5 kV. For fiber diameter and porosity measurements, 6-8 fields of view at 1,000× or 2,000× magnification (depending on fiber size) were made per sample. Three different sample runs at least were used.

### Rheology Measurements

Rheology studies were conducted to measure viscosity profiles of HA solutions of different concentrations (1-4% w/v). Briefly, rheological properties were determined using a TA Instruments Discovery Hybrid 3 Rheometer with a cone plate geometry. The cone had a 40 mm diameter, 1° angle, and 26 μm truncation gap. The plate was temperature controlled to 25° C. and a solvent trap was used to ensure the sample did not lose solvent during testing. All materials in contact with the sample were aluminum. To load the sample, the cone was brought to a height above the plate defined by the truncation gap. After trimming the sample, the cone was raised and then brought back to the truncation gap. This repetition was employed to reduce normal forces generated during loading. After loading, a 300 s soak time ensured the sample reached equilibrium. The solution was sampled at a rate of 10 points per decade over 10⁻³ to 10⁴ (1/s). To ensure the solution reached equilibrium during each of these samplings, steady state sensing was used over 180 s of testing. If subsequent 30 second sample periods were with 5% tolerance of one another, then the sample was determined to have reached steady state and the next point was sample. Testing revealed that below 10⁻¹ (1/s) shear rates, the solution-rheometer system was dominated by surface forces while above 10⁻⁴ (1/s) shear rates the system was dominated by momentum. As these shear rates were not dominated by viscous force, they were not included in the date presented.

### X-Ray Micro-Computed Tomography (μCT)

μCT was performed with an X-Tek HMXST225 system (Nikon Metrology, Inc.) equipped with a 225 kV microfocus X-ray source with 3 μm focal spot size. Nanofiber fiber samples were incubated for 24 hrs on a shaker in a 1:10 dilution of Lugols's iodine solution to improve contrast upon imaging. An aluminum target and 115 kV accelerating voltage were used. Image acquisition and reconstruction was performed with InspectX (X-ray imaging and CT acquisition), CT Pro 3D (volume reconstruction) and VG Studio MAX 2.2 (3D volume visualization, rendering and analysis).

### Fourier Transform Infrared Spectroscopy (FTIR)

ATR-FTIR (Bruker) was performed to obtain infrared spectra of HA nanofibers and raw lyophilized powder over 600-4000 cm-1 at a resolution of 2 cm-1 with 16 scans. Measurements were normalized from 0 to 1. Graph plotting and analysis was performed using OriginPro 8.6 software (Origin Lab Corporation). For statistical analysis, at least 3 different areas were measured on each sample.

### Swelling Ratio and Degradation Kinetics

Lyophilized HA nanofiber samples were cut into ˜5 mg samples. Water absorption was calculated using the swelling ratio commonly used for hydrogels. The swelling ratio (SR) is defined as SR=(Wₕ−W_{d})/W_{d}, where W_{d} is weight of dry sample and Wₕ is weight of hydrated sample. Nanofiber samples were hydrated in diH₂O for 5 min before measurements. Degradation was evaluated by measuring loss in weight of hydrated samples in diH₂O over time (up to 10,000 min˜1 week).

### Mechanical Testing

Mechanical properties were measured in extension using a CellScale biaxial tensile tester (0.5 N load cells, Biotester, CellScale), and in compression using an Instron universal testing machine (Model 5566, Instron). Briefly, for tensile testing, samples were cut in rectangle shapes (5×10 mm) with a thickness of 2 mm, mounted for uniaxial testing, and tested using a 50% strain at 10% strain rate. Strain was applied parallel to fiber orientation. Measurements were performed at 37° C. in PBS. Mechanical testing in compression was performed with square samples (5×5 mm) with a thickness of 2 mm. Strain was set at 40% with 10% strain rate. Measurements were performed at RT in PBS. For both testing experiments, stress-strain curves were calculated for each sample and modulus was extracted.

### In Vitro Cell Infiltration Studies

GFP-expressing human dermal neonatal fibroblasts (GFP-HNDFs, Angioproteomie) were seeded on fiber HA fiber scaffolds (100,000 cells per sample) and imaged 30 min later using a confocal microscope (Olympus) under controlled culture conditions (37° C. and 95% humidity). Z-stack images were taken from the scaffolds surface to depths exceeding 100 μm. Image analysis, 3D reconstruction renderings, and infiltration intensity values were performed and quantified using ImageJ analysis software. GFP-HNDFs were cultured in cell growth medium consisting of Dulbecco's modified eagle medium (DMEM, ThermoFisher Scientific), 5% fetal bovine serum and 1% antibiotics (penicillin-streptomycin, ThermoFisher Scientific). Passages were made before cells reached 80% confluency and used for experiments until passage number 15.

### In Vivo Wound Healing Studies

All animal studies were performed following approved procedures by the Harvard University Institutional Animal Care and Use Committee (IACUC). Protocol follows previously established excisional wound splinting model that enables wound closure by reepithelialization instead of by wound contraction (Galiano, R. D., Michaels, J. t., Dobryansky, M., Levine, J. P. & Gurtner, G. C. Wound repair and regeneration: official publication of the Wound Healing Society [and] the European Tissue Repair Society 12, 485-492, doi:10.1111/j.1067-1927.2004.12404.x (2004)). Briefly, C57BL/6 male mice (8-10 weeks old) (Charles River Laboratories, Wilmington, Mass.) were anesthetized and maintained on surgical plane of anesthesia with isoflurane. After a toe pinch test confirmed, the back of the mice were first prepared by shaving with an electric razor (Kent Scientific, BravMini Pro, CL7300). The surgical area was then sterilized with alcohol and betadine (at least 2× each). A line across the centerline of the back was made with a surgical marker to facilitate positioning. Two full-thickness wounds were made on the back, lateral to the spin on both sides using 6 mm biopsy punches. Silicon splinting rings (OD: 10 mm, ID: 6 mm), sterilized in ethanol and under UV overnight, were applied and set in place with instant-bonding adhesive glue and sutured with 4 surgical knots. Nanofiber wound dressings were then applied to the wound with 5 μL of PBS to facilitate adherence and covered with Tegaderm silicon patches. Mice were monitored daily. Photographic images of the wounds were performed every 3 days. Tissues were collected on day 6 to assess granulation tissue formation, reepithelialization and scaffold integration. Treatments and controls application was randomized

### Histological Analysis

Histology was performed by HMS Rodent Histopathology Core following standard protocols. Tissues were harvested at days 6 and 28 after wounding and fixed with 4% paraformaldehyde for 15 min Samples were then washed and stored in PBS before PFA embedding, sectioning and staining. Whole-slide imaging was performed using a slide scanner (Virtual Slide Microscope VS120, Olympus) with a 20× objective. Granulation tissue formation and reepithelialization were analyzed with using FIJI image analysis software (ImageJ, NIH).

### Statistical Analysis

Statistical analyses were conducted using SigmaPlot (v12.0, Systat Software, Inc., CA). One-way ANOVA on ranks with post hoc multiple comparisons Dunn's test, or Holm-Sidak's test, and Student's t-test were used where appropriate. Quantitative data are presented as mean±SEM and significance was considered for p<0.05.

### Example 3B: Production-Scale Manufacture of Biological Polymer Nanofibers Using iRJS

Using fetal-inspired extracellular matrix nanofiber scaffolds, biomimetic pro-regenerative nanofiber scaffolds, for use as a ‘soil’ strategy to stimulate endogenous repair were prepared. These protein-based nanofiber scaffolds recapitulate the multiscale fibrous structure and biochemistry of fetal ECM and promote faster wound closure and enhance skin tissue restoration.

In the current automated setup of the iRJS system, a polymer solution is continuously channeled in the rotating reservoir, accelerated through two 350 micrometer-wide orifices via high centrifugal forces, ejected across an air-gab and into a precipitation bath (FIGS. 61A and 62). As the polymer jet hits the bath, the carrier solution rapidly dissipates, leaving an aggregated and stable fiber whirling in the vortex40. The polymer fiber then gradually and continuously wraps around a cylindrical collector (in gray), forming a non-woven thick sheet (in white) (FIGS. 61B and 61C). A 5-liter vortexed precipitation bath and a large cylindrical collector enabled the manufacture of centimeter-wide thick nanofiber scaffolds.

To first illustrate the versatility of this approach, the fabrication of several different ECM proteins and GAGs was investigated. Typically, engineering biological fibers has been enabled or enhanced by using synthetic carrier polymers (such as, polycaprolactone or polyethylene glycol) that facilitate jet elongation and fiber formation (Badrossamay, M. R. et al. Biomaterials 35, 3188-3197 (2014)). Although incorporation of such materials may prove critical in certain applications where for example superior mechanical properties are required (i.e. tissue engineered heart valves (Capulli, A. K. et al. Biomaterials 133, 229-241, doi:10.1016/j.biomaterials.2017.04.033 (2017)) and ventricles (MacQueen, L. A. et al. Nature Biomedical Engineering, doi:10.1038/s41551-018-0271-5 (2018))), designing entirely biological fibrous materials remains relevant for a variety regenerative medicine applications (Pashuck, E. T. & Stevens, M. M. Science translational medicine 4, 160sr164, doi:10.1126/scitranslmed.3002717 (2012); Xia, H. et al. Nature Reviews Materials 3, 174-193, doi:10.1038/s41578-018-0027-6 (2018)). The GAG: chondroitin sulfate (CS), two ECM proteins: fibrinogen and collagen type I, as well as gelatin, the denatured form of collagen were all spun directly from aqueous solutions. SEM images reveal the formation of fibrous structures for all these materials (FIG. 61D), and higher magnification micrographs detail their respective ultrastructures (FIG. 62). Wide ranges of polymer concentrations and blends were furthermore permitted (FIG. 63) as detailed in Table 1.

To next demonstrate the high-throughput caliber of this technology and the proceeding tunability of the scaffolds, the production of hyaluronic acid (HA) was focused on. Notably, fabrication of HA nanofiber scaffolds was possible at production rates far exceeding alternative manufacturing methods that depend on electrical fields for fiber formation (i.e. electrospinning (Um, I. C., Fang, D., Hsiao, B. S., Okamoto, A. & Chu, B. Biomacromolecules 5, 1428-1436, doi:10.1021/bm034539b (2004))), whether quantified by polymer solution volume or mass (FIG. S3).

In particular, a polymer solution comprising 1% w/v, or 2% w/v, 3% w/v or 4% w/v hyaluronic acid (HA) was placed into the reservoir of an immersed rotary jet spinning (iRJS) device and was extruded through tan orifice in a rotating reservoir rotated at about 15,000 rpm into a collection device comprising a precipitation bath of about 80% ethanol, e.g., a reservoir and a collection device positioned such that the one or more orifices of the reservoir are positioned in an air gap of a liquid vortex in the collection device created by causing the liquid in the collection device to rotate; and wherein the ejection of the polymer into the air gap and subsequently into the liquid of the liquid vortex in the collection device causes formation of one or more micron, submicron or nanometer dimension polymeric fibers.

The formed scaffolds comprising the polymeric fibers were post-processed by drying, e.g., lyophilization, for subsequent analyses.

As depicted in FIG. 65, a wide range of polymer concentrations (from 1 to 4 percent) could be consistently spun into uniform and robust scaffolds, thus offering the ability to tailor fiber structure and mechanics to specific applications. This increased flexibility on polymer concentration is caused by a reduced reliance on traditional spinning parameters. Indeed, the use of non-volatile solvents decreased surface tension instabilities at any given jet-elongating time-point, while the introduction of a precipitation bath abbreviated the jet-elongating phase altogether. Additionally, the use of high centrifugal forces, causing high shear strain rates in the reservoir channel, decreased dependency on solvent viscosity—a common hindrance of traditional spinning or 3D-printing techniques. This was confirmed with rheological measurements of HA dopes that revealed shear-thinning behaviors, where viscosity curves significantly decreased at high shear rates and showed convergent trajectories for all different concentrations (FIG. 66). Beading or fiber breakage could thus be minimized for a variety of dope concentrations, while spinning capabilities were retained or even increased.

The reproducibility and uniformity that was furthermore achieved is exemplified by the SEM image taken at the center of a centimeter-thick scaffold (FIG. 67) and X-ray Micro Computed Tomography (μCT) renderings of a millimeter-thick scaffold (FIG. 65B). This readily addresses a limitation of previously described HA fiber wound dressings (Uppal, R., Ramaswamy, G. N., Arnold, C., Goodband, R. & Wang, Y. Journal of biomedical materials research. Part B, Applied biomaterials 97, 20-29, doi:10.1002/jbm.b.31776 (2011)), particularly relevant if these scaffolds are for clinical and regulatory approval.

### Example 3C: Investigating and Tuning Fiber Structure and Mechanics

The architectural and biophysical properties are, along with a microenvironment's unique biochemical makeup, critical mediators of tissue function and regeneration. Designing potent pro-regenerative scaffolds must therefore require the ability to tailor these specific properties—whether mechanical or structural—to a specific organ for optimal integration and subsequent regenerative instruction.

It was sought here to further explore these biomimetic and instructive material properties using the model material: hyaluronic acid. First, iRJS induced HA assembly into fibrous internally-aligned structures (FIG. 64), often observed in ECM proteins in vivo (Hynes, R. O. Science (New York, N.Y.) 326, 1216-1219, doi:10.1126/science.1176009 (2009)). Fourier-transform infrared spectroscopy (FTIR) next revealed a decrease of the hydroxyl- and C—O—C— groups of HA fibers compared to the raw lyophilized powder, suggesting an intra-fiber molecular packing (FIG. 62). Individual fibers ranged from ˜1 μm to ˜3 μm for dope concentrations of 1-4 percent weight/volume (w/v) (FIG. 68), while lower concentrations further decreased the range of attainable fiber sizes to nanometer scales (˜600 nm for 0.5%) (FIG. 69). Conversely, varying reservoir speed rotation, thus modulating the shear forces that form the polymer jet, likewise modified fiber diameter (FIGS. 68B and 70).

The porosity of these HA scaffolds was next investigated, as tissue integration can be severely hampered by often minimally-porous nanofiber scaffolds (Baker, B. M. et al. Proceedings of the National Academy of Sciences of the United States of America 109, 14176-14181, doi:10.1073/pnas.1206962109 (2012). Remarkably, porosities between 65 and 75% were measured for all our tested conditions (FIG. 68C), contrasting the significantly lower percent-range (40-55%) of dry-spinning techniques (Capulli, A. K. et al. Biomaterials 133, 229-241, doi:10.1016/j.biomaterials.2017.04.033 (2017)) (FIG. 71). The collection method—a wet rotating bath—supports a looser scaffold assembly, and concomitantly prevents inter-fiber stacking or bonding, which may occur in traditional dry-spinning setups. Notably, it was also observed that fiber sheet dehydration at room temperature post-spinning and prior to further storage in a precipitation solution exhibited decreased porosities. The effect of dehydration on HA scaffolds was thus investigated and it was discovered that there was an evident dependency with time, as porosities could be significantly reduced with drying times of 15 min or above, while other parameters remained unchanged (FIGS. 68D and 68E). The faster evaporation of ethanol compared to that of water in the precipitation solution (80% ethanol/20% water) likely caused a gradual increase in water content that facilitated fiber dissolution, thus catalyzing inter-fiber packing or bonding, and subsequently, a decrease in scaffold porosity.

Next, water absorbent dressings have demonstrated strong ability in removing wound exudates, while providing a hydrated environment for cell viability and growth. As such, the swelling ratios of the scaffolds were measured, exhibiting highly absorbent properties (˜1500-3000%) within the first minutes of water contact (FIGS. 68 and 72). For comparison, this is an order of magnitude higher than previous cellulose-based fiber scaffolds that supported tissue restoration in a murine model (Ahn, S. et al. Advanced healthcare materials, doi:10.1002/adhm.201701175 (2018)). Prolonged measurements however revealed a rapid degradation of their fibrous architecture, indicative of non-cross-linked HA polymer chains.

In order to provide additional mechanical and structural stability to the HA fiber scaffold without loss of the desirable structural characteristics of the formed fibers and scaffolds, the formed scaffolds were covalently cross-linked via ester bond formation by contacting the scaffolds with a solution of ethyl(dimethylaminopropyl) carbodiimide (EDC)/N-hydroxysuccinimide (NHS) (10 mM/4 mM) for 24 hours, with shaking.

Ester bond formation was induced via an EDC/NHS catalyst, linking the hydroxyl- and carboxyl-groups of the HA molecule, thus significantly decelerating degradation kinetics (FIGS. 68 and 72). After a week, the scaffolds still retained 80% or more of their initial weight. Concomitantly, an increase in water absorbance for these cross-linked scaffolds was observed, reaching a ratio close to 6000% for the 1% HA samples (FIG. 68F). Fiber diameter and porosity were, however, unaffected by this cross-linking process.

Mechanical properties finally demonstrated stiffness regimes in pare with mammalian soft tissue mechanics—theoretically a prerequisite for bio-mimetic scaffold design. Measurements were performed in compression and extension (along the fiber axis), exhibiting storage moduli ranging from ˜450 to 1,500 Pa (in compression) and ˜5 to 100 kPa (in extension) (FIG. 68G). These data additionally revealed that scaffold mechanics are significantly influenced by fiber size, as indicated by the higher properties with increased HA concentrations.

### Example 3D: Highly Porous HA Scaffolds Enable Direct Cellular Infiltration

The ability to manipulate HA scaffold properties was leveraged to investigate the influence of porosity on cell infiltration. It was hypothesized that highly porous scaffolds should enable rapid ingress of cells, when compared to denser scaffolds that are more representative of existing pro-regenerative fibrous materials (Baker, B. M. et al. Proceedings of the National Academy of Sciences of the United States of America 109, 14176-14181, doi:10.1073/pnas.1206962109 (2012)). Three different groups were thus tested in an in vitro assay, and were termed: porous HA (pHA; ˜75% porosity), standard HA (sHA; ˜65% porosity), and dense HA (dHA; ˜55% porosity). Other parameters were kept unchanged (precursor solution of 1% HA spun at 15 k rpm) with fibers in the ˜1 micron range, low stiffness regimes and high water absorbency.

In this assay, HA scaffolds of ˜0.5 mm in thickness were seeded with GFP-human neonatal dermal fibroblasts (GFP-HNDF) and tracked under live confocal microscopy 30 min following seeding. Initial observations with images at varying depths and 3D reconstructions confirmed the hypothesized influence of porosity on cellular infiltration (FIGS. 73A and 73B). Dermal fibroblasts in the dense dHA scaffolds were indeed constrained to the surface, where a compact network of fibers likely acted as an almost impermeable barrier to entry. Intensity measurements supported these observations, evident by a rapid decrease of signal 15 microns through (FIG. 73C). In contrast, the more porous sHA scaffolds allowed penetration of cells in the sample, while the highly porous pHA supported a close to homogenous diffusion of cells through over 100 microns of scaffold (FIGS. 73D and 73E). Quantification of the average infiltration (based on intensity values) and the infiltration at the 100 μm depth position further revealed higher values for the pHA scaffolds, when compared to the other conditions. These data reiterate the relevance of appropriately tailoring scaffold properties for applications in tissue engineering and regenerative medicine, and underscore in particular the importance of porosity.

### Example 3E: Accelerated Tissue Integration and Repair Through Increased Scaffold Porosity

It was next hypothesized that these porous HA scaffolds (pHA) should potentiate rapid tissue integration and subsequent tissue repair, when tested in vivo. sHA scaffolds were used as the denser controls, despite being as or more porous than materials fabricated using other spinning techniques. Three different groups were thus tested on full-thickness wounds in mice, following established excisional splinting protocols: pHA and sHA wound dressings, and a non-treated controls. All wounds were additionally covered with a Tegaderm film dressing to secure nanofiber scaffolds and limit entry of external pathogens (FIGS. 74A and 74B).

Macroscopically, both HA-treated wounds exhibited clear formation of a scab 4-6 days post-wounding, contrasting the lack of any tissue in the non-treated controls (FIGS. 74C and 74D). Histological analysis via trichrome staining at day 6 further revealed marked differences in wound morphologies (FIG. 74E). pHA and sHA demonstrated indeed robust reepithelialization (in red, highlighted with arrows), while formation of a granulation tissue was apparent underneath the entire wounded area. Quantification of new epidermis formation displayed an upregulated trend for the two HA treatments, with a significant difference measured between the control and the pHA specimen (FIG. 74F, top). Presence of remnant HA fibers over the epidermis further suggests an efficient cellular infiltration, thus supporting neogenesis of dermal and epidermal tissues. This was in particular emphasized by the marked differences in granulation tissue formation between all groups tested (FIG. 74F, bottom), heralding porosity as a key regulatory property. By contrast, non-treated wounds, covered only with the Tegaderm film, showed sparse reepithelialization, with wounds that were typically void of any scab or granulation tissue. These underscore how changes in the material structural properties—here the scaffold's porosity—can have potent influences on wound healing and tissue formation.

### Example 3F: Porous HA Scaffold Reduces Scar Formation

Finally, to verify the influence of these biomimetic and porous HA scaffolds (pHA) on the long-term outcome of wound healing, the formation of scar tissue 28 days post-wounding was examined (FIG. 75A). Photographic images revealed smaller scar sizes for the treatment group, while a reduced red pigmentation suggests faster recovery of normal capillary density levels⁵⁹—corroborating our data on accelerated wound healing (see FIG. 74). When measured as a percentage of original wound size, control scars averaged at ˜19.5%, while pHA-treated specimen showed a decrease with an average at ˜11% (FIG. 75B). Differences at this healing endpoint underscore how influencing early-stage tissue integration and wound closure can lead to long-standing effects. Importantly, these results were achieved by relying entirely on the nanofibrous structure of these HA scaffolds and their inherent biochemical makeup, suggesting promise for strategies that would integrate additional cell binding moieties or morphogen cues.

Designing organ-specific pro-regenerative materials requires the ability to precisely tune biophysical and biochemical properties to support and stimulate an endogenous response^{7,60}. In this context, the versatility of a nanofiber manufacturing method—termed immersed rotary jet spinning (iRJS)—was investigated for engineering tunable hyaluronic acid scaffolds, while achieving similar fabrication flexibility with a wide range of other ECM molecules. Either in pure form or as hybrids, these engineered materials formed fibrous scaffolds at production rates readily amenable to clinical translation, while being fashioned from entirely aqueous solutions. Overcome the need to rely on organic solvents may prove advantageous as these chemicals were shown to denature ECM proteins, and effectively reduce their biological functionality. The reliable manufacturing capabilities furthermore spurred the establishment of a comprehensive structural and mechanical parameter framework, achievable within an iRJS system. Notably, it was observed that fiber diameters could range from hundreds of nanometers to several microns, while high porosity, water absorbency and tissue-level mechanics were inherent features of all HA-based scaffolds. Degradation kinetics and porosity could likewise be tuned, thus offering a holistic approach for designing the structural and mechanical properties of biomimetic materials.

These HA scaffolds were collected into large centimeter-wide sheets and cut into 500 micron-thick circular dressings for studies in vitro and applications in an excisional splinting wound mouse model in vivo. It was first sought to understand the effect of highly porous scaffolds (pHA, ˜75%) on cellular infiltration. Porosity remains indeed a critical regulator in supporting rapid scaffold integration, which subsequently facilitates downstream tissue repair mechanisms. In vitro, rapid and in-depth ingress of seeded dermal fibroblasts was measured, with a roughly homogenous distribution. By contrast, the denser sHA and dHA scaffolds (of ˜65% and ˜55% porosity, respectively)—while remaining porous in comparison to other nanofiber scaffolds—demonstrated stronger accumulation of cells at the scaffold's surface and concomitant poorer infiltration.

It was next investigated whether these biomimetic HA scaffolds could potentiate wound closure and tissue repair in a wound mouse model, and, notably, how porosity was affecting these reparative mechanisms. Our data first revealed that both HA scaffold significantly supported the wound closure process, contributing to the rapid formation of scabs over the wounds. Histological analysis then underscored the relevance of higher porosity, exemplified by the rapid formation of granulation tissues and long protruding epithelial tongues days after injury in the pHA specimen. By contrast, the sHA dressing initiated tissue repair, yet at a lower level, while the controls showed close to no wound closure and tissue restoration, illustrated by the large gaps remaining between the wound edges. In a long-term study, pHA treatments further revealed a trend of reduced scar formation and more mature regenerated tissues 28 days post-wounding, suggesting promise for further investigation.

Altogether, these data reveal how designing materials with faithful biomimetic features, such as mechanical and structural properties, and that are amenable to rapid tissue integration through a porous interface, can potentiate tissue repair. The influence of porosity, highlighted in vitro and in vivo, was in particular made evident by the poor cellular ingress and slow tissue formation in denser scaffolds. Remarkably, without relying on integrated cell-binding moieties or additional morphogenic cues, these HA scaffolds caused marked differences within the first week of treatment, embodied by faster scab formation, re-epithelialization, and granulation tissue formation.

### Example 4: Biomimetic and Estrogenic Alfalfa-Polycaprolactone Composite Nanofibers as Aligned Bioscaffolds

Once damaged, it is challenging for human tissues to completely regenerate their original structure and function due to their lack of intrinsic regenerative capacity. Accordingly, there is a great need for developing biocompatible tissue scaffolds in an effort to support and facilitate tissue reconstruction (Griffith, L. G.; Naughton, G. Science 2002, 295 (5557), 1009-1014).

There is a wide variety of materials that can be used for the production of engineered scaffolds that provide a backbone and/or present bioactive moieties, however, there are numerous drawbacks associated with such materials. For example, synthetic polymers, such as polycaprolactone (PCL) or polyurethane, are capable of forming fibrous networks due to high polymer chain entanglements and can therefore recapitulate the native fibrous architecture of tissues (Ma, P. X. Adv. Drug Del. Rev. 2008, 60 (2), 184-198). However, these polymers alone lack bioactive domains that enhance cell adhesion and growth, requiring these materials to be functionalized with additional bioactive moieties. In contrast, animal-derived proteins (e.g., collagen) are rich in cell-binding domains, but are expensive, have poor mechanical properties, may be immunogenic, and are associated with ethical concerns (Ma, P. X. Adv. Drug Del. Rev. 2008, 60 (2), 184-198; Chan, G.; Mooney, D. J. Trends Biotechnol. 2008, 26 (7), 382-392 Plant-derived materials provide an alternative because they are biocompatible, renewable, and primarily non-immunogenic and are not associated with ethical issues (Reddy, N.; Yang, Y. Trends Biotechnol. 2011, 29 (10), 490-498; Liu, W.; Burdick, J. A.; van Osch, G. J. Tissue Eng., Part A 2013, 19, 1489-1490). They also include bioactive molecules similar to extracellular matrix (ECM) proteins or hormones that control cell fates (Ahn, S., et al. Adv. Healthcare Mater. 2018, 7 (9), e1701175). However, engineering plant-based scaffolds remains largely unexplored due to the limited choices of materials.

From ancient times, humans have utilized herbal medicines to cure diseases. Amongst various medicinal herbs that have been used, alfalfa (“father of all foods” or Medicago sativa) is one of the most primitive and the most used plants. Historically, oral and topical applications of alfalfa have been known to treat central nervous system (CNS) disorders, diabetes, kidney pain, fever, ulcers, arthritis, breast cancer, urinary, cutaneous wound, menopausal symptoms etc. And it has been found that alfalfa possesses many bioactive chemicals which could be beneficial to human health (Bora, K. S.; Sharma, A. Pharm. Biol. 2011, 49 (2), 211-220). For instance, alfalfa contains proteins that can have human ECM protein-mimetic structure and integrin-like function to control cell responses and cell fate (Garcia-Gomez, B. I., et al. The Plant Journal 2000, 22 (4), 277-288; Bardor, M., et al. Plant Biotechnol. J. 2003, 1 (6), 451-462).

In addition, alfalfa contains phytoestrogens that are structurally and functionally similar to estrogen (Bora, K. S.; Sharma, A. Pharm. Biol. 2011, 49 (2), 211-220). Estrogen, a primary female hormone, affects multiple organs in humans by binding to estrogen receptors (ERs) in the cells. Oral or topical estrogen therapies have revealed potentials to reverse diseases in post-menopausal women due to the estrogen deficiency. 10 For cutaneous wound healing, estrogen facilitates wound closure via ER β and transforming growth factor-β1 (TGF-β1) (Ashcroft, G. S. et al. Nat. Med. 1997, 3 (11), 1209; Campbell, L., et al. J. Exp. Med. 2010, 207 (9), 1825-1833. Cardioprotective roles of estrogen against coronary heart diseases and ischemia have been well explained by utilizing animal models with estrogen treatment (Moolman, J. A., Cardiovasc. Res. 2006, 69 (4), 777-780). Like estrogen, phytoestrogens can also bind to ERs and trigger ER-related pathways to influence human organs (Patisaul, H. B.; Jefferson, W. Front. Neuroendocrinol. 2010, 31 (4), 400-419). Although clinical potentials and bioactive contents of alfalfa have been reported over the past centuries, alfalfa has not been explored as a building block to design and engineer biomaterials.

In this study, the fabrication of alfalfa-based nanofibers is presented and their functionality as a bioscaffoldis demonstrated. Nanofibers have shown significant potentials as engineered tissue substrates. They can easily recapitulate structural cues of native ECM microenvironments vital for healthy tissue functions. Nanofibers provide high surface area-to-volume ratio, controlled geometry (fiber size, alignment, porosity, and thickness), and high production rate. In addition, aligned nanofibers can guide anisotropic tissue formation (for cardiac tissue engineering) and accelerate cellular migration (for neurite outgrowth and wound healing application). Recent studies have highlighted that plant-based scaffolds can provide ECM-mimetic microenvironments while delivering phytoestrogens and/or other bioactive molecules for enhanced tissue regeneration. Therefore, the goal is to develop estrogenic and aligned nanofiber scaffolds by using a natural and beneficial biomaterial, like alfalfa, as a building block. Here, it is hypothesized that alfalfa nanofibers can provide ECM-mimetic nanostructures and to deliver bioactive molecules (proteins and phytoestrogens) that will enable a faster rate of regeneration of functionally-mature tissues. To stabilize these bioactive components, PCL/alfalfa composite nanofibers were engineered using PCL as a co-spinning polymer in a pull spinning system. Polymer concentrations were varied to optimize for continuous fiber formation. Additionally, physical (fiber diameter and stiffness) and chemical (presence and distribution of bioactive components) properties of nanofibers was investigated to find an optimal polymer composition for bioactive scaffolds and to confirm the delivery of bioactive chemicals from nanofibers. In vitro cell culture on PCL/alfalfa nanofiber scaffolds was also performed, which showed good biocompatibility, cellular growth, and maturation of anisotropic tissue. Finally, it was confirmed the feasibility of this scaffold for regenerative applications by evaluating its effect on in vivo wound healing.

### Example 4A: Materials and Methods

The following Materials and Methods were used in Example 4.

### Materials

Polycaprolactone (PCL) (Mₙ 70,000; Sigma-Aldrich, USA), alfalfa (powdered alfalfa leaf; Frontier Natural Products Co-op, USA), and 1,1,1,3,3,3-hexafluoro-2-propanol (HFIP; Oakwood Chemical, USA).

### Fiber Spinning

Pull spinning was used to produce nanofibers as described previously (Deravi, L. F., et al. Macromol. Mater. Eng. 2017, 302 (3); Ahn, S., et al. Anal. Bioanal. Chem. 2018). Briefly, different concentrations of alfalfa were dissolved in HFIP with 6 wt/v % of PCL. The solution was stirred overnight. The prepared solution was pumped at 0.3 mL/min and contacted with the rotating bristle at 25,000 RPM, forming nanofibers. The spun nanofibers were dried in a chemical hood overnight to remove excess HFIP before further characterization. For in vitro cell culture studies, the nanofibers were directly spun on the coverslips.

### Scanning Electron Microscopy (SEM)

The spun nanofibers were mounted on the SEM stubs. Pt/Pd (5 nm thickness, Denton Vacuum, USA) was sputter-coated on the nanofibers before imaging. The samples were imaged using field emission scanning electron microscopy (FESEM, Zeiss, USA).

### Fiber Diameter, Alignment, and Porosity Analysis

SEM images of nanofibers were used to determine the fiber diameter, alignment, and porosity. The analysis was performed by utilizing ImageJ software (NIH) with the DiameterJ plug-in (Hotaling, N. A., et al. Biomaterials 2015, 61, 327-338). For the fiber alignment analysis, Gaussian fitting was applied to the raw data to show the anisotropic distribution of fiber alignment.

### Fourier Transform Infrared Spectroscopy

Attenuated Total Reflectance-Fourier Transform Infrared spectroscopy (ATR-FTIR, Lumos, Bruker, USA) was used to obtain FT-IR spectra of samples. The raw spectra were normalized from 0 to 1. OriginPro 8.6 software (Origin Lab Corporation) was used to plot the normalized spectra.

### Ultraviolet-Visible (UV-Vis) Absorption Spectroscopy

The nanofiber membranes were placed in the spectrometer (Cary 60 UV-Vis, Agilent, USA). The absorption spectra were collected from 400 nm to 800 nm.

### Hyperspectral Imaging

PCL and PCL/alfalfa fibers cast on silicon wafers were imaged under reflectance mode using a darkfield hyperspectral microscope (Cytoviva) integrated with a confocal Raman microscope (Horiba XploRA PLUS). Hyperspectral maps were processed using ENVI data analysis software the (ENVI Classic 5.4) to reconstruct the spectral information multiple regions of interest per fiber. The corresponding darkfield images were obtained using a 50x objective under a halogen lamp (International Light Technologies Part L1090, USA).

### Contact Angle Measurement

To measure contact angles, the cast films were prepared by pouring and drying the polymer solution in a Petri dish overnight at room temperature. 10 μL of water was dropped on the surface of the samples. The droplet formation was photographed. ImageJ software with the Drop Shape Analysis plug-in was used to calculate contact angle (Stalder, A., et al. Colloids Surf. Physicochem. Eng. Aspects 2006, 286 (1-3), 92-103; Stalder, A. F., et al. Physicochem. Eng. Aspects 2010, 364 (1-3), 72-81).

### Mechanical Property Testing

Single fiber standard ASTM D3822M-14 was adapted to determine the modulus of fiber sheets. A frame, cut from 130 μm thick polycarbonate sheet, was employed to ensure no fiber slippage at the fiber clamp interface. The frame had a gauge length of 2.5 mm to match the length of the cantilever. Fiber samples were cut to 10 mm length and secured to the frame using a primer (Loctite® 770, USA) followed by the application of an adhesive (Loctite® 401) to ensure no slippage between the frame and the fiber (Wang, H., et al. In The Effectiveness of Combined Gripping Method in Tensile Testing of Uhmwpe Single Yarn, IOP Conf. Ser.: Mater. Sci. Eng., IOP Publishing: 2015; p 012109). After preparation, a frame loaded with a sample was placed into pneumatic grips of an Instron Model 5566 equipped with a 10 N Load Cell. After loading, the frame was cut to allow for extension of the fiber sheets. The sample was then strained at a rate of 240% per min until sample break. The specific modulus (modulus divided by specific density) was also calculated to account for the effect of porosity on the sample properties.

### Liquid Chromatography-Mass Spectrometry

The amount of genistein in alfalfa powder and nanofiber was measured by using Liquid Chromatography-Mass Spectrometry (LC-MS, Agilent 1290/6140, USA). Samples were prepared in dimethyl sulfoxide (DMSO, HPLC grade, Sigma-Aldrich, USA). A gradient of H₂O and acetonitrile (ACN) with a flow rate of 0.25 mL/min was selected as a mobile phase for C18 LC column (ZORBAX RRHD C18, USA). The gradient was as follows; 95% H₂O and 5% ACN were maintained for first 2 min. Then, the ratio increased to 100% B in 10 min 100% B was retained for 2 min and decreased to 95% A and 5% B in 1 min After the chromatographic separation, electrospray ionization (ESI) was applied to ionize molecules and thus detect each ions based on their molecular weights. For genistein detection, negative ESI-MS scan at 269 (m/z) was performed.

### Cell Culture

Green fluorescent protein (GFP)-expressing human neonatal dermal fibroblasts (HNDFs, Angio-Proteomie, USA) and primary neonatal rat ventricular myocytes (NRVMs) were cultured on nanofibers as described previously (Ahn, S., et al. Adv. Healthcare Mater. 2018, 7 (9), e1701175; Grosberg, A., et al. Lab Chip 2011, 11 (24), 4165-4173). Briefly, for HNDF culture, cells were delivered at passage 3 and subcultured to passage 7 in Dulbecco's modified eagle medium (DMEM, ThermoFisher Scientific, USA) with 5% fetal bovine serum (FBS) and 1% antibiotics (penicillin/streptomycin, ThermoFisher Scientific, USA). The cells at passage 7 were isolated by using trypsin/ethylenediaminetetraacetic acid solution (trypsin/EDTA, Lonza, USA). 100,000 cells per sample were seeded. Cell culture media (DMEM without FBS) were replaced every 2 days. For NRVM culture, cells were extracted from two-day-old Sprague-Dawley rats followed by previously established and IACUC approved protocols (Grosberg, A., et al. Lab Chip 2011, 11 (24), 4165-4173). 1,000,000 cells per sample were seeded. Cells were cultured in M199 culture media with 10% heat-inactivated fetal bovine serum (FBS), 10 mM HEPES, 0.1 mM MEM nonessential amino acids, 20 mM glucose, 2 mM L-glutamine, 1.5 μM vitamin B-12 and 50 U/mL penicillin After 48 h of cell culture, concentration of FBS in the media decreased to 2%. After 5 days of cell culture, cells were fixed. All animal protocols performed in this study were approved by Institutional Animal Care and Use Committee (IACUC) at Harvard University. Primary cortical neurons were harvested from 2-day-old Sprague-Dawley rat pups (Charles River Laboratories) as described previously (Dauth, S., et al. J. Comp. Neurol. 2016, 524 (7), 1309-1336; Dauth, S., et al. J. Neurophysiol. 2016, 117 (3), 1320-1341. Briefly, pups were euthanized via decapitation, and surgically removed whole brains, except the cerebellum and olfactory bulbs, were minced in warmed HABG media (Hibernate-A with B-27 and GlutaMax supplements; all GIBCO Life Technologies, Grand Island, N.Y.). Minced tissue was digested for 30 mins at 37° C. with papain (Worthington Biochemical Corporation, Lakewood, N.J.) prior to mechanical trituration with silane treated, fire polished glass Pasteur pipettes. Cell-containing supernatant was collected, filtered through a 40 μm cell strainer (BD Bioscience, San Jose, Calif.), and centrifuged at 250 rcf for 5 mins After the supernatant was aspirated, the resulting cell pellet was re-suspended in warmed NBA media (Neurobasal A with added B-27, GlutaMax, and gentamycin; all GIBCO). Cells were counted using a hemocytometer (SKC Inc, Covington, USA) and diluted in NBA media prior to seeding on nanofiber-covered glass coverslips at a density of 3000 cells/mm². After 1 h, samples were washed with fresh NBA medium to remove debris and non-adherent cells. All samples were incubated under standard conditions of 20% O₂ and 5% CO₂ at 37° C., with half-volume media changes every 3 days until experiments were conducted.

### Cytotoxicity Measurement

Cytotoxicity of nanofibers was investigated using a commercial lactic acid dehydrogenase (LDH) assay (Promega, USA). Cell culture media at Day 5 of HNDF culture was collected. The collected media was incubated with the reagent for 30 min at room temperature. Then, stop solution was added to samples and absorbance of the solutions was measured at 490 nm using a microplate reader (BioTek, USA).

### Neurite Outgrowth Analysis

Neurons cultured for 7 days were fixed by 4% paraformaldehyde (PFA) and permeabilized by 0.05% Triton X-100 for 10 min. The fixed samples were incubated with 5% bovine serum albumin (BSA) for 2 h at room temperature to block non-specific binding. After blocking, samples were incubated with a primary antibody (anti βIII tubulin, Abcam, USA) in 0.5% BSA for 1 h at 37° C., followed by 3 times PBS wash and Alexa Fluor 488-conjugated mouse IgG (H+L) secondary antibody (Invitrogen, USA) and 4′,6-diamidino-2-phenylindole dihydrochloride (DAPI, Invitrogen, USA) for 1 h at 37° C. Samples were mounted on glass slides and imaged immediately using a spinning disc confocal microscope (Olympus ix83, USA). Neurite outgrowth was measured by using ImageJ software (NIH) with the NeuriteTracer plug-in (Pool, M., et al. J. Neurosci. Methods 2008, 168 (1), 134-139).

### Cell Coverage Analysis and 3D Reconstruction

GFP-expressing HNDFs on nanofibers at Day 7 of cell culture was imaged using confocal microscopy. The coverage of HNDFs was analyzed using ImageJ to calculate the area percentage of GFP-positive area from the confocal images. For 3D reconstruction of z-stack images, NRVMs cultured for 5 days were fixed by 4% PFA and 0.05% Triton-X for 10 min. The fixed samples were incubated with a primary antibody (anti α-actinin, Sigma-Aldrich, USA) for 1 h, followed by Alexa Fluor 546-conjugated rabbit IgG (H+L) secondary antibody (Invitrogen, USA) and DAPI for 1 h. 3D reconstruction of z-stack images from DAPI and anti α-actinin stains was performed by using Zeiss Zen microscope software (Zeiss, USA).

### Optogenetics and Optical Mapping Experiments

Photosensitive electrophysiological properties of ChR2-expressing cardiomyocytes cultured on PCL/Alfalfa nanofiber scaffolds were measured by optical mapping system with X-Rhod-1 (Invitrogen, USA), a Ca²⁺ sensitive fluorescent dye, using a modified tandem-lens microscope (Scimedia Ltd., USA). The microscope was equipped with a high speed camera (MiCAM Ultima, Scimedia Ltd., USA), a plan APO 1.0x objective, a collimator (Lumencor, USA), and a 200 mW mercury lamp for epifluorescence illumination (X-Cite exacte, Lumen Dynamics, Canada). In order to stimulate the ChR2 and collect the X-Rhod-1 fluorescent signal, we utilized excitation filter (580/14 nm), dichroic mirror (593 nm cut-off), and emission filter (641/75) (Semrock, USA). The recombinant lentiviruses, containing cardiac troponin T (cTnT) promoter and ChR2-EYFP, were purchased from VectorBuilder Inc (CA, USA) to drive the cardiac specific expression. The ChR2-expressing NRVMs (1 million cells per sample) were seeded on PCL/Alfalfa nanofiber scaffolds in 6-well plates. After 1 day of cell culture, the scaffolds were washed 2 times with PBS and incubated in culture medium with 10% FBS and lentiviral vectors encoding for ChR2-eYFP. Lentivirus was used to transduce NRVMs at Multiplicity of infection (MOI) of 5. On day 2 of cell culture (post-transduction day 1), the scaffolds were washed 2 times with PBS and then incubated with culture medium containing 2% FBS. For optical mapping measurements, cardiomyocytes on PCL/Alfalfa nanofiber scaffolds were incubated with 2 μM X-Rhod-1 for 40 min at 37° C. and rinsed, and incubated in media for 15 min at 37° C. Before measuring the Ca²⁺ optical propagation, rinsed, and incubated with Tyrode's buffer for 5 min at 37° C. For the optogenetic stimulation, LED optical fibers (Doric Lenses, Canada) were used. The light sources of the LED were controlled by custom software written in LabVIEW (National Instruments, USA). For post-imaging processing and analysis, we used MiCAM imaging software (BV_Ana, SciMedia, USA).

### Mouse Excisional Wound Splinting Model

All animal experiments for wound healing study were approved by IACUC. As previously reported, we utilized the mouse splinting model to limit wound contraction in the mouse skin in an effort to investigate human-like wound healing. Briefly, C57BL/6 male mice (8 week old, Charles River Laboratories, USA) were anesthetized using isoflurane during all procedure. Hairs on the dorsal side of mice were shaved using electric razor. After shaving, betadine (Santa Cruz Biotechnology, USA) and ethanol (70% vol/vol) were used to clean the skin. The full thickness wounds were made by utilizing a 6-mm diameter sterile biopsy punch (Integra Miltex, USA). The splinting rings were attached to skin near the wound sites with an adhesive (Krazy glue, USA) and sutures (Ethicon, USA). We applied nanofiber scaffolds to the wounds and then covered the wounds with Tegaderm (Nexcare, USA) patches. For control samples, wounds had no treatment, but were covered with Tegaderm patches. Wound closure was monitored on day 0 and 14 after the surgery. Tissues were harvested on day 14 post surgery. The harvest tissues were fixed by 4% PFA, embedded in paraffin, sectioned, deparaffinized, and stained with Masson's trichrome. The Masson's trichrome-stained samples were imaged by slide scanner (Olympus VS120, USA). For immunochemistry, the sections were deparaffinized and incubated with 5% BSA for 2 h. Then, the sections were incubated with primary antibody (anti cytokeratin 14 or K14, Abcam, USA) in 1% BSA overnight at 4° C. Next day, the samples were washed by PBS 3 times and incubated with secondary antibodies (Alexa Fluor 488-conjugated mouse IgG (H+L) secondary antibody and DAPI) for 1 h. After the incubation, the samples were washed by PBS 3 times and imaged using a spinning disc confocal microscope. Epithelial gap and granulation tissue formation were analyzed from Masson's trichrome images following the established methods (Wang, X., et al. Nat. Protoc. 2013, 8 (2), 302; Martino, M. M., et al. Sci. Transl. Med. 2011, 3 (100), 100ra89-100ra89).

### Statistical Analysis

All data are presented as mean±standard error (SEM) and box plots with all data point overlap. The edges of box plots were defined as 25^{th} and 75^{th} percentiles. The middle bar is the median and the whiskers are 5^{th} and 95^{th} percentiles. The statistical comparisons were evaluated by using One-way analysis of variance (ANOVA) with the post-hoc Tukey's test in OriginPro 8.6 software (Plodinec, M., et al. Nat. Nanotechnol. 2012, 7 (11), 757). Statistical significance was determined at *p<0.05.

### Example 4B: Nanofiber Fabrication and Structural Properties

Nanofibers were fabricated using a pull spinning system under high centrifugal forces (FIG. 76) (Deravi, L. F., et al. Macromol. Mater. Eng. 2017, 302 (3)). As described below, alfalfa was co-spun with PCL, which is agood carrier polymer fro nanofiber production due to its fiber-forming capability, its biocompatibility and biostability (Suwantong, O. Polym. Adv. Technol. 2016, 27 (10), 1264-1273). Specifically, 6 wt/v % of PCL was used as a carrier polymer because it showed the least % beading with the nanoscale fiber radius in the pull spinning system. HFIP was used as a volatile solvent since it can dissolve both PCL and the biomolecular contents of alfalfa such as phytoestrogens and chlorophylls. The concentration of alfalfa was varied (0, 0.5, and 1 wt/v %) with a fixed ratio (6 wt/v %) of PCL in HFIP (Table 2).

TABLE 2
Spinnability of PCL and alfalfa in HFIP

| Material | | | |
|---|---|---|---|
| Carrier polymer | Alfalfa | | Corresponding |
| (w/v %) | (w/v %) | Morphology | image |
| PCL (6%) | None | Continuous fibers | FIG 1a |
| PCL (6%) | 0.5% | Continuous fibers | FIG 1c |
| PCL (6%) | 1% | Continuous fibers | FIG 1e |
| PCL (6%) | 1.5% | Fiber with beads | FIG S2b |
| PCL (6%) | 2% | Fiber with beads | FIG S2c |
| None | 1% | No fiber | FIG S2a |

Without the co-spinning polymer, alfalfa alone cannot form fibers (FIG. 77a) due to its low chain entanglement. When co-spun with PCL, the spinning conditions generated continuous nanofibers (FIGS. 78a-78c) with diameters of 345.3±52.5 for PCL 6 wt/v %, 394.3±70.7 for PCL 6 wt/v %/alfalfa 0.5 wt/v %, and 408.6±56.1 nm PCL 6 wt/v %/alfalfa 1 wt/v % (FIGS. 78d-78f). When the doping concentration was 1.5 wt/v % or higher, the spun nanofibers exhibited extreme bead formation (FIGS. 77b-77c). The fiber diameter increased when the ratio of alfalfa was increased in the polymer dope. The spun nanofibers were also highly aligned, showing a unidirectional distribution of fiber orientation (FIG. 78g). The alignment of nanofibers plays an important role in facilitating cell migration and laminar tissue formation (such as cardiac tissues) (Schnell, E. et al. Biomaterials 2007, 28 (19), 3012-3025; Badrossamay, M. R., et al. Biomaterials 2014, 35 (10), 3188-3197; Ahn, S., et al. Anal. Bioanal. Chem. 2018). Furthermore, the nanofiber scaffolds exhibited similar porosity regardless of doping concentrations (FIG. 78h). In addition to the topographical cue provided by aligned nanofibers, stiffness also plays a crucial role in determining cell behavior (Discher, D. E., et al. Science 2005, 310 (5751), 1139-1143; Wells, R. G., Hepatology 2008, 47 (4), 1394-1400). Accordingly, mechanical matching of scaffolds to the tissue is an important factor for tissue engineering applications because the stiffness of human tissues varies according to their structure and function—ranging from a few hundred Pa (brain) to a few GPa (bone) (Barnes, J. M., et al. J. Cell Sci. 2017, 130 (1), 71-82). Mechanical uniaxial testing was employed in an effort to study the mechanical properties of our scaffolds. The Young's modulus of the nanofiber scaffolds significantly decreased with an increase of alfalfa doping concentration (FIG. 78i). To correct the effect of the scaffold density, specific modulus was calculated by dividing Young's modulus by the density of nanofiber scaffolds. There was no significant difference between PCL (6 wt/v %) and PCL/alfalfa (6 wt/v %/0.5 wt/v %) nanofiber scaffolds in the specific modulus (FIG. 78j). On the other hand, the specific modulus of PCL/alfalfa (6 wt/v %/1 wt/v %) nanofiber scaffolds was significantly lower than that of PCL (6 wt/v %) and PCL/alfalfa (6 wt/v %/0.5 wt/v %) nanofiber scaffolds. Therefore, mechanical properties of the scaffolds become softer when the concentration of alfalfa increases. This is potentially due to the scaffolds having higher contents of hydrophilic compounds and higher degree of hydration as the alfalfa concentration increases (Ahn, S., et al. Adv. Healthcare Mater. 2018, 7 (9), e1701175; Ahn, S., et al. Anal. Bioanal. Chem. 2018; Joy, A., et al. Langmuir 2011, 27 (5), 1891-1899). The Young's moduli of PCL/alfalfa scaffolds were 24.9±4.4 kPa (with 0.5 wt/v % of alfalfa) and 9.0±1.8 kPa (with 1 wt/v % of alfalfa). The mechanical property of the scaffolds could be ideal for soft tissue engineering such as skin (5-600 kPa) and cardiac ventricle (15-100 kPa) (Agache, P., et al. Arch. Dermatol. Res. 1980, 269 (3), 221-232; Liang, X., et al. IEEE Trans. Biomed. Eng. 2010, 57 (4), 953-959; Capulli, A., et al. Adv. Drug Del. Rev. 2016, 96, 83-102).

### Example 4C: Chemical Characterization of Fiber Components

Alfalfa is composed of various biomacromolecular components, including phytoestrogens and chlorophylls. In order to check if these alfalfa components remained stable within the spun nanofibers, FT-IR spectra of the nanofibers were recorded (FIG. 79a). FT-IR spectra showed a major peak at 1723 cm⁻¹ that is indicative of carbonyl stretching (C═0) of PCL (Badrossamay, M. R., et al. Biomaterials 2014, 35 (10), 3188-3197). All spectra were normalized to the PCL peak (1732 cm⁻¹) to see relative changes in IR peaks. To verify the existence of alfalfa in the nanofibers, amide peaks were monitored since PCL has no peak in the amide I and II regions (1500-1700 cm⁻¹) sensitive to protein secondary structures (Kong, J., et al. Acta Biochim. Biophys. Sin. 2007, 39 (8), 549-559). The amide peaks at 1540, 1578, and 1660 cm⁻¹ increased with higher alfalfa concentration. The optical properties of

PCL/alfalfa composite nanofibers were also characterized to confirm whether the distinctive green color due to the high chlorophyll content in the native state of alfalfa was maintained (FIGS. 79b and 79c). The UV-Vis absorption spectra of PCL/alfalfa nanofibers showed peaks at ˜450 and ˜650 nm (Lichtenthaler, H. K., et al. Current Protocols in Food Analytical Chemistry 2001) which are indicative of chlorophyll content, while no peaks were detected for PCL nanofibers (FIG. 79d). Nanofibers with higher alfalfa concentration resulted in stronger peak intensities at 435 and 663 nm. This is further supported by hyperspectral imaging (FIGS. 79e-79h), whereby the average map of absorbance was collected from multiple regions of samples. In line with UV-Vis measurement, alfalfa cast film showed distinctive peaks (at ˜435 and 663 nm) due to chlorophyll content of alfalfa (FIGS. 79e and 79h), which are consistent with the peaks detected in different regions of PCL/alfalfa nanofiber (FIGS. 79g and 79h) and are not present in the spectra for PCL nanofiber (FIGS. 79f and 79h). Altogether, we confirmed that alfalfa was successfully integrated within the scaffolds.

### Example 4D: Surface Wettability

Because the hydrophilicity of a material affects its efficacy as a bioscaffold, the wettability of the alfalfa-based scaffolds was also characterized. Contact angle (θ) has been used to classify the surface wettability as follows: superhydrophilic (θ<25°), high hydrophilic (25°<0<90°), low hydrophilic (90°<θ<150°), and superhydrophobic (θ>150°) (Xu, X., et al. ACS Appl. Mater. Interfaces 2012, 4 (8), 4331-4337; Donaldson, E. C.; Alam, W., Wettability. Elsevier: 2013). The contact angle was assessed by calculating angles between water droplet and surface of the samples. Wettability of both cast films and nanofiber scaffolds was tested. Contact angle on the cast film is a traditional way to investigate a static contact angle (FIGS. 80a and 80b). The contact angle on the PCL cast film was 86.4°±2.3 that is close to low hydrophilicity due to the hydrophobic nature of the PCL polymer. With addition of alfalfa, the cast films became more hydrophilic. Especially, superhydrophilic property was achieved by the PCL/alfalfa (6 wt/v %/1 wt/v %) cast film (θ=17.9±1.7°). Furthermore, contact angle of the spun nanofiber scaffolds was investigated (FIGS. 80c and 80d). It should be noted that contact angles on the scaffolds do not represent the conventional static contact angle, but rather explain the degree of spreading and absorption of the droplet on the scaffolds (Xu, X., et al. ACS Appl. Mater. Interfaces 2012, 4 (8), 4331-4337). The initial contact angles in all conditions were alike regardless of chemical compositions. However, within the same time frame (25 s), water droplet on PCL/alfalfa (6 wt/v %/1 wt/v %) nanofiber scaffold was completely spread and absorbed, resulting in a superhydrophilic contact angle (˜0°). On the other hand, PCL only and PCL/alfalfa (6 wt/v %/0.5 wt/v %) nanofiber scaffolds retained water droplets on their surfaces at 25 s with high contact angles (θ>70°). Since nanofiber scaffolds are absorptive materials and have higher roughness compared to cast films, contact angles of nanofiber scaffolds at a later time point are lower than those of cast films. Moreover, polar groups from alfalfa (such as proteins and phytoestrogens) increase the wettability by facilitating interaction between the surface and the polar water droplet. Superhydrophilic scaffolds play a vital role in tissue engineering since they promote cell adhesion, proliferation, and infiltration (Jiao, Y.-P., et al. Biomed. Mater. 2007, 2 (4), R24; Yoo, H. S., et al. Adv. Drug Del. Rev. 2009, 61 (12), 1033-1042). Therefore, in the following studies, the superhydrophilic PCL/alfalfa (6 wt/v %/1 wt/v %) nanofiber was selected as the sample and PCL (6 wt/v %) nanofiber as a control.

### Example 4E. Phytoestrogen Content Analysis

Phytoestrogen is a chemical in plants that is structurally and functionally similar to estrogen. Once delivered to a target organ, phytoestrogens bind to estrogen receptors (ERs; ER α or ER β) in cells with higher affinity to ER β than ER α. By triggering the ER β signaling pathways, phytoestrogens benefit human health (such as wound healing and breast cancer) (Patisaul, H. B.; Jefferson, W. Front. Neuroendocrinol. 2010, 31 (4), 400-419). For example, phytoestrogens promote re-epithelialization, new hair follicle formation, and adipose tissue regeneration during wound healing (Emmerson, E., et al. Mol. Cell. Endocrinol. 2010, 321 (2), 184-193; Zhao, J., et al. J. Nutr. Biochem. 2011, 22 (3), 227-233; Zanella, I., et al. Eur. J. Nutr. 2015, 54 (7), 1095-1107. Additionally, proliferation of breast cancer cells can be prevented by phytoestrogens (Sotoca, A., et al. J. Steroid Biochem. Mol. Biol. 2008, 112 (4-5), 171-178; Rajah, T. T., et al. Pharmacology 2009, 84 (2), 68-73). One of major phytoestrogens that are advantageous to human health is genistein, which is known to be present in alfalfa (Hwang, J., et al. J. Agric. Food Chem. 2001, 49 (1), 308-314). In an effort to see whether our scaffolds can deliver genistein, LC-MS analysis for genistein was performed (FIG. 81). Accordingly, a signal at m/z=269 was detected using a selected ion monitoring (SIM) mode to quantify the amount of genistein. A genistein standard solution produced a peak at 7.8 min. The genistein peak at 7.8 min was also found in alfalfa powder and PCL/Alfalfa nanofiber, but not in PCL nanofiber. The amount of genistien in PCL/Alfalfa nanofiber was further quantified. It was observed that PCL/Alfalfa nanofiber possesses 2.48±1.02 (mg/L) of genistein (analyzed from 5 samples). Consequently, this data shows that genistein can be delivered by using PCL/alfalfa nanofiber.

### Example 4F: In Vitro Cell Culture

In the previous sections, it was demonstrated that PCL/alfalfa scaffolds have nanofibrous structure, bioactive molecules, and superhydrophilic property that are crucial for biomedical applications. Due to such properties, it was hypothesized that PCL/alfalfa nanofiber scaffolds can support cell adhesion, proliferation, and ultimately—mature tissue formation. To test if these scaffolds can facilitate tissue maturation, three types of cells (dermal fibroblasts, cardiomyocytes, and neurons) were cultured on PCL/alfalfa nanofiber. In human neonatal dermal fibroblast (HNDF) culture, PCL nanofiber was used as a control to see if the existence of alfalfa in the nanofiber can enhance cell growth. First, biocompatibility of PCL/alfalfa nanofiber was investigated utilizing a traditional LDH assay to measure the cytotoxic LDH release from dead cells (Korzeniewski, C.; Callewaert, D. M., J. Immunol. Methods 1983, 64 (3), 313-320.). At day 7 post cell culture, cells on PCL and PCL/alfalfa nanofibers released a similar amount of lactate dehydrogenase (LDH) without a significant difference, demonstrating a good biocompatibility of PCL/alfalfa nanofiber (FIG. 82). Moreover, coverage of HDNFs on scaffolds was analyzed. HNDF coverage was significantly higher on PCL/alfalfa nanofiber than on PCL nanofiber, owing to the increased hydrophilicity and the existence of bioactive components of the alfalfa-containing fibers (FIGS. 83a-83c). Additionally, the effect of fiber anisotropy on the alignment and growth of primary rat cortical neurons was investigated. At day 7, it was observed that neurons on the nanofiber scaffolds were highly aligned along the fiber axis due to the surface anisotropy (FIGS. 83d-83e). The degree of neurite outgrowth was further quantified to determine the effect of alfalfa in the nanofibers on neuronal development in vitro. It was found that total neurite length on PCL/alfalfa nanofibers was significantly higher than that on simple PCL nanofibers (FIG. 83h).

The growth and maturation of neonatal rat ventricular myocytes (NRVM) on the alfalfa-based scaffolds was also invetigated. NRVMs grown on PCL/alfalfa nanofiber scaffolds were spontaneously beating after 5 days of cell culture. NRVMs on PCL/alfalfa nanofiber formed anisotropic tissues due to the high alignment of the nanofiber (FIG. 84a). Three-dimensional (3D) reconstruction of z-stack images of NRVMs on PCL/alfalfa nanofiber (FIG. 84b) exhibited three-dimensionally aligned cell growth infiltrating through the scaffolds (z-depth is about 30 μm) owing to the porous and three-dimensional architecture of the nanofiber scaffolds. Furthermore, Ca²⁺ waves locally activated by optical stimulation were directionally propagated through the fiber alignment (FIG. 84c). Ca²⁺ transients extracted from Ca²⁺ imaging at different regions indicated the synchronized Ca²⁺ propagation throughout the scaffolds (FIG. 84d).

Taken together, these in vitro cell culture tests support that PCL/alfalfa nanofiber scaffolds can promote cell growth and new tissue formation for the diverse cell type-specific behaviors (skin fibroblasts for wound healing, neurons for CNS disorders, and cardiomyocytes for CVDs) from different species (human and rat). Furthermore, directional cues from the aligned scaffolds guide anisotropic tissue formation that are beneficial for engineering other muscular tissues (such as skeletal muscles) (Choi, J. S., et al. Biomaterials 2008, 29 (19), 2899-2906; Younesi, M., et al. Adv. Funct. Mater. 2014, 24 (36), 5762-5770) and be potentially used as a nerve conduit to accelerate neuronal differentiation and remove brain tumor cells (Xie, J., et al. Biomaterials 2009, 30 (3), 354-362; Jain, A., et al. Nat. Mater. 2014, 13 (3), 308).

### Example 4G: In Vivo Tissue Regeneration

In an effort to verify the regenerative effects of PCL/alfalfa scaffolds, we utilized the excisional mice splinting wound model to study how our scaffolds affect tissue regeneration in vivo (FIG. 85a). This model limits wound contraction in mice and thus provides human-like healing results (Wang, X., et al. Nat. Protoc. 2013, 8 (2), 302). The control wounds had no treatment, but were covered with Tegaderm dressing. After 14 days healing processes, wounds treated with PCL/alfalfa nanofiber were closed faster than those treated with PCL nanofiber or control (FIGS. 85b-85c). To further investigate in vivo tissue generation, we performed Masson's trichrome stating of day 14 tissues (FIGS. 85d-85f). Once wounded, epithelial cells migrate to the wound site to enclose the wounds and fibroblasts and inflammatory cells deposit new extracellular matrix called as granulation tissue to fill the wound. The re-epithelialization was measured by calculating distance among newly formed epithelial layers. In a line with the macroscopic wound closure analysis (FIG. 85c), epithelial gaps in PCL/alfalfa nanofiber-treated wounds were significantly smaller than those in control and PCL only nanofiber-treated wounds (FIG. 85g). In addition, more granulation tissue was synthesized in PCL/alfalfa scaffolds-treated wounds compared to control and PCL scaffolds-treated wounds (FIG. 85h). Without any treatment, normal healing processes in human and mice causes a scar with a lack of hair follicles (Plikus, M. V., et al. Science 2017, 355 (6326), 748-752). In order to further study effects of alfalfa scaffolds on new hair follicle formation, cytokeratin 14 (K14) staining was performed (FIG. 86). K14 is highly expressed in the basal keratinocyte layer and the outer layer of the hair follicle (Nijhof, J. G., et al. Development 2006, 133 (15), 3027-3037; Pastar, I., et al. Adv. Wound Care 2014, 3 (7), 445-464). In control and PCL nanofiber-treated wounds, formation of new hair follicle or hair was not found. On the other hand, wounds treated with PCL/alfalfa nanofiber exhibited new hair germ and follicle formation in the wound bed with K14-positive stains. The enhanced re-epithelialization, granulation tissue formation, and hair follicle regeneration by PCL/alfalfa scaffolds possibly attributes to the existence of bioactive components in alfalfa such as ECM-mimetic peptides and phytoestrogens.

In summary, this is the first report of an engineered alfalfa-based nanofiber composite material. PCL and HFIP were used as the carrier polymer and solvent, respectively. Using the optimal concentrations for spinning the alfalfa and PCL composite, we generated nanofiber bioscaffolds. The pull-spun PCL/alfalfa composite bioscaffold is comprised of hydrophilic nanofibers and bioactive molecules (such as proteins and genistein). Owing to these components, aligned PCL/alfalfa nanofiber scaffolds have improved in vitro cell adhesion, growth, sustained biocompatibility and mature tissue formation for various cell types (dermal fibroblasts, cardiomyocytes, and neurons) from different origins (rat and human). Additionally, the data demonstrated that the anisotropic topography from PCL/alfalfa scaffolds helps to synchronize and guide directional calcium wave propagation in the engineered cardiac tissue that is vital for muscle tissue function. Lastly, the in vivo functionality of PCL/alfalfa scaffolds was assessed using a human-like mouse wound model. PCL/alfalfa scaffolds accelerated re-epithelialization and granulation tissue formation. Interestingly, new hair germ and follicle formation were also discovered when PCL/alfalfa scaffolds were applied to the wounds. These data demonstrate the usefulness of PCL/alfalfa nanofibrous scaffolds for diverse and tissue engineering applications.

### Example 5: Biomimetic and Estrogenic Soy-Based Nanofibrous Wound Dressings

More than 6 million patients annually suffer from severe cutaneous wounds. During the process of wound healing, just before the inflammatory phase is initiated, the clotting cascade occurs in order to achieve hemostasis, or stop blood loss by way of a fibrin clot. Thereafter, various soluble factors (including chemokines and cytokines) are released to attract cells that phagocytise debris, bacteria, and damaged tissue, in addition to releasing signaling molecules that initiate the proliferative phase of wound healing.

About two or three days after the wound occurs, fibroblasts begin to enter the wound site, marking the onset of the proliferative phase even before the inflammatory phase has ended. As in the other phases of wound healing, steps in the proliferative phase do not occur in a series but rather partially overlap in time.

When the levels of collagen production and degradation equalize, the maturation phase of tissue repair is said to have begun. During maturation, type III collagen, which is prevalent during proliferation, is replaced by type I collagen. Originally disorganized collagen fibers are rearranged, cross-linked, and aligned along tension lines. The onset of the maturation phase may vary extensively, depending on the size of the wound and whether it was initially closed or left open, ranging from approximately 3 days to 3 weeks. The maturation phase can last for a year or longer, similarly depending on wound type.

As discussed above, estrogen and phytoestrogen promote wound healing via the ER β pathway. However, estrogen also has a high affinity to ER α, which can trigger ER α-positive breast cancer (nearly 70% of breast tumors). On the other hand, phytoestrogens preferentially bind to ER β resulting in less risk for ER α-positive cancers. In addition, soy protein has bioactive peptides similar to extracellular matrix (ECM) proteins, present in human tissues. Specifically in cutaneous wound healing, it has been shown that cryptic peptides in soy protein improved wound healing by increasing dermal ECM synthesis and stimulating re-epithelialization. Soy phytoestrogens have demonstrated to accelerate the healing process via ER-mediated signaling pathways. They also possess anti-bacterial, anti-inflammatory, and anti-oxidant properties that support and enhance wound healing. It has also been reported that oral intake of soy (both protein and phytoestrogens) accelerates skin regeneration in aged women and burn patients. Because of these pro-regenerative traits, phytoestrogens in soy can promote cutaneous wound healing, with low risk of ER α-mediated carcinogenic pathway. Current methods for engineering soy protein nanofibers require the use of synthetic polymers as carriers, due to the low molecular weight of soy protein that inhibits the production of nanofibers alone, and high-voltage for use in electrospinning to prepare the fibers. Accordingly, there is a need in the art for scaffolds, wound dressings, and methods to promote and accelerate cutaneous wound closure and to restore cutaneous wounds to their original native configuration.

Using the iRJS system (described above), polymeric fiber scaffolds comprising soy protein isolate (SPI) and hyaluronic acid (HA) were produced as described above in Example 3. In order to provide additional mechanical and structural stability to the HA/SPI fiber scaffolds without loss of the desirable structural characteristics of the formed fibers and scaffolds, the formed scaffolds were covalently cross-linked via ester bond formation by contacting the scaffolds with a solution of ethyl(dimethylaminopropyl) carbodiimide (EDC)/N-hydroxysuccinimide (NHS) (10 mM/4 mM) for 24 hours, with shaking (FIG. 87). The solutions of HA and SPI used were 2% HA (w/v %); 2% HA (w/v %)/2% SPI (w/v %); or 2% HA (w/v %)/4% SPI (w/v %). In some embodiments, the aqueous solution comprising hyaluronic acid further comprises DMSO (dimethyl sulfoxide) to assist in fully dissolving the phytoestrogens present in SPI. In some embodiments, the aqueous solution comprising DMSO comprises a water to DMSO ration of about 6:1. As depicted in FIG. 88a, solutions comprising % HA (w/v %)/2% SPI (w/v %) and cross-linked with EDC/NHS enabled the formation of bead-free fibers.

Phytoestrogen analysis of the formed fibers and scaffolds was performed as described above and, as depicted in FIG. 88b, left-hand, a signal at m/z=271 (corresponding to genistein) was detected using a selected ion monitoring (SIM) mode to quantify the amount of genistein. A genistein standard solution produced a peak at 7.1 min. The genistein peak at 7.1 min was also found in SPI powder and HA/SPI, but not in HA nanofiber. The amount of genistien in HA/SPI nanofiber was further quantified. It was observed that HA/SPI nanofiber possesses 3.2153±0.62603 (mg/L) of genistein (2% HA (w/v %)/2% SPI (w/v %) fiber samples). Consequently, this data shows that genistein can be delivered by using HA/SPI nanofiber.

Soy protein isolate is composed of various biomacromolecular components, including phytoestrogens and proteins. In order to determine if these SPI components remained stable within the spun fibers and that cross-linking with EDC/NHS did not affect the stability of the active compounds, FT-IR spectra of the nanofibers were recorded (FIG. 89). FT-IR spectra showed a major peak at 1040 cm⁻¹ that is indicative of C—O—C stretching of HA (Ji et al. (2006) Biomaterials). All spectra were normalized to the HA peak (1040 cm⁻¹) to see relative changes in IR peaks. To verify the existence of

SPI in the nanofibers, amide peaks were monitored in the amide I region (1600-1700 cm⁻¹) sensitive to protein secondary structures (Kong, J., et al. Acta Biochim. Biophys. Sin. 2007, 39 (8), 549-559). The amide peaks at around 1626 cm⁻¹ increased with higher SPI concentration. The occurrence of the peak at 1693 cm⁻¹ indicated new ester bond formation by EDC/NHS crosslinking

As depicted in FIG. 90a, the HA and HA/SPI formed fibers have micron-scale diameters. Specifically, fibers formed in an iRJS system using a 2% w/v solution of HA were between about 1 and about 2 micrometers (average diameter of about 1.58128±0.02278 micrometers) and fibers formed in an iRJS system using a solution comprising 2% w/v HA and 2% w/v SPI were between about 1.25 to about 2.25 micrometers (average diameter of about 1.73765±0.03278 micrometers). After crosslinking of the fibers with EDC/NHS, the fiber diameters increased slightly. In particular, fibers formed in an iRJS system using a 2% w/v solution of HA and crosslinked in EDC/NHS were between about 1.5 and about 2.5 micrometers (average diameter of about 1.58128±0.02278 micrometers) and fibers formed in an iRJS system using a solution comprising 2% w/v HA and 2% w/v SPI and crosslinked with EDC/NHS were between about 1.5 to about 2.5 micrometers (average diameter of about 2.04206±0.05726 micrometers).

The mechanical strength of the HA/SPI fiber scaffolds was determined by uniaxially stretching the fibers along the length of the fibers. As depicted in FIG. 91a, the Young's modulus of fibers increased after crosslinking and fibers formed from a solution comprising about 2% w/v HA/2% w/v SPI that were cross-linked had a Young's modulus range of about 4 kPa to about 10 kPa which is similar to the stiffness of human skin.

The stability of the fibers formed from a solution comprising about 2% w/v HA/2% w/v SPI were also examined. As depicted in FIG. 91b, without crosslinking, the spun fibers were quickly dissolved in both PBS and DMEM. However, after crosslinking, the fibers were stable in PBS for up to 2 weeks and a few days in DMEM. This data shows that the biostability of the fibers was improved by crosslinking

The porosity of the fiber scaffolds formed from a solution comprising about 2% w/v HA was examined and as depicted in FIG. 92, regardless of the addition of/2% w/v SPI in the HA solution or EDC/NHS crosslinking of formed fibers, all formed fibers had a porosity of between about 40%-60%, e.g., about 50%, without no significant differences between.

The effect of polymeric fiber scaffolds formed from a solution comprising 2% w/v HA and 2% w/v SPI and cross-linked with EDC/NHS on wound closure in mouse skin as compared to the effect of polymeric fiber scaffolds formed from a solution comprising 2% w/v HA cross-linked with EDC/NHS or a no treatment control was examined. In particular, 6 mm skin wounds were created in ovariectomized and soy-free diet fed mice and the wound was covered with a scaffold on Day 0. A splinting model was used to prevent skin contraction. The extent of wound closure was examined at days 3, 7, 14, and 20 post-surgery. As demonstrated in FIGS. 93a and 93b, the polymeric fiber scaffolds formed from a solution comprising 2% w/v HA and 2% w/v SPI and cross-linked with EDC/NHS accelerated wound closure as compared to polymeric fiber scaffolds formed from a solution comprising 2% w/v HA cross-linked with EDC/NHS or a no treatment control.

Animals were sacrificed on Day 22 and histological analyses of the tissues were performed. As demonstrated in FIGS. 94a and 94b, the polymeric fiber scaffolds formed from a solution comprising 2% w/v HA and 2% w/v SPI and cross-linked with EDC/NHS statistically significantly reduced the epidermal thickness and scar index as well as increases the new hair follicle regeneration compared to HA fibers and control samples. (*p<0.05). In addition, as demonstrated in FIG. 95, immunoflouresence staining of the tissues reveals that wounds treated with polymeric fiber scaffolds formed from a solution comprising 2% w/v HA and 2% w/v SPI and cross-linked with EDC/NHS had larger areas with higher expression of ER β and K14-positive in the center of wounds when compared to wounds treated with polymeric fiber scaffolds formed from a solution comprising 2% w/v HA and cross-linked with EDC/NHS and control samples.

An ex vivo analyses of the effect of the polymeric fiber scaffolds formed formed from a solution comprising 2% w/v HA and 2% w/v SPI and cross-linked with EDC/NHS as compared to polymeric fiber scaffolds formed from a solution comprising 2% w/v HA cross-linked with EDC/NHS or a no treatment control was also performed using human skin biopsies with 2 mm diameter wounds. At Day 7 post-wounding, histological analyses of the tissues demonstrated wound healing results similar to those observed in mouse tissues. Specifically, as demonstrated in FIGS. 96a and 96b, wounds treated with polymeric fiber scaffolds formed formed from a solution comprising 2% w/v HA and 2% w/v SPI and cross-linked with EDC/NHS had accelerated re-epithelialization (or newly formed epidermal layers shown in medium gray) as compared to polymeric fiber scaffolds formed formed from a solution comprising 2% w/v HA and cross-linked with EDC/NHS and control samples. Furthermore, when PHTPP (an ER β antagonist which specifically blocks ER β signaling pathways) was added to the culture medium, the re-epithelialization of the tissues treated with polymeric fiber scaffolds formed formed from a solution comprising 2% w/v HA and 2% w/v SPI and cross-linked with EDC/NHS was abolished. These data demonstrate that polymeric fiber scaffolds formed formed from a solution comprising 2% w/v HA and 2% w/v SPI and cross-linked with EDC/NHS promote and accelerate the wound healing processes via ER β pathways stimulated by the presence of active phytoestrogens in the formed fibers and scaffolds.

### EQUIVALENTS

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

The contents of all references, including patents and patent applications, cited throughout this application are hereby incorporated herein by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

As may be recognized by those of ordinary skill in the pertinent art based on the teachings herein, numerous changes and modifications may be made to the above-described and other embodiments of the present disclosure without departing from the spirit of the invention as defined in the appended claims. Accordingly, this detailed description of embodiments is to be taken in an illustrative, as opposed to a limiting, sense. Those skilled in the art will recognize, or be able to ascertain using no more than routine experimentation, many equivalents to the specific embodiments of the described herein. Such equivalents are intended to be encompassed by the following claims.

1. A polymeric fiber scaffold comprising:
a plurality of polymeric polymric fibers, each polymeric fiber independently comprising cellulose acetate and soy protein hydrolysate.

2.-16. (canceled)

17. A polymeric fiber scaffold comprising:
a plurality of polymeric fibers, each polymeric fiber independently comprising a protein selected from the group consisting of collagen type I, fibrinogen, fibronectin, gelatin, chondroitin sulfate, and hyaluronic acid, and combinations thereof.

18.-49. (canceled)

50. A polymeric fiber scaffold comprising:
a plurality of polymeric fibers, each polymeric fiber independently comprising polycaprolactone (PCL) and alfalfa.

51.-62. (canceled)

63. A polymeric fiber scaffold comprising:
a plurality of polymeric fibers, each polymeric fiber independently comprising hyaluronic acid and soy protein isolate.

64.-79. (canceled)

80. A method of forming a polymeric fiber scaffold comprising cellulose acetate and soy protein hydrosylate, the method comprising:
providing a solution comprising:
a polymer comprising cellulose acetate; and
soy protein hydrolysate;
forming a plurality of polymeric fibers by ejecting or flinging the solution from a reservoir; and
collecting the plurality of polymeric fibers on a collection surface to form the polymeric fiber scaffold.

81. (canceled)

82. (canceled)

83. A method of forming a polymeric fiber scaffold, the method comprising:
providing a solution comprising:
an extracellular matrix protein selected from the group consisting of cola protein selected from the group consisting of collagen type I, fibrinogen, fibronectin, gelatin, and hyaluronic acid, and combinations thereof;
rotating the polymer in solution about an axis of rotation to cause ejection of the polymer solution in one or more jets; and
collecting the one or more jets of the polymer in a liquid to cause formation of one or more polymeric fibers, thereby forming the polymeric fiber scaffold.

84.-103. (canceled)

104. A method of forming a polymeric fiber scaffold, the method comprising:
providing a solution comprising:
a polymer comprising polycaprolactone (PCL); and
alfalfa;
forming a plurality of polymeric fibers by ejecting or flinging the solution from a reservoir; and
collecting the plurality of polymeric fibers on a collection surface to form the polymeric fiber scaffold.

105. (canceled)

106. (canceled)

107. A method of forming a polymeric fiber scaffold, the method comprising:
providing a solution comprising:
hyaluronic acid and soy protein isolate;
rotating the polymer in solution about an axis of rotation to cause ejection of the polymer solution in one or more jets; and
collecting the one or more jets of the polymer in a liquid to cause formation of one or more polymeric fibers, thereby forming the polymeric fiber scaffold.

108.-111. (canceled)

112. A wound dressing comprising the polymeric fiber scaffold of any one of claims 1, 17, 50 and 63.

113. A method for treating a subject having a cutaneous wound, the method comprising:
providing the polymeric fiber scaffold of any one of claims 1, 17, 50, and 63; and
disposing the polymeric fiber scaffold on, over, or in the wound, thereby treating the subject.

114.-122. (canceled)
